(19) United States
(12) Patent Application Publication
(10) Pub. No.: US 2011/0053931 A1
(43) Pub. Date: Mar. 3, 2011

(54) QUINOLINE COMPOUNDS AND METHODS OF USE

(76) Inventors: John Gaudino, Longmont, CO (US); Steven Armen Boyd, Longmont, CO (US); Allison L. Marlow, Louisville, CO (US); Tomas Kaplan, Broomfield, CO (US); Kin Chiu Fong, Longmont, CO (US); Jeongbeob Seo, Broomfield, CO (US); Hongqi Tian, Longmont, CO (US); James Blake, Longmont, CO (US); Kevin Koch, Boulder, CO (US)

(21) Appl. No.: 12/303,930

(22) PCT Filed: Jun. 8, 2007
(86) PCT No.: PCT/US07/70787

§ 371 Date: Nov. 11, 2010

Related U.S. Application Data
(60) Provisional application No. 60/811,909, filed on Jun. 8, 2006.

(51) Int. Cl. A61K 31/5377; C07D 413/14; C12N 9/99; A61P 35/00; A61P 29/00; A61P 37/00; A61P 9/10; A61P 31/12; A61P 3/10; A61P 25/28; A61P 17/06; A61P 19/08; A61P 35/04; C07D 401/12; A61K 31/4709; C07D 401/14; A61K 31/506; A61K 31/501

(52) U.S. Cl. 514/234.5; 544/123; 514/235.2; 435/184; 544/128; 546/153; 514/312; 544/319; 514/269; 544/295; 514/252.18; 544/116; 544/114; 514/236.5; 514/252.02; 544/238; 514/252.04; 514/272; 544/331

(57) ABSTRACT

Compounds of Formula (I), and stereoisomers, geometric isomers, tautomers, solvates, metabolites, salts and pharmaceutically acceptable prodrugs thereof, are useful for inhibiting receptor tyrosine kinases and for treating hyperproliferative disorders mediated thereby. Methods of using compounds of Formula (I), and stereoisomers, geometric isomers, tautomers, solvates and pharmaceutically acceptable salts thereof, for in vitro, in situ, and in vivo diagnosis, prevention or treatment of such disorders in mammalian cells, or associated pathological conditions are disclosed.

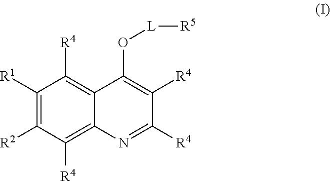

### FIELD OF THE INVENTION

The invention relates to quinoline compounds having protein tyrosine kinase activity. The quinoline compounds may be useful in the treatment of hyperproliferative disorders, such as cancer, in mammals. The invention also relates to pharmaceutical compositions and formulations, methods of synthesis, and methods of use such as treating hyperproliferative disorders.

### BACKGROUND OF THE INVENTION

Met tyrosine kinase is a high-affinity transmembrane receptor for the hepatocyte growth factor (HGF, Bottaro et al (1991) Science 251:802-804). Met was cloned, named (Cooper et al (1984) 311:29-33) and identified as an oncogene (Park et al (1986) Cell 45:895-904). When deregulated by overexpression or mutations, Met receptor tyrosine kinase leads to tumor growth and invasion (Cristiani et al (2005) Biochem. 44:14110-14119). Stimulation of Met by the ligand HGF, also known as Scatter Factor, initiates numerous physiological processes, including cell proliferation, scattering, morphogenic differentiation, angiogenesis, wound healing, tissue regeneration, and embryological development (Parr et al (2004) Clin. Cancer Res. 10(1, Pt. 1) 202-211; Comoglio et al (2002) J. Clin. Invest. 109:857-862; Maulik et al (2002) Cytokine Growth Factor Reviews 13:41-59; Hecht et al (2004) Cancer Res. 64(17):6109-6118). Receptor c-Met is rapidly internalized via clathrin-coated vesicles and traffics through an early endosomal compartment after hepatocyte growth factor stimulation. c-Met accumulates progressively in perinuclear compartments, which in part include the Golgi (Kermorgant et al (2003) J. of Biol. Chem. 278(31):28921-28929).

The phenomena of: deregulation or dysregulation of Met and/or HGF; Met overexpression; and Met mutations are implicated in uncontrolled cell proliferation and survival. Such factors play key roles in early-stage tumorigenesis, invasive growth of cancer cells, and metastasis (Danilkovitch-Miagkova et al (2002) J. Clin. Invest. 109(7):863-867; Di Renzo et al (1994) Int. J. Cancer 58:658-662; Matsumoto et al (1994) J. Biol. Chem. 269:31807-31813; Tusolino et al (1998) J. Cell Biol. 142:1145-1156; Jeffers et al (1996) Mol. Cell. Biol. 16:1115-1125; Wong et al (2004) Exper. Cell Res. 299(1):248-256; Konda et al (2004) J. Urology 171(6), Pt. 1:2166-2170; Heideman et al (2004) J. Gene Med. 6(3):317-327; Ma et al (2003) Cancer Res. 63(19):6272-6281; Maulik et al (2002) Clin. Cancer Res. 8:620-627), making Met an important target for anticancer drug development (Cohen, P. (2002) Nat. Rev. Drug Discovery 1:309-315). Overexpression of Met and HGF is associated with poor prognosis.

Much evidence supports the role of HGF as a regulator of carcinogenesis, cancer invasion and metastasis (for review see: Herynk, M. H., and Radinsky, R. (2000) In Vivo 14:587-596; Jiang et al (1999) Crit. Rev. Oncol. Hematol. 29:209-248; Longati (2001) Curr. Drug Targets 2:41-55; Maulik et al, (2002) Cytokine Growth Factor Rev. 13:41-59; Parr, C., and Jiang, W. G., (2001) Histol. Histopathol. 16:251-268). Recent data demonstrating the suppression of cancer cell proliferation, survival, and invasion upon inhibition of Met binding to HGF and Met receptor dimerization (Furge et al (2001) Proc. Natl. Acad. Sci. USA 98:10722-10727; Michieli et al (2004) Cancer Cell 6:61-73) confirm the relevance of Met in neoplasia and provide further proof of concept for the development of small-molecule compounds for antineoplastic therapy, e.g. against multiple myeloma (Hov et al. (2004) Clin. Cancer Res. 10(19):6686-6694). Inhibition of Met results in slowing tumor growth in tumor xenograft mouse models. Antibodies specific for c-Met have been expressed to block binding of HGF to c-Met (US 2005/0037431; US 2004/0166544). c-Met is also over-expressed in both non-small cell lung cancer and small cell lung cancer cells, in lung, breast, colon and prostate tumors (Herynk et al (2003) Cancer Res. 63(11):2990-2996; Maulik et al (2002) Clin. Cancer Res. 8:620-627). Since c-Met appears to play an important role in oncogenesis of a variety of tumors, various inhibition strategies have been employed to therapeutically target this receptor tyrosine kinase. The usefulness of inhibiting the protein-tyrosine kinase c-Met for inhibiting tumor growth and invasion has been shown in many well documented preclinical experiments (Abounader et al (1999) J. Natl. Cancer Inst. 91:1548-1556; Laterra et al (1997) Lab. Invest. 76:565-577; Tomioka, D. (2001) Cancer Res. 61:7518-7524; Wang et al (2001) J. Cell Biology 153:1023-1033).

Protein kinases (PK) are enzymes that catalyze the phosphorylation of hydroxy groups on tyrosine, serine and threonine residues of proteins by transfer of the terminal (gamma) phosphate from ATP. Through signal transduction pathways, these enzymes modulate cell growth, differentiation and proliferation, i.e., virtually all aspects of cell life in one way or another depend on PK activity. Furthermore, abnormal PK activity has been related to a host of disorders, ranging from relatively non-life threatening diseases such as psoriasis to extremely virulent diseases such as glioblastoma (brain cancer). Protein kinases include two classes; protein tyrosine kinases (PTK) and serine-threonine kinases (STK).

One of the prime aspects of PTK activity is their involvement with growth factor receptors which are cell-surface proteins. When bound by a growth factor ligand, growth factor receptors are converted to an active form which interacts with proteins on the inner surface of a cell membrane. This leads to phosphorylation on tyrosine residues of the receptor and other proteins and to the formation inside the cell of complexes with a variety of cytoplasmic signaling molecules that, in turn, effect numerous cellular responses such as cell division (proliferation), cell differentiation, cell growth, expression of metabolic effects to the extracellular microenvironment, etc. For a more complete discussion, see Schlessinger and Ullrich, (1992) Neuron 9:303-391, which is incorporated by reference, including any drawings, as if fully set forth herein.

Growth factor receptors with PTK activity are known as receptor tyrosine kinases (RTK, Plowman et al (1994) DN&P, 7(6):334-339), which comprise a large family of transmembrane receptors with diverse biological activity. Met is one member of the tyrosine kinase growth factor receptor family, and often referred to as c-Met or human hepatocyte growth factor receptor tyrosine kinase (hHGFR). The expression of c-Met is thought to play a role in primary tumor growth and metastasis (Kim et al. Clin. Cancer Res. (2003) 9(14):5161-5170).

Modulation of the HGF/c-met signaling pathway may be effected by regulating binding of HGF beta chain to c-Met. In particular embodiments, the zymogen-like form of HGF beta mutant was shown to bind Met with 14-fold lower affinity than the wild-type serine protease-like form, suggesting optimal interactions result from conformational changes upon cleavage of the single-chain form (US 2005/0037431). Extensive mutagenesis of the HGF beta region corresponding to the active site and activation domain of serine proteases showed that 17 of the 38 purified two-chain HGF mutants resulted in impaired cell migration or Met phosphorylation but no loss in Met binding. However, reduced biological activities were well correlated with reduced Met binding of corresponding mutants of HGF beta itself in assays eliminating dominant alpha-chain binding contributions.

Protein-tyrosine kinases (PTK) are critical components of signaling pathways that control cellular proliferation and differentiation. PTK are subdivided into two large families, receptor tyrosine kinases (RTK) and non-receptor tyrosine kinases (NRTK). RTK span the plasma membrane and contain an extra-cellular domain, which binds ligand, and an intracellular portion, which possesses catalytic activity and regulatory sequences. Most RTK, like the hepatocyte growth factor receptor c-Met, possess a single polypeptide chain and are monomeric in the absence of ligand. Ligand binding to the extracellular portion of RTK, dimerizes monomeric receptors, resulting in autophosphorylation of specific tyrosine residues in the cytoplasmic portion (for review see: Blume-Jensen, P., and Hunter, T., Nature (2001) 411:355-365; Hubbard, S. R., et al, J. Biol. Chem. 273 (1998) 11987-11990; Zwick, E., et al, Trends Mol. Med. (2002) 8:17-23). In general, tyrosine autophosphorylation either stimulates the intrinsic catalytic kinase activity of the receptor or generates recruitment sites for downstream signaling proteins containing phosphotyrosine-recognition domains, such as the Src homology 2 (SH2) domain or the phosphotyrosine-binding (PTB) domain.

c-Met inhibitors have been reported (U.S. Pat. No. 5,792,783; U.S. Pat. No. 5,834,504; U.S. Pat. No. 5,880,141; U.S. Pat. No. 6,297,238; U.S. Pat. No. 6,599,902; U.S. Pat. No. 6,790,852; US 2003/0125370; US 2004/0242603; US 2004/0198750; US 2004/0110758; US 2005/0009845; US 2005/0009840; US 2005/0245547; US 2005/0148574; US 2005/0101650; US 2005/0075340; US 2006/0009453; US 2006/0009493; WO 98/007695; WO 2003/000660; WO 2003/087026; WO 2003/097641; WO 2004/076412; WO 2005/004808; WO 2005/121125; WO 2005/030140; WO 2005/070891; WO 2005/080393; WO 2006/014325; WO 2006/021886; WO 2006/021881). PHA-665752 is a small molecule, ATP-competitive, active-site inhibitor of the catalytic activity of c-Met, as well as phenotypes such as cell growth, cell motility, invasion, and morphology of a variety of tumor cells (Ma et al (2005) Clin. Cancer Res. 11:2312-2319; Christensen et al (2003) Cancer Res. 63:7345-7355).

### SUMMARY OF THE INVENTION

In one aspect, the invention relates to quinoline compounds that are inhibitors of receptor tyrosine kinases (RTK), including c-Met. Certain hyperproliferative disorders are characterized by the overactivation of c-Met kinase function, for example by mutations or overexpression of the protein. Accordingly, the compounds of the invention are useful in the treatment of hyperproliferative disorders such as cancer.

More specifically, one aspect of the invention provides quinoline compounds of Formula I:

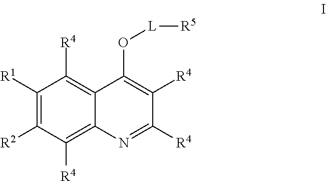

and stereoisomers, geometric isomers, tautomers, solvates, metabolites, salts, and pharmaceutically acceptable prodrugs thereof, wherein R¹, R², R⁴, R⁵ and L are as defined herein.

Another aspect of the invention provides a pharmaceutical composition comprising a quinoline compound of Formula I and a pharmaceutically acceptable carrier. The pharmaceutical composition may further comprise one or more additional therapeutic agents selected from anti-proliferative agents, anti-inflammatory agents, immunomodulatory agents, neurotropic factors, agents for treating cardiovascular disease, agents for treating liver disease, anti-viral agents, agents for treating blood disorders, agents for treating diabetes, and agents for treating immunodeficiency disorders.

Another aspect of the invention provides methods of inhibiting c-Met kinase activity, comprising contacting a c-Met kinase with an effective inhibitory amount of a quinoline compound of Formula I, or a stereoisomer, geometric isomer, tautomer, solvate, metabolite, or pharmaceutically acceptable salt or prodrug thereof.

Another aspect of the invention provides methods of preventing or treating a disease or disorder modulated by c-Met kinases, comprising administering to a mammal in need of such treatment an effective amount of a compound of Formula I, or a stereoisomer, geometric isomer, tautomer, solvate, metabolite, or pharmaceutically acceptable salt or prodrug thereof. Examples of such diseases, conditions and disorders include, but are not limited to, hyperproliferative disorders (e.g., cancer, including melanoma and other cancers of the skin), neurodegeneration, cardiac hypertrophy, pain, migraine, neurotraumatic diseases, stroke, diabetes, hepatomegaly, cardiovascular disease, Alzheimer's disease, cystic fibrosis, viral diseases, autoimmune diseases, atherosclerosis, restenosis, psoriasis, allergic disorders, inflammation, neurological disorders, hormone-related diseases, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders.

Another aspect of the invention provides methods of preventing or treating a hyperproliferative disorder, comprising administering to a mammal in need of such treatment an effective amount of a compound of Formula I, or a stereoisomer, geometric isomer, tautomer, solvate, metabolite, or pharmaceutically acceptable salt or prodrug thereof, alone or in combination with one or more additional compounds having anti-hyperproliferative properties.

In a further aspect the present invention provides a method of using a compound of this invention to treat a disease or condition modulated by c-Met in a mammal.

An additional aspect of the invention is the use of a compound of this invention in the preparation of a medicament for the treatment or prevention of a disease or condition modulated by c-Met in a mammal.

Another aspect of the invention includes kits comprising a compound of Formula I, or a stereoisomer, geometric isomer, tautomer, solvate, metabolite, or pharmaceutically acceptable salt or prodrug thereof a container, and optionally a package insert or label indicating a treatment.

Another aspect of the invention includes methods of preparing, methods of separating, and methods of purifying compounds of Formula I.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### DEFINITIONS

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (t-Bu, t-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂ CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like.

The term “alkenyl” refers to linear or branched-chain monovalent hydrocarbon radical of two to twelve carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like.

The term “alkynyl” refers to a linear or branched monovalent hydrocarbon radical of two to twelve carbon atoms with at least one site of unsaturation, i.e., a carbon-carbon, sp triple bond, wherein the alkynyl radical may be optionally substituted independently with one or more substituents described herein. Examples include, but are not limited to, ethynyl (—C≡CH), propynyl (propargyl, —CH₂C≡CH), and the like.

The terms “carbocycle”, “carbocyclyl”, “carbocyclic ring” and “cycloalkyl” refer to a monovalent or multivalent non-aromatic, saturated or partially unsaturated ring having 3 to 12 carbon atoms as a monocyclic ring or 7 to 12 carbon atoms as a bicyclic ring. Bicyclic carbocycles having 7 to 12 atoms can be arranged, for example, as a bicyclo[4,5], [5,5], [5,6] or [6,6] system, and bicyclic carbocycles having 9 or 10 ring atoms can be arranged as a bicyclo[5,6] or [6,6] system, or as bridged systems such as bicyclo[2.2.1]heptane, bicyclo[2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

“Aryl” means a monovalent or multivalent aromatic hydrocarbon radical of 6-20 carbon atoms derived by the removal of one or more hydrogen atoms from a carbon atom of a parent aromatic ring system. Some aryl groups are represented in the exemplary structures as “Ar”. Aryl includes bicyclic radicals comprising an aromatic ring fused to a saturated, partially unsaturated ring, or aromatic carbocyclic or heterocyclic ring. Typical aryl groups include, but are not limited to, radicals derived from benzene, substituted benzenes, naphthalene, anthracene, biphenyl, indenyl, indanyl, 1,2-dihydronapthalene, 1,2,3,4-tetrahydronapthyl, and the like.

Examples of aryl fused to a heterocyclic ring include, but are not limited to, the structure:

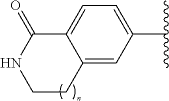

wherein n is 0, 1 or 2. Examples of aryl fused to a carbocylic ring include, but are not limited to, the structures:

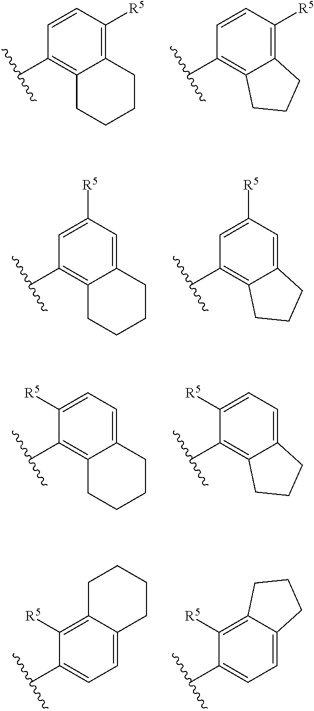

wherein R⁵ is as defined herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic, monovalent or multivalent radical of 3 to 20 carbon atoms, and in which at least one ring atom is a heteroatom selected from nitrogen, oxygen and sulfur, the remaining ring atoms being C, where one or more ring atoms is optionally substituted independently with one or more substituents described below. A heterocycle may be a monocycle having 3 to 7 ring members (2 to 6 carbon atoms and 1 to 3 heteroatoms selected from N, O, P, and S, wherein the S is optionally substituted with one or more oxo to provide the group SO or SO₂) or a bicycle having 7 to 10 ring members (4 to 9 carbon atoms and 1 to 3 heteroatoms selected from N, O, P, and S, wherein the S is optionally substituted with one or more oxo to provide the group SO or SO₂), for example: a bicyclo[4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. The heterocyclyl may be a carbon radical or heteroatom radical. The term “heterocycle” includes heterocycloalkoxy. “Heterocyclyl” also includes radicals where heterocycle radicals are fused with a saturated, partially unsaturated ring, or aromatic carbocyclic or heterocyclic ring. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 1,2,3,4-tetrahydroisoquinolinyl, 3-azabicyclo[3.1.0]hexanyl, 3-azabicyclo[4.1.0]heptanyl, azabicyclo[2.2.2]hexanyl, 3H-indolyl quinolizinyl and N-pyridyl ureas. Spiro moieties are also included within the scope of this definition. Examples of a heterocyclic group wherein 2 ring carbon atoms are substituted with oxo (═O) moieties are pyrimidindionyl and 1,1-dioxo-thiomorpholinyl. The heterocycle groups herein are optionally substituted independently with one or more substituents described herein.

The term “heteroaryl” refers to a monovalent or multivalent aromatic radical of 5-, 6-, or 7-membered rings, and includes fused ring systems (at least one of which is aromatic) of 1 to 20 carbon atoms, and containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

Examples of heteroaryl fused to an aryl ring include, but are not limited to:

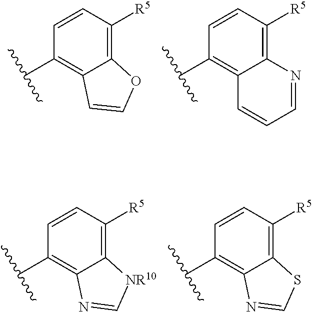

wherein R⁵ and R¹⁰ are as defined herein.

The heterocycle or heteroaryl groups may be C-attached or N-attached where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

“Substituted alkyl”, “substituted alkenyl”, “substituted alkynyl”, “substituted aryl”, “substituted heteroaryl”, “substituted heterocyclyl” and “substituted cycloalkyl” mean alkyl, alkenyl, alkynyl, aryl, heteroaryl, heterocyclyl and cycloalkyl, respectively, in which one or more hydrogen atoms are each independently replaced with a substituent. Typical substituents include, but are not limited to, F, Cl, Br, I, CN, CF₃, OR, R, ═O, ═S, ═NR, ═N⁺(O)(R), ═N(OR), ═N⁺(O)(OR), ═N—NRR′, —C(═O)R, —C(═O)OR, —C(═O)NRR′, —NRR′, —N⁺RR′R″, —N(R)C(═O)R′, —N(R)C(═O)OR′, —N(R)C(═O)NR′R″, —SR, —OC(═O)R, —OC(═O)OR, —OC(═O)NRR′, —OS(O)₂(OR), —OP(═O)(OR)(OR′), —OP(OR)(OR′), —P(═O)(OR)(OR′), —P(═O)(OR)NR′R″, —S(O)R, —S(O)₂R, —S(O)₂NR, —S(O)(OR), —S(O)₂(OR), —SC(═O)R, —SC(═O)OR, ═O and —SC(═O)NRR′; wherein each R, R′ and R″ is independently selected from H, C₁-C₁₂ alkyl, C₂-C₈ alkenyl, C₂-C₈ alkynyl, C₆-C₂₀ aryl and C₂-C₂₀ heterocyclyl. Substituents may also be combinations of alkyl, alkenyl, alkynyl, carbocycle, aryl, and heteroaryl radicals, such as cyclopropylmethyl, cyclohexylethyl, benzyl, and N-ethylmorpholino, and substituted forms thereof.

The terms “treat” and “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), lapatinib (TYKERB®, GlaxoSmithKline PLC), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma11 and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb, Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (docetaxel; Rhone-Poulenc Rorer, Antony, France); chloranbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XEL)DA®, Hoffman LaRoche Inc); ibandronate; CPT-11; topoisomerase inhibitor RFS 2000; difluoromethylornithine (DMFO); retinoids such as retinoic acid; capecitabine; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN® and LUCENTIS®, Genentech); (x) therapeutic antibodies such as HERCEPTIN®, AVASTIN®, LUCENTIS®; (xi) antibody-drug conjugates such as MYLOTARG®; and (xii) pharmaceutically acceptable salts, acids and derivatives of any of the above.

The term “prodrug” as used in this application refers to a precursor or derivative form of a compound of the invention that is less cytotoxic to cells compared to the parent compound or drug and is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al, “Prodrugs: A Chemical Approach to Targeted Drug Delivery,” Directed Drug Delivery, Borchardt et al, (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, compounds of the invention and chemotherapeutic agents such as described above.

A “metabolite” is a product produced through metabolism in the body of a specified compound or salt thereof. Metabolites of a compound may be identified using routine techniques known in the art and their activities determined using tests such as those described herein. Such products may result for example from the oxidation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

A “liposome” is a small vesicle composed of various types of lipids, phospholipids and/or surfactant which is useful for delivery of a drug (such as the c-Met inhibitors disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

A “salt,” as used herein, refers to organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion or other counter ion. The counter ion may be any organic or inorganic moiety that stabilizes the charge on the parent compound. Furthermore, a salt may have more than one charged atom in its structure. Instances where multiple charged atoms are part of the salt can have multiple counter ions. Hence, a salt can have one or more charged atoms and/or one or more counter ion.

If the compound of the invention is a base, the desired salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In certain embodiments, the salt is a pharmaceutically acceptable salt. The phrase “pharmaceutically acceptable” indicates that the substance or composition must be compatible chemically and/or toxicologically, with the other ingredients comprising a formulation, and/or the mammal being treated therewith.

The compounds of Formula I also include salts of such compounds which are not necessarily pharmaceutically acceptable salts, and which may be useful as intermediates for preparing and/or purifying compounds of Formula I and/or for separating enantiomers of compounds of Formula I.

A “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The terms “compound of this invention,” and “compounds of the present invention” and “compounds of Formula I” include compounds of Formula I and stereoisomers, geometric isomers, tautomers, solvates, metabolites, salts and pharmaceutically acceptable prodrugs thereof.

The term “mammal” includes, but is not limited to, humans, dogs, cats, horses, cows, pigs, and sheep, and poultry.

c-Met Inhibitor Compounds

The present invention provides quinoline compounds, and pharmaceutical formulations thereof, that are potentially useful in the treatment of diseases, conditions and/or disorders modulated by c-Met. More specifically, the present invention provides compounds of Formula I:

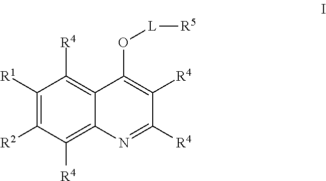

and stereoisomers, geometric isomers, tautomers, solvates, metabolites, salts, and pharmaceutically acceptable prodrugs thereof, wherein:

R¹, R² and R⁴ are independently selected from H, F, Cl, Br, I, CN, —(CR¹⁴R¹⁵)ₜNR¹⁰R¹¹, —C(═Y)R¹⁰, —C(═Y)OR¹⁰, —C(═Y)NR¹⁰R¹¹, —C(═O)NR¹²(CR¹⁴R¹⁵)ₜNR¹⁰R¹¹, —NO₂, —NR¹⁰R¹¹, —NR¹⁰C(═Y)R¹¹, —NR¹⁰C(═Y)OR¹¹, —NR¹²C(═Y)NR¹⁰R¹¹, —NR¹²SO₂NR¹⁰R¹¹, —OR¹⁰, —OC(═Y)R¹⁰, —OC(═Y)OR¹⁰, —OC(═Y)NR¹⁰R¹¹, —OP(═Y)(OR¹⁰)(OR¹¹), —OP(OR¹⁰)(OR¹¹), —P(═Y)(OR¹⁰)(OR¹¹), —SR¹⁰, —S(O)R¹⁰, —S(O)₂R¹⁰, —S(O)₂NR¹⁰R¹¹, —SC(═Y)R¹⁰, —SC(═Y)(OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl, where said alkyl, alkenyl, alkynyl, carbocyclyl, heterocyclyl, aryl and heteroaryl are optionally substituted with one or more groups independently selected from F, Cl, Br, I, CN, CF₃, —NO₂, oxo, —C(═Y)R¹⁰, —C(═Y)OR¹⁰, —C(═Y)NR¹⁰R¹¹, —(CR¹⁴R¹⁵)ₙ—NR¹⁰R¹¹, —NR¹⁰C(═Y)R¹⁰, —NR¹⁰C(═Y)OR¹¹, —NR¹²C(═Y)NR¹⁰R¹¹, —NR¹²SO₂R¹⁰, ═NR¹⁰, —OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰, —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, C₁-C₂₀ heteroaryl, —(CR¹⁴R¹⁵)ₜ—NR¹²C(═O)(CR¹⁴R¹⁵)NR¹⁰R¹¹, and —(CR¹⁴R¹⁵)ₜ—NR¹⁰R¹¹,

with the proviso that at least one of R¹ and R² is not H;

L is C₃-C₁₂ carbocyclyl, C₂-C₂₀ heterocyclyl, C₆-C₂₀ aryl or C₁-C₂₀ heteroaryl, wherein said carbocyclyl, heterocyclyl, aryl and heteroaryl are optionally substituted with one or more groups independently selected from R⁴ and R¹⁰, with the proviso that L is not naphthyl;

R⁵ is —C(═Y)R¹³, —C(═Y)NR¹⁰R¹³, —NR¹⁰R¹³, —NR¹⁰C(═Y)R¹³, —NR¹⁰C(═Y)OR¹³, —NR¹²SO₂R¹⁰, —NR¹²C(═Y¹)(CR¹⁴R¹⁵)C(═Y²)NR¹⁰R¹¹, C₃-C₁₂ carbocyclyl, C₂-C₂₀ heterocyclyl, C₆-C₂₀ aryl, or C₁-C₂₀ heteroaryl, wherein said carbocyclyl, heterocyclyl, aryl, and heteroaryl are optionally substituted with one or more groups independently selected from oxo, F, Cl, Br, I, SO₂R^{c}, CN, OR^{a}, (CH₂)ₙ—NR^{a}R^{b}, C(═O)NR^{a}R^{b}, C(═O)R^{a}, CR^{a}C(═O)R^{b}, NHSO₂R^{c}, CF₃, C₁-C₁₂ alkyl, C₂-C₈ alkenyl, C₂-C₈ alkynyl, (CH₂)ₙ—(C₆-C₂₀ aryl), (CH₂)ₙ-cycloalkyl, CH(OH)-aryl, CH(CO₂CH₃)aryl, and (CH₂)ₙ—(C₁-C₂₀ heteroaryl), and wherein any aryl or heteroaryl of the one or more groups is optionally substituted with one or more R^{d};

R¹⁰, R¹¹ and R¹² are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more groups independently selected from F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₃-C₁₂ carbocyclyl, C₂-C₂₀ heterocyclyl optionally substituted with C₁-C₆ alkyl, CH₂OH or SO₂Me, C₆-C₂₀ aryl, and C₁-C₂₀ heteroaryl optionally substituted with C₁-C₆ alkyl,

or R¹⁰ and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₙOR^{a}, NR^{a}R^{b}, CF₃, F, Cl, Br, I, SO₂R^{a}, C(═O)R^{a}, NR¹⁰C(═Y)R¹¹, C(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl and C₁-C₂₀ heteroaryl;

R¹³ is H, C₁-C₆ alkyl, —(CR¹⁴R¹⁵)ₙ-cycloalkyl, —(CR¹⁴R¹⁵)ₙ-heterocyclyl, —(CR¹⁴R¹⁵)ₙ-aryl, —(CR¹⁴R¹⁵)ₙ-heteroaryl, (CR¹⁴R¹⁵)ₙ—O—(CR¹⁴R¹⁵)ₘ-aryl, (CR¹⁴R¹⁵)—N(SO₂R^{a})—(CR¹⁴R¹⁵)R¹¹, (CR¹⁴R¹⁵)ₙ-heterocyclyl-(CR¹⁴R¹⁵)ₜ-aryl, or (CR¹⁴R¹⁵)—NR¹⁰C(═O)aryl, where said cycloalkyl, heterocyclyl, aryl, and heteroaryl portions are optionally substituted with one or more groups independently selected from F, Cl, Br, I, oxo, SO₂R^{c}, CN, OR^{a}, C(═O)R^{a}, C(═O)OR^{a}, NR^{a}R^{b}, NR^{a}C(═O)R^{b}, O—(CH₂)-aryl, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;

each R¹⁴ and R¹⁵ is independently H, C₁-C₁₂ alkyl, or (CH₂)ₜ-aryl,

or R¹⁴ and R¹⁵ together with the atoms to which they are attached form a saturated or partially unsaturated C₃-C₁₂ carbocyclic ring,

or R¹⁰ and R¹⁵ together with the atoms to which they are attached form a saturated or partially unsaturated C₂-C₁₂ heterocyclic ring,

or R¹⁴ is null and R¹⁰ and R¹⁵ together with the atoms to which they are attached form a 5-6 membered heteroaryl ring,

or R¹² and R¹⁴ together with the atoms to which they are attached form a saturated or partially unsaturated C₂-C₁₂ heterocyclic ring;

R^{a} and R^{b} are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more alkyl groups;

R^{c} is C₁-C₁₂ alkyl or C₆-C₂₀ aryl, wherein said alkyl and aryl are optionally substituted with one or more groups independently selected from F, Cl, Br, I, OR^{a} and C(═O)NR^{a}R^{b};

R^{d} is F, Cl, Br, I, CF₃, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, or C₁-C₂₀ heteroaryl;

Y, Y¹ and Y² are independently O or S;

t is 1, 2, 3, 4, 5 or 6; and

n and m are independently 0, 1, 2, 3, 4, 5 or 6.

In certain embodiments, one or both of R¹ and R² is —OR¹⁰ where R¹⁰ is C₁-C₁₂ alkyl. For example, in one embodiment one or both of R¹ and R² are methoxy.

In other embodiments, one or both of R¹ and R² is —OR¹⁰ where R¹⁰ is C₁-C₁₂ alkyl substituted with NR^{a}R^{b} or C₂-C₂₀ heterocyclyl, wherein said heterocyclyl is optionally substituted with C₁-C₆ alkyl, CH₂OH or SO₂Me.

Exemplary embodiments include the structures:

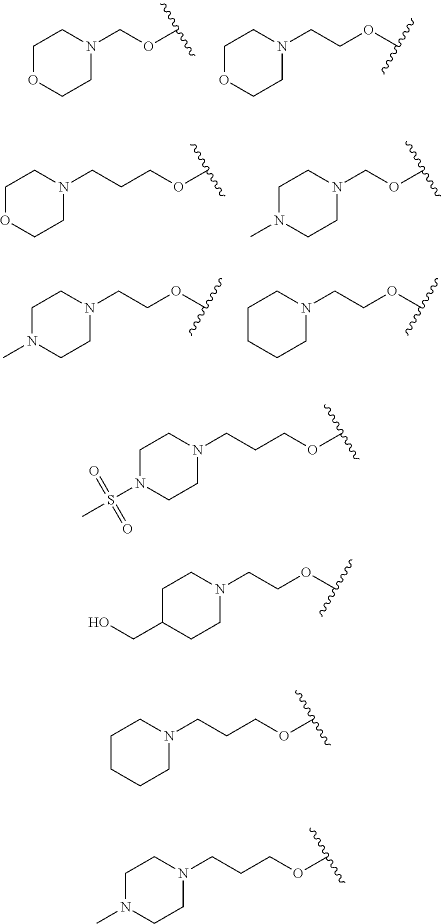

where the wavy line is the attachment site to the quinoline ring.

In other embodiments, R¹ is methoxy and R² is selected from

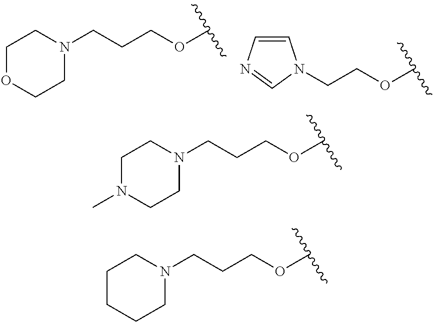

In other embodiments, one or both of R¹ and R² is —OR¹⁰ wherein R¹⁰ is C₁-C₁₂ alkyl substituted with C₁-C₂₀ heteroaryl, wherein said heteroaryl is optionally substituted with C₁-C₆ alkyl. Exemplary embodiments include the structures:

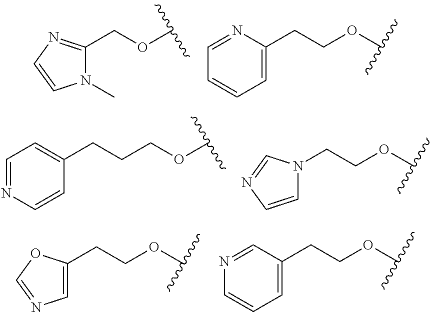

where the wavy line is the attachment site to the quinoline ring.

In other embodiments, one or both of R¹ and R² are independently selected from C₂-C₈ alkynyl substituted by —(CR¹⁴R¹⁵)ₜNR¹⁰R¹¹, including the exemplary structures:

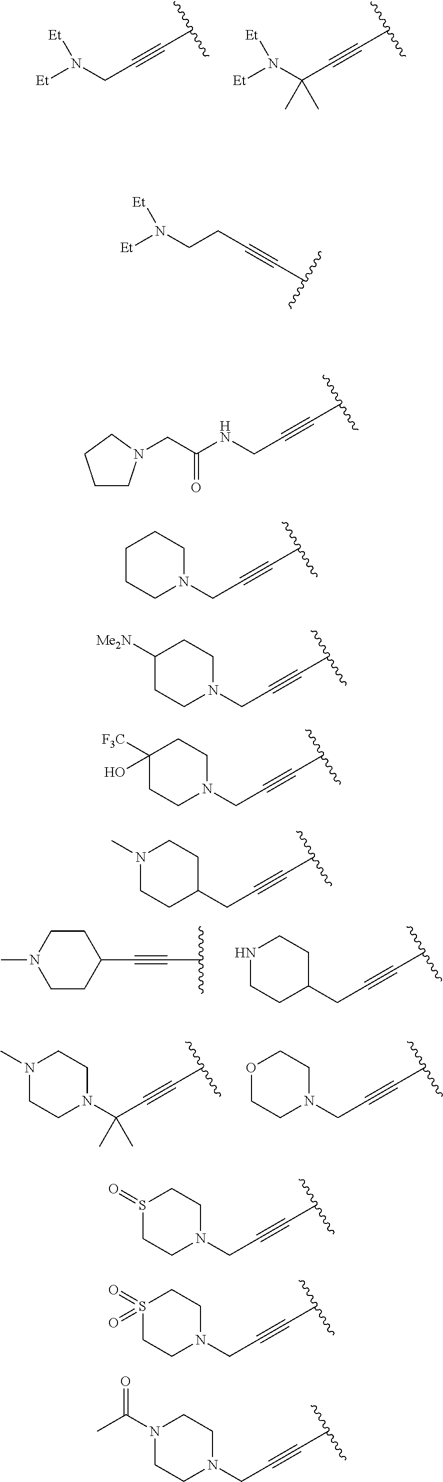

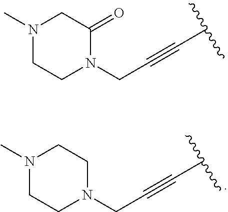

In other embodiments, one or both of R¹ and R² are independently selected from optionally substituted aryl or heteroaryl, including the exemplary structures:

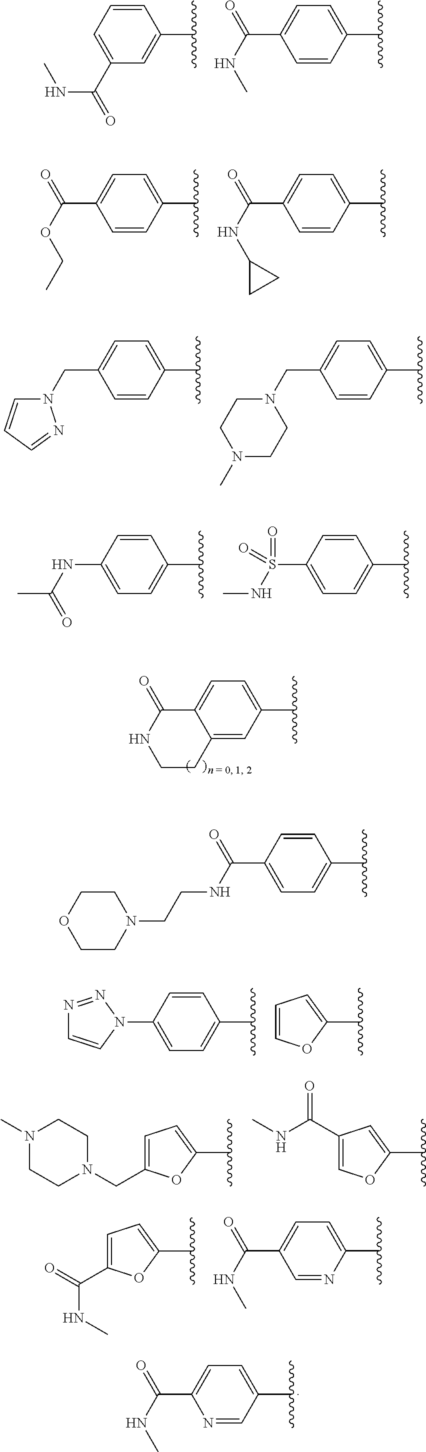

In other embodiments, one or both of R¹ and R² may be independently selected from —C(═O)NR¹⁰R¹¹ or —(CR¹⁴R¹⁵)ₜNR¹⁰R¹¹.

In other embodiments, one or both of R¹ and R² is independently alkyl optionally substituted with one or more groups independently selected from OR¹⁰, NR¹⁰R¹¹, and heteroaryl. Examples include, but are not limited to, methyl, —CH₂OH, —CH₂CH₂OH, —CH₂CH₂CH₂OH, and —CH(OH)CH₂OH.

In other embodiments, one or both of R¹ and R² are independently —OR¹⁰, including the exemplary structure:

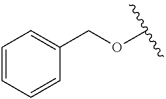

In exemplary embodiments, each R⁴ is H.

In certain embodiments, L-R⁵ is (C₃-C₁₂ carbocyclyl)-R⁵, including the exemplary structures:

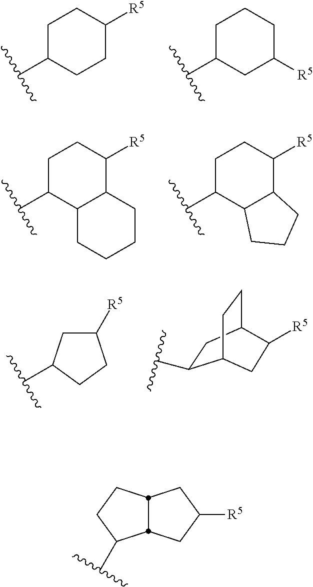

where the wavy line indicates the point of attachment to the 4-oxy position of the quinoline ring.

In certain embodiments, L-R⁵ is (C₂-C₂₀ heterocyclyl)-R⁵ wherein said heterocyclyl is optionally substituted, including the exemplary structures:

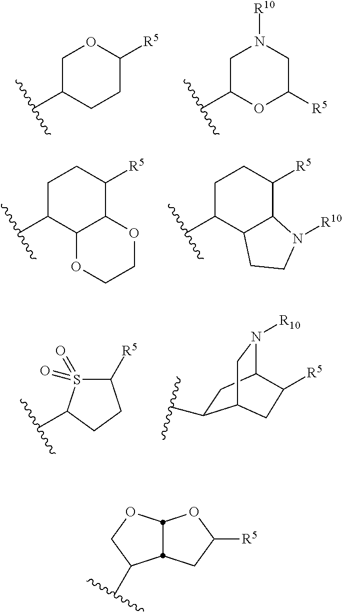

where the wavy line indicates the point of attachment to the 4-oxy position of the quinoline ring.

In certain embodiments, L-R⁵ is (C₆-C₂₀ aryl)-R⁵ wherein said aryl is optionally substituted, including the exemplary structures:

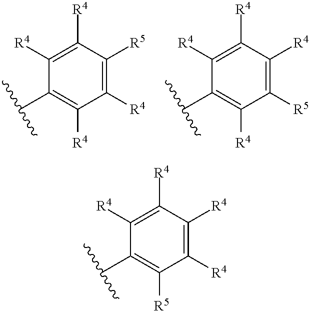

where the wavy line indicates the point of attachment to the 4-oxy position of the quinoline ring and each R⁴ is independent of the other.

Exemplary embodiments where L-R⁵ is (C₆-C₂₀ aryl)-R⁵ include the structures:

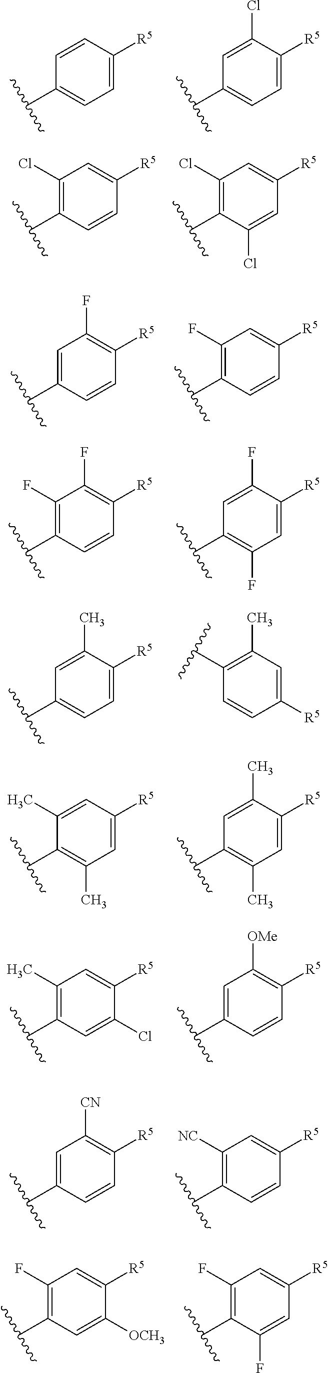

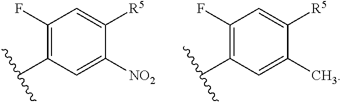

Other exemplary embodiments where L-R⁵ is (C₆-C₂₀ aryl)-R⁵, include the structures:

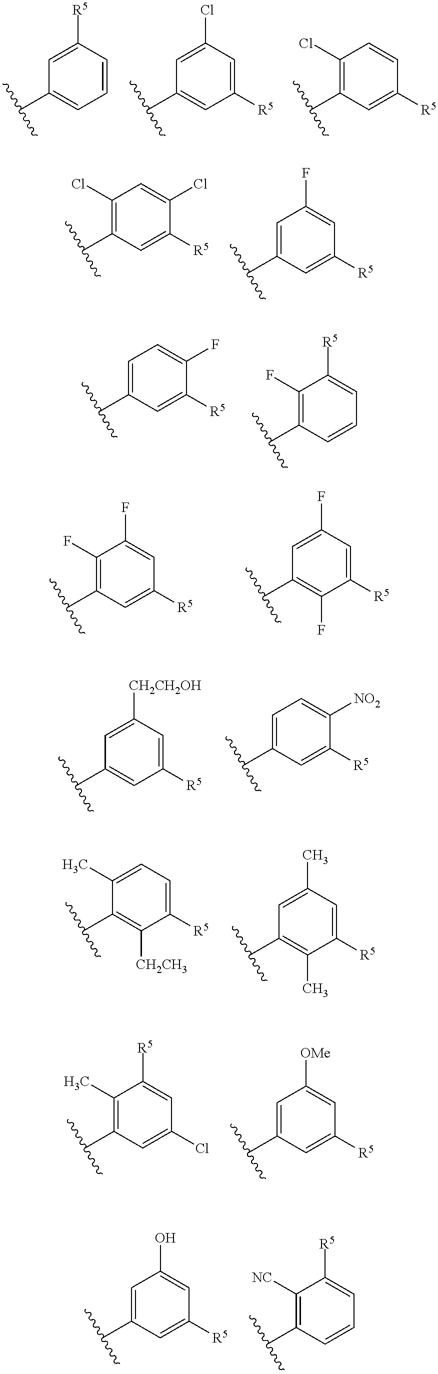

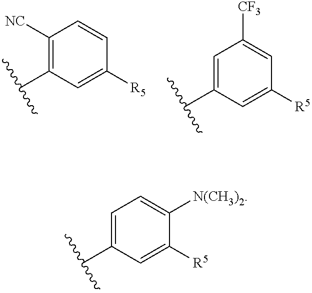

Other exemplary embodiments where L-R⁵ is (C₆-C₂₀ aryl)-R⁵ include the structures:

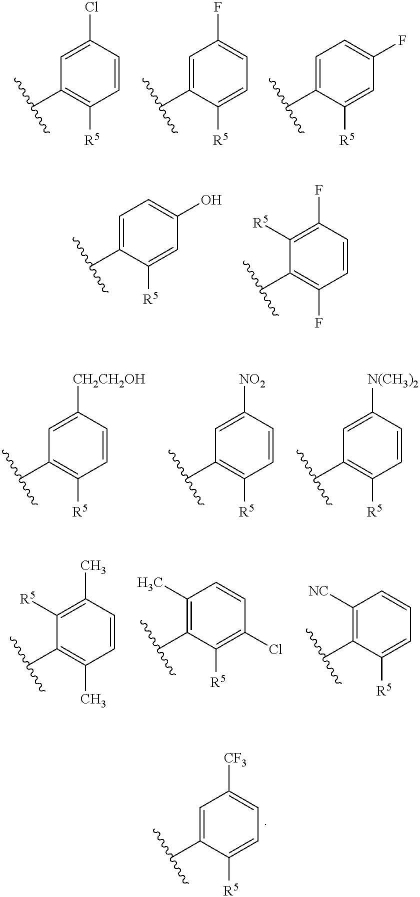

Other exemplary embodiments where L-R⁵ is (C₆-C₂₀ aryl)-R⁵ include the structures:

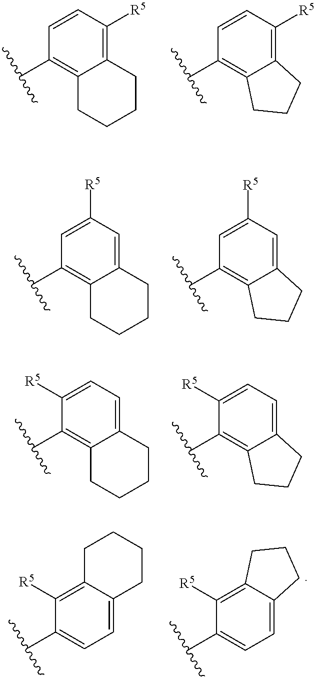

In certain embodiments, L-R⁵ is (C₁-C₂₀ heteroaryl)-R⁵. Exemplary embodiments include the structures:

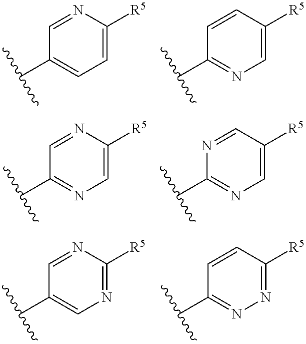

where the wavy line indicates the point of attachment to the 4-oxy position of the quinoline ring.

Exemplary embodiments where L-R⁵ is (C₁-C₂₀ heteroaryl)-R⁵ also include the structures:

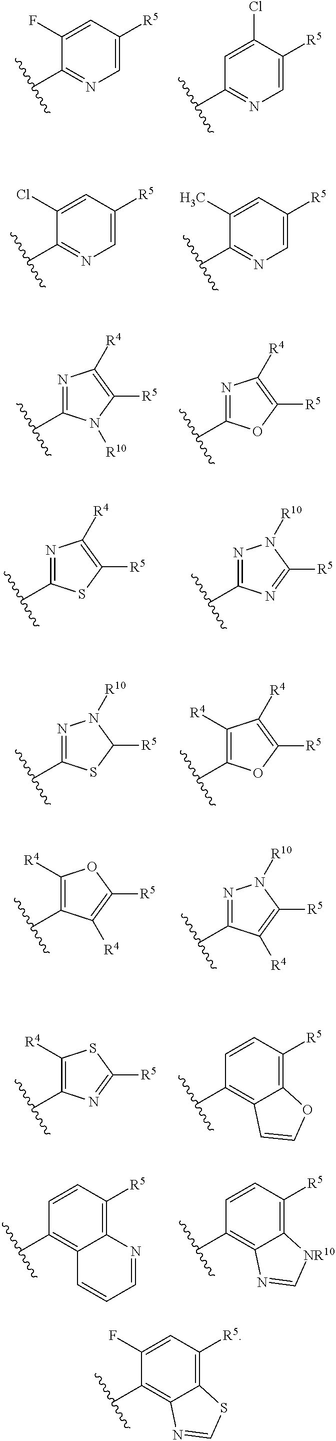

In certain embodiments, R⁵ is —C(═Y)R¹³. In certain embodiments, R¹³ is —(CR¹⁴R¹⁵)ₙ-cycloalkyl, CR¹⁴R¹⁵)aryl, —(CR¹⁴R¹⁵)ₙ—O—(CR¹⁴R¹⁵)ₘ-aryl, or —(CR¹⁴R¹⁵)ₙ-heterocyclyl-(CR¹⁴R¹⁵)ₜ-aryl, wherein said heterocyclyl portion is optionally substituted with SO₂R^{c} or C₁-C₁₂ alkyl. Exemplary embodiments include the structures:

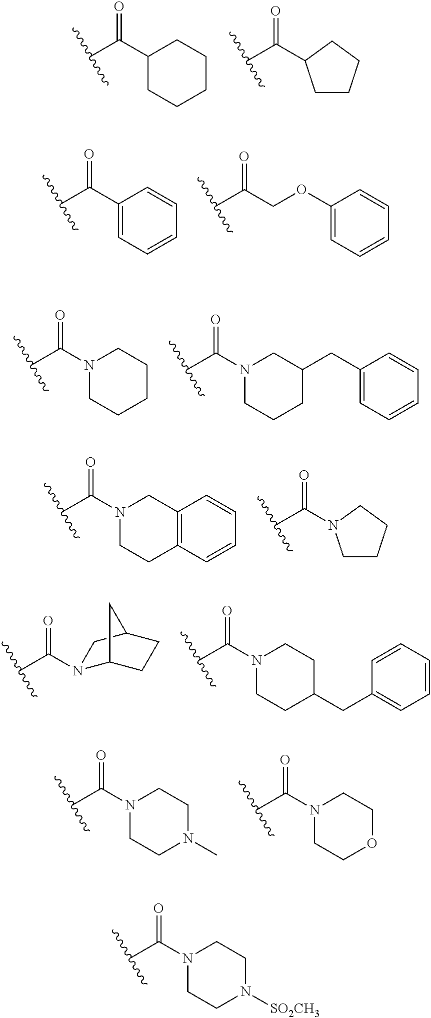

where the wavy line indicates the point of attachment to L.

In certain embodiments, R⁵ is —C(═Y)NR¹⁰R¹³. In certain embodiments, R¹⁰ is H or C₁-C₁₂ alkyl, and R¹³ is H, C₁-C₆ alkyl, —(CR¹⁴R¹⁵)ₙ-cycloalkyl, or —(CR¹⁴R¹⁵)ₙ-aryl, wherein said alkyl, cycloalkyl, and aryl portions are optionally substituted with F or OR^{a}. Exemplary embodiments of R⁵ include the structures:

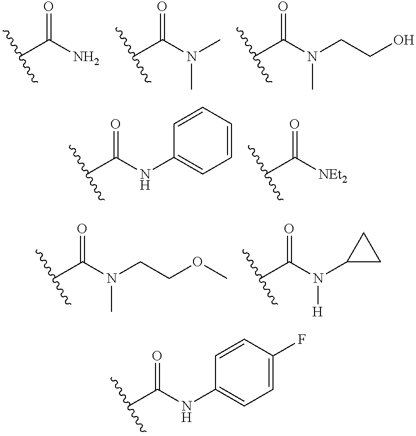

where the wavy line indicates the point of attachment to L.

In certain embodiments, R⁵ is —NR¹⁰R¹³. In certain embodiments, R¹⁰ is H or C₁-C₁₂ alkyl, and R¹³ is —(CR¹⁴R¹⁵)ₙ-heterocyclyl or —(CR¹⁴R¹⁵)ₙ-heteroaryl, wherein said heterocyclyl and heteroaryl are optionally substituted with OR^{a}, SO₂R^{c} or O—(CH₂)-aryl. Exemplary embodiments of R⁵ include the structures:

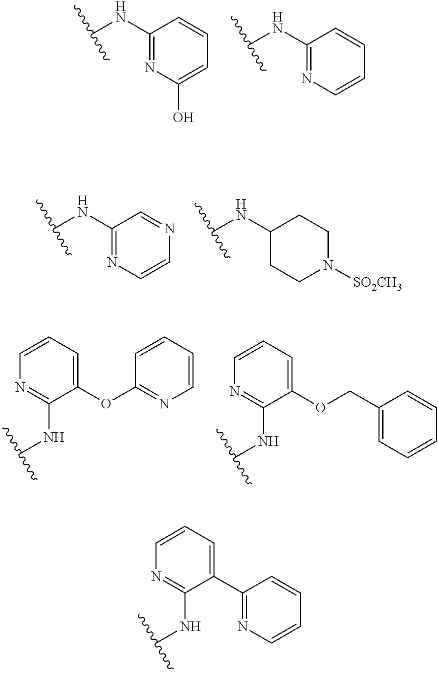

where the wavy line indicates the point of attachment to L.

In certain embodiments, R⁵ is —NR¹²C(═Y¹)(CR¹⁴R¹⁵)C(═Y²)NR¹⁰R¹¹, wherein R¹⁵ and R¹⁰ optionally together with the atoms to which they are attached form a 5-6 membered heterocyclic ring, and wherein R¹⁴ and the adjacent saturated ring carbon together with the atoms to which they are attached optionally form a fused cyclopropyl ring. Exemplary embodiments include the structures:

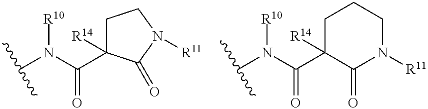

where the wavy line indicates the point of attachment to L. Exemplary embodiments of R⁵ include the structures:

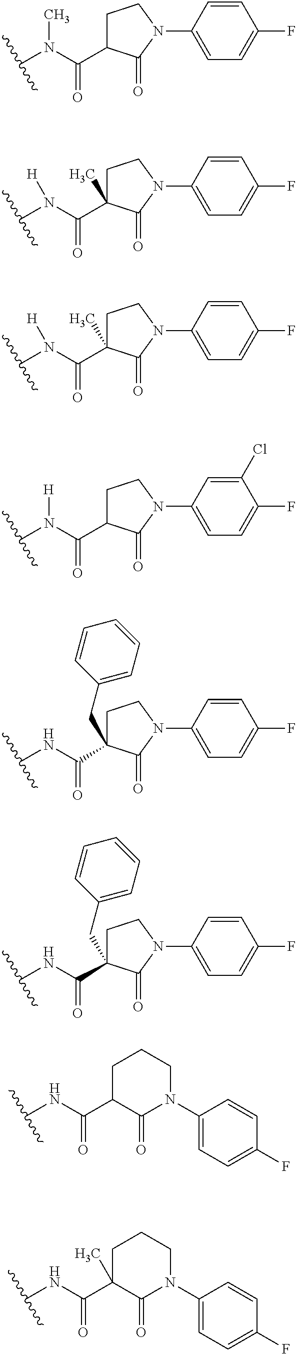

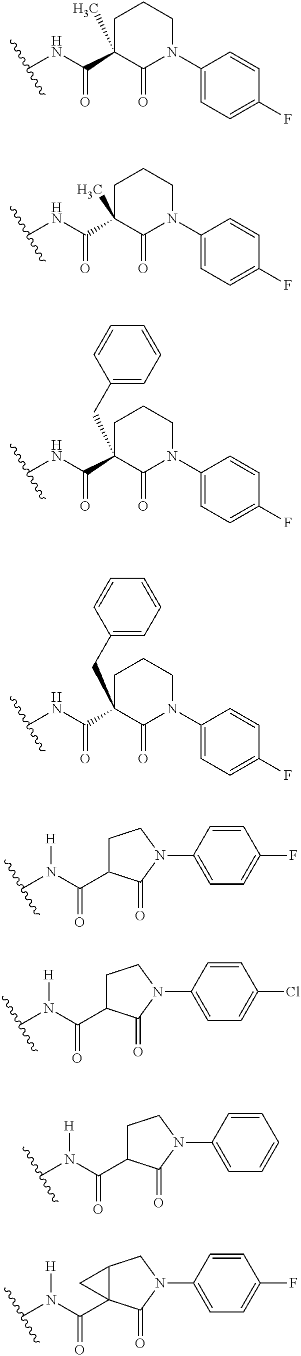

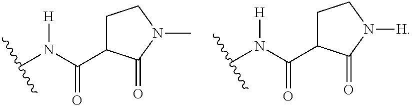

In other embodiments, R⁵ is —NR¹²C(═Y¹)(CR¹⁴R¹⁵)C(═Y²)NR¹⁰R¹¹ wherein R⁴ is null and R¹⁰ and R¹⁵ together with the nitrogen atom to which they are attached form a heteroaryl ring optionally having an additional ring nitrogen atom. Exemplary embodiments of R⁵ include the structures:

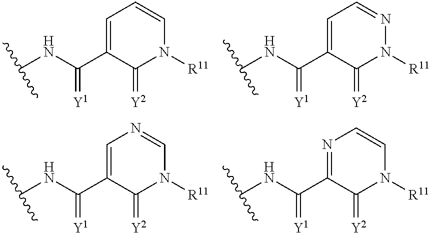

where Y¹ and Y² are independently selected from O and S; and where the wavy line indicates the point of attachment to L. In certain embodiments, R¹¹ is aryl or a C₁-C₁₂ alkyl substituted with aryl, wherein said aryl portions are optionally substituted. Particular embodiments include the structures:

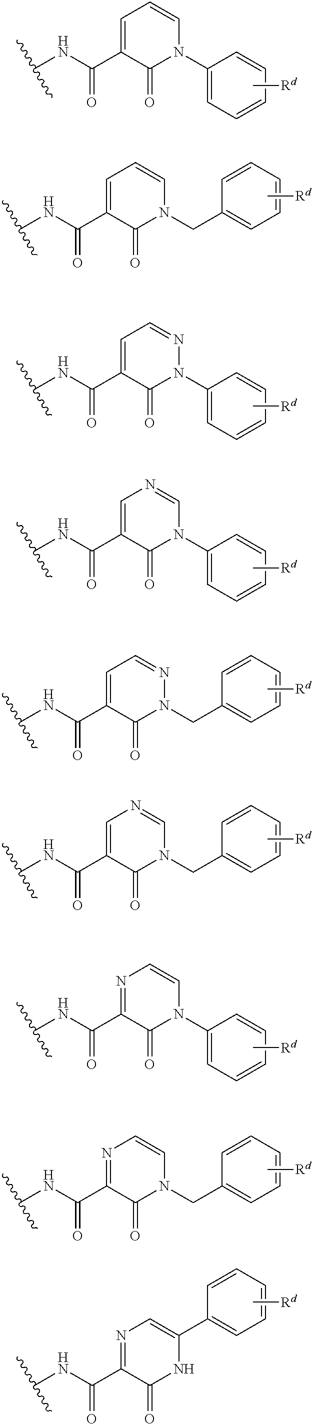

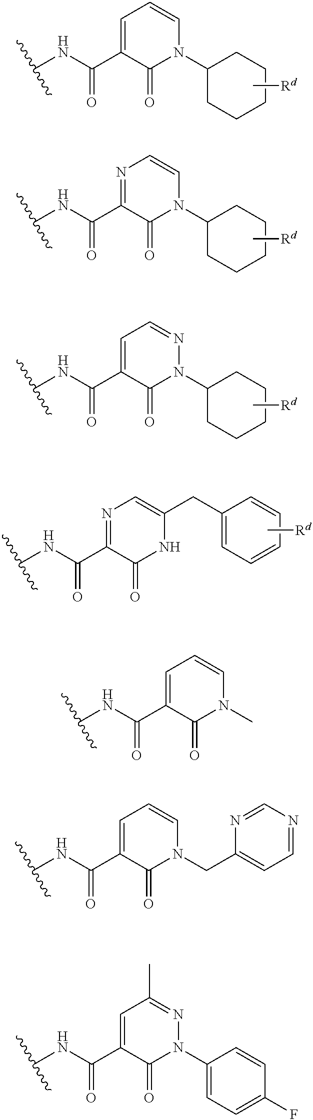

wherein the cyclohexyl and phenyl groups are optionally substituted with one or more R^{d} groups independently selected from F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, and C₁-C₂₀ heteroaryl.

In certain embodiments, R⁵ is —NR¹²C(═Y¹)(CR¹⁴R¹⁵)C(═Y²)NR¹⁰R¹¹, wherein R¹² and R¹⁴ together with the atoms to which they are attached form a 5-6 membered heterocyclic ring. Exemplary embodiments include, but are not limited to

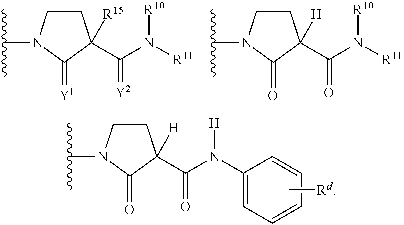

A particular example includes

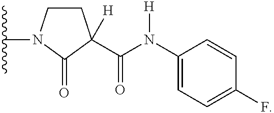

In other embodiments, R⁵ is —NR¹⁰C(═Y)R¹³. In certain embodiments, R¹³ is C₁-C₆ alkyl, (CR¹⁴R¹⁵)ₙ—O—(CR¹⁴R¹⁵)ₘ-aryl, (CR¹⁴R¹⁵)-aryl, (CR¹⁴R¹⁵)-heteroaryl, (CR¹⁴R¹⁵)-heterocyclyl, (CR¹⁴R¹⁵)—N(SO₂R^{a})(CR¹⁴R¹⁵)R¹¹, or (CR¹⁴R¹⁵)NR¹⁰C(═O)-aryl, wherein said alkyl, aryl, heteroaryl and heterocyclyl portions are optionally substituted. Exemplary embodiments of R⁵ include the structures:

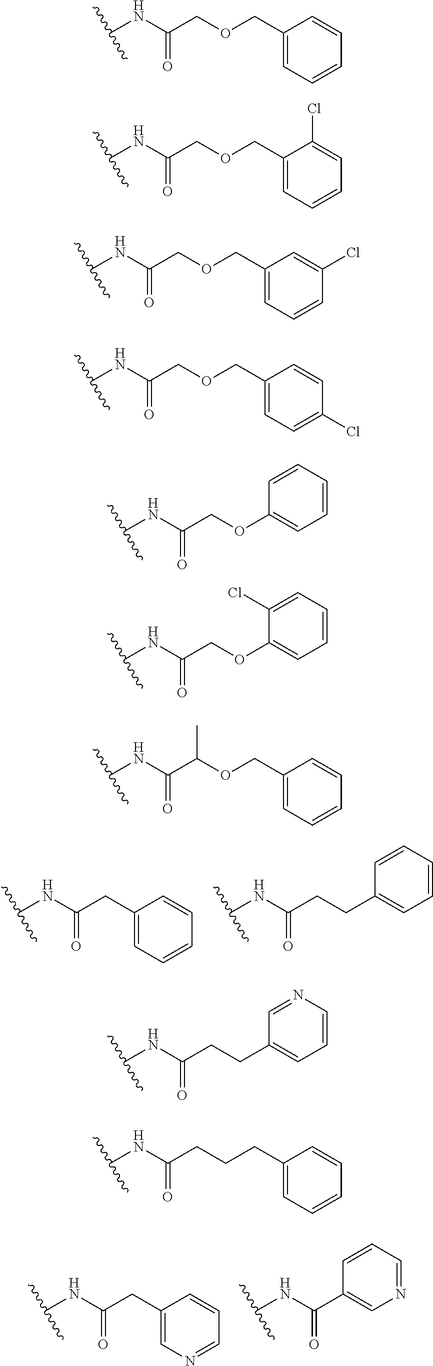

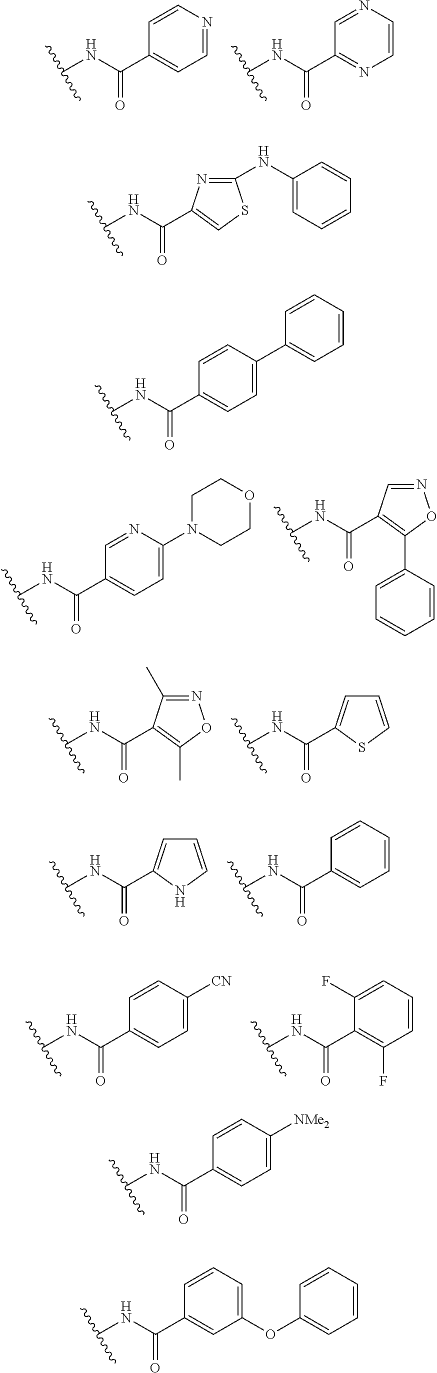

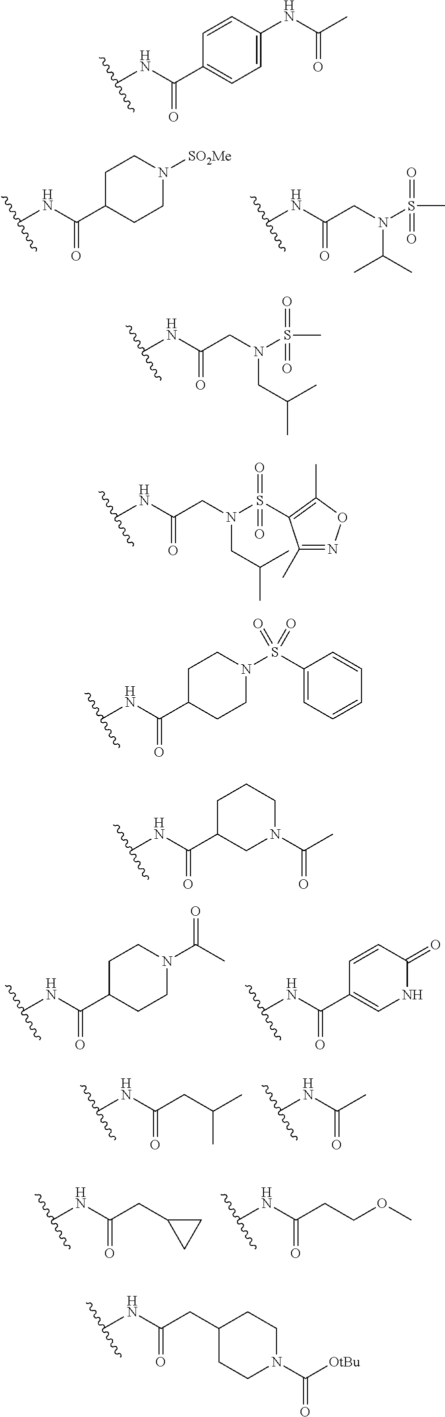

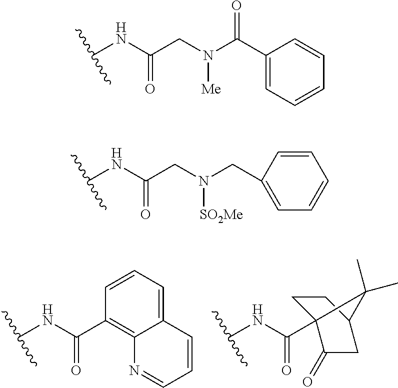

where the wavy line indicates the point of attachment to L. In certain embodiments, R⁵ is —NR¹⁰C(═Y)OR¹³, including the exemplary structures:

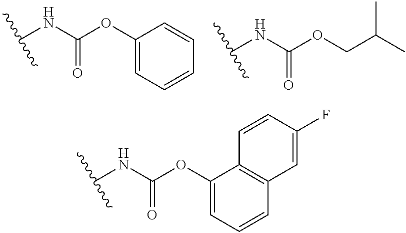

where the wavy line indicates the point of attachment to L. In certain embodiments, R⁵ is —NR¹²SO₂R¹⁰, including where R¹⁰ is alkyl or optionally substituted aryl. Exemplary embodiments include the structures:

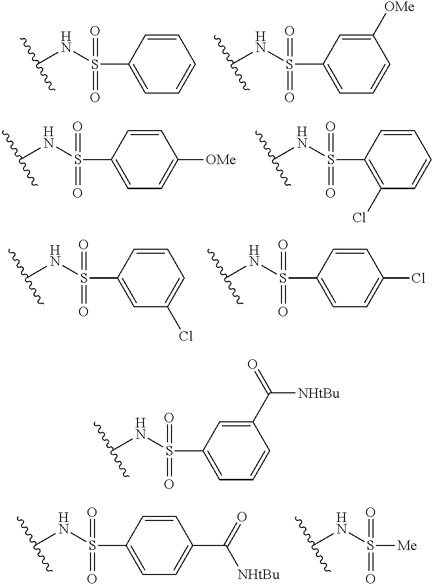

where the wavy line indicates the point of attachment to L.

In certain embodiments, R⁵ is an optionally substituted carbocyclyl, including the exemplary structures:

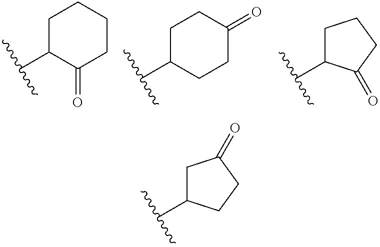

where the wavy line indicates the point of attachment to L.

In certain embodiments, R⁵ is an optionally substituted heterocyclyl, including the exemplary structures:

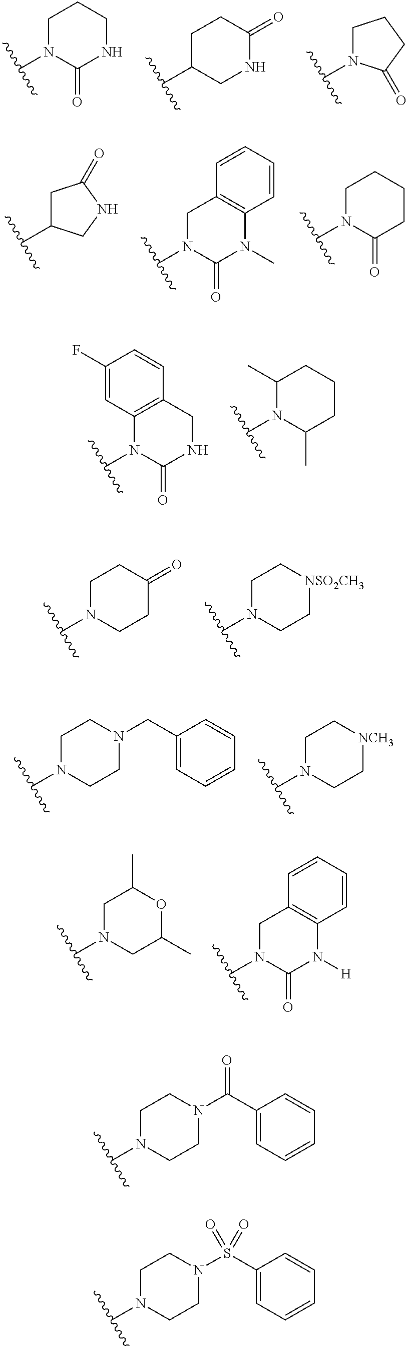

where the wavy line indicates the point of attachment to L.

In certain embodiments, R⁵ is an optionally substituted aryl, including the exemplary structures:

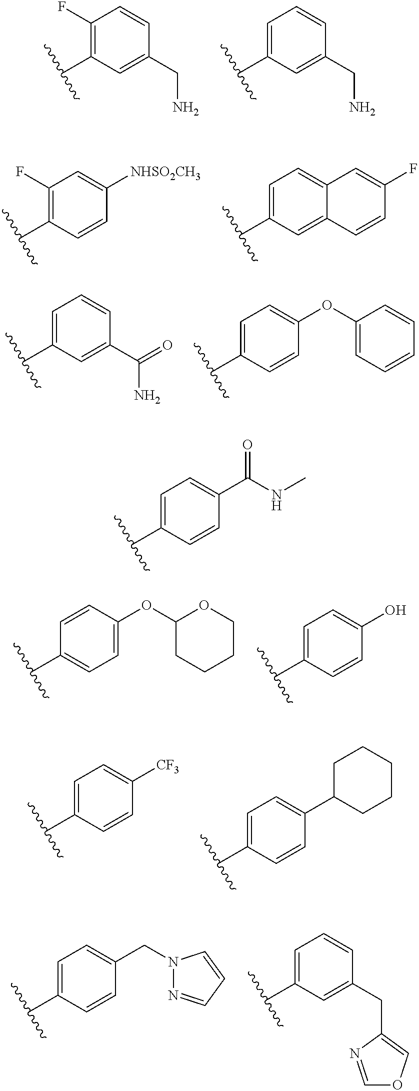

where the wavy line indicates the point of attachment to L.

In certain embodiments, R⁵ is an optionally substituted heteroaryl, including the exemplary structures:

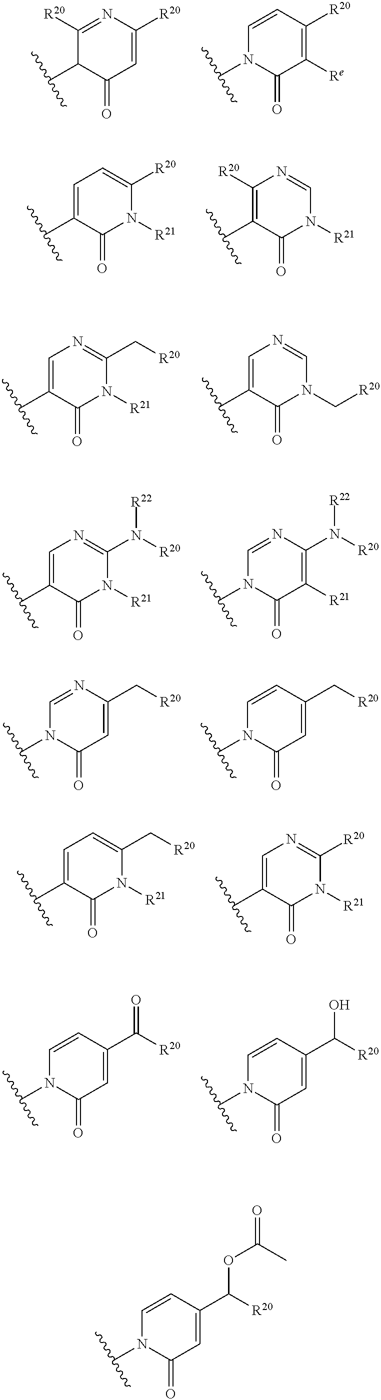

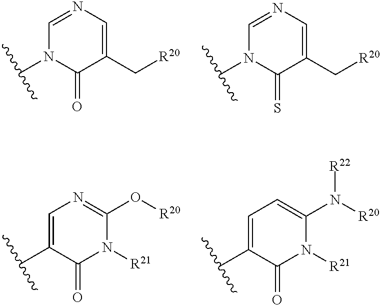

where R²⁰ is H, C₁-C₁₂ alkyl, C₃-C₁₂ cycloalkyl, C₆-C₂₀ aryl, or C₁-C₂₀ heteroaryl, and R²¹ and R²² are independently selected from H or C₁-C₁₂ alkyl, wherein said alkyl, cycloalkyl, aryl, heteroaryl are optionally substituted with one or more groups independently selected from F, Cl, Br, I and C₁-C₁₂ alkyl; and where the wavy line indicates the point of attachment to L. In certain embodiments, R²⁰ is H.

In certain embodiments, R⁵ is an optionally substituted heteroaryl, including the exemplary structures:

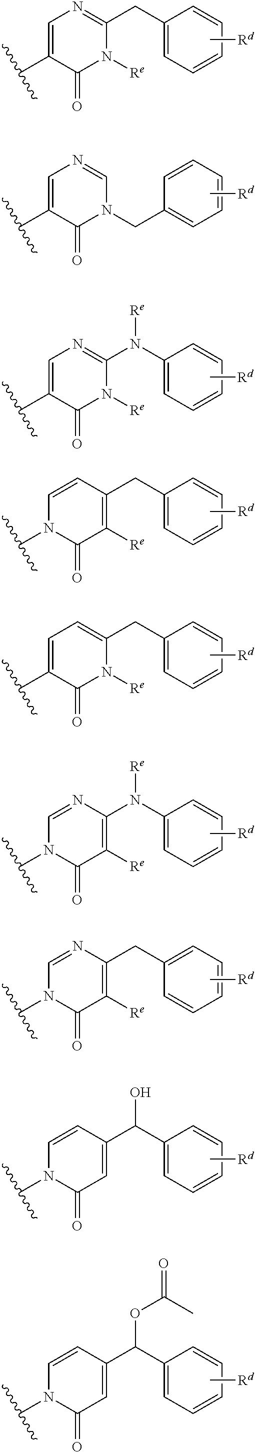

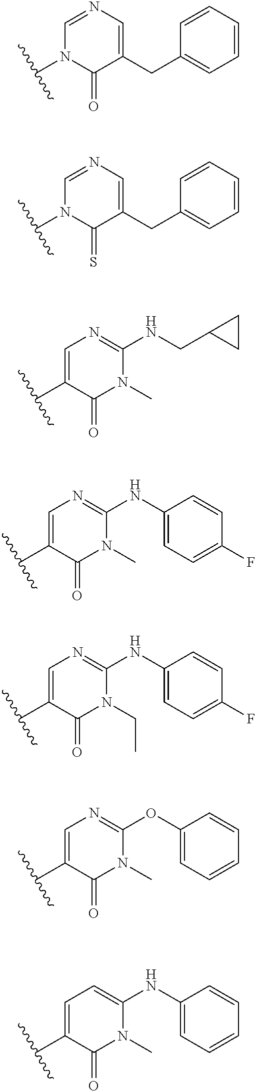

where the phenyl groups are optionally substituted with one or more R^{d} groups independently selected from F, Cl, Br, I, CF₃, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, and C₁-C₂₀ heteroaryl; and each R^{e} is independently H, C₁-C₄ alkyl or NHC(═O)-aryl wherein said aryl is optionally substituted with halogen.

In certain embodiments, R⁵ is an optionally substituted heteroaryl, including the exemplary structures:

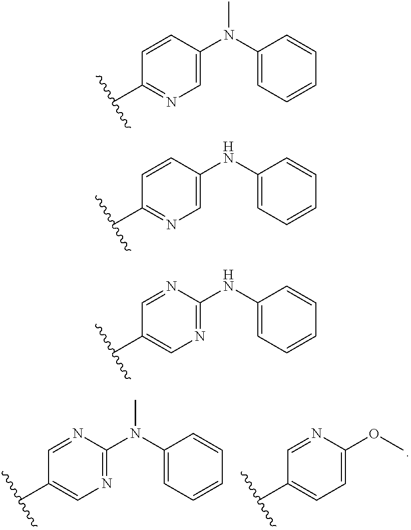

The quinoline compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In addition, the present invention embraces all geometric and positional isomers. For example, if a quinoline compound of the present invention incorporates a double bond or a fused ring, the cis- and trans-forms, as well as mixtures thereof, are embraced within the scope of the invention. Both the single positional isomers and mixture of positional isomers, e.g., resulting from the N-oxidation of the pyrimidine and pyrazine rings, are also within the scope of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The compounds of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The present invention also embraces isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (3H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Synthesis of cMET Inhibitor Compounds

Quinoline compounds of Formula I of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database).

In certain embodiments, compounds of Formula I may be readily prepared using procedures well-known to prepare quinoline compounds; and other heterocycles, which are described in: Comprehensive Heterocyclic Chemistry, Editors Katritzky and Rees, Pergamon Press, 1984; Klemm et al (1970) J. Hetero. Chem. 7(2):373-379; Klemm et al (1974) J. Hetero. Chem. 11(3): 355-361; Klemm et al (1976) J. Hetero. Chem. 13:273-275; Klemm et al (1985) J. Hetero. Chem. 22(5):1395-1396; Bisagni et al (1974) Bull. Soc. Chim. Fr. (3-4, Pt. 2):515-518; Frehel et al (1984) Heterocycles 22(5):1235-1247; WO 93/13664; WO 2004/012671; WO 2005/061476; U.S. Application Publication Nos. 2003/0045540, US 2003/0105089, and 2004/0024210; and U.S. Pat. Nos. 5,252,581, 6,232,320, and 6,579,882.

Compounds of Formula I may be prepared singly or as compound libraries comprising at least 2, for example 5 to 1,000 compounds, or 10 to 100 compounds. Libraries of compounds of Formula I may be prepared by a combinatorial ‘split and mix’ approach or by multiple parallel syntheses using either solution phase or solid phase chemistry, by procedures known to those skilled in the art. Thus according to a further aspect of the invention there is provided a compound library comprising at least 2 compounds, or pharmaceutically acceptable salts thereof.

For illustrative purposes, Schemes 1-20 show general methods for preparing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In preparing compounds of Formulas I, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

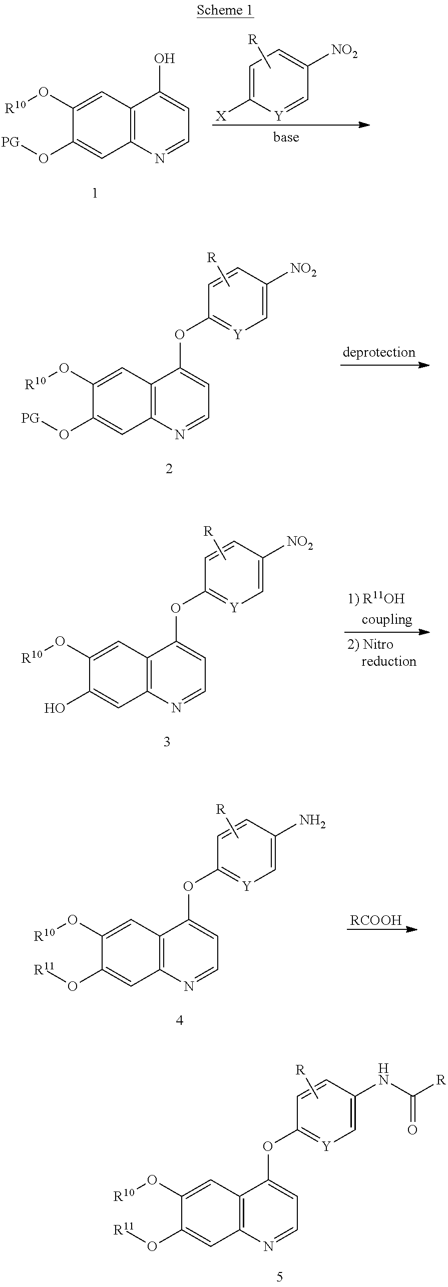

Scheme 1 shows a general method for the synthesis of intermediate compound 4, which is useful for the synthesis of compounds of Formula I. Syntheses of 4-phenoxy-6,7-dialkoxyquinolines have been previously reported in US 2004/0242603; US 2005/0002326; WO 2005/030140; J. Med. Chem. (2005) 48:1359-1366. As shown in Scheme 1, reaction of a 4-hydroxy-6,7-dialkoxyquinoline 1 with a variably substituted p-halonitroarene or heteroarene wherein X is F or Cl and Y is N or CH using an appropriate base (e.g. Cs₂CO₃, NaH, KOt-Bu, or the like) provides intermediate 2. The protecting group PG can then be removed (in the case where PG=benzyl, HBr or TFA can be used for the deprotection) to give intermediate 3 and a new 7-alkoxy substituent introduced, typically using Mitsunobu conditions or alkylation with an alkyl halide and base. Nitro reduction under hydrogenation conditions or with zinc in acetic acid gave key intermediate 4. Alternatively, exchange of the protecting group PG exchange for R¹¹ could be omitted from the sequence and thus provide compound 4 with the 6,7-alkoxy substituents originally contained in intermediate 1. Compound 4 can then be coupled with an appropriate acid (as prepared according to Schemes 13-17 below) using standard amide bond construction methods to provide compound 5.

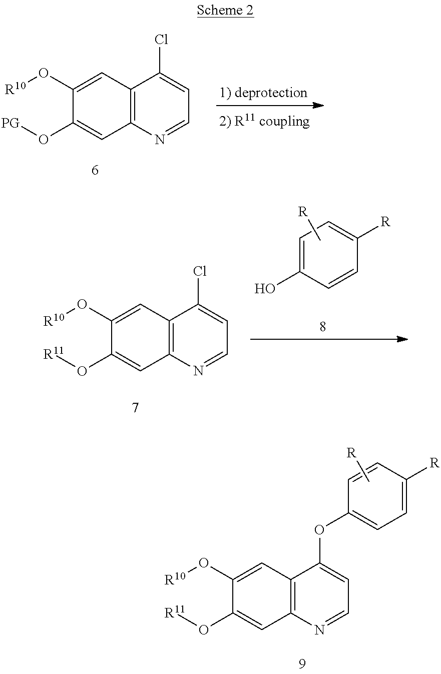

Scheme 2 shows a general synthetic route for the synthesis of compound 9, which is useful for the synthesis of compounds of Formula I. A 4-chloro-6,7-dialkoxyquinoline, such as compound 6, can be prepared by chlorination of the corresponding hydroxyquinoline (compound I, Scheme 1) typically using POCl₃, MeSO₂Cl and the like. The protecting group PG can then be removed (in the case where PG=benzyl, HBr, or TFA can be used for the deprotection) and a new 7-alkoxy substituent introduced, typically using Mitsunobu conditions or alkylation with an alkyl halide and base to give compound 7. Compound 7 can then be reacted under basic conditions, typically DMAP in bromobenzene or Cs₂CO₃ in DMF, with a functionalized phenol 8 (general schemes for synthesis of preferred functionalized phenols are shown below) to give rise to compound 9. Compound 9 can optionally be further manipulated depending on the phenol functionalization. Alternatively, PG exchange for R¹¹ can be omitted from the sequence and thus provide intermediate 9 with the 6,7-alkoxy substituents originally contained in intermediate 6.

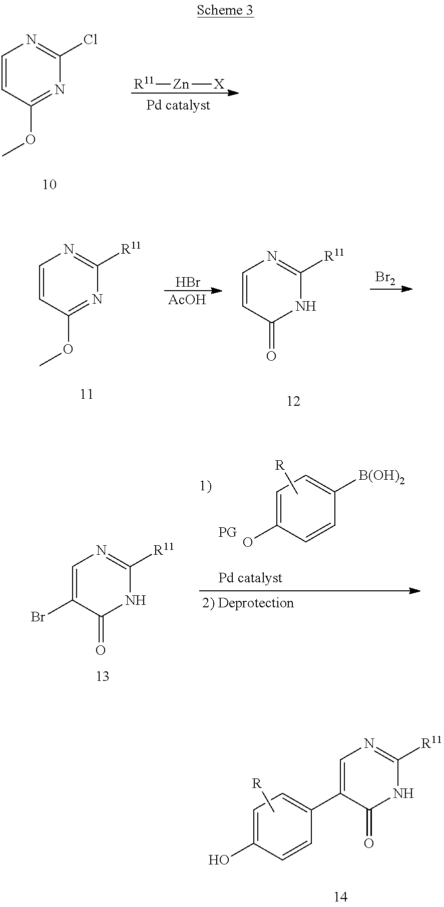

Scheme 3 shows a route for the preparation of phenol compound 14. Commercially available 2-chloro-4-methoxypyrimidine 10 is reacted with the appropriate zinc reagent and palladium catalyst to give 2-substituted 4-methoxypyrimidine 11. Deprotection of the methoxypyrimidine with HBr in acetic acid provides 2-substituted pyrimidinone 12. Bromination in the 5-position gives pyrimidinone intermediate 13. Suzuki coupling of compound 13 to an appropriate boronic acid gives a bicyclic intermediate, which after final deprotection of the phenol gives compound 14 which can be reacted with the appropriate core intermediate 7 as in Scheme 2 to provide compound 9.

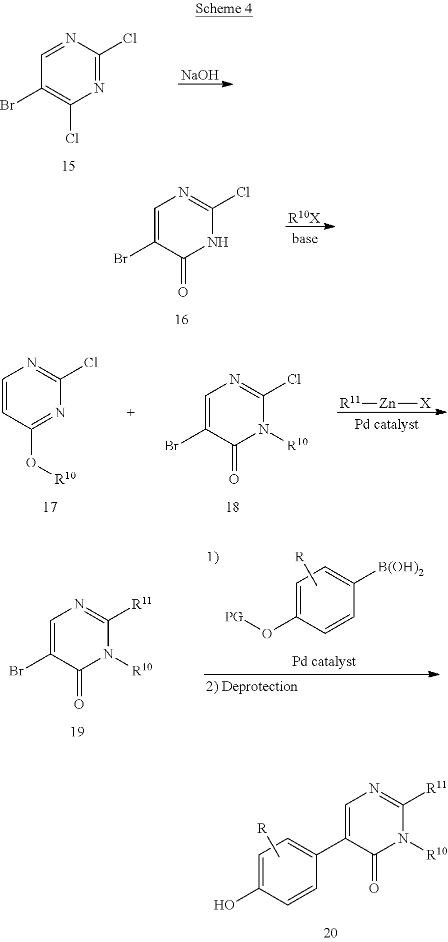

Scheme 4 shows a route for the preparation of the 1-substituted pyrimidinone intermediate 20 (wherein R¹⁰ is independently selected from H, alkyl, aryl and heteroaryl). 5-bromo-2,4-dichloropyrimidine 15 is hydrolyzed with NaOH to give 5-bromo-2-chloropyrimidin-4(3H)-one 16 as described in EP1506967 A1. Alkylation of compound 16 to provide the 1-substituted pyrimidinone can be accomplished with an alkylation agent (e.g. iodomethane, or the like) mediated by an appropriate base (e.g. sodium alkoxides, lithium or sodium hydride, or the like) providing a mixture of isomers 17 and 18. Isomers 17 and 18 can be separated using purification techniques known to those skilled in the art (e.g. flash chromatography, reverse phase HPLC, or the like). Compound 18 is reacted with the appropriate zinc reagent and palladium catalyst to give 2-substituted intermediate 19. Suzuki coupling of compound 19 to an appropriate boronic acid followed by final deprotection of the phenol gives compound 20, which can be reacted with the appropriate core intermediate 7 as in Scheme 2 to provide compound 9.

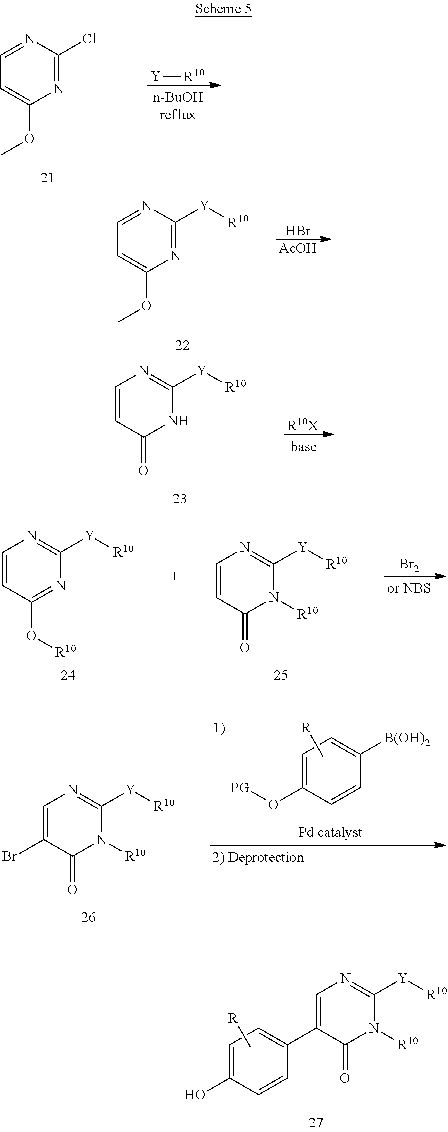

Scheme 5 shows a method for preparing phenol intermediate 27 (wherein R¹⁰ is independently selected from H, alkyl, aryl and heteroaryl). Nucleophilic substitution of 2-chloro-4-methoxypyrimidine 21 with a compound of the formula HY—R¹⁰, (wherein Y is O, N or S) can be accomplished in an appropriate solvent such as n-butanol, at refluxing temperature to give intermediate 22. Deprotection of the methoxypyrimidine with HBr in acetic acid provides 2-substituted pyrimidinone 23. Alkylation of 23 to provide the 1-substituted pyrimidinone can be accomplished with an alkylation agent (e.g. iodomethane, or the like) mediated by an appropriate base (e.g. sodium alkoxides, lithium or sodium hydride, or the like) providing a mixture of isomers 24 and 25. Isomers 24 and 25 can be separated using purification techniques known to those skilled in the art (e.g. flash chromatography, reverse phase HPLC, or the like). Bromination in the 5-position with a brominating agent such as Br₂ or NBS gives compound 26. Suzuki coupling of compound 26 to an appropriate boronic acid gives a bicyclic intermediate which after final deprotection of the phenol gives compound 27. Compound 27 can be reacted with the appropriate core intermediate 7 as in Scheme 2 to provide compound 9.

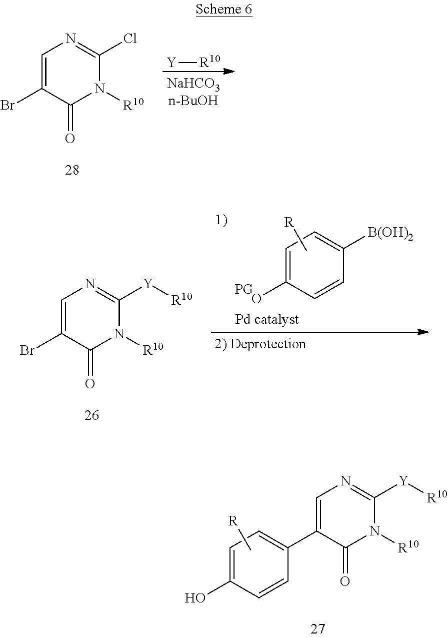

Scheme 6 shows an alternative route to compound 27 (wherein R¹⁰ is independently selected from H, alkyl, aryl and heteroaryl). Nucleophilic substitution of compound 28 with a compound of the formula HY—R¹⁰ (wherein Y is O, N or S) can be accomplished at elevated temperature with a base such as NaHCO₃ in an appropriate solvent such as n-butanol to give intermediate 26. Suzuki coupling of compound 26 to an appropriate boronic acid gives a bicyclic intermediate which after final deprotection of the phenol gives compound 27. Intermediate 27 can then be reacted with the appropriate core intermediate 7 as in Scheme 2 to give compound 9.

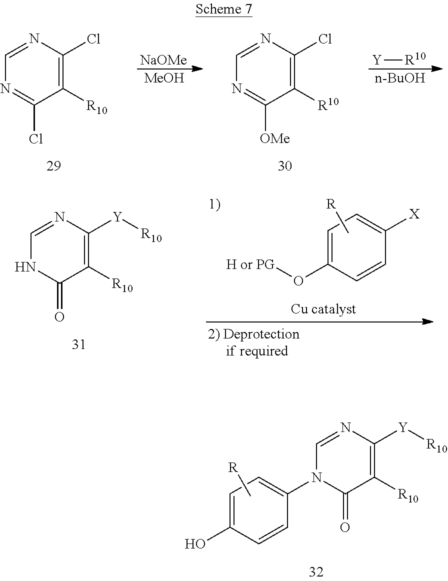

Scheme 7 shows a route to phenol intermediate 32 (wherein R¹⁰ is independently selected from H, alkyl, aryl and heteroaryl). Nucleophilic substitution of compound 29 with NaOMe can be accomplished at elevated temperature in an appropriate solvent such as methanol. Nucleophilic substitution of compound 30 with a compound of the formula HY—R¹⁰, (wherein Y is O, N or S), to form the 5-substituted pyrimidinone 31 can be accomplished at elevated temperature with a base such as NaHCO₃ in an appropriate solvent such as n-butanol. Under these reaction conditions, deprotection of the methoxypyrimidine to the pyrimidinone can also be achieved. Alternatively, deprotection of the methoxypyrimidine can be accomplished with HBr in acetic acid. Copper (I)-mediated coupling of compound 31 to an appropriate halide provides compound 32. In some instances, the halide used in the coupling reaction contains a standard protecting group. In those cases, the protecting group can be removed by standard conditions known in the art. Compound 32 can then be reacted with the appropriate core intermediate 7 as in Scheme 2 to provide compound 9.

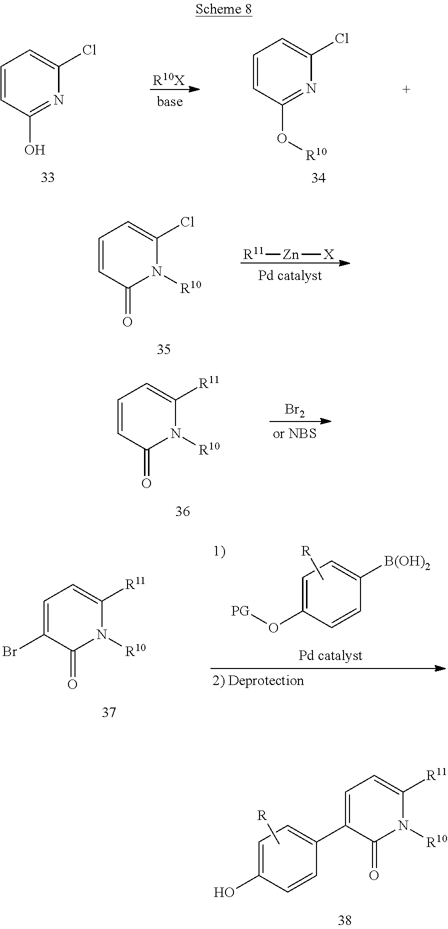

Scheme 8 shows a route for the preparation of the 1-substituted pyridone intermediate 38 (wherein R¹⁰ is independently selected from H, alkyl, aryl and heteroaryl). Alkylation of 6-chloropyridin-2-ol 33 to provide the 1-substituted pyridone 35 can be accomplished with an alkylation agent (e.g. iodomethane, or the like) mediated by an appropriate base (e.g. potassium carbonate, sodium alkoxides, lithium or sodium hydride, or the like) providing a mixture of isomers 34 and 35. Isomers 34 and 35 can be separated using purification techniques known to those skilled in the art (e.g. flash chromatography, reverse phase HPLC, or the like). Compound 35 is reacted with the appropriate zinc reagent and palladium catalyst to give 6-substituted compound 36. Bromination of the 3-position with a brominating agent such as Br₂ or NBS gives pyridone intermediate 37. Suzuki coupling of compound 37 to an appropriate boronic acid followed by final deprotection of the phenol gives compound 38, which can then be reacted with the appropriate core intermediate 7 as in Scheme 2 to give compound 9.

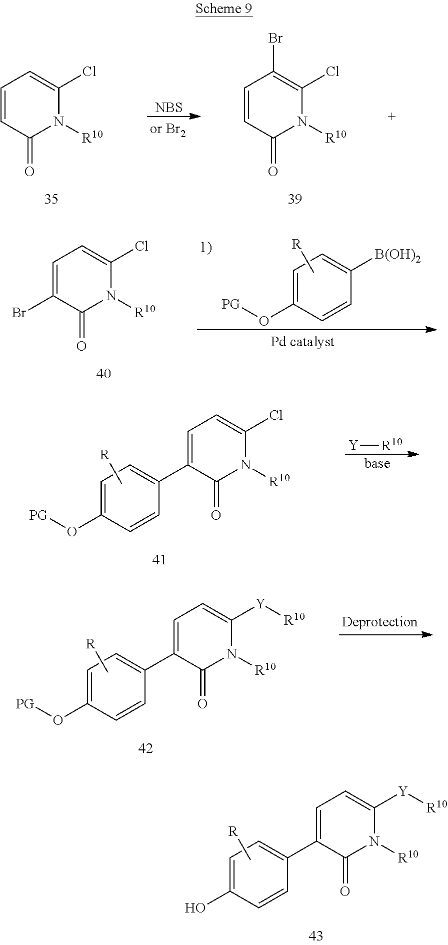

Scheme 9 shows a method for preparing phenol intermediate 43 (wherein R¹⁰ is independently selected from H, alkyl, aryl and heteroaryl). 1-Substituted pyridone 35, which can be synthesized as shown in Scheme 8, is brominated with a brominating agent such as Br₂ or NBS providing a mixture of isomers 39 and 40. Isomers 39 and 40 can be separated using purification techniques known to those skilled in the art (e.g. flash chromatography, reverse phase HPLC, or the like). Suzuki coupling of compound 40 with an appropriate boronic acid gives compound 41. Nucleophilic substitution of compound 41 with a compound of the formula HY—R¹⁰, (wherein Y is O, N or S) can be accomplished in an appropriate solvent such as THF, mediated by an appropriate base such as LDA, LiHMDS, NaHMDS, or KHMDS at appropriate temperatures (−78° C. to room temperature) to give compound 42. Final deprotection of the phenol gives compound 43, which can then be reacted with appropriate core intermediate 7 as in Scheme 2 to give compound 9.

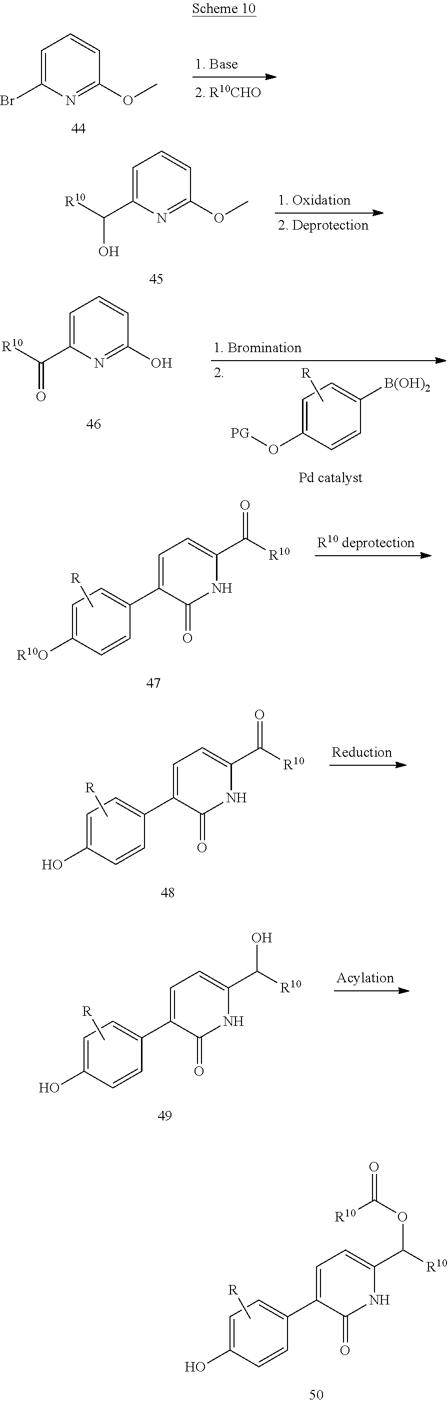

Scheme 10 shows a route for the preparation of the 6-acyl pyridin-2(1H)-one phenol compound 48. Base-mediated halogen exchange of the commercially available bromopyridine 44 followed by quenching with an aldehyde gives the secondary alcohol compound 45. Oxidation of the alcohol followed by demethylation gives compound 46. Bromination of compound 46 followed by a Suzuki coupling with an appropriate boronic acid gives a coupling compound 47. Final deprotection of the phenol gives compound 48, which can then be reacted with appropriate core intermediate 7 as in Scheme 2 to give compound 9. Sodium borohydride reduction of this compound gives compound 49, and acetylation of compound 49 gives intermediate 50. Compounds 49 and 50 can also be reacted with appropriate compound 7 as in Scheme 2 to provide compound 9.

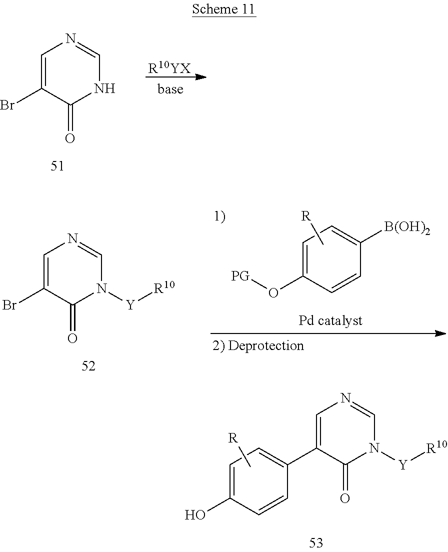

Scheme 11 shows a route for the preparation of 3-benzyl substituted pyrimidin-4(3H)-one phenol compound 53 which is useful for the synthesis of compounds of Formula I. 5-Bromopyrimidin-4(3H)-one 51 is reacted with a base such as NaH, and an appropriate bromide or chloride of formula R¹⁰—Y—X to give the corresponding 3-benzyl-5-bromopyrimidin-4(3H)-one 52. Suzuki coupling of compound 52 with an appropriate boronic acid gives a coupling intermediate, which after final deprotection of the phenol gives compound 53, which can be reacted with appropriate core intermediate 7 as in Scheme 2 to provide compound 9.

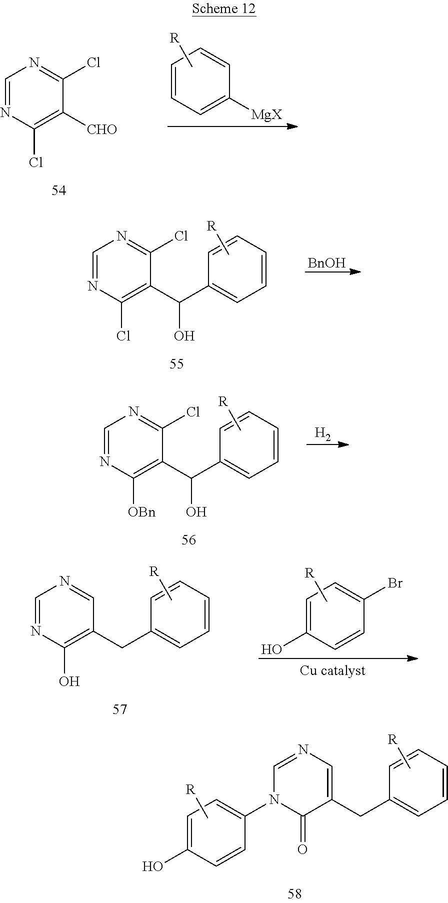

Scheme 12 shows a route for the preparation of the 5-benzyl-3-(4-hydroxyphenyl)pyrimidin-4(3H)-one phenol intermediate 58 which is useful for the synthesis of compounds of Formula I. Commercially available 4,6-dichloropyrimidine-5-carbaldehyde 54 is reacted with the appropriate substituted phenyl magnesium halide to give the secondary alcohol 55. Monobenzylation gives compound 56, which is subjected to hydrogenation to provide compound 57. Copper (I)-mediated coupling of compound 57 to an appropriate phenol provides the desired compound 58, which can be reacted with appropriate core intermediate 7 as in Scheme 2 to provide compound 9.

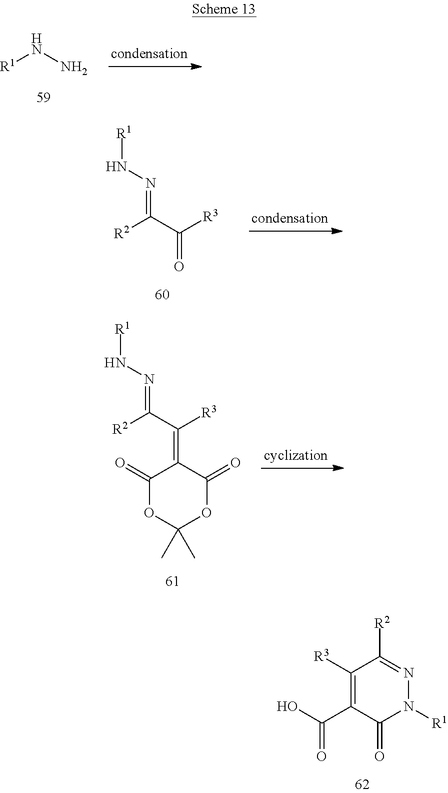

The pyridazino carboxylic acid compound 62 can be prepared using methods described by McNab H. et al (1982) J. Chem. Soc. Perkin Trans. 1:1845 as depicted in Scheme 13. Substituted hydrazine 59 can be converted to hydrazono acetaldehyde 60 with standard dehydrating conditions such as acetic acid at room temperature. The carbonyl group condensation product 61 is prepared in a suitable organic solvent such as toluene, benzene or dioxane at room temperature using piperidinium acetate as catalyst. Carboxylic acid pyridazinone 62 is prepared from hydrazono ethylidene 61 by cyclization under basic conditions (sodium methoxide in methanol) at 70° C. When R² or R³═CH₃ or alkyl, the desired product 62 can be obtained in a one-pot reaction through condensation and cyclization of compound 60. Compound 62 may then be used to acylate aniline intermediate 4 whose preparation is described in Scheme 1 to prepare compounds of Formula I.

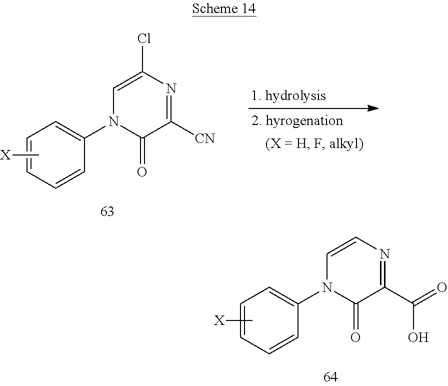

Scheme 14 shows a route for the preparation of oxo-4-phenyl-3,4-dihydropyrazine-2-carboxylic acids. The pyrazine-2-carbonitrile 63 was prepared using methods described by Hoornaert, G., et al (1983) J. Heterocyclic Chem. 20:919 and Hoornaert, G., et al (1990) Tetrahedron 46:5715. The pyrazine-2-carboxylic acid 64 can be prepared by hydrolysis to the carboxylic acid followed by removal of the chloro group under hydrogenolysis conditions to give the desired 3-oxo-4-phenyl-3,4-dihydropyrazine-2-carboxylic acid 64. The acid 64 can then be coupled via standard amide bond forming techniques to an aniline bearing core 4, prepared according to Scheme 1 to provide final compound 5.

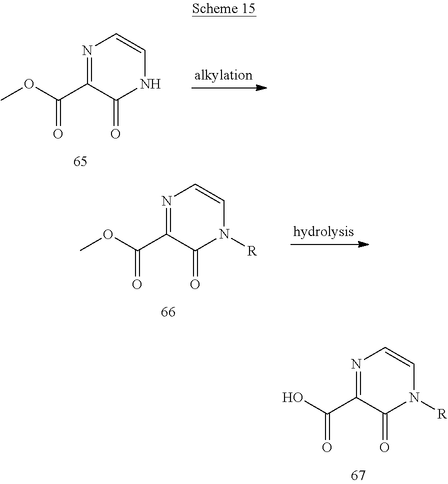

The substituted pyrazino carboxylic acids 67 can be prepared as in Scheme 15. Methyl 3-oxo-3,4-dihydropyrazine-2-carboxylate 65 can be converted to alkylpyrazino carboxylate 66 by standard basic alkylation conditions with alkyl halides. These conditions include but are not limited to treatment with K₂CO₃ in acetone or DMF at room or elevated temperature, or NaH in THF at ambient or elevated temperature, followed by addition of the alkyl halide. In certain embodiments, this alkylation is achieved with LiH in DMF at 0° C., followed by addition of alkyl chloride or alkyl bromide or alkyl iodide and warming to room temperature. Carboxylic acid 67 can then be prepared using standard saponification conditions such as LiOH or NaOH in standard mixed aqueous/organic solvent systems. The acid 67 can then be coupled via standard amide bond forming techniques to an aniline bearing core 4 constructed according to Scheme 1 to provide final compound 5.

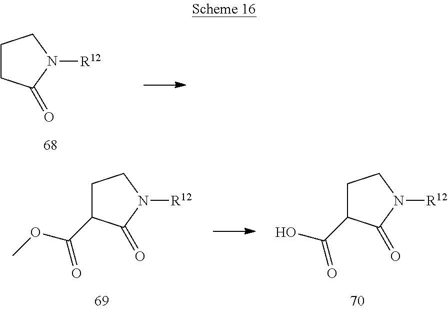

Scheme 16 shows a method for preparing pyrrolidin-2-one intermediate 70 (wherein R¹² is independently selected from H, alkyl, aryl and heteroaryl). Carbonylation of the N-substituted pyrrolidin-2-one 68 can be completed by treatment with LDA, followed by quenching with methyl carbonochloridate to give ester 69. Hydrolysis of the ester with appropriate base, such as TMSOK, KOH, etc, yields the corresponding acid 70. The acid 70 can then be coupled via standard amide bond forming techniques to an aniline bearing core such as 4 according to Scheme 1 to provide compound 5.

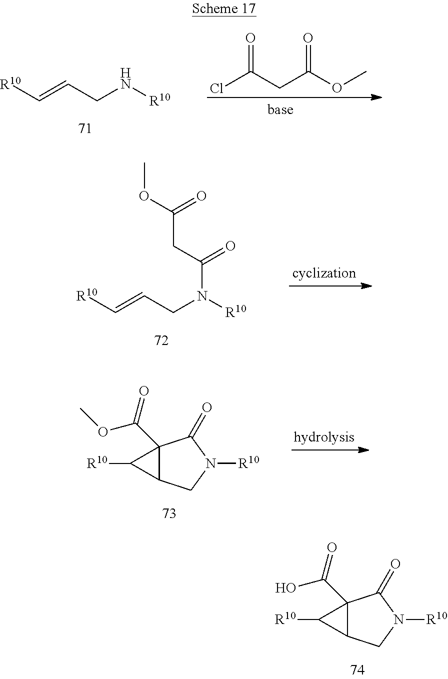

Scheme 17 shows a method for the preparation of the fused bicyclic cyclopropane lactam ester 74. An optionally substituted allylic amine 71 is acylated with a malonyl chloride ester under basic conditions to give the allylic amide intermediate 72. Cyclization under conditions which generate the malonyl carbine (preferably manganese III acetate catalyzed) provide the fused cyclopropyllactam compound 73. Deprotection under basic conditions (typically LiOH or NaOH in an aqueous/organic solvent mixture) provides the intermediate acid 74. The acid 74 can then be coupled via standard amide bond forming techniques to aniline bearing cores such as 4 constructed according to Scheme 1 to provide final compound 5.

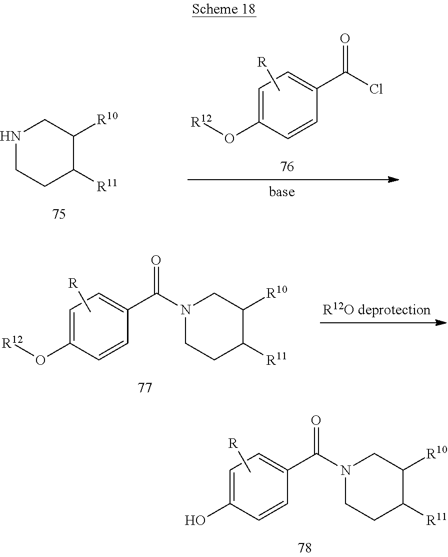

Scheme 18 shows a route for the preparation of (piperidin-1-yl)methanone phenol intermediate 78 which is useful for the synthesis of compounds of Formula I. Substituted and O-protected benzoyl chlorides of type 76 are reacted with an appropriate amine 75 to form the corresponding amide 77, which after final deprotection of the phenol gives compound 78. Compound 78 can then be reacted with the appropriate core intermediate 7 as in Scheme 2 to provide compound 9.

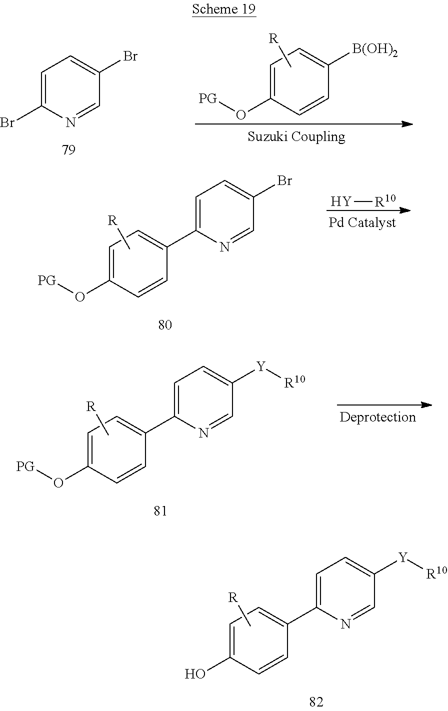

Scheme 19 shows a method for the preparation of phenolic intermediate 82. 2,5-dibromopyridine 79 is treated with the appropriate boronic acid under Suzuki type reaction conditions to give selective coupling at the pyridine 2-position to provide compound 80. Buchwald type palladium coupling of compound 80 with an appropriate heteroatom bearing an R¹⁰ group gives the protected compound 81. Final deprotection of compound 81 gives compound 82 which can be reacted with an appropriate core intermediate 7 as in Scheme 2 to provide compound 9.

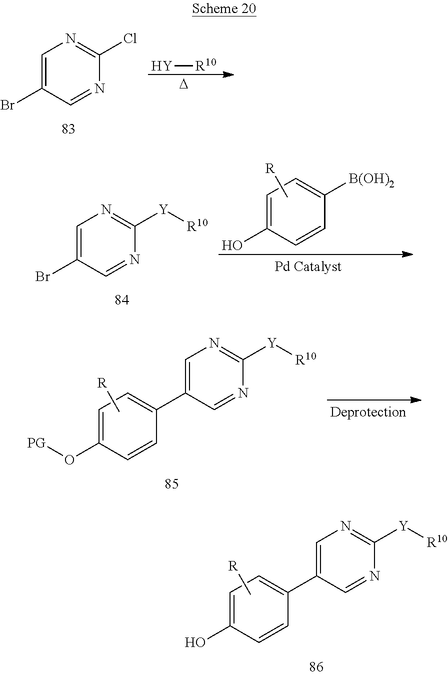

Scheme 20 shows a method for the preparation of phenolic intermediate 86. 2,5-Dibromopyrimidine 83 is treated with the appropriate heteroatom bearing an R¹⁰ group with heating in an appropriate solvent such as 1-propanol. Reaction occurs selectively at the 2-position to give the bromopyrimidine intermediate 84. Suzuki coupling to the appropriately substituted boronic acid gives intermediate 85, which after deprotection gives the phenolic compound 86. Compound can be reacted with appropriate core intermediate 7 as in Scheme 2 to provide compound 9.

### Methods of Separation

In the methods of preparing the compounds of this invention, it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point and molecular weight in distillation and sublimation, presence or absence of polar functional groups in chromatography, stability of materials in acidic and basic media in multiphase extraction, and the like. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine(amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−)menthyl chloroformate in the presence of base, or Mosher ester, α-methoxy-α-(trifluoromethyl)phenyl acetate (Jacob III (1982) J. Org. Chem. 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. Chromatogr., (1990) 513:375-378). Enriched or purified enantiomers can be distinguished by methods used to distinguish other chiral molecules with asymmetric carbon atoms, such as optical rotation and circular dichroism.

Exemplary compounds of this invention include compounds 101-205 as described in Examples 1-105.

### Biological Evaluation

Determination of the activity of c-Met kinase activity of a compound of Formula I is possible by a number of direct and indirect detection methods. One example of an assay used for the determination of c-Met kinase activity is based on an enzyme linked immunosorbant assay (ELISA). The assay includes a compound of Formula I, c-Met (His-tagged recombinant human Met (amino acids 974-end), expressed by baculovirus), and ATP in assay buffer, as described in Example 106.

In MKN45 cells, the activity of c-Met inhibitors of Formula I was determined by the in vitro fluorescence assay as described in Example 107.

Exemplary compounds described herein were prepared, characterized, and assayed for their c-Met binding activity and in vitro activity against tumor cells. The range of c-Met binding activities was less than 1 nM to about 10 μM. Certain exemplary compounds of the invention had c-Met binding activity IC₅₀ values less than 10 nM. Certain compounds of the invention had MKN45 cell-based activity IC₅₀ values less than 100 nM.

### Administration of Compounds of Formula I

The compounds of the invention may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

Methods of Treatment with Compounds of Formula I

Compounds of the present invention are useful for treating diseases, conditions and/or disorders including, but not limited to, those characterized by over expression of receptor tyrosine kinases (RTK), e.g. c-Met kinase. Accordingly, another aspect of this invention includes methods of treating or preventing diseases or conditions that can be treated or prevented by inhibiting receptor tyrosine kinases (RTK), including c-Met. In one embodiment, the method comprises administering to a mammal in need thereof a therapeutically effective amount of a compound of Formula I, or a stereoisomer, geometric isomer, tautomer, solvate, metabolite, or pharmaceutically acceptable salt or prodrug thereof.

Diseases and conditions treatable according to the methods of this invention include, but are not limited to, cancer, stroke, diabetes, hepatomegaly, cardiovascular disease, Alzheimer's disease, cystic fibrosis, viral disease, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders in a patient. In one embodiment, a human patient is treated with a compound of Formula I and a pharmaceutically acceptable carrier, adjuvant, or vehicle, wherein said compound of Formula I is present in an amount to detectably inhibit c-Met kinase activity.

Cancers which can be treated according to the methods of this invention include, but are not limited to, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

Cardiovascular diseases which can be treated according to the methods of this invention include, but are not limited to, restenosis, cardiomegaly, atherosclerosis, myocardial infarction, and congestive heart failure.

Neurodegenerative disease which can be treated according to the methods of this invention include, but are not limited to, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

Inflammatory diseases which can be treated according to the methods of this invention include, but are not limited to, rheumatoid arthritis, psoriasis, contact dermatitis, and delayed hypersensitivity reactions.

Another aspect of this invention provides a compound of this invention for use in the treatment of the diseases or conditions described herein in a mammal, for example, a human, suffering from such disease or condition. Also provided is the use of a compound of this invention in the preparation of a medicament for the treatment of the diseases and conditions described herein in a warm-blooded animal, such as a mammal, for example a human, suffering from such disorder.

### Pharmaceutical Formulations

In order to use a compound of this invention for the therapeutic treatment (including prophylactic treatment) of mammals including humans, it is normally formulated in accordance with standard pharmaceutical practice as a pharmaceutical composition. According to this aspect of the invention there is provided a pharmaceutical composition comprising a compound of this invention in association with a pharmaceutically acceptable diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of Formula I having the desired degree of purity may optionally be mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compound of this invention for use herein is preferably sterile. In particular, formulations to be used for in vivo administration must be sterile. Such sterilization is readily accomplished by filtration through sterile filtration membranes.

The compound ordinarily can be stored as a solid composition, a lyophilized formulation or as an aqueous solution.

The pharmaceutical compositions of the invention will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

As a general proposition, the initial pharmaceutically effective amount of the inhibitor administered parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of compounds of Formulas I may be prepared. Suitable examples of sustained-release preparations include semipermeable matrices of solid hydrophobic polymers containing a compound of Formula I, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate) and poly-D-(−)-3-hydroxybutyric acid.

The formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound of Formula I suitable for oral administration may be prepared as discrete units such as pills, capsules, cachets or tablets each containing a predetermined amount of a compound of Formula I.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of Formula I intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include TWEEN® 60, Span® 80, cetostearyl alcohol, benzyl alcohol, myristyl alcohol, glyceryl mono-stearate and sodium lauryl sulfate.

Aqueous suspensions of Formula I compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of Formula I may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The compounds of Formula I may be employed alone or in combination with other therapeutic agents for the treatment of a disease or disorder described herein, such as a hyperproliferative disorder (e.g., cancer). In certain embodiments, a compound of Formula I is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of Formula I such that they do not adversely affect each other. Such compounds are suitably present in combination in amounts that are effective for the purpose intended. In one embodiment, a composition of this invention comprises a compound of Formula I, or a stereoisomer, geometric isomer, tautomer, solvate, metabolite, or pharmaceutically acceptable salt or prodrug thereof, in combination with a chemotherapeutic agent such as described herein.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In a particular embodiment of anti-cancer therapy, a compound of Formula I, or a stereoisomer, geometric isomer, tautomer, solvate, metabolite, or pharmaceutically acceptable salt or prodrug thereof, may be combined with other chemotherapeutic, hormonal or antibody agents such as those described herein, as well as combined with surgical therapy and radiotherapy. Combination therapies according to the present invention thus comprise the administration of at least one compound of Formula I, or a stereoisomer, geometric isomer, tautomer, solvate, metabolite, or pharmaceutically acceptable salt or prodrug thereof, and the use of at least one other cancer treatment method. The amounts of the compound(s) of Formula I and the other pharmaceutically active chemotherapeutic agent(s) and the relative timings of administration will be selected in order to achieve the desired combined therapeutic effect.

### Metabolites of Compounds of Formula I

Also falling within the scope of this invention are the in vivo metabolic products of quinoline compounds of Formula I described herein. Such products may result for example from the oxidation, reduction, hydrolysis, amidation, deamidation, esterification, deesterification, enzymatic cleavage, and the like, of the administered compound. Accordingly, the invention includes metabolites of compounds of Formula I, including compounds produced by a process comprising contacting a compound of this invention with a mammal for a period of time sufficient to yield a metabolic product thereof.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

### Articles of Manufacture

In another embodiment of the invention, an article of manufacture, or “kit”, containing materials useful for the treatment of the diseases and disorders described above is provided. In one embodiment, the kit comprises a container comprising a quinoline compound of Formula I, or a stereoisomer, geometric isomer, tautomer, solvate, metabolite, or pharmaceutically acceptable salt or prodrug thereof. The kit may further comprise a label or package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of Formula I or a formulation thereof which is effective for treating the condition and may have a sterile access port (for example, the container may be an intravenous solution bag or a vial having a stopper pierceable by a hypodermic injection needle). At least one active agent in the composition is a compound of Formula I. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising a compound of Formula I can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of Formula I and, if present, the second pharmaceutical formulation. For example, if the kit comprises a first composition comprising a compound of Formula I and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formula I, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a compound of Formula I contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a composition of Formula I and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other c-Met inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was conducted on a Biotage system (Manufacturer: Dyax Corporation) having a silica gel column or on a silica SEP PAK® cartridge (Waters). ¹H NMR spectra were recorded on a Varian instrument operating at 400 MHz. ¹H NMR spectra were obtained as CDCl₃, d₆-DMSO, CH₃OD or d₆-acetone solutions (reported in ppm), using chloroform as the reference standard (7.25 ppm). When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

### Example 1

### Preparation of 3-(3-fluoro-4-(6-methoxy-7-(3-morpholino-propoxy)quinolin-4-yloxy)phenyl)-5-methyl-6-(2-methylbenzyl)pyrimidin-4(3H)-one 101

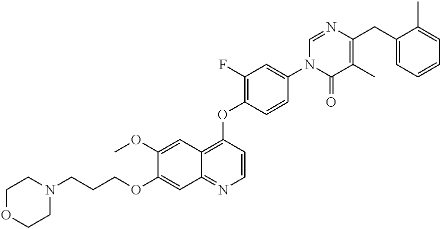

Step A: Preparation of 4-chloro-5-methyl-6-(2-methylbenzyl)pyrimidine: 2-Methylbenzylzinc chloride (25 ml of 0.5 M THF solution, 12 mmol) was added to a solution of 4,6-dichloro-5-methylpyrimidine (2.0 g, 12 mmol) and bis(triphenylphosphine) palladium(II) chloride (0.4 g. 0.6 mmol) in THF (20 mL). The reaction mixture was heated to reflux for 2 hours, cooled to room temperature, and then poured onto water (10 mL). The reaction mixture was extracted with ethyl acetate, and the organic layer was washed with brine, dried over sodium sulfate, filtered, and concentrated. The residue was purified by silica gel flash column chromatography (1:10 Et₂O/Hexane) to yield the product (1.0 g, 35%) as a white solid. ¹H NMR (CDCl₃, 400 MHz) δ 8.75 (s, 1H), 7.09-7.22 (m, 4H), 6.84 (d, J=7.81 Hz, 1H), 4.15 (s, 1H), 2.38 (s, 3H), 2.32 (s, 3H).

Step B: Preparation of 4-(benzyloxy)-5-methyl-6-(2-methylbenzyl)pyrimidine: Potassium hydroxide (0.48 g, 8.6 mmol) was added to a solution of 4-chloro-5-methyl-6-(2-methylbenzyl)pyrimidine (1.0 g, 4.3 mmol), 18-crown-6 (0.11 g, 0.43 mmol) and benzyl alcohol (0.45 ml, 4.3 mmol) in toluene (20 mL). The reaction mixture was heated to reflux for 2 hours, cooled to room temperature, and then poured into water (10 mL). The reaction mixture was extracted with ethyl acetate, and the organic layer was washed with brine, dried over sodium sulfate, filtered, and concentrated. The residue was purified by silica gel flash column chromatography (1:1 Et₂O/Hexane) to yield the product (1.5 g, 92%) as a white solid. ¹H NMR (CDCl₃, 400 MHz) δ 8.60 (s, 1H), 7.44-7.48 (m, 2H), 7.30-7.42 (m, 3H), 7.06-7.20 (m, 3H), 6.88 (d, J=7.42 Hz, 1H), 5.45 (s, 2H), 2.33 (s, 3H), 2.15 (s, 3H).

Step C: Preparation of 5-methyl-6-(2-methylbenzyl)pyrimidin-4-ol: 4-(benzyloxy)-5-methyl-6-(2-methylbenzyl)pyrimidine (1.5 g, 4.9 mmol) was dissolved in trifluoroacetic acid (10 mL). The reaction mixture was heated at 60° C. for 4 hours, cooled to room temperature, and then solvent was evaporated to yield the product (1.5 g, 98%) as white solid. ¹H NMR (DMSO-d₆, 400 MHz) δ 8.12 (s, 1H), 7.30-7.40 (m, 1H), 7.10-7.15 (m, 1H), 7.00-7.10 (m, 1H), 6.90 (d, J=7.42 Hz, 1H), 3.82 (s, 2H), 2.45 (s, 3H), 2.22 (s, 3H). LRMS (ESI pos) m/e 215 (M+1).

Step D: Preparation of 3-(3-fluoro-4-hydroxyphenyl)-5-methyl-6-(2-methylbenzyl)pyrimidin-4(3H)-one: Copper(I) iodide (90 mg, 0.5 mmol) was added to a solution of 5-methyl-6-(2-methylbenzyl)pyrimidin-4-ol (1.0 g, 5.0 mmol), 4-bromo-2-fluorophenol (0.90 g, 5.0 mmol), N,N′-dimethylethylenediamine (80 mg, 0.90 mmol) and potassium phosphate (2.0 g, 9.0 mmol). The reaction mixture was heated to reflux for 12 hours, cooled to room temperature, and then filtered through a pad of celite. The filtrate was concentrated and the residue was purified by silica gel flash column chromatography (2:1 EtOAc/hexanes) to yield the product (0.6 g, 40%) as a white solid. ¹H NMR (CDCl₃, 400 MHz) δ 7.98 (s, 1H), 7.15-7.24 (m, 3H), 7.03-7.08 (m, 2H), 6.88-6.94 (m, 2H), 3.98 (2, 2H), 2.37 (s, 3H), 2.21 (s, 3H). LRMS (ESI pos) m/e 325 (M+1).

Step E: Preparation of 3-(3-fluoro-4-(6-methoxy-7-(3-morpholino-propoxy)quinolin-4-yloxy)phenyl)-5-methyl-6-(2-methylbenzyl)pyrimidin-4(3H)-one: DMAP (0.75 mg, 0.0062 mmol) was added to a suspension of 3-(3-fluoro-4-hydroxyphenyl)-5-methyl-6-(2-methylbenzyl)pyrimidin-4(3H)-one (20 mg, 0.062 mmol) and 4-chloro-6-methoxy-7-(3-morpholinopropoxy)quinoline (prepared according to WO 01/55116, Example 2, 21 mg, 0.062 mmol). The reaction mixture was heated at 150° C. for 12 hours, cooled to room temperature and purified directly by silica gel flash column chromatography (1:10 MeOH/EtOAc) to yield 101 (10 mg, 26%) as a light brown solid. ¹H NMR (CDCl₃, 400 MHz) δ 8.54 (s, 1H), 8.05 (s, 1H), 7.52 (s, 1H), 7.46 (s, 1H), 7.34-7.42 (m, 2H), 7.12-7.24 (m, 4H), 7.05-7.12 (m, 1H), 6.50-6.56 (m, 1H), 4.24-4.32 (m, 2H), 4.04 (s, 3H), 3.94-4.02 (m, 2H), 3.68-3.80 (m, 4H), 2.56-2.62 (m, 2H), 2.44-2.52 (m, 4H), 2.38 (s, 3H), 2.22 (s, 3H), 2.10-2.18 (m, 2H). LRMS (ESI pos) m/e 625 (M+1).

### Example 2

### Preparation of 6-benzyl-3-(3-fluoro-4-(6-methoxy-7-(3-morpholinopropoxy)quinolin-4-yloxy)phenyl)pyrimidin-4(3H)-one 102

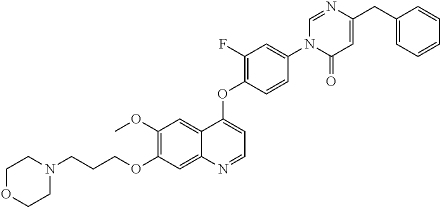

Step A: Preparation of 4-benzyl-6-chloropyrimidine: Prepared from 4,6-dichloropyrimidine (2.0 g, 13 mmol) and benzyl zinc chloride (0.5 M solution in THF, 27 mL, 13 mmol) according to the procedure described for Example 1, Step A. The crude product was purified by silica gel flash column chromatography (1:10 Et₂O/Hexane) to yield the product (1.3 g, 47%) as a yellow liquid. ¹H NMR (CDCl₃, 400 MHz) δ 8.86 (s, 1H), 7.33-7.38 (m, 2H), 7.28-7.32 (m, 1H), 7.24-7.28 (m, 2H), 7.13 (d, J=0.78 Hz, 1H), 4.11 (s, 2H).

Step B: Preparation of 4-benzyl-6-(benzyloxy)pyrimidine: Prepared from 4-benzyl-6-chloropyrimidine (1.1 g, 5.4 mmol) according to the procedure described for Example 1, Step B. The crude was purified by silica gel flash column chromatography (1:1 Et₂O/Hexane) to yield the product (1.3 g, 88%) as a colorless liquid. ¹H NMR (CDCl₃, 400 MHz) δ 8.76 (s, 1H), 7.36-7.46 (m, 3H), 7.29-7.35 (m, 3H), 7.24-7.27 (m, 4H), 6.52 (s, 1H), 5.40 (s, 2H), 4.20 (s, 2H).

Step C: Preparation of 6-benzylpyrimidine-4-ol: Prepared from 4-benzyl-6-(benzyloxy)pyrimidine was (1.0 g, 3.6 mmol) according to the procedure described for Example 1, Step C, to yield the product (0.63 g, 94%) as a white solid. ¹H NMR (CDCl₃, 400 MHz) δ 8.06 (s, 1H), 7.30-7.36 (m, 2H), 7.23-7.29 (m, 3H), 6.24 (s, 1H), 3.90 (s, 3H).

Step D: Preparation of 6-benzyl-3-(3-fluoro-4-hydroxyphenyl)pyrimidin-4(3H)-one: Prepared from 6-benzylpyrimidin-4-ol (0.50 g, 2.7 mmol) according to the procedure described for Example 1, Step D. The crude was purified by silica gel flash column chromatography (1:1 EtOAc/Hexane) to yield the product (0.50 g, 63%) as a white solid. ¹H NMR (DMSO-d₆, 400 MHz) δ 10.30 (s, 1H), 8.32 (s, 1H), 7.30-7.38 (m, 4H), 7.20-7.30 (m, 1H), 7.00-7.10 (m, 2H), 6.32 (s, 1H), 5.76 (s, 1H), 3.83 (s, 2H). LRMS (ESI pos) m/e 297 (M+1).

Step E: Preparation of 6-benzyl-3-(3-fluoro-4-(6-methoxy-7-(3-morpholinopropoxy)quinolin-4-yloxy)phenyl)pyrimidin-4(3H)-one: Prepared from 6-benzyl-3-(3-fluoro-4-hydroxyphenyl)pyrimidin-4(3H)-one (18 mg, 0.059 mmol) according to the procedure described for Example 1, Step E. The crude product was purified by silica gel flash column chromatography (1:10 MeOH/EtOAc) to yield 102 (10 mg, 28%) as a white solid. ¹H NMR (CDCl₃, 400 MHz) δ 8.54 (d, J=5.01 Hz, 1H), 8.14 (s, 1H), 7.52 (s, 1H), 7.46 (s, 1H), 7.34-7.42 (m, 4H), 7.28-7.34 (m, 3H), 7.20-7.24 (m, 1H), 6.54 (d, J=5.47 Hz, 1H), 4.26 (m, 2H), 4.04 (s, 3H), 3.90-3.94 (m, 2H), 3.70-3.76 (m, 4H), 2.54-2.60 (m, 2H), 2.44-2.52 (m, 4H), 2.10-2.18 (m, 2H). LRMS (ESI pos) m/e 597 (M+1).

### Example 3

### Preparation of 6-benzyl-3-(3-fluoro-4-(6-methoxy-7-(3-morpholinopropoxy)quinolin-4-yloxy)phenyl)-5-methylpyrimidin-4(3H)-one 103

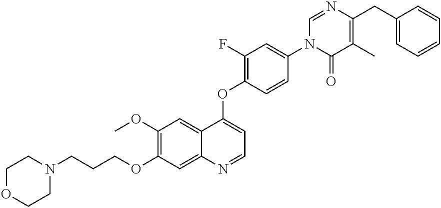

Step A: Preparation of 4-benzyl-6-chloro-5-methylpyrimidine: Prepared from benzyl zinc bromide (0.5 M solution in THF, 25 mL, 12 mmol) and 4,6-dichloro-5-methylpyrimidine (2.0 g, 12 mmol) according to the procedure described for Example 1, Step A. The crude was purified by silica gel flash column chromatography (1:5 EtOAc/Hexane) to yield the product (0.86 g, 32%) as a colorless oil. ¹H NMR (CDCl₃, 400 MHz) δ 8.78 (s, 1H), 7.28-7.33 (m, 2H), 7.18-7.26 (m, 3H), 4.19 (s, 2H), 2.35 (s, 3H).

Step B: Preparation of 4-benzyl-6-(benzyloxy)-5-methylpyrimidine: Prepared from 4-benzyl-6-chloro-5-methylpyrimidine (0.8 g, 4.0 mmol) according to the procedure described for Example 1, Step B. The crude product was purified by silica gel flash column chromatography (1:9 Et₂O/Hexane) to yield the product (1.0 g, 94%) as a colorless oil. ¹H NMR (CDCl₃, 400 MHz) δ 8.62 (s, 1H), 7.42-7.45 (m, 2H), 7.32-7.40 (m, 3H), 7.27-7.30 (m, 2H), 7.18-7.24 (m, 3H), 5.42 (s, 2H), 4.20 (s, 2H), 2.20 (s, 3H).

Step C: Preparation of 6-benzyl-5-methylpyrimidin-4-ol: Prepared from 4-benzyl-6-(benzyloxy)-5-methylpyrimidine (1.0 g, 3.0 mmol) according to the procedure described for Example 1, Step C, to yield the product (0.50 g, 73%) as a white solid. ¹H NMR (DMSO-d₆, 400 MHz) δ 8.20 (s, 1H), 7.18-7.33 (m, 5H), 3.91 (s, 2H), 1.99 (s, 3H).

Step D: Preparation of 6-benzyl-3-(3-fluoro-4-hydroxyphenyl)-5-methylpyrimidin-4(3H)-one: Prepared from 6-benzyl-5-methylpyrimidin-4-ol (0.16 g, 0.80 mmol) according to the procedure described for Example 1, Step D. The crude product was purified by silica gel flash column chromatography (1:1 EtOAc/Hexane) to yield the product (0.10 g, 64%) as a white solid. ¹H NMR (DMSO-d₆, 400 MHz) δ 8.21 (s, 1H), 7.34-7.38 (m, 1H), 7.28-7.32 (m, 4H), 7.20-7.24 (m, 1H), 7.00-7.10 (m, 2H), 3.94 (s, 2H), 2.08 (s, 3H). LRMS (ESI pos) m/e 311 (M+1).

Step E: Preparation of 6-benzyl-3-(3-fluoro-4-(6-methoxy-7-(3-morpholinopropoxy)quinolin-4-yloxy)phenyl)-5-methylpyrimidin-4(3H)-one: Prepared from 6-benzyl-3-(3-fluoro-4-hydroxyphenyl)-5-methylpyrimidin-4(3H)-one (18 mg, 0.06 mmol) according to the procedure described for Example 1, Step E. The crude product was purified by silica gel flash column chromatography (1:10 MeOH/EtOAc) to yield 103 (10 mg, 28%) as a white solid. ¹H NMR (CDCl₃, 400 MHz) δ8.52 (s, 1H), 8.06 (s, 1H), 7.52 (s, 1H), 7.46 (s, 1H), 7.20-7.40 (m, 8H), 6.52 (s, 1H), 4.24-4.32 (m, 2H), 4.04 (s, 3H), 3.98-4.02 (m, 2H), 3.70-3.78 (m, 4H), 2.54-2.62 (m, 2H), 2.42-2.54 (m, 4H), 2.24 (s, 3H), 2.10-2.18 (m, 2H). LRMS (ESI pos) m/e 611 (M+1).

### Example 4

### Preparation of (3-benzylpiperidin-1-yl)(3-fluoro-4-(6-methoxy-7-(3-morpholinopropoxy)quinolin-4-yloxy)phenyl)methanone 104

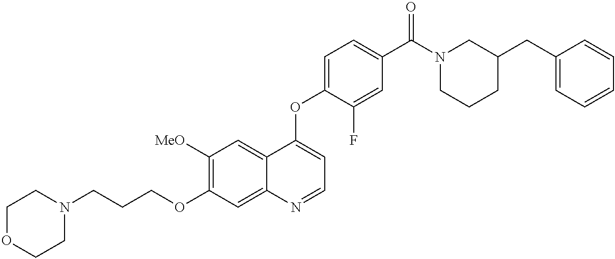

Step A: Preparation of (3-benzylpiperidin-1-yl)(3-fluoro-4-methoxyphenyl)methanone: Triethylamine (2 mL, 0.01 mol) was added into a solution of 3-fluoro-4-methoxybenzoyl chloride (500 mg, 2.65 mmol) and 3-benzylpiperidine hydrochloride (561 mg, 2.65 mmol) in CH₂Cl₂ (20 mL). The reaction mixture was stirred for 30 minutes at room temperature and then poured into water (10 mL). The reaction mixture was extracted with CH₂Cl₂, and the organic layer was washed with brine, dried over sodium sulfate, filtered, and concentrated to yield the product (0.80 g, 92%) as a white solid. LRMS (ESI pos) m/e 328 (M+1).

Step B: Preparation of (3-benzylpiperidin-1-yl)(3-fluoro-4-hydroxyphenyl)methanone: Boron tribromide (0.5 ml, 6.57 mmol) was added into a solution of (3-benzylpiperidin-1-yl)(3-fluoro-4-methoxyphenyl)methanone (0.86 g, 2.63 mmol) in CH₂Cl₂ (2 mL) at 0° C. The reaction mixture was stirred for 30 minutes at room temperature and then poured into water (10 mL). The reaction mixture was extracted with CH₂Cl₂, and the organic layer was washed with brine, dried over sodium sulfate, filtered, and concentrated to yield the product (0.74 g, 90% yield) as a white solid. LRMS (ESI pos) m/e 314 (M+1).

Step C: Preparation of (3-benzylpiperidin-1-yl)(3-fluoro-4-(6-methoxy-7-(3-morpholinopropoxy)quinolin-4-yloxy)phenyl)methanone: Prepared from (3-benzylpiperidin-1-yl)(3-fluoro-4-hydroxyphenyl)methanone (30 mg, 0.10 mmol) according to the procedure described for Example 1, Step E. The crude product was purified by silica gel flash column chromatography (1:10 MeOH/EtOAc) to yield 104 (10 mg, 28%) as a white solid. ¹H NMR (CDCl₃, 400 MHz) δ 8.50 (s, 1H), 7.50 (s, 1H), 7.40 (s, 1H), 7.00-7.30 (m, 9H), 4.50-4.60 (m, 2H), 4.20-4.30 (m, 4H), 4.00 (s, 3H), 3.70-3,8- (m, 6H), 2.70-2.80 (m, 2H), 2.60-2.70 (m, 4H), 2.40-2.60 (m, 4H), 2.10-2.20 (m, 2H), 1.80-1.90 (m, 1H). LRMS (ESI pos) m/e 614 (M+1).

### Example 5

### Preparation of (3-benzylpiperidin-1-yl)(4-(6,7-dimethoxyquinolin-4-yloxy)-3-fluorophenyl)methanone 105

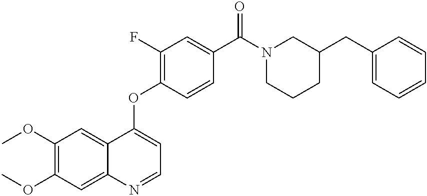

Prepared from 4-chloro-6,7-dimethoxyquinoline (prepared according to Kazuo Kubo (2005) Journal of Medicinal Chemistry 48:1359-1366, 70 mg, 0.31 mmol) and (3-benzylpiperidin-1-yl)(3-fluoro-4-hydroxyphenyl)methanone (Example 4, Step B, 98 mg, 0.31 mmol) according to the procedure described for Example 1, Step E. The crude product was purified by silica gel flash column chromatography (1:10 MeOH/EtOAc) to yield 105 (40 mg, 26%) as a white solid. ¹H NMR (CDCl₃, 400 MHz) δ 8.52-8.56 (m, 1H), 7.55 (s, 1H), 7.45 (s, 1H), 7.00-7.30 (m, 8H), 6.20-6.50 (m, 1H), 4.40-4.70 (m, 1H), 4.06 (s, 6H), 3.50-3.80 (m, 1H, 2.40-3.20 (m, 4H), 1.60-2.00 (m, 2H), 1.40-1.60 (m, 1H), 1.20-1.40 (m, 2H). LRMS (ESI pos) m/e 501 (M+1).

### Example 6

### Preparation of (4-benzylpiperidin-1-yl)(4-(6,7-dimethoxy-quinolin-4-yloxy)-3-fluorophenyl)methanone 106

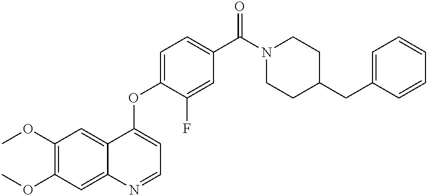

Step A: Preparation of (4-benzylpiperidin-1-yl)(3-fluoro-4-methoxyphenyl)methanone: Prepared from 3-fluoro-4-methoxybenzoyl chloride (570 mg, 3.02 mmol) and 4-benzylpiperidine (530 mg, 3.02 mmol) according to the procedure described for Example 4, Step A, to yield the product (920 mg, 93%) as a white solid. ¹H NMR (CDCl₃, 400 MHz) δ 7.25-7.30 (m, 2H), 7.12-7.23 (m, 5H), 6.93-6.98 (m, 1H), 3.91 (s, 3H), 2.60-2.90 (m, 1H0, 2.50-2.60 (m, 3H), 1.58-1.82 (m, 4H), 1.10-1.30 (m, 2H), 1.00-1.10 (m, 1H). LRMS (ESI pos) m/e 328 (M+1).

Step B: Preparation of (4-benzylpiperidin-1-yl)(3-fluoro-4-hydroxyphenyl)methanone: Prepared from (4-benzylpiperidin-1-yl)(3-fluoro-4-methoxyphenyl)methanone (130 mg, 0.40 mmol) according to the procedure described for Example 4, Step B, to yield the product (120 mg, 96%) as a white solid. LRMS (ESI pos) m/e 314 (M+1).

Step C: (4-benzylpiperidin-1-yl)(4-(6,7-dimethoxyquinolin-4-yloxy)-3-fluorophenyl)methanone: Prepared from 4-chloro-6,7-dimethoxyquinoline (for preparation see reference in Example 5) (79 mg, 0.35 mmol) and (4-benzylpiperidin-1-yl)(3-fluoro-4-hydroxyphenyl)methanone (110 g, 0.35 mmol) according to the procedure described for Example 1, Step E. The crude product was purified by silica gel flash column chromatography (1:10 MeOH/EtOAc) to yield 106 (20 mg, 11%) as a white solid. ¹H NMR (CDCl₃, 400 MHz) δ 8.50-8.55 (m, 1H), 7.55 (s, 1H), 7.44 (s, 1H), 7.10-7.40 (m, 8H), 6.40-6.50 (m, 1H), 4.60-4.80 (m, 1H), 4.06 (s, 6H), 3.70-3.82 (m, 1H), 2.40-3.20 (m, 4H), 1.60-1.90 (m, 3H), 1.20-1.30 (m, 1H). LRMS (ESI pos) m/e 501 (M+1).

### Example 7

### Preparation of 3-benzyl-5-(3-fluoro-4-(6-methoxy-7-(3-(piperidin-1-yl)propoxy)quinolin-4-yloxy)phenyl)pyrimidin-4(3H)-one 107

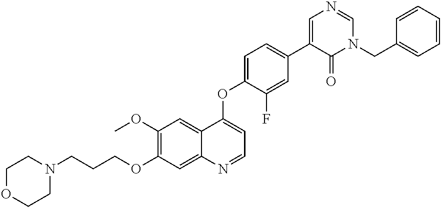

Step A: Preparation of 3-benzyl-5-(4-(benzyloxy)-3-fluorophenyl)pyrimidin-4(3H)-one: Tetrakis(triphenylphosphine)palladium(0) (0.65 g, 0.57 mmol) was added into a suspension of 3-benzyl-5-bromopyrimidin-4(3H)-one (prepared according to Gurnos Jones Journal of the Chemical Society, Perkin Transactions 1: Organic and Bio-Organic Chemistry (1972-1999) 1983, 11:2645-8, 3.0 g, 11 mmol), 4-benzyloxy-3-fluorobenzeneboronic acid (3.3 g, 14 mmol) and lithium chloride (2.4 g, 57 mmol) in dioxane (100 mL) and 2M aqueous sodium carbonate solution (50 mL). The reaction mixture was heated at 100° C. for 2 hours, cooled and poured into water (10 mL). The reaction mixture was extracted with ethyl acetate, and the organic layer was washed with brine, dried over sodium sulfate, filtered, and concentrated. The residue was purified by silica gel flash column chromatography (2:1 EtOAc/Hexane) to yield the product (1.4 g, 32%) as a white solid. ¹H NMR (CDCl₃, 400 MHz) δ 8.15 (s, 1H), 8.01 (s, 1H), 7.53 (dd, J=12.5, 2.34 Hz, 1H), 7.43-7.47 (m, 2H), 7.30-7.42 (m, 9H), 7.00-7.05 (m, 1H), 5.18 (s, 2H), 5.17 (s, 2H). LRMS (ESI pos) m/e 387 (M+1).

Step B: Preparation of 3-benzyl-5-(3-fluoro-4-hydroxyphenyl)pyrimidin-4(3H)-one: Prepared from 3-benzyl-5-(4-(benzyloxy)-3-fluorophenyl)pyrimidin-4(3H)-one (0.3 g, 0.8 mmol) according to the procedure described for Example 1, Step C, to yield the product (0.2 g, 87%) as a white solid. LRMS (ESI pos) m/e 297 (M+1).

Step C: Preparation of 3-benzyl-5-(4-(7-(benzyloxy)-6-methoxyquinolin-4-yloxy)-3-fluorophenyl)pyrimidin-4(3H)-one: Prepared from 7-(benzyloxy)-4-chloro-6-methoxyquinoline (prepared according to WO2005030140, Example 32, 200 mg, 0.67 mmol) and 3-benzyl-5-(3-fluoro-4-hydroxyphenyl)pyrimidin-4(3H)-one (198 mg, 0.67 mmol) according to the procedure described for Example 1, Step E. The crude product was purified by silica gel flash column chromatography (1:10 MeOH/EtOAc) to yield the product (100 mg, 37%) as a white solid. LRMS (ESI pos) m/e 560 (M+1).

Step D: Preparation of 3-benzyl-5-(3-fluoro-4-(7-hydroxy-6-methoxyquinolin-4-yloxy)phenyl)pyrimidin-4(3H)-one: Prepared from 3-benzyl-5-(4-(7-(benzyloxy)-6-methoxyquinolin-4-yloxy)-3-fluorophenyl)pyrimidin-4(3H)-one (90 mg, 0.16 mmol) according to the procedure described for Example 1, Step C, to yield the product (50 mg, 66%) as a white solid. LRMS (ESI pos) m/e 470 (M+1).

Step E: Preparation of 3-benzyl-5-(3-fluoro-4-(6-methoxy-7-(3-morpholinopropoxy)quinolin-4-yloxy)phenyl)pyrimidin-4(3H)-one: Cesium carbonate (6.9 mg, 0.021 mmol) was added to a solution of 3-benzyl-5-(3-fluoro-4-(7-hydroxy-6-methoxyquinolin-4-yloxy)phenyl)pyrimidin-4(3H)-one (10 mg, 0.02 mmol) and 4-(3-chloropropyl)morpholine (3.5 mg, 0.021 mmol). The reaction mixture was heated to 50° C. for 1 hour, and then poured onto water (1 mL). The reaction mixture was extracted with EtOAc, and the organic layer was washed with brine, dried over sodium sulfate, filtered, and concentrated. The residue was purified by silica gel flash column chromatography (1:10 MeOH/EtOAc) to yield 107 (6 mg, 47%) as a yellow solid. ¹H NMR (CDCl₃ 400 MHz) δ 8.46 (m, 1H), 8.23 (s, 1H), 8.13 (s, 1H), 7.70-7.76 (m, 1H), 7.50-7.60 (m, 2H), 7.30-7.48 (m, 2H), 7.20-7.30 (m, 3H), 6.44-6.50 (m, 1H), 5.30 (s, 2H), 5.20 (s, 2H), 4.24-4.34 (m, 2H), 4.04 (s, 3H), 3.66-3.80 (m, 4H), 2.44-2.64 (m, 4H), 2.10-2.20 (m, 2H). LRMS (ESI pos) m/e 597 (M+1).

### Example 8

### Preparation of 5-(4-(7-(2-(1H-imidazol-1-yl)ethoxy)-6-methoxyquinolin-4-yloxy)-3-fluorophenyl)-3-benzylpyrimidin-4(3H)-one 108

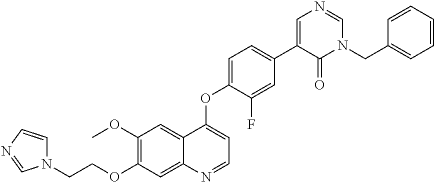

Prepared from 3-benzyl-5-(3-fluoro-4-(7-hydroxy-6-methoxyquinolin-4-yloxy)phenyl)pyrimidin-4(3H)-one (Example 7, Step D, 10 mg, 0.02 mmol) and 1-(2-chloroethyl)-1H-imidazole hydrochloride (10 mg, 0.06 mmol) according to the procedure described for Example 7, Step E. The crude product was purified by silica gel flash column chromatography (1:10 MeOH/EtOAc) to yield 108 (5 mg, 52%) as a white solid. LRMS (ESI pos) m/e 564 (M+1).

### Example 9

### Preparation of 3-benzyl-5-(3-fluoro-4-(6-methoxy-7-(3-(4-methylpiperazin-1-yl)propoxy)quinolin-4-yloxy)phenyl)pyrimidin-4(3H)-one 109

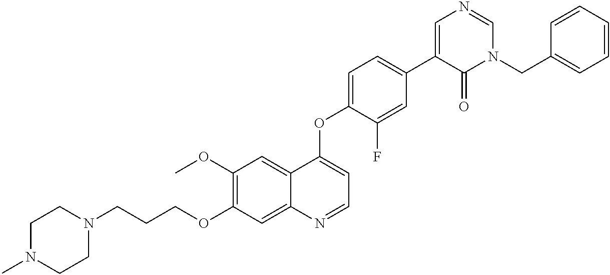

Prepared from 3-benzyl-5-(3-fluoro-4-(7-hydroxy-6-methoxyquinolin-4-yloxy)phenyl)pyrimidin-4(3H)-one (Example 7, Step D, 22 mg, 0.05 mmol) and 1-(3-chloropropyl)-4-methylpiperazine hydrochloride (45 mg, 0.21 mmol) according to the procedure described for Example 7, Step E. The crude product was purified by silica gel flash column chromatography (1:10 MeOH/EtOAc) to yield 109 (20 mg, 70%) as a yellow solid. LRMS (ESI pos) m/e 610 (M+1).

### Example 10

### Preparation of 3-benzyl-5-(3-fluoro-4-(6-methoxy-7-(3-(4-methylpiperazin-1-yl)propoxy)quinolin-4-yloxy)phenyl)pyrimidin-4(3H)-one Hydrogen Chloride 110

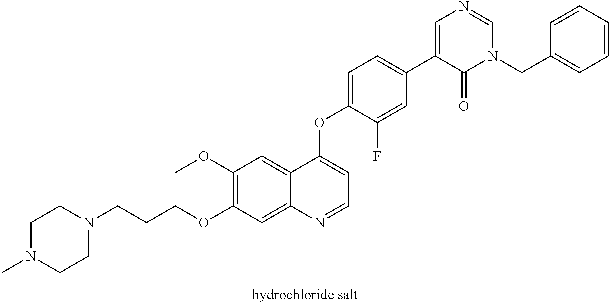

HCl (2.0 M in ether, 1 mL) was added in a solution of 3-benzyl-5-(3-fluoro-4-(6-methoxy-7-(3-(4-methylpiperazin-1-yl)propoxy)quinolin-4-yloxy)phenyl)pyrimidin-4(3H)-one (12 mg, 0.02 mmol) in ether (1 mL). The reaction mixture was stirred for 20 minutes and the solvent was evaporated to yield 110 (12 mg, 81%) as a white solid. ¹H NMR (DMSO-d₆, 400 MHz) δ 11.50-12.50 (s, br, 4H), 8.80-9.00 (m, 2H), 8.40 (s, 1H), 7.90-8.10 (m, 1H), 7.60-7.80 (m, 2H), 7.20-7.60 (m, 5H), 6.90-7.10 (m, 1H), 5.20-5.25 (m, 2H), 4.20-5.00 (m, 4H), 4.00 (s, 3H), 3.60-4.00 (m, 4H), 3.20-3.60 (m, 6H), 300 (m, 3H). LRMS (ESI pos) m/e 610 (M+1).

### Example 11

### Preparation of 3-benzyl-5-(3-fluoro-4-(6-methoxy-7-(3-(piperidin-1-yl)propoxy)quinolin-4-yloxy)phenyl)pyrimidin-4(3H)-one 111

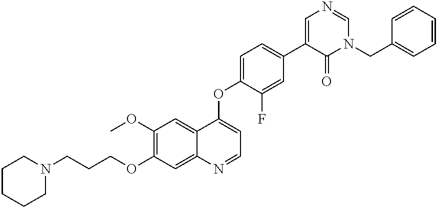

Prepared from 1-(3-chloropropyl)piperidine hydrochloride (46 mg, 0.05 mmol) and 3-benzyl-5-(3-fluoro-4-(7-hydroxy-6-methoxyquinolin-4-yloxy)phenyl)pyrimidin-4(3H)-one (Example 7, Step D, 22 mg, 0.05 mmol) according to the procedure described for Example 7, Step E. The crude product was purified by silica gel flash column chromatography (1:10 MeOH/EtOAc) to yield 111 (10 mg, 36%) as a yellow solid. ¹H NMR (DMSO-d₆, 400 MHz) δ 8.80 (s, 1H), 8.47-8.52 (m, 1H), 8.36 (s, 1H), 7.90-7.92 (m, 1H), 7.70-7.75 (m, 1H), 7.45-7.55 (m, 2H), 7.30-7.42 (m, 6H), 6.48-6.52 (m, 1H), 5.22 (s, 2H), 4.15-4.30 (m, 2H), 3.94 (s, 3H), 3.35-3.40 (m, 2H), 2.62-2.70 (m, 2H), 2.30-2.32 (m, 2H), 1.45-1.60 (m, 4H), 1.30-1.42 (m, 2H). LRMS (ESI pos) m/e 595 (M+1).

### Example 12

### Preparation of 3-benzyl-5-(3-fluoro-4-(6-methoxy-7-(3-(piperidin-1-yl)propoxy)quinolin-4-yloxy)phenyl)pyrimidin-4(3H)-one Hydrogen Chloride 112

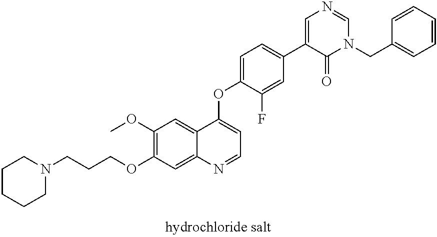

Prepared from 3-benzyl-5-(3-fluoro-4-(6-methoxy-7-(3-(piperidin-1-yl)propoxy)quinolin-4-yloxy)phenyl)pyrimidin-4(3H)-one (Example 11, 9 mg, 0.02 mmol) according to the procedure described for Example 10, to yield 112 (1.2 mg, 90%) as a white solid. ¹H NMR (DMSO-d₆, 400 MHz) δ 9.20 (s, 1H), 8.80 (s, 1H), 8.40 (s, 1H), 7.90-8.00 (m, 1H), 7.75-7.80 (m, 1H), 7.65 (s, 1H), 7.50-7.60 (m, 1H), 7.35-7.40 (m, 5H), 7.30-7.35 (m, 2H), 6.70 (s, 1H), 5.40 (s, 2H), 4.50-4.60 (m, 2H), 4.00 (s, 3H), 3.20-3.30 (m, 2H), 2.80-3.00 (m, 2H), 2.20-2.40 (m, 4H), 1.80-1.90 (m, 2H), 1.60-1.70 (m, 2H), 1.30-1.50 (m, 2H). LRMS (ESI pos) m/e 595 (M+1).

### Example 13

### Preparation of 3-(4-chlorobenzyl)-5-(3-fluoro-4-(6-methoxy-7-(3-morpholinopropoxy)quinolin-4-yloxy)phenyl)pyrimidin-4(3H)-one 113

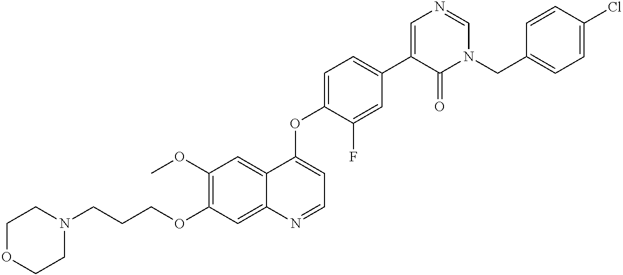

Step A: Preparation of 5-bromo-3-(4-chlorobenzyl)pyrimidin-4(3H)-one: Sodium hydride (0.34 g, 8.6 mmol) was added into a solution of 5-bromopyrimidin-4(3H)-one (prepared according to Thomas J Kress (1985) J. Org. Chem. 50:3073-6, 1.5 g, 8.57 mmol) in THF (10 mL) and DMF (6 mL). The reaction was stirred for 10 minutes and 4-chlorobenzyl bromide (1.76 g, 8.57 mmol) was added. The reaction was stirred for 30 minutes, poured into water (10 mL), and diluted with ethyl acetate. The reaction mixture was extracted with ethyl acetate, and the organic layer was washed with brine, dried over sodium sulfate, filtered, and concentrated. The residue was purified by silica gel flash column chromatography (2:1 EtOAc/Hexane) to yield the product (0.39 g, 15%) as a white solid. ¹H NMR (CDCl₃, 400 MHz) δ 8.20 (s, 1H), 8.10 (s, 1H), 7.29-7.38 (m, 4H), 5.10 (s, 2H). LRMS (ESI pos) m/e 300 (M+1).

Step B: Preparation of 5-(4-(benzyloxy)-3-fluorophenyl)-3-(4-chlorobenzyl)pyrimidin-4(3H)-one: Prepared from 5-bromo-3-(4-chlorobenzyl)pyrimidin-4(3H)-one (0.39 g, 1.3 mmol) according to the procedure described for Example 7, Step A. The crude was purified by silica gel flash column chromatography (1:1 EtOAc/Hexane) to yield the product (0.17 g, 31%) as a white solid. ¹H NMR (CDCl₃, 400 MHz) δ 8.15 (s, 1H), 8.02 (s, 1H), 7.50-7.54 (m, 1H), 7.42-7.46 (m, 2H), 7.35-7.42 (m, 2H0, 7.30-7.35 (m, 5H), 7.00-7.05 (m, 1H0, 5.18 (s, 2H), 5.12 (s, 2H). LRMS (ESI pos) m/e 421 (M+1).

Step C: Preparation of 3-(4-chlorobenzyl)-5-(3-fluoro-4-hydroxyphenyl)pyrimidin-4(3H)-one: Prepared from 5-(4-(benzyloxy)-3-fluorophenyl)-3-(4-chlorobenzyl)pyrimidin-4(3H)-one (0.17 g, 0.40 mmol) according to the procedure described for Example 1, Step C, to yield the product (0.1 g, 75%) as a yellow solid. LRMS (ESI pos) m/e 331 (M+1).

Step D: Preparation of 3-(4-chlorobenzyl)-5-(3-fluoro-4-(6-methoxy-7-(3-morpholinopropoxy)quinolin-4-yloxy)phenyl)pyrimidin-4(3H)-one: Prepared from 3-(4-chlorobenzyl)-5-(3-fluoro-4-hydroxyphenyl)pyrimidin-4(3H)-one (27 mg, 0.08 mmol) according to the procedure described for Example 1, Step E. The crude was purified by silica gel flash column chromatography (1:10 MeOH/EtOAc) to yield 113 (10 mg, 36%) as a yellow solid. ¹H NMR (CDCl₃, 400 MHz) δ 8.40-8.60 (m, 1H), 8.20-8.40 (m, 2H), 7.70-7.80 (m, 1H), 7.50-7.60 (m, 2H), 7.40-7.50 (m, 6H), 6.40-6.60 (m, 1H), 5.10-5.30 (m, 2H), 4.20-4.40 (m, 2H), 4.00 (s, 3H), 3.60-3.80 (m, 4H), 2.30-2.70 (m, 4H), 2.00-2.30 (m, 2H), 1.50-1.80 (m, 2H). LRMS (ESI pos) m/e 631 (M+1).

### Example 14

### Preparation of 6-benzyl-3-(4-(6,7-dimethoxyquinolin-4-yloxy)-3-fluorophenyl)pyridin-2(1H)-one 114

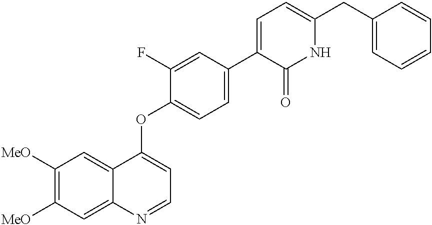

Step A: Preparation of (6-(benzyloxy)pyridin-2-yl)(phenyl)methanol: nBuLi (2.5 M in hexanes, 18.2 ml, 45.4 mmol) was added into a solution of 2-(benzyloxy)-6-bromopyridine (10 g, 37.9 mmol) in THF (200 mL) at −78° C. for 30 minutes. Benzaldehyde (4.59 ml, 45.4 mmol) was added, the reaction was stirred for 20 minutes at that temperature and poured onto water (10 mL). The reaction mixture was extracted with EtOAc, and the organic layer was washed with brine, dried over sodium sulfate, filtered, and concentrated. The residue was purified by silica gel flash column chromatography (1:2 EtOAc/Hexane) to yield the product (9.9 g, 90%) as a colorless oil. ¹H NMR (CDCl₃, 400 MHz) δ 7.50-7.55 (m, 1H), 7.44-7.47 (m, 2H), 7.35-7.41 (m, 2H), 7.30-7.35 (m, 5H), 6.68-6.72 (m, 2H), 5.66 (d, J=4.69 Hz, 2H), 5.44 (s, 2H), 4.70 (d, J=5.08 Hz, 1H).

Step B: Preparation of 6-benzylpyridin-2-ol: Palladium on carbon (10%, 1.5 g, 1.4 mmol) was added into a solution of (6-(benzyloxy)pyridin-2-yl)(phenyl)methanol (4 g, 14 mmol) in MeOH (20 mL). The reaction was pressurized with hydrogen using a balloon, stirred for 2 hours and filtered through a pad of celite. The filtrate was concentrated to yield the product (2 g, 79% yield) as a white solid. ¹H NMR (DMSO-d₆, 400 MHz) δ 7.28-7.35 (m, 5H), 7.18-7.26 (m, 1H), 6.10-6.16 (m, 1H), 5.92-5.98 (m, 1H), 3.80 (s, 2H).

Step C: Preparation of 6-benzyl-3-bromopyridin-2-ol: Bromine (0.14 mL, 2.7 mmol) was added into a solution of 6-benzylpyridin-2-ol (0.5 g, 2.7 mmol) in CH₂Cl₂ (5 mL). The reaction was stirred for 20 minutes at room temperature and then poured into 10% aqueous sodium bisulfate solution (10 mL). The reaction mixture was extracted with CH₂Cl₂ and the organic layer was washed with brine, dried over sodium sulfate, filtered, and concentrated to yield the product (0.59 g, 82%) as a yellow solid. LRMS (ESI pos) m/e 263 (M+1).

Step D: Preparation of 6-benzyl-3-(4-(benzyloxy)-3-fluorophenyl)pyridin-2(1H)-one: Prepared from 6-benzyl-3-bromopyridin-2-ol (0.63 g, 2.39 mmol) according to the procedure described for Example 7, Step A. The crude product was purified by silica gel flash column chromatography (1:1 EtOAc/Hexane) to yield the product (200 mg, 22%) as a white solid. ¹H NMR (DMSO-d₆, 400 MHz) δ 12.00 (s, 1H), 7.68-7.74 (m, 1H), 7.62 (d, J=7.42 Hz, 1H), 7.42-7.52 (m, 3H), 7.38-7.44 (m, 2H), 7.30-7.38 (m, 5H), 7.20-7.28 (m, 2H0, 6.04-6.10 (m, 1H), 5.20 (s, 2H), 3.80 (s, 2H). LRMS (ESI pos) m/e 386 (M+1).

Step E: Preparation of 6-benzyl-3-(3-fluoro-4-hydroxyphenyl)pyridin-2(1H)-one: Prepared from 6-benzyl-3-(4-(benzyloxy)-3-fluorophenyl)pyridin-2(1H)-one (150 mg, 0.39 mmol) according to the procedure described for Example 14, Step B. The crude product was purified by silica gel flash column chromatography (1:1 EtOAc/Hexane) to yield the product (100 mg, 87%) as a white solid. ¹H NMR (DMSO-d₆, 400 MHz) δ 11.90 (s, 1H), 10.00 (s, 1H), 7.60-7.66 (m, 1H), 7.54-7.58 (m, 1H), 7.32-7.40 (m, 5H), 7.22-7.30 (m, 1H), 6.92-6.96 (m, 1H0, 6.02-6.08 (m, 1H), 3.80 (s, 2H). LRMS (ESI pos) m/e 296 (M+1).

Step F: Preparation of 6-benzyl-3-(4-(6,7-dimethoxyquinolin-4-yloxy)-3-fluorophenyl)pyridin-2(1H)-one: Prepared from 4-chloro-6,7-dimethoxyquinoline (prepared according to reference in Example 5) (85 mg, 0.38 mmol) and 6-benzyl-3-(3-fluoro-4-hydroxyphenyl)pyridin-2(1H)-one (93 mg, 0.31 mmol) according to the procedure described for Example 1, Step E. The crude product was purified by silica gel flash column chromatography (1:10 MeOH/EtOAc) to yield 114 (50 mg, 33%) as a white solid. ¹H NMR (DMSO-d₆, 400 MHz) δ 10.40 (s, 1H), 8.45-8.48 (m, 1H0, 7.70-7.76 (m, 1H), 7.52-7.80 (m, 2H), 7.40 (s, 1H), 7.28-7.36 (m, 2H), 7.20-7.36 (m, 5H), 6.44-6.50 (m, 1H0, 6.14-6.20 (m, 1H0, 4.03 (s, 3H), 4.02 (s, 3H), 3.91 (s, 2H)). LRMS (ESI pos) m/e 483 (M+1).

### Example 15

### Preparation of 6-benzyl-3-(3-fluoro-4-(6-methoxy-7-(3-morpholinopropoxy)quinolin-4-yloxy)phenyl)pyridin-2(1H)-one 115

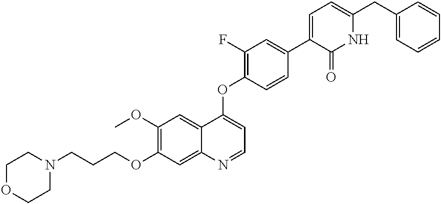

Prepared from 3-benzyl-5-(3-fluoro-4-hydroxyphenyl)pyrimidin-4(3H)-one (Example 14, Step E, 30 mg, 0.10 mmol) according to the procedure described for Example 1,

Step E. The crude product was purified by silica gel flash column chromatography (1:10 MeOH/EtOAc) to yield 115 (32 mg, 53%) as a white solid. ¹H NMR (DMSO-d₆, 400 MHz) δ 12.00 (s, 1H), 8.40-8.45 (m, 1H), 8.02-8.08 (m, 1H), 7.88-7.96 (m, 1H), 7.66-7.76 (m, 1H), 7.46-7.50 (m, 1H), 7.26-7.40 (m, 4H), 7.18-7.26 (m, 1H), 7.02-7.14 (m, 1H), 6.52-6.58 (m, 1H0, 6.40-6.46 (m, 1H), 6.04-6.12 (m, 1H), 4.10-4.20 (m, 2H), 3.90 (s, 3H0, 3.78-3.84 (m, 2H), 3.50-3.58 (m, 4H), 2.90 (s, 2H), 2.20-2.35 (m, 4H), 1.85-2.00 (m, 2H). LRMS (ESI pos) m/e 596 (M+1).

### Example 16

### Preparation of 4-benzyl-1-(3-fluoro-4-(6-methoxy-7-(3-morpholinopropoxy)quinolin-4-yloxy)phenyl)pyridin-2(1H)-one 116

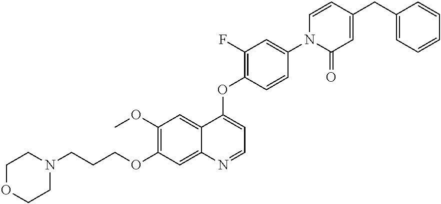

Step A: Preparation of (2-(benzyloxy)-5-bromopyridin-4-yl)(phenyl)methanol: Prepared from 2-(benzyloxy)-5-bromopyridine (1 g, 3.8 mmol) according to the procedure described for Example 14, Step A. The crude product was purified by silica gel flash column chromatography (1:9 Et₂O/Hexane) to yield the product (1.4 g, 29%) as a colorless oil. ¹H NMR (CDCl₃, 400 MHz) δ 8.19 (s, 1H), 7.43-7.47 (m, 2H), 7.30-7.40 (m, 8H), 7.18 (s, 1H), 5.99 (d, J=3.90 Hz, 1H), 5.31-5.40 (m, 2H).

Step B: Preparation of 4-benzylpyridin-2(1H)-one: Prepared from (2-(benzyloxy)-5-bromopyridin-4-yl)(phenyl)methanol (0.40 g, 1.1 mmol) according to the procedure described for Example 14, Step B, to yield the product (0.20 g, 100%) as a white solid. ¹H NMR (CDCl₃, 400 MHz) δ 7.80 (s, 1H), 7.26-7.36 (m, 4H), 7.10-7.20 (m, 2H), 6.80 (s, 1H0, 6.60 (s, 1H), 3.93 (s, 2H).

Step C: Preparation of 4-benzyl-1-(3-fluoro-4-hydroxyphenyl)pyridin-2(1H)-one: Prepared from 4-benzylpyridin-2(1H)-one (0.20 g, 1.1 mmol) according to the procedure described for Example 1, Step D. The crude product was purified by silica gel flash column chromatography (EtOAc) to yield the product (0.29 g, 91%) as a white solid. ¹H NMR (DMSO-d₆, 400 MHz) δ 10.17 (s, 1H), 7.50 (d, J=7.0H), 7.28-7.38 (m, 4H), 7.20-7.38 (m, 2H), 6.96-7.04 (m, 2H), 6.29-6.30 (m, 1H), 6.12-6.16 (m, 1H), 3.80 (s, 2H). LRMS (ESI pos) m/e 296 (M+1).

Step D: Preparation of 4-benzyl-1-(3-fluoro-4-(6-methoxy-7-(3-morpholinopropoxy)quinolin-4-yloxy)phenyl)pyridin-2(1H)-one: Prepared from 4-benzyl-1-(3-fluoro-4-hydroxyphenyl)pyridin-2(1H)-one (30 mg, 0.10 mmol) according to the procedure described for Example 1, Step E. The crude product was purified by silica gel flash column chromatography (1:10 MeOH/EtOAc) to yield 116 (41 mg, 68%) as a white solid. ¹H NMR (DMSO-d₆, 400 MHz) δ 8.45-8.50 (m, 1H), 7.62-7.68 (m, 1H0, 7.56-7.60 (m, 1H), 7.48-7.54 (m, 1H), 7.46-7.48 (m, 1H), 7.36-7.38 (m, 1H), 7.28-7.39 (m, 5H), 7.18-7.24 (m, 1H), 6.46-6.50 (m, 1H), 6.30 (s, 1H), 6.16-6.20 (m, 1H), 4.12-4.20 (m, 2H), 3.90 (s, 1H), 3.80 (s, 1H), 3.50-3.58 (M, 4H)), 2.40 (s, 2H), 2.30-2.38 (m, 4H), 1.85-2.00 (m, 2H). LRMS (ESI pos) m/e 596(M+1).

### Example 17

### Preparation of 4-benzyl-1-(4-(6,7-dimethoxyquinolin-4-yloxy)-3-fluorophenyl)pyridin-2(1H)-one 117

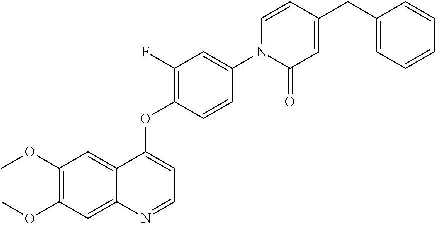

Prepared from 4-chloro-6,7-dimethoxyquinoline (prepared according to reference procedure in Example 5) (0.13 g, 0.57 mmol) and 4-benzyl-1-(3-fluoro-4-hydroxyphenyl)pyridin-2(1H)-one (Example 16, Step C, 0.14 g, 0.34 mmol) according to the procedure described for Example 1, Step E. The crude product was purified by silica gel flash column chromatography (1:10 MeOH/EtOAc) to yield 117 (82 mg, 50%) as a white solid. ¹H NMR (DMSO-d₆, 400 MHz) δ 8.50 (d, 1H), 7.67-7.72 (m, 1H0, 7.62-7.64 (m, 1H), 7.54-7.60 (m, 1H0, 7.52 (s, 1H0, 7.42 (s, 1H), 7.32-7.40 (m, 5H0, 7.24-7.30 (m, 1H), 6.52-6.56 (m, 1H0, 6.36-6.38 (m, 1H0, 6.22-6.26 (m, 1H), 3.96 (s, 3H), 3.95 (s, 3H), 3.84 (s, 2H). LRMS (ESI pos) m/e 483 (M+1).

### Example 18

### Preparation of 3-benzyl-5-(4-(6,7-dimethoxyquinolin-4-yloxy)-3-fluorophenyl)pyrimidin-4(3H)-one 118

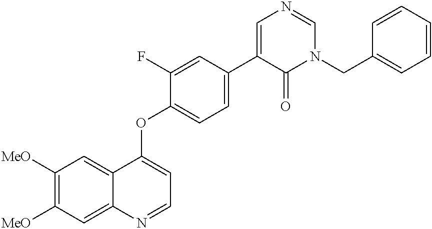

Prepared from 4-chloro-6,7-dimethoxyquinoline (prepared according to reference procedure in Example 5) (91 mg, 0.41 mmol) and 3-benzyl-5-(3-fluoro-4-hydroxyphenyl)pyrimidin-4(3H)-one (Example 7, Step D, 100 mg, 0.34 mmol) according to the procedure described for Example 1, Step E. The crude product was purified by silica gel flash column chromatography (1:10 MeOH/EtOAc) to yield 118 (50 mg, 61%) as a white solid. ¹H NMR (DMSO-d₆, 400 MHz) δ 8.80 (s, 1H), 8.50 (d, J=5.08 Hz, 1H), 8.36 (s, 1H), 7.88-7.94 (m, 1H), 7.71-7.78 (m, 1H), 7.46-7.56 (m, 2H), 7.30-7.42 (m, 5H), 6.48-6.54 (m, 1H), 5.22 (s, 2H), 3.95 (s, 6H). LRMS (ESI pos) m/e 484 (M+1).

### Example 19

### Preparation of 3-(2-chlorobenzyl)-5-(4-(6,7-dimethoxyquinolin-4-yloxy)-3-fluorophenyl)pyrimidin-4(3H)-one 119

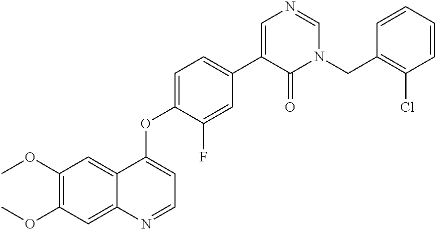

Step A: Preparation of 5-bromo-3-(2-chlorobenzyl)pyrimidin-4(3H)-one: Prepared from 1-(bromomethyl)-2-chlorobenzene (1.0 g, 5.7 mmol) according to the procedure described for Example 13, Step A. The crude product was purified by silica gel flash column chromatography (1:1 EtOAc/Hexane) to yield the product (0.37 g, 22%) as a white solid. ¹H NMR (CDCl₃, 400 MHz) δ 8.24 (s, 1H), 8.20 (s, 1H), 7.50-7.54 (m, 1H0, 7.42-7.44 (m, 1H), 7.28-7.35 (m, 2H), 5.25 (s, 1H). LRMS (ESI pos) m/e 299 (M+1).

Step B: Preparation of 5-(4-(benzyloxy)-3-fluorophenyl)-3-(2-chlorobenzyl)pyrimidin-4(3H)-one: Prepared from 5-bromo-3-(2-chlorobenzyl)pyrimidin-4(3H)-one (0.37 g, 1.2 mmol) according to the procedure described for Example 7, Step A. The crude product was purified by silica gel flash column chromatography (1:1 EtOAc/Hexane) to yield the product (0.30 g, 58%) as a white solid. ¹H NMR (CDCl₃, 400 MHz) δ 8.25 (s, 1H), 8.02 (s, 1H), 7.26-7.55 (m, 11H), 7.00-7.05 (m, 1H), 5.28 (s, 1H), 5.18 (s, 1H). LRMS (ESI pos) m/e 421 (M+1).

Step C: Preparation of 3-(2-chlorobenzyl)-5-(3-fluoro-4-hydroxyphenyl)pyrimidin-4(3H)-one: Prepared from 5-(4-(benzyloxy)-3-fluorophenyl)-3-(2-chlorobenzyl)pyrimidin-4(3H)-one (0.4 g, 1.0 mmol) according to the procedure described for Example 1, Step C, to yield the product (0.20 g, 64%) as a white solid. LRMS (ESI pos) m/e 331 (M+1).

Step D: Preparation of 3-(2-chlorobenzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (60 mg, 0.27 mmol) and 3-(2-chlorobenzyl)-5-(3-fluoro-4-hydroxyphenyl)pyrimidin-4(3H)-one (89 mg, 0.27 mmol) according to the procedure described for Example 1, Step E. The crude product was purified by silica gel flash column chromatography (1:10 MeOH/EtOAc) to yield 119 (20 mg, 14%) as a white solid. ¹H NMR (DMSO-d₆, 400 MHz) δ 8.70 (s, 1H), 8.47-8.51 (m, 1H), 8.41 (s, 1H), 7.90-7.95 (m, 1H), 7.73-7.26 (m, 1H), 7.50-7.55 (m, 3H), 7.41-7.44 (m, 1H), 7.34-7.40 (m, 2H), 7.14-7.16 (m, 1H), 6.49-6.52 (m, 1H), 5.75 (s, 2H), 3.95 (s, 6H). LRMS (ESI pos) m/e 518 (M+1).

### Example 20

### Preparation of 5-(4-(6,7-dimethoxyquinolin-4-yloxy)-3-fluorophenyl)-3-(2-methylbenzyl)pyrimidin-4(3H)-one 120

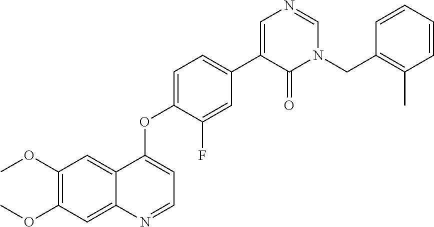

Step A: Preparation of 5-bromo-3-(2-methylbenzyl)pyrimidin-4(3H)-one: Prepared from 5-bromo-3-(2-methylbenzyl)pyrimidin-4(3H)-one (1.0 g, 5.7 mmol) according to the procedure described for Example 13, Step A. The crude product was purified by silica gel flash column chromatography (1:1 EtOAc/Hexane) to yield the product (0.28 g, 18%) as a white solid. ¹H NMR (CDCl₃, 400 MHz) δ 8.21 (s, 1H), 7.90 (s, 1H), 7.21-7.30 (m, 3H), 7.12-7.15 (m, 1H), 5.16 (s, 2H), 2.32 (s, 3H). LRMS (ESI pos) m/e 281 (M+1).

Step B: Preparation of 5-(4-(benzyloxy)-3-fluorophenyl)-3-(2-methylbenzyl)pyrimidin-4(3H)-one: Prepared from 5-bromo-3-(2-chlorobenzyl)pyrimidin-4(3H)-one (280 mg, 1.0 mmol) according to the procedure described for Example 7, Step A. The crude product was purified by silica gel flash column chromatography (1:1 EtOAc/Hexane) to yield the product (0.31 g, 77%) as a white solid. ¹H NMR (CDCl₃, 400 MHz) δ 8.04 (s, 1H), 7.95 (s, 1H), 7.53-7.58 (m, 1H), 7.43-7.47 (m, 2H), 7.35-7.44 (m, 2H), 7.20-7.30 (m, 6H), 7.01-7.06 (m, 1H), 5.19 (s, 2H), 5.18 (s, 2H), 2.35 (s, 3H). LRMS (ESI pos) m/e 421 (M+1).

Step C: Preparation of 5-(3-fluoro-4-hydroxyphenyl)-3-(2-methylbenzyl)pyrimidin-4(3H)-one: Prepared from 5-(4-(benzyloxy)-3-fluorophenyl)-3-(2-methylbenzyl)pyrimidin-4(3H)-one (0.31 g, 0.77 mmol) according to the procedure described for Example 1, Step C, to yield the product (0.20 g, 84%) as a white solid. LRMS (ESI pos) m/e 331 (M+1).

Step D: Preparation of 5-(4-(6,7-dimethoxyquinolin-4-yloxy)-3-fluorophenyl)-3-(2-methylbenzyl)pyrimidin-4(3H)-one: Prepared from 4-chloro-6,7-dimethoxyquinoline (prepared according to reference procedure in Example 5) (54 mg, 0.24 mmol) and 5-(3-fluoro-4-hydroxyphenyl)-3-(2-methylbenzyl)pyrimidin-4(3H)-one (75 mg, 0.24 mmol) according to the procedure described for Example 1, Step E. The crude product was purified by silica gel flash column chromatography (1:10 MeOH/EtOAc) to yield 120 (30 mg, 25%) as a white solid. ¹H NMR (DMSO-d₆, 400 MHz) δ 8.64 (s, 1H), 8.50 (d, J=5.08 Hz, 1H), 8.40 (s, 1H), 7.90-7.94 (m, 1H), 7.74-7.78 (m, 1H), 7.48-7.56 (m, 2H), 7.42 (s, 1H), 7.14-7.26 (m, 3H), 6.94-6.98 (m, 1H), 6.50-6.54 (m, 1H0, 5.20 (s, 2H), 3.96 (s, 3H), 3.95 (s, 3H), 2.40 (s, 3H). LRMS (ESI pos) m/e 498 (M+1).

### Example 21

### Preparation of 3-(3-chlorobenzyl)-5-(4-(6,7-dimethoxyquinolin-4-yloxy)-3-fluorophenyl)pyrimidin-4(3H)-one 121

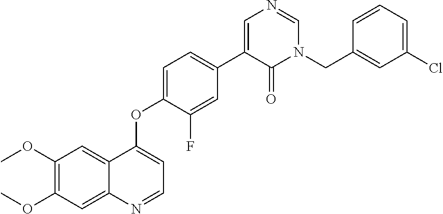

Step A: Preparation of 5-bromo-3-(3-chlorobenzyl)pyrimidin-4(3H)-one: Prepared from 1-(bromomethyl)-3-chlorobenzene (1.5 g, 8.6 mmol) according to the procedure described for Example 13, Step A. The crude product was purified by silica gel flash column chromatography (1:1 EtOAc/Hexane) to yield the product (0.41 g, 16%) as a white solid. ¹H NMR (CDCl₃, 400 MHz) δ 8.21 (s, 1H), 8.12 (s, 1H), 7.28-7.35 (m, 3H), 7.23-7.25 (m, 1H), 5.11 (s, 2H). LRMS (ESI pos) m/e 301 (M+1).

Step B: Preparation of 5-(4-(benzyloxy)-3-fluorophenyl)-3-(3-chlorobenzyl)pyrimidin-4(3H)-one: Prepared from 5-bromo-3-(3-chlorobenzyl)pyrimidin-4(3H)-one (0.41 g, 1.38 mmol) according to the procedure described for Example 7, Step A. The crude product was purified by silica gel flash column chromatography (1:1 EtOAc/Hexane) to yield the product (0.42 g, 72%) as a white solid. ¹H NMR (CDCl₃, 400 MHz) δ 8.15 (s, 1H), 8.04 (s, 1H), 7.50-7.55 (m, 1H), 7.43-7.47 (m, 2H), 7.30-7.42 (m, 7H), 7.28-7.30 (m, 1H), 7.00-7.06 (m, 1H), 5.18 (s, 2H), 5.12 (s, 2H). LRMS (ESI pos) m/e 421 (M+1).

Step C: Preparation of 3-(3-chlorobenzyl)-5-(3-fluoro-4-hydroxyphenyl)pyrimidin-4(3H)-one: Prepared from 5-(4-(benzyloxy)-3-fluorophenyl)-3-(3-chlorobenzyl)pyrimidin-4(3H)-one (0.42 g, 1.0 mmol) according to the procedure described for Example 1, Step C, to yield the product (0.20 g, 61%) as a white solid. LRMS (ESI pos) m/e 331 (M+1).

Step D: Preparation of 3-(3-chlorobenzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (64 mg, 0.29 mmol) and 3-(3-chlorobenzyl)-5-(3-fluoro-4-hydroxyphenyl)pyrimidin-4(3H)-one (95 mg, 0.30 mmol) according to the procedure described for Example 1, Step E. The crude product was purified by silica gel flash column chromatography (1:10 MeOH/EtOAc) to yield 121 (20 mg, 13%) as a white solid. ¹H NMR (DMSO-d₆, 400 MHz) δ 8.80 (s, 1H), 8.50 (d, J=5.08 Hz, 1H), 8.36 (s, 1H), 7.88-7.94 (m, 1H), 7.72-7.76 (m, 1H), 7.48-7.54 (m, 3H), 7.36-7.44 (m, 4H), 6.50-6.52 (m, 1H), 5.20 (s, 3H), 3.96 (s, 3H), 3.95 (s, 3H). LRMS (ESI pos) m/e 518 (M+1).

### Example 22

### Preparation of 3-(4-chlorobenzyl)-5-(4-(6,7-dimethoxyquinolin-4-yloxy)-3-fluorophenyl)pyrimidin-4(3H)-one 122

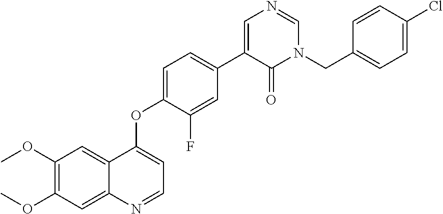

Prepared from 4-chloro-6,7-dimethoxyquinoline (prepared according to reference procedure in Example 5) (60 mg, 0.27 mmol) and 3-(4-chlorobenzyl)-5-(3-fluoro-4-hydroxyphenyl)pyrimidin-4(3H)-one (Example, Step C, 89 mg, 0.20 mmol) according to the procedure described for Example 1, Step E. The crude product was purified by silica gel flash column chromatography (1:10 MeOH/EtOAc) to yield 122 (37 mg, 36%) as a white solid. ¹H NMR (DMSO-d₆, 400 MHz) δ 8.80 (s, 1H), 8.50 (d, J=5.5 Hz, 1H), 8.36 (s, 1H), 7.88-7.94 (m, 1H), 7.70-7.76 (m, 1H), 7.48-7.54 (m, 2H), 7.40-7.46 (m, 5H), 6.48-6.52 (m, 1H), 5.20 (s, 2H), 3.96 (s, 3H), 3.95 (s, 3H). LRMS (ESI pos) m/e 518 (M+1).

### Example 23

### Preparation of 5-(4-(6,7-dimethoxyquinolin-4-yloxy)-3-fluorophenyl)-3-(2-fluorobenzyl)pyrimidin-4(3H)-one 123

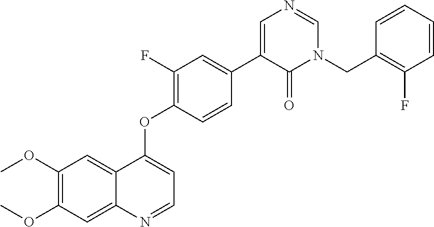

Step A: Preparation of 5-bromo-3-(2-fluorobenzyl)pyrimidin-4(3H)-one: Prepared from 1-(bromomethyl)-2-fluorobenzene (1.58 g, 8.4 mmol) according to the procedure described for Example 13, Step A. The crude product was purified by silica gel flash column chromatography (1:1 EtOAc/Hexane) to yield the product (0.55 g, 23%) as a white solid. LRMS (ESI pos) m/e 283 (M+1).

Step B: Preparation of 5-(4-(benzyloxy)-3-fluorophenyl)-3-(3-chlorobenzyl)pyrimidin-4(3H)-one: Prepared from 5-bromo-3-(2-fluorobenzyl)pyrimidin-4(3H)-one (0.55 g, 1.9 mmol) according to the procedure described for Example 7, Step A. The crude product was purified by silica gel flash column chromatography (1:1 EtOAc/Hexane) to yield the product (0.35 g, 44%) as a white solid. LRMS (ESI pos) m/e 405 (M+1).

Step C: Preparation of 5-(3-fluoro-4-hydroxyphenyl)-3-(2-fluorobenzyl)pyrimidin-4(3H)-one: Prepared from 5-(4-(benzyloxy)-3-fluorophenyl)-3-(3-chlorobenzyl)pyrimidin-4(3H)-one (0.35 g, 8.6 mmol) according to the procedure described for Example 1, Step C, to yield the product (0.20 g, 74%) as a white solid. LRMS (ESI pos) m/e 315 (M+1).

Step D: Preparation of 5-(4-(6,7-dimethoxyquinolin-4-yloxy)-3-fluorophenyl)-3-(2-fluorobenzyl)pyrimidin-4(3H)-one: Prepared from 4-chloro-6,7-dimethoxyquinoline (prepared according to reference procedure in Example 5) (100 mg, 0.45 mmol) and 3-(3-chlorobenzyl)-5-(3-fluoro-4-hydroxyphenyl)pyrimidin-4(3H)-one (92 mg, 0.29 mmol) according to the procedure described for Example 1, Step E. The crude product was purified by silica gel flash column chromatography (1:10 MeOH/EtOAc) to yield 123 (41 mg, 28%) as a white solid. ¹H NMR (DMSO-d₆, 400 MHz) δ 8.73 (s, 1H), 8.50 (d, J=5.1 Hz, 1H), 8.38 (s, 1H), 7.88-7.94 (m, 1H), 7.70-7.76 (m, 1H), 7.46-7.56 (m, 2H), 7.32-7.44 (m, 3H), 7.18-7.30 (m, 2H), 6.48-6.54 (m, 1H), 5.26 (s, 2H), 3.95 (s, 3H), 3.94 (s, 3H). LRMS (ESI pos) m/e 502 (M+1).

### Example 24

### Preparation of 5-(4-(6,7-dimethoxyquinolin-4-yloxy)-3-fluorophenyl)-3-(4-fluorobenzyl)pyrimidin-4(3H)-one 124

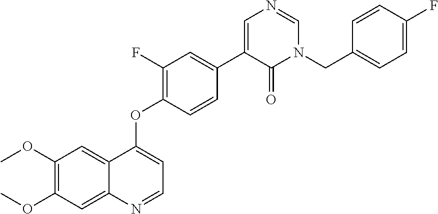

Step A: Preparation of 5-bromo-3-(4-fluorobenzyl)pyrimidin-4(3H)-one: Prepared from 1-(bromomethyl)-4-fluorobenzene (1.58 g, 8.30 mmol) according to the procedure described for Example 13, Step A. The crude product was purified by silica gel flash column chromatography (1:1 EtOAc/Hexane) to yield the product (0.42 g, 17%) as a white solid. LRMS (ESI pos) m/e 283 (M+1).

Step B: Preparation of 5-(4-(benzyloxy)-3-fluorophenyl)-3-(4-fluorobenzyl)pyrimidin-4(3H)-one: Prepared from 5-bromo-3-(4-fluorobenzyl)pyrimidin-4(3H)-one (0.42 g, 1.5 mmol) according to the procedure described for Example 7, Step A. The crude product was purified by silica gel flash column chromatography (1:1 EtOAc/Hexane) to yield the product (0.33 g, 55%) as a white solid. LRMS (ESI pos) m/e 405 (M+1).

Step C: Preparation of 5-(3-fluoro-4-hydroxyphenyl)-3-(4-fluorobenzyl)pyrimidin-4(3H)-one: Prepared from 5-(4-(benzyloxy)-3-fluorophenyl)-3-(4-fluorobenzyl)pyrimidin-4(3H)-one (0.33 g, 0.82 mmol) according to the procedure described for Example 1, Step C, to yield the product (0.20 g, 78%) as a white solid. LRMS (ESI pos) m/e 315 (M+1).

Step D: Preparation of 5-(4-(6,7-dimethoxyquinolin-4-yloxy)-3-fluorophenyl)-3-(4-fluorobenzyl)pyrimidin-4(3H)-one: Prepared from 4-chloro-6,7-dimethoxyquinoline (prepared according to reference procedure in Example 5) (105 mg, 0.47 mmol) and 5-(3-fluoro-4-hydroxyphenyl)-3-(4-fluorobenzyl)pyrimidin-4(3H)-one (90 mg, 0.29 mmol) according to the procedure described for Example 1, Step E. The crude product was purified by silica gel flash column chromatography (1:10 MeOH/EtOAc) to yield 124 (35 mg, 24%) as a white solid. ¹H NMR (DMSO-d₆, 400 MHz) δ 8.80 (s, 1H), 8.50 (d, J=5.47 Hz, 1H), 8.35 (s, 1H), 7.88-7.94 (m, 1H), 7.71-7.76 (m, 1H), 7.46-7.54 (m, 4H), 7.42 (s, 1H), 7.18-7.24 (m, 2H), 6.50 (d, J=4.3 Hz, 1H), 5.20 (s, 2H), 3.96 (s, 3H), 3.95 (s, 3H). LRMS (ESI pos) m/e 502 (M+1).

### Example 25

### Preparation of 5-(4-(6,7-dimethoxyquinolin-4-yloxy)-3-fluorophenyl)-3-(4-methylbenzyl)pyrimidin-4(3H)-one 125

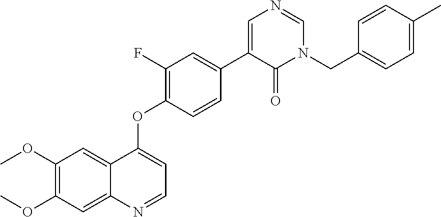

Step A: Preparation of 5-bromo-3-(4-methylbenzyl)pyrimidin-4(3H)-one: Prepared from 1-(bromomethyl)-4-fluorobenzene (1.55 g, 8.3 mmol) according to the procedure described for Example 13, Step A. The crude product was purified by silica gel flash column chromatography (1:1 EtOAc/Hexane) to yield the product (0.14 g, 6%) as a white solid. LRMS (ESI pos) m/e 281 (M+1).

Step B: Preparation of 5-(4-(benzyloxy)-3-fluorophenyl)-3-(4-methylbenzyl)pyrimidin-4(3H)-one: Prepared from 5-bromo-3-(4-methylbenzyl)pyrimidin-4(3H)-one (0.14 g, 0.5 mmol) according to the procedure described for Example 7, Step A. The crude product was purified by silica gel flash column chromatography (1:1 EtOAc/Hexane) to yield the product (0.15 g, 75%) as a white solid. LRMS (ESI pos) m/e 401 (M+1).

Step C: Preparation of 5-(3-fluoro-4-hydroxyphenyl)-3-(4-methylbenzyl)pyrimidin-4(3H)-one: Prepared from 5-(4-(benzyloxy)-3-fluorophenyl)-3-(4-methylbenzyl)pyrimidin-4(3H)-one (0.15 g, 0.38 mmol) according to the procedure described for Example 1, Step C, to yield the product (0.10 g, 86%) as a white solid. LRMS (ESI pos) m/e 311 (M+1).

Step D: 5-(4-(6,7-dimethoxyquinolin-4-yloxy)-3-fluorophenyl)-3-(4-methylbenzyl)pyrimidin-4(3H)-one: Prepared from 4-chloro-6,7-dimethoxyquinoline (prepared according to reference procedure in Example 5) (105 mg, 0.50 mmol) and 5-(3-fluoro-4-hydroxyphenyl)-3-(4-methylbenzyl)pyrimidin-4(3H)-one (90 g, 0.29 mmol) according to the procedure described for Example 1, Step E. The crude product was purified by silica gel flash column chromatography (1:10 MeOH/EtOAc) to yield 125 (44 mg, 30%) as a white solid. ¹H NMR (DMSO-d₆, 400 MHz) δ 8.78 (s, 1H), 8.50 (d, J=5.1 Hz, 1H), 8.34 (s, 1H), 7.88-7.94 (m, 1H), 7.71-7.76 (m, 1H), 7.53 (s, 1H), 7.48-7.54 (m, 1H), 7.42 (s, 1H), 7.28-7.34 (m, 2H), 7.16-7.20 (m, 2H), 6.50-6.52 (m, 1H), 5.17 (s, 2H), 3.96 (s, 3H), 3.95 (s, 3H). LRMS (ESI pos) m/e 498 (M+1).

### Example 26

### Preparation of 3-(3,4-dichlorobenzyl)-5-(4-(6,7-dimethoxyquinolin-4-yloxy)-3-fluorophenyl)pyrimidin-4(3H)-one 126

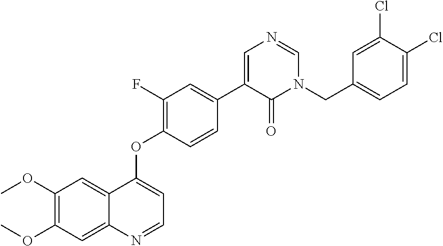

Step A: Preparation of 5-bromo-3-(3,4-dichlorobenzyl)pyrimidin-4(3H)-one: Prepared from 4-(bromomethyl)-1,2-dichlorobenzene (2.0 g, 8.4 mmol) according to the procedure described for Example 13, Step A. The crude product was purified by silica gel flash column chromatography (1:1 EtOAc/Hexane) to yield the product (0.63 g, 22%) as a white solid. LRMS (ESI pos) m/e 335 (M+1).

Step B: Preparation of 5-(4-(benzyloxy)-3-fluorophenyl)-3-(3,4-dichlorobenzyl)pyrimidin-4(3H)-one: Prepared from 5-bromo-3-(3,4-dichlorobenzyl)pyrimidin-4(3H)-one (0.63 g, 0.20 mmol) according to the procedure described for Example 7, Step A. The crude product was purified by silica gel flash column chromatography (1:1 EtOAc/Hexane) to yield the product (0.11 g, 13%) as a white solid. LRMS (ESI pos) m/e 455 (M+1).

Step C: Preparation of 3-(3,4-dichlorobenzyl)-5-(3-fluoro-4-hydroxyphenyl)pyrimidin-4(3H)-one: Prepared from 5-(4-(benzyloxy)-3-fluorophenyl)-3-(3,4-dichlorobenzyl)pyrimidin-4(3H)-one (0.11 g, 0.24 mmol) according to the procedure described for Example 1, Step C, to yield the product (50 mg, 56%) as a white solid. LRMS (ESI pos) m/e 365 (M+1).

Step D: 3-(3,4-dichlorobenzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (60 mg, 0.14 mmol) and 3-(3,4-dichlorobenzyl)-5-(3-fluoro-4-hydroxyphenyl)pyrimidin-4(3H)-one (50 mg, 0.1 mmol) according to the procedure described for Example 1, Step E. The crude product was purified by silica gel flash column chromatography (1:10 MeOH/EtOAc) to yield 126 (10 mg, 13%) as a white solid. ¹H NMR (DMSO-d₆, 400 MHz) δ 8.78-8.82 (m, 1H), 8.48-8.52 (m, 1H), 8.34-8.40 (m, 1H), 7.86-7.94 (m, 1H), 7.70-7.76 (m, 2H), 7.62-7.68 (m, 1H), 7.46-7.56 (m, 2H), 7.38-7.46 (m, 2H), 6.48-6.54 (m, 1H), 5.20 (s, 2H), 3.96 (s, 3H), 3.95 (s, 3H). LRMS (ESI pos) m/e 536 (M+1).

### Example 27

### Preparation of 5-(4-(6,7-dimethoxyquinolin-4-yloxy)-3-fluorophenyl)-3-(4-(trifluoromethyl)benzyl)pyrimidin-4(3H)-one 127

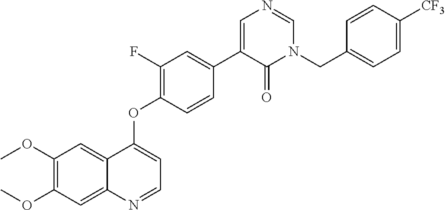

Step A: Preparation of 5-bromo-3-(4-(trifluoromethyl)benzyl)pyrimidin-4(3H)-one: Prepared from 1-(bromomethyl)-4-(trifluoromethyl)benzene (2.0 g, 8.3 mmol) according to the procedure described for Example 13, Step A. The crude product was purified by silica gel flash column chromatography (1:1 EtOAc/Hexane) to yield the product (0.28 g, 9.8%) as a white solid. LRMS (ESI pos) m/e 333 (M+1).

Step B: Preparation of 5-(4-(benzyloxy)-3-fluorophenyl)-3-(4-(trifluoromethyl)benzyl)pyrimidin-4(3H)-one: Prepared from 5-bromo-3-(4-(trifluoromethyl)benzyl)pyrimidin-4(3H)-one (0.28 g, 0.25 mmol) according to the procedure described for Example 7, Step A. The crude product was purified by silica gel flash column chromatography (1:1 EtOAc/Hexane) to yield the product (0.34 g, 90%) as a white solid. LRMS (ESI pos) m/e 455 (M+1).

Step C: Preparation of 5-(3-fluoro-4-hydroxyphenyl)-3-(4-(trifluoromethyl)benzyl)pyrimidin-4(3H)-one: Prepared from in 5-(4-(benzyloxy)-3-fluorophenyl)-3-(4-(trifluoromethyl)benzyl)pyrimidin-4(3H)-one (0.34 g, g, 0.75 mmol) according to the procedure described for Example 1, Step C, to yield the product (0.2 g, 72%) as a white solid. LRMS (ESI pos) m/e 365 (M+1).

Step D: Preparation of 5-(4-(6,7-dimethoxyquinolin-4-yloxy)-3-fluorophenyl)-3-(4-(trifluoromethyl)benzyl)pyrimidin-4(3H)-one: Prepared from 4-chloro-6,7-dimethoxyquinoline (prepared according to reference procedure in Example 5) (100 mg, 0.45 mmol) and 5-(3-fluoro-4-hydroxyphenyl)-3-(4-(trifluoromethyl)benzyl)pyrimidin-4(3H)-one (100 mg, 0.22 mmol) according to the procedure described for Example 1, Step E. The crude product was purified by silica gel flash column chromatography (1:10 MeOH/EtOAc) to yield 127 (38 mg, 31%) as a white solid. ¹H NMR (DMSO-d₆, 400 MHz) δ 8.82 (s, 1H), 8.50 (d, J=5.5 Hz, 1H), 8.40 (s, 1H), 7.89-7.94 (m, 1H), 7.72-7.78 (m, 3H), 7.59-7.64 (m, 2H), 7.48-7.54 (m, 2H), 7.42 (s, 1H), 6.48-6.52 (m, 1H), 5.30 (s, 2H), 3.96 (s, 3H), 3.95 (s, 3H). LRMS (ESI pos) m/e 552 (M+1).

### Example 28

### Preparation of 3-(4-chloro-2-fluorobenzyl)-5-(4-(6,7-dimethoxyquinolin-4-yloxy)-3-fluorophenyl)pyrimidin-4(3H)-one 128

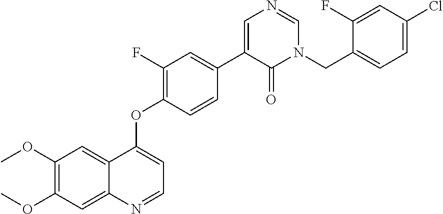

Step A: Preparation of 5-bromo-3-(4-chloro-2-fluorobenzyl)pyrimidin-4(3H)-one: Prepared from 1-(bromomethyl)-4-chloro-2-fluorobenzene (1.87 g, 8.3 mmol) according to the procedure described for Example 13, Step A. The crude product was purified by silica gel flash column chromatography (1:1 EtOAc/Hexane) to yield the product (0.81 g, 30%) as a white solid. LRMS (ESI pos) m/e 319 (M+1).

Step B: Preparation of 5-(4-(benzyloxy)-3-fluorophenyl)-3-(4-chloro-2-fluorobenzyl)pyrimidin-4(3H)-one: Prepared from 5-bromo-3-(4-chloro-2-fluorobenzyl)pyrimidin-4(3H)-one (0.81 g, 2.5 mmol) according to the procedure described for Example 7, Step A. The crude product was purified by silica gel flash column chromatography (EtOAc) to yield the product (0.74 g, 66%) as a white solid. LRMS (ESI pos) m/e 439 (M+1).

Step C: Preparation of 3-(4-chloro-2-fluorobenzyl)-5-(3-fluoro-4-hydroxyphenyl)pyrimidin-4(3H)-one: Prepared from 5-(4-(benzyloxy)-3-fluorophenyl)-3-(4-chloro-2-fluorobenzyl)pyrimidin-4(3H)-one (0.74 g, 1.7 mmol) according to the procedure described for Example 1, Step C, to yield the product (0.5 g, 85%) as a white solid. LRMS (ESI pos) m/e 349 (M+1).

Step D: Preparation of 3-(4-chloro-2-fluorobenzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (50 mg, 0.22 mmol) and in 3-(4-chloro-2-fluorobenzyl)-5-(3-fluoro-4-hydroxyphenyl)pyrimidin-4(3H)-one (78 mg, 0.22 mmol) according to the procedure described for Example 1, Step E. The crude product was purified by silica gel flash column chromatography (1:10 MeOH/EtOAc) to yield 128 (8.3 mg, 7%) as a white solid. ¹H NMR (DMSO-d₆, 400 MHz) δ 8.68 (s, 1H), 8.44 (s, 1H), 8.33 (s, 1H), 7.80-8.00 (m, 2H), 7.60-7.70 (m, 1H), 7.40-7.50 (m, 3H), 7.30-7.40 (m, 2H), 7.20-7.30 (m, 1H), 6.40-6.50 (m, 1H), 5.19 (s, 2H), 3.90 (s, 6H). LRMS (ESI pos) m/e 536 (M+1).

### Example 29

### Preparation of 3-(2-chloro-4-fluorobenzyl)-5-(4-(6,7-dimethoxyquinolin-4-yloxy)-3-fluorophenyl)pyrimidin-4(3H)-one 129

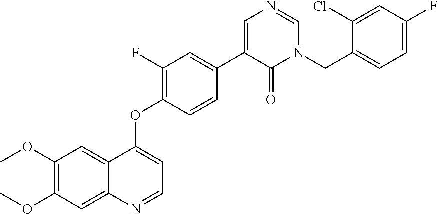

Step A: Preparation of 5-bromo-3-(2-chloro-4-fluorobenzyl)pyrimidin-4(3H)-one: Prepared from 1-(bromomethyl)-2-chloro-4-fluorobenzene (1.87 g, 8.4 mmol) according to the procedure described for Example 13, Step A. The crude product was purified by silica gel flash column chromatography (1:1 EtOAc/Hexane) to yield the product (0.66 g, 24%) as a white solid. LRMS (ESI pos) m/e 319 (M+1).

Step B: Preparation of 5-(4-(benzyloxy)-3-fluorophenyl)-3-(2-chloro-4-fluorobenzyl)pyrimidin-4(3H)-one: Prepared from 5-bromo-3-(2-chloro-4-fluorobenzyl)pyrimidin-4(3H)-one (0.66 g, 2.1 mmol) according to the procedure described for Example 7, Step A. The crude product was purified by silica gel flash column chromatography (1:1 EtOAc/Hexane) to yield the product (0.84 g, 92%) as a white solid. LRMS (ESI pos) m/e 439 (M+1).

Step C: Preparation of 3-(2-chloro-4-fluorobenzyl)-5-(3-fluoro-4-hydroxyphenyl)pyrimidin-4(3H)-one: Prepared from 5-(4-(benzyloxy)-3-fluorophenyl)-3-(2-chloro-4-fluorobenzyl)pyrimidin-4(3H)-one (0.84 g, 1.5 mmol) according to the procedure described for Example 1, Step C, to yield the product (0.5 g, 75%) as a white solid. LRMS (ESI pos) m/e 349 (M+1).

Step D: Preparation of 3-(2-chloro-4-fluorobenzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (80 mg, 0.36 mmol) and 3-(2-chloro-4-fluorobenzyl)-5-(3-fluoro-4-hydroxyphenyl)pyrimidin-4(3H)-one (125 mg, 0.36 mmol) according to the procedure described for Example 1, Step E. The crude product was purified by silica gel flash column chromatography (1:10 MeOH/EtOAc) to yield 129 (4.2 mg, 2.2%) as a white solid. ¹H NMR (DMSO-d₆, 400 MHz) δ 8.64 (s, 1H), 8.45 (s, 1H), 8.35 (s, 1H), 7.80-7.90 (m, 1H). 7.60-7.70 (m, 1H), 7.40-7.60 (m, 3H), 7.30-7.40 (m, 1H), 7.10-7.30 (m, 2H), 6.40-6.50 (m, 1H), 5.20 (s, 2H), 3.90 (s, 6H). LRMS (ESI pos) m/e 536 (M+1).

### Example 30

### Preparation of 3-(4-chloro-2,6-difluorobenzyl)-5-(4-(6,7-dimethoxyquinolin-4-yloxy)-3-fluorophenyl)pyrimidin-4(3H)-one 130

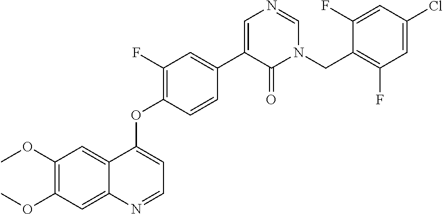

Step A: Preparation of 5-bromo-3-(4-chloro-2,6-difluorobenzyl)pyrimidin-4(3H)-one: Prepared from 2-(bromomethyl)-5-chloro-1,3-difluorobenzene (2.0 g, 8.3 mmol) according to the procedure described for Example 13, Step A. The crude product was purified by silica gel flash column chromatography (1:1 EtOAc/Hexane) to yield the product (0.76 g, 26%) as a white solid. LRMS (ESI pos) m/e 335 (M+1).

Step B: Preparation of 5-(4-(benzyloxy)-3-fluorophenyl)-3-(4-chloro-2,6-difluorobenzyl)pyrimidin-4(3H)-one: Prepared from 5-bromo-3-(4-chloro-2,6-difluorobenzyl)pyrimidin-4(3H)-one (0.76 g, 2.2 mmol) according to the procedure described for Example 7, Step A. The crude product was purified by silica gel flash column chromatography (1:1 EtOAc/Hexane) to yield the product (0.52 g, 50%) as a white solid. LRMS (ESI pos) m/e 457 (M+1).

Step C: Preparation of 3-(4-chloro-2,6-difluorobenzyl)-5-(3-fluoro-4-hydroxyphenyl)pyrimidin-4(3H)-one: Prepared from 5-(4-(benzyloxy)-3-fluorophenyl)-3-(4-chloro-2,6-difluorobenzyl)pyrimidin-4(3H)-one (0.52 g, 1.14 mmol) according to the procedure described for Example 1, Step C, to yield the product (0.4 g, 97%) as a white solid. LRMS (ESI pos) m/e 367 (M+1).

Step D: Preparation of 3-(4-chloro-2,6-difluorobenzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (100 mg, 0.45 mmol) and 3-(4-chloro-2,6-difluorobenzyl)-5-(3-fluoro-4-hydroxyphenyl)pyrimidin-4(3H)-one (164 mg, 0.45 mmol) according to the procedure described for Example 1, Step E. The crude product was purified by silica gel flash column chromatography (1:10 MeOH/EtOAc) to yield 130 (1.4 mg, 1%) as a white solid. ¹H NMR (DMSO-d₆, 400 MHz) δ 8.76(s, 1H), 8.49 (s, 1H), 8.35 (s, 1H), 7.82-7.90 (m, 1H), 7.66-7.74 (m, 1H), 7.45-7.55 (m, 2H), 7.35-7.40 (m, 3H), 6.44-6.52 (m, 1H), 5.24 (s, 2H), 3.95 (s, 6H). LRMS (ESI pos) m/e 554 (M+1).

### Example 31

### Preparation of 5-(4-(6,7-dimethoxyquinolin-4-yloxy)-3-fluorophenyl)-3-(3,4-dimethylbenzyl)pyrimidin-4(3H)-one 131

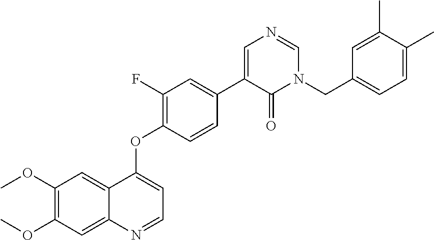

Step A: Preparation of 5-bromo-3-(3,4-dimethylbenzyl)pyrimidin-4(3H)-one: Prepared from 4-(chloromethyl)-1,2-dimethylbenzene (1.3 g, 8.4 mmol) according to the procedure described for Example 13, Step A. The crude product was purified by silica gel flash column chromatography (1:1 EtOAc/Hexane) to yield the product (0.68 g, 27%) as a white solid. LRMS (ESI pos) m/e 295 (M+1).

Step B: Preparation of 5-(4-(benzyloxy)-3-fluorophenyl)-3-(3,4-dimethylbenzyl)pyrimidin-4(3H)-one: Prepared from 5-bromo-3-(3,4-dimethylbenzyl)pyrimidin-4(3H)-one (0.68 g, 2.3 mmol) according to the procedure described for Example 7, Step A. The crude product was purified by silica gel flash column chromatography (1:1 EtOAc/Hexane) to yield the product (0.17 g, 17%) as a white solid. LRMS (ESI pos) m/e 415 (M+1).

Step C: Preparation of 3-(3,4-dimethylbenzyl)-5-(3-fluoro-4-hydroxyphenyl)pyrimidin-4(3H)-one: Prepared from 5-(4-(benzyloxy)-3-fluorophenyl)-3-(3,4-dimethylbenzyl)pyrimidin-4(3H)-one (0.17 g, 0.4 mmol) according to the procedure described for Example 1, Step C, to yield the product (0.1 g, 77%) as a white solid. LRMS (ESI pos) m/e 325 (M+1).

Step D: Preparation of 5-(4-(6,7-dimethoxyquinolin-4-yloxy)-3-fluorophenyl)-3-(3,4-dimethylbenzyl)pyrimidin-4(3H)-one: Prepared from 4-chloro-6,7-dimethoxyquinoline (prepared according to reference procedure in Example 5) (100 mg, 0.45 mmol) and 3-(3,4-dimethylbenzyl)-5-(3-fluoro-4-hydroxyphenyl)pyrimidin-4(3H)-one (145 mg, 0.45 mmol) according to the procedure described for Example 1, Step E. The crude product was purified by silica gel flash column chromatography (1:10 MeOH/EtOAc) to yield 131 (2 mg, 1%) as a white solid. ¹H NMR (DMSO-d₆, 400 MHz) δ 8.56 (s, 1H), 8.51 (s, 1H), 8.40 (s, 1H), 7.90-8.00 (m, 1H), 7.70-7.80 (m, 2H), 7.48-7.60 (m, 2H), 7.38-7.46 (m, 1H), 7.02-7.20 (m, 2H), 6.76-6.84 (m, 1H), 6.48-6.56 (m, 1H), 5.20 (s, 2H), 3.96 (s, 3H), 3.95 (s, 3H), 2.50 (s, 6H). LRMS (ESI pos) m/e 512 (M+1).

### Example 32

### Preparation of 5-(4-(6,7-dimethoxyquinolin-4-yloxy)-3-fluorophenyl)-3-(4-fluoro-3-methylbenzyl)pyrimidin-4(3H)-one 132

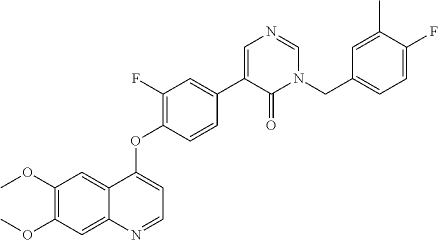

Step A: Preparation of 5-bromo-3-(4-fluoro-3-methylbenzyl)pyrimidin-4(3H)-one: Prepared from 4-(bromomethyl)-1-fluoro-2-methylbenzene (0.57 g, 2.8 mmol) according to the procedure described for Example 13, Step A. The crude product was purified by silica gel flash column chromatography (1:1 EtOAc/Hexane) to yield the product (0.46 g, 54%) as a white solid. LRMS (ESI pos) m/e 296 (M+1).

Step B: Preparation of 5-(4-(benzyloxy)-3-fluorophenyl)-3-(4-fluoro-3-methylbenzyl)pyrimidin-4(3H)-one: Prepared from 5-bromo-3-(4-fluoro-3-methylbenzyl)pyrimidin-4(3H)-one (0.46 g, 1.5 mmol) according to the procedure described for Example 7, Step A. The crude product was purified by silica gel flash column chromatography (1:1 EtOAc/Hexane) to yield the product (86 mg, 13%) as a white solid. LRMS (ESI pos) m/e 419 (M+1).

Step C: Preparation of 3-(4-fluoro-3-methylbenzyl)-5-(3-fluoro-4-hydroxyphenyl)pyrimidin-4(3H)-one: Prepared 5-(4-(benzyloxy)-3-fluorophenyl)-3-(4-fluoro-3-methylbenzyl)pyrimidin-4(3H)-one (86 mg, 0.2 mmol) according to the procedure described for Example 1, Step C, to yield the product (50 mg, 74%) as a white solid. LRMS (ESI pos) m/e 329 (M+1).

Step D: Preparation of 5-(4-(6,7-dimethoxyquinolin-4-yloxy)-3-fluorophenyl)-3-(4-fluoro-3-methylbenzyl)pyrimidin-4(3H)-one: Prepared from 4-chloro-6,7-dimethoxyquinoline (prepared according to reference procedure in Example 5) (40 mg, 0.18 mmol) and 3-(4-fluoro-3-methylbenzyl)-5-(3-fluoro-4-hydroxyphenyl)pyrimidin-4(3H)-one (59 mg, 0.18 mmol) according to the procedure described for Example 1, Step E. The crude product was purified by silica gel flash column chromatography (1:10 MeOH/EtOAc) to yield 132 (1.6 mg, 2%) as a white solid. ¹H NMR (DMSO-d₆, 400 MHz) δ 8.79 (s, 1H), 8.50 (d, J=5.5 Hz, 1H), 8.35 (s, 1H), 7.89-7.95 (m, 1H), 7.72-7.75 (m, 1H), 7.46-7.60 (m, 2H), 7.45 (s, 1H), 7.25-7.40 (m, 2H), 7.10-7.16 (m, 1H), 6.48-6.54 (m, 1H), 5.16 (s, 2H), 5.96 (s, 3H), 5.95 (s, 3H), 2.22 (s, 3H). LRMS (ESI pos) m/e 516 (M+1).

### Example 33

### Preparation of 4-benzoyl-1-(4-(6,7-dimethoxyquinolin-4-yloxy)-3-fluorophenyl)pyridin-2(1H)-one 133

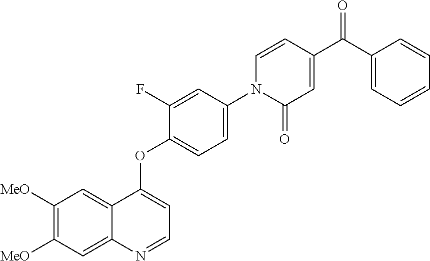

Step A: Preparation of (5-bromo-2-methoxypyridin-4-yl)(phenyl)methanol: Prepared from 5-bromo-2-methoxypyridine (10 g, 51 mmol) according to the procedure described for Example 12, Step A. The crude product was purified by silica gel flash column chromatography (1:1 EtOAc/Hexane) to yield the product (8.9 g, 60%) as a colorless liquid. ¹H NMR (CDCl₃, 400 MHz) δ 8.18 (s, 1H), 7.30-7.40 (m, 5H), 7.12 (s, 1H), 5.99 (d, J=3.9 Hz, 1H), 3.93 (s, 3H), 2.34 (d, J=3.9 Hz, 1H).

Step B: Preparation of (5-bromo-2-methoxypyridin-4-yl)(phenyl)methanone: PCC (2.93 g, 13.6 mmol) and 4 A molecular sieves (2 g) were added into a solution of (5-bromo-2-methoxypyridin-4-yl)(phenyl)methanol (2.0 g, 6.80 mmol) in CH₂Cl₂ (50 mL). The reaction was stirred for 1 hour, was then filtered with a pad of silica gel. The filtrate was concentrated the filtrate to yield the product (1.9 g, 99%) as a yellow liquid. ¹H NMR (CDCl₃, 400 MHz) δ 8.34 (s, 1H), 7.80-7.84 (m, 2H), 7.61-7.66 (m, 1H), 7.46-7.52 (m, 2H). 6.72 (s, 1H), 3.97 (s, 3H).

Step C: Preparation of 4-benzoyl-5-bromopyridin-2(1H)-one: (5-bromo-2-methoxypyridin-4-yl)(phenyl)methanone (1.3 g, 4.5 mmol) and pyridine hydrochloride (2 g, 17 mmol) were heated at 150° C. for 1 hour. CH₂Cl₂ (30 mL) was added into the hot mixture. The mixture was cooled and the solvent was evaporated. The residue was purified by silica gel flash column chromatography (2:1 EtOAc/Hexane) to yield the product (0.3 g, 24%) as a white solid. ¹H NMR (DMSO-d₆, 400 MHz) δ 12.00 (s, 1H), 7.91 (s, 1H), 7.82-7.86 (m, 2H), 7.72-7.78 (m, 1H), 7.56-7.64 (m, 2H), 6.53 (s, 1H).

Step D: Preparation of 4-benzoyl-1-(3-fluoro-4-hydroxyphenyl)pyridin-2(1H)-one: Prepared from 4-benzoyl-5-bromopyridin-2(1H)-one (170 mg, 0.61 mmol) according to the procedure described for Example 1, Step D. The crude product was purified by silica gel flash column chromatography (EtOAc) to yield the product (170 mg, 90%) as a brown solid. ¹H NMR (DMSO-d₆, 400 MHz) δ 10.28 (s, 1H), 7.85-7.90 (m, 2H), 7.79 (d, J=7.0 Hz, 1H), 7.72-7.77 (m, 1H), 7.59-7.65 (m, 2H), 7.35-7.40 (m, 1H), 7.04-7.14 (m, 2H), 6.60 (d, J=2.0 Hz, 1H), 6.46 (dd, J=7.0, 2.0 Hz, 1H). LRMS (ESI pos) m/e 310 (M+1).

Step E: Preparation of 4-benzoyl-1-(4-(6,7-dimethoxyquinolin-4-yloxy)-3-fluorophenyl)pyridin-2(1H)-one: Prepared from 4-chloro-6,7-dimethoxyquinoline (prepared according to reference procedure in Example 5) (123 mg, 0.55 mmol) and 4-benzoyl-1-(3-fluoro-4-hydroxyphenyl)pyridin-2(1H)-one (170 mg, 0.55 mmol) according to the procedure described for Example 1, Step E. The crude product was purified by silica gel flash column chromatography (1:10 MeOH/EtOAc) to yield 133 (151 mg, 55%) as a white solid. ¹H NMR (DMSO-d₆, 400 MHz) δ 8.55 (d, J=5.5 Hz, 1H), 7.88-7.94 (m, 3H), 7.74-7.84 (m, 2H), 7.60-7.66 (m, 3H), 7.55 (s, 1H), 7.48-7.52 (m, 1H0, 7.45 (s, 1H), 6.66-6.68 (m, 1H), 6.54-6.62 (m, 2H). LRMS (ESI pos) m/e 497 (M+1).

### Example 34

### Preparation of N-(4-(7-(3-morpholinopropoxy)-6-methoxyquinolin-4-yloxy)-3-fluorophenyl)-2-(pyridin-2-yl)acetamide 134

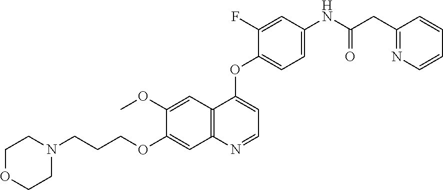

A mixture of 3-fluoro-4-(6-methoxy-7-(3-morpholinopropoxy)quinolin-4-yloxy)aniline (prepared in example 72, steps C-F) (10.0 mg, 0.0234 mmol), 2-(pyridin-2-yl)acetic acid (16.0 mg, 0.117 mmol), N¹-((ethylimino)methylene)-N³,N³-dimethylpropane-1,3-diamine hydrochloride (22.4 mg, 0.117 mmol), 1H-benzo[d][1,2,3]triazol-1-ol (15.8 mg, 0.117 mmol) and N-ethyl-N-isopropylpropan-2-amine (0.0204 ml, 0.117 mmol) in THF (10 mL) was stirred at room temperature for 2 days. Water (10 mL) was added and the aqueous was extracted with CH₂Cl₂ (3×50 mL). The organic layers were combined and dried over Na₂SO₄. Concentration and purification by silica gel chromatography afforded 134 (1.3 mg, 10.2%). ¹H NMR (400 MHz, CDCl₃) δ 10.37 (s, 1H, NH), 8.65 (d, J=4.8 Hz, 1H), 8.46 (d, J=5.2 Hz, 1H), 7.72-7.79 (m, 2H), 7.57 (s, 1H), 7.43 (s, 1H), 7.30-7.34 (m, 3H), 7.21 (t, J=8.6 Hz, 1H), 6.37 (d, J=5.6 Hz, 1H), 4.27 (t, J=6.8 Hz, 2H), 4.04 (s, 3H), 3.91 (s, 2H), 3.73 (t, J=4.6 Hz, 4H), 2.58 (t, J=7.2 Hz, 2H), 2.44-2.53 (m, 4H), 2.10-2.17 (m, 2H). LRMS (APCI neg) m/z 545 (M−1).

### Example 35

### Preparation of 4-(2-fluoro-4-(6-methoxypyridin-3-yl)phenoxy)-6,7-dimethoxyquinoline 135

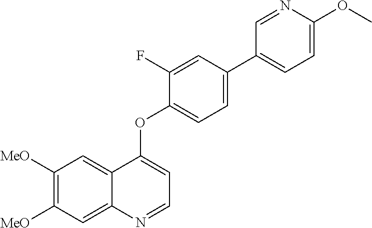

Prepared from 2-methoxy-5-pyridine boronic acid (24 mg, 0.16 mmol) and 4-(4-bromo-2-fluorophenoxy)-6,7-dimethoxyquinoline (Example 34, 60 mg, 0.16 mmol) according to the procedure described for Example 7, Step A. The crude product was purified by silica gel flash column chromatography (EtOAc) to yield 135 (30 mg, 47%) as a white solid. ¹H NMR (DMSO-d₆, 400 MHz) δ 8.60 (s, 1H0, 8.51 (s, 1H), 8.10-8.18 (m, 1 h), 7.80-7.90 (m, 1H), 7.60-7.70 (m, 1H), 7.50-7.60 (m, 1H), 7.40 (s, 1H), 6.90-7.00 (m, 1H), 6.50-6.60 (m, 1H), 3.96 (s, 6H), 3.92 (s, 3H). LRMS (ESI pos) m/e 407 (M+1).

### Example 36

### Preparation of 4-(3-fluoro-4′-phenoxybiphenyl-4-yloxy)-6,7-dimethoxyquinoline 136

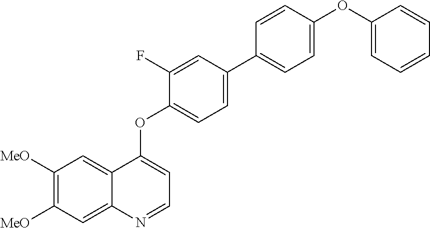

Prepared from 4-phenoxyphenyl boronic acid (109 mg, 0.51 mmol) and 4-(4-bromo-2-fluorophenoxy)-6,7-dimethoxyquinoline (Example 34, 64 mg, 0.17 mmol) according to the procedure described for Example 7, Step A. The crude product was purified by silica gel flash column chromatography (EtOAc) to yield 136 (40 mg, 51%) as a white solid. ¹H NMR (DMSO-d₆, 400 MHz) δ 8.51 (d, J=5.1 Hz, 1H), 7.78-7.86 (m, 3H), 7.62-7.68 (m, 1H), 7.50-7.58 (m, 2H), 7.40-7.46 (m, 3H), 7.16-7.22 (m, 1H), 7.06-7.14 (m, 4H), 6.54 (d, J=5.1 HZ, 1H), 3.88 (s, 6H). LRMS (ESI pos) m/e 468 (M+1).

### Example 37

### Preparation of N-(4′-(6,7-dimethoxyquinolin-4-yloxy)-3′-fluorobiphenyl-4-yl)methanesulfonamide 137

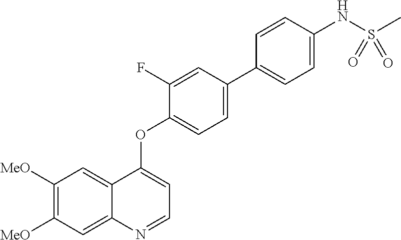

Prepared from 4-(methylsulfonylamino)phenyl boronic acid (109 mg, 0.51 mmol) and 4-(4-bromo-2-fluorophenoxy)-6,7-dimethoxyquinoline (Example 34, 64 mg, 0.17 mmol) according to the procedure described for Example 7, Step A. The crude product was purified by silica gel flash column chromatography (EtOAc) to yield 137 (55 mg, 69%) as a white solid. ¹H NMR (DMSO-d₆, 400 MHz) δ 9.93 (s, 1H), 8.51 (s, 1H), 7.56-7.82 (m, 3H), 7.50-7.65 (m, 3H), 7.43 (s, 1H), 7.24-7.38 (m, 2H), 6.50-6.60 (m, 1H), 3.96 (m, 6H), 3.05 (s, 3H). LRMS (ESI pos) m/e 469 (M+1).

### Example 38

### Preparation of N-cyclopropyl-4′-(6,7-dimethoxyquinolin-4-yloxy)-3′-fluorobiphenyl-4-carboxamide 138

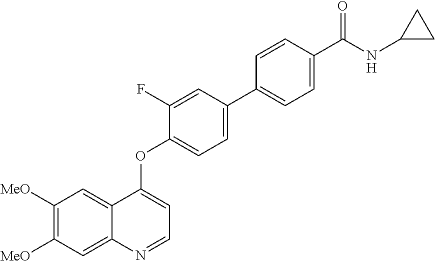

Prepared from 4-(cyclopropylcarbamoyl)phenyl boronic acid (81 mg, 0.40 mmol) and 4-(4-bromo-2-fluorophenoxy)-6,7-dimethoxyquinoline (Example 34, 50 mg, 0.13 mmol) according to the procedure described for Example 7, Step A. The crude product was purified by silica gel flash column chromatography (EtOAc) to yield 138 (30 mg, 49%) as a white solid. ¹H NMR (DMSO-d₆, 400 MHz) δ 8.50-8.54 (m, 2H), 7.92-7.96 (m, 3H), 7.85-7.89 (m, 1H), 7.72-7.76 (m, 1H), 7.55-7.60 (m, 2H), 7.43 (s, 1H), 6.55-6.58 (m, 1H), 2.84-2.92 (m, 1H), 0.69-0.74 (m, 2H), 0.58-0.64 (m, 2H). LRMS (ESI pos) m/e 459 (M+1).

### Example 39

### Preparation of 4-(3-fluoro-4′-(tetrahydro-2H-pyran-2-yloxy)biphenyl-4-yloxy)-6,7-dimethoxyquinoline 139

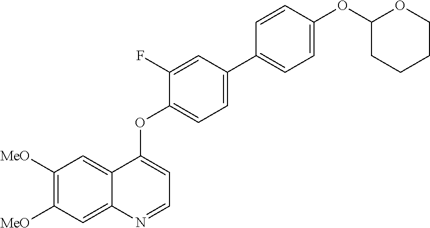

Prepared from 4-(tetrahydro-2H-pyran-2-yloxy)phenyl boronic acid (106 mg, 0.48 mmol) and 4-(4-bromo-2-fluorophenoxy)-6,7-dimethoxyquinoline (Example 34, 60 mg, 0.16 mmol) according to the procedure described for Example 7, Step A. The crude product was purified by silica gel flash column chromatography (EtOAc) to yield 139 (40 mg, 53%) as a white solid. ¹H NMR (DMSO-d₆, 400 MHz) δ 8.51 (s, 1H), 8.50 (s, 1H), 7.78-7.82 (m, 1H), 7.68-7.72 (m, 2H), 7.58-7.62 (m, 1H), 7.55 (s, 1H), 7.48-7.52 (m, 1H), 7.43 (s, 1H), 7.10-7.15 (m, 2H), 5.52-5.56 (m, 1H), 3.75-3.82 (m, 1H), 1.70-1.94 (m, 3H), 1.50-1.70 (m, 3H). LRMS (ESI pos) m/e 476 (M+1).

### Example 40

### Preparation of 1-(4-(6,7-dimethoxyquinolin-4-yloxy)-3-fluorophenyl)-4-methylpyridin-2(1H)-one 140

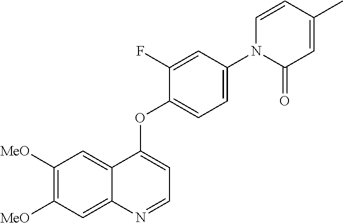

Step A: Preparation of 1-(3-fluoro-4-hydroxyphenyl)-4-methylpyridin-2(1H)-one: Prepared from 2-hydroxy-4-methylpyridine (2.37 g, 21.7 mmol) according to the procedure described for Example 1, Step D. The crude reaction mixture was filtered with a pad of celite and the filtrate was evaporated to yield the product as a brown solid (1.0 g, 21%). ¹H NMR (DMSO-d₆, 400 MHz) δ 10.17 (s, 1H), 7.48 (d, J=7.0 Hz, 1H), 7.20-7.27 (m, 1H), 6.95-7.05 (m, 2H), 6.25 (s, 1H), 6.14 (dd, J=7.0, 1.6 Hz, 1H), 2.16 (s, 3H). LRMS (ESI pos) m/e 220 (M+1).

Step B: Preparation of 1-(4-(6,7-dimethoxyquinolin-4-yloxy)-3-fluorophenyl)-4-methylpyridin-2(1H)-one: Prepared from 1-(3-fluoro-4-hydroxyphenyl)-4-methylpyridin-2(1H)-one (0.1 g, 0.46 mmol) and 4-chloro-6,7-dimethoxyquinoline (prepared according to reference procedure in Example 5) (0.12 g, 0.55 mmol) using the procedure described for Example 1, Step E. The reaction mixture was purified by silica gel flash column chromatography (1:10 MeOH/EtOAc) to yield 140 (50 mg, 27%) as a white solid. ¹H NMR (CDCl₃, 400 MHz) δ 8.52 (s, 1H), 7.70 (dd, 1H), 7.62 (d, 1H), 7.56-7.60 (m, 1H), 7.54 (s, 1H), 7.44 (s, 1H), 6.24 (dd, 1H), 3.96 (s, 3H), 3.95 (s, 3H), 2.20 (s, 1H). LRMS (ESI pos) m/e 407 (M+1).

### Example 41

### Preparation of 1-(4-(6,7-dimethoxyquinolin-4-yloxy)-3-fluorophenyl)-4-(hydroxy(phenyl)methyl)pyridin-2(1H)-one 141

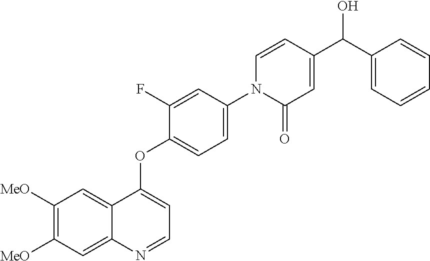

Sodium borohydride (11 mg, 0.30 mmol) was added into a solution of 4-benzoyl-1-(4-(6,7-dimethoxyquinolin-4-yloxy)-3-fluorophenyl)pyridin-2(1H)-one (Example 33, 30 mg, 0.060 mmol) in MeOH (2 mL) at 0° C. The reaction was warmed up to room temperature and was stirred for 20 minutes. The reaction was diluted with ethyl acetate (10 mL), washed with sodium bicarbonate and brine, dried with Na₂SO₄, filter and concentrated. The residue was purified by silica gel flash column chromatography (1:10 MeOH/EtOAc) to yield 141 (30 mg, 95%) as a yellow solid. ¹H NMR (DMSO-d₆, 400 MHz) δ 8.53 (d, 1H), 7.70 (dd, 1H), 7.64 (d, 1H), 7.54-7.58 (m, 1H), 7.52 (s, 1H), 7.44-7.48 (m, 1H), 7.43 (s, 1H), 7.34-7.42 (m, 3H), 7.25-7.30 (m, 1H), 6.60 (s, 1H), 6.54 (d, 1H), 6.28 (d, 1H), 6.12 (s, 1H), 5.75 (s, 1H), 3.96 (s, 3H), 3.95 (s, 3H). LRMS (ESI pos) m/e 499 (M+1).

### Example 42

### Preparation of (1-(4-(6,7-dimethoxyquinolin-4-yloxy)-3-fluorophenyl)-2-oxo-1,2-dihydropyridin-4-yl)(phenyl)methyl acetate 142

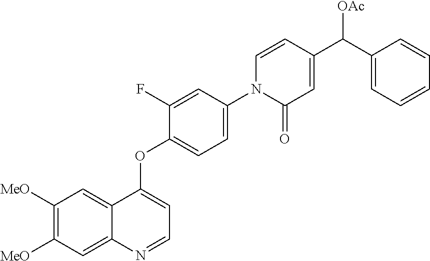

Triethylamine (0.1 mL, 0.7 mmol) and acetyl chloride (2.7 mg, 0.034 mmol) was into a solution of 1-(4-(6,7-dimethoxyquinolin-4-yloxy)-3-fluorophenyl)-4-(hydroxy(phenyl)methyl)pyridin-2(1H)-one (Example, 41, 17 mg, 0.034 mmol) in CH₂Cl₂ (2 mL) and was stirred for 10 minutes. Water (1 mL) and ethyl acetate (2 mL) was added. The reaction mixture was extracted with ethyl acetate, and the organic layer was washed with brine, dried over sodium sulfate, filtered, and concentrated. The residue was purified by silica gel flash column chromatography (1:10 MeOH/EtOAc) to yield 142 (10 mg, 43% yield) as a yellow solid. LRMS (ESI pos) m/e 541 (M+1).

### Example 43

### Preparation of 5-(4-(6,7-dimethoxyquinolin-4-yloxy)-3-fluorophenyl)pyrimidin-4(3H)-one 143

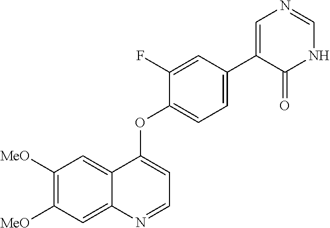

Step A: Preparation of 5-(4-(benzyloxy)-3-fluorophenyl)pyrimidin-4(3H)-one: Prepared from 5-bromopyrimidin-4(3H)-one (300 mg, 1.71 mmol) and 4-(benzyloxy)-3-fluorophenylboronic acid (844 mg, 3.43 mmol) according to the procedure described for Example 7, Step A. The product was crashed out from the solution while the crude was concentrating. The solid was collected to yield the product (305 mg, 60%) as white solid. LRMS (ESI pos) m/e 297 (M+1).

Step B: Preparation of 5-(3-fluoro-4-hydroxyphenyl)pyrimidin-4(3H)-one: Prepared from 5-(4-(benzyloxy)-3-fluorophenyl)pyrimidin-4(3H)-one (300 mg, 1.01 mmol) in MeOH (2 mL) and acetic acid (2 mL) according to the procedure described for Example 14, Step B, to yield the product (140 mg, 67%) as white solid. LRMS (ESI pos) m/e 207 (M+1).

Step C: Preparation of 5-(4-(6,7-dimethoxyquinolin-4-yloxy)-3-fluorophenyl)pyrimidin-4(3H)-one: Prepared from 5-(3-fluoro-4-hydroxyphenyl)pyrimidin-4(3H)-one (40 mg, 0.19 mmol) and 4-chloro-6,7-dimethoxyquinoline (prepared according to reference procedure in Example 5) (43 mg, 0.19 mmol) according to the procedure described for Example 1, Step E, to yield 143 (1 mg, 1%) as white solid. ¹H NMR (DMSO-d₆, 400 MHz) δ 12.93 (s, 1H), 8.84-8.54 (m, 1H), 8.34 (s, 1H), 8.26 (s, 1H), 7.96 (d, 1H), 7.76 (d, 1H), 7.48-7.58 (m, 2H), 7.42 (s, 1H), 6.50-6.54 (m, 1H), 3.96 (s, 6H). LRMS (ESI pos) m/e 394 (M+1).

### Example 44

### Preparation of 4-(4′-((1H-pyrazol-1-yl)methyl)-3-fluorobiphenyl-4-yloxy)-6,7-dimethoxyquinoline 144

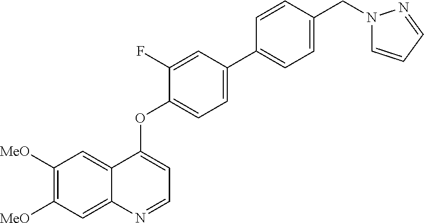

Prepared from 4-(4-bromo-2-fluorophenoxy)-6,7-dimethoxyquinoline (Example 35, 60 mg, 0.16 mmol) and 1H-pyrazole-1-benzyl-4-boronic acid (96 mg, 0.48 mmol) according to the procedure described for Example 7, Step A. The crude product was purified by silica gel flash column chromatography (EtOAc) to yield 144 (40 mg, 55%) as a white solid. ¹H NMR (DMSO-d₆, 400 MHz) δ 8.46-8.56 (m, 1H), 8.40 (s, 1H), 8.02 (s, 1H), 7.50 (d, 1H), 7.50-7.65 (m, 2H), 7.24-7.48 (m, 7H), 6.46-6.52 (m, 1H), 5.36 (s, 2H), 3.96 (s, 6H). LRMS (ESI pos) m/e 456 (M+1).

### Example 45

### Preparation of 3-(4-(7-(3-morpholinopropoxy)-6-methoxyquinolin-4-yloxy)-3-fluorophenyl)-3,4-dihydroquinazolin-2(1H)-one 145

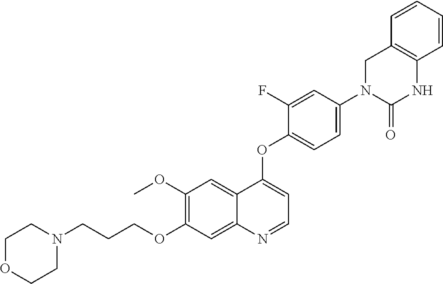

Step A: Preparation of 2-amino-N-(3-fluoro-4-methoxyphenyl)benzamide: To a stirred suspension of isatoic anhydride (1.63 g, 10 mmol) in 15 mL dioxane at room temperature under nitrogen was added powdered sodium hydroxide (40 mg, 1 mmol) followed by 3-fluoro-4-methoxyaniline (1.41 g, 10 mmol). The mixture was immersed in a room temperature oil bath and slowly heated to reflux. Carbon dioxide gas evolution was evident. After stirring at reflux for 2 hours, the reaction was cooled to room temperature and inorganics were filtered off with dioxane. The filtrate was concentrated to dryness to a brown solid. The crude product was dissolved in a minimum of hot 95% EtOH and with cooling, crystals formed. The crystals were filtered off and rinsed with a minimum of ice cold 95% EtOH to give a tan solid (1.0 g, 39%). ¹H-NMR (400 MHz, CDCl₃) δ 7.66 (br s, 1H), 7.50 (dd, 1H), 7.44 (dd, 1H), 7.26 (m, 1H), 7.17 (m, 1H), 6.95 (m, 1H), 6.71 (m, 2H), 5.50 (br s, 2H), 3.89 (s, 3H).

Step B: Preparation of N-(2-aminobenzyl)-3-fluoro-4-methoxyaniline: To a stirred suspension of lithium aluminum hydride (121 mg, 3.2 mmol) in 2 mL dioxane at reflux under nitrogen was added 2-amino-N-(3-fluoro-4-methoxyphenyl)benzamide (260 mg, 1 mmol) as a solution in 2 mL dioxane. A vigorous reaction was evident. After refluxing overnight the reaction was cooled to room temperature and quenched by sequential treatment with H₂O (150 uL), 15% NaOH (150 uL) and H₂O (450 uL). After stirring for several minutes, the heterogeneous mixture was filtered through GF/F filter paper with dioxane and concentrated to a brown residue (246 mg, 100%). ¹H-NMR (400 MHz, CDCl₃) δ 7.14 (m, 2H), 6.86 (m, 1H), 6.74 (m, 2H), 6.60 (dd, 1H), 6.42 (dd, 1H), 4.15 (d, 2H), 4.12 (br s, 2H), 3.83 (s, 3H), 3.54 (br s, 1H).

Step C: Preparation of 3-(3-fluoro-4-methoxyphenyl)-3,4-dihydroquinazolin-2(1H)-one: To a stirred suspension of crude N-(2-aminobenzyl)-3-fluoro-4-methoxyaniline (246 mg, 1 mmol) in 10 mL toluene at 0° C. under a drying tube was added phosgene solution (20% in toluene, 683 uL, 1.30 mmol). Immediately a bright orange color appeared. The cooling bath was removed and the reaction allowed to warm to room temperature over 30 minutes. The solution was then warmed to reflux. After 1 hour, the reaction was concentrated to dryness and the residue dissolved in a minimum of hot 95% EtOH. A precipitate formed which was isolated by filtration with 95% EtOH and dried to give a tan solid (65 mg, 24%). ¹H-NMR (400 MHz, CDCl₃) δ 7.23 (m, 1H), 7.14 (m, 1H), 7.08 (m, 2H), 7.01 (m, 2H), 6.81 (d, 1H), 4.80 (s, 2H), 3.91 (s, 3H).

Step D: Preparation of 3-(3-fluoro-4-hydroxyphenyl)-3,4-dihydroquinazolin-2(1H)-one: To a stirred solution of 3-(3-fluoro-4-methoxyphenyl)-3,4-dihydroquinazolin-2(1H)-one (60 mg, 0.22 mmol) in 2.2 mL dichloromethane at 0° C. under a drying tube was added boron tribromide (104 uL, 1.1 mmol) neat by syringe. The solution turned yellow. After 5 minutes, the reaction was quenched by pouring into saturated NaHCO₃ (30 mL) with stirring. 9/1 Dichloromethane/methanol (30 mL) was added and the mixture stirred rapidly. The layers were separated and the organics were dried (MgSO₄), filtered, and concentrated to a white solid (40 mg, 70%). LRMS (APCI pos) m/e 259 (M+1). ¹H-NMR (400 MHz, CDCl₃) δ7.40 (m, 1H), 7.22 (m, 1H), 7.07 (m, 2H), 6.98 (m, 2H), 6.83 (m, 1H), 4.78 (s, 2H).

Step E: Preparation of 3-(3-fluoro-4-(6-methoxy-7-(3-morpholinopropoxy)quinolin-4-yloxy)phenyl)-3,4-dihydroquinazolin-2(1H)-one. Prepared according to the procedure of Example 1, step E substituting 3-(3-fluoro-4-hydroxyphenyl)-3,4-dihydroquinazolin-2(1H)-one for 3-(3-fluoro-4-hydroxyphenyl)-5-methyl-6-(2-methylbenzyl)pyrimidin-4(3H)-one to give 145 after silica gel chromatography as a white foam (23 mg, 53% yield). ¹H-NMR (400 MHz, CDCl₃) δ 8.51 (d, 1H), 7.57 (s, 1H), 7.45 (s, 1H), 7.39 (m, 1H), 7.32-7.24 (m, 5H), 7.14 (d, 1H), 7.04 (m, 1H), 6.80 (d, 1H), 6.49 (d, 1H), 4.89 (s, 2H), 4.28 (m, 2H), 4.05 (s, 3H), 3.73 (m, 4H), 2.58 (m, 2H), 2.49 (m, 4H), 2.14 (m, 2H). LRMS (apcipos) m/e 559 (M+1).

### Example 46

### Preparation of (4-(4-(6,7-dimethoxyquinolin-4-yloxy)-3-fluorophenyl)piperazin-1-yl)(phenyl)methanone 146

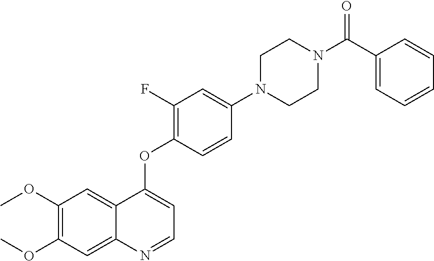

Step A: Preparation of 4-(2-fluoro-4-(piperazin-1-yl)phenoxy)-6,7-dimethoxyquinoline: Pd2(dba)3 (20 mg) was added into a suspension of 4-(4-bromo-2-fluorophenoxy)-6,7-dimethoxyquinoline (Example 34, 100 mg, 0.264 mmol), piperazine (228 mg, 2.64 mmol), Xanthphos (100 mg) and potassium phosphate (200 mg) in toluene (10 mL). The reaction mixture was heated to 100° C. for 10 hours. The reaction mixture was filtered through a pad of silica gel. The filtrate was concentrated and the residue was purified by silica gel flash column chromatography (1:1 MeOH/EtOAc) to yield the product (41 mg, 41% yield) as a brown solid. LRMS (ESI pos) m/e 384 (M+1).

Step B: Preparation of (4-(4-(6,7-dimethoxyquinolin-4-yloxy)-3-fluorophenyl)piperazin-1-yl)(phenyl)methanone: Benzoyl chloride (11 mg, 0.078 mmol) and triethylamine (0.5 mL) were added into a solution of 4-(2-fluoro-4-(piperazin-1-yl)phenoxy)-6,7-dimethoxyquinoline (30 mg, 0.078 mmol) in CH₂Cl₂. After a few minutes, water (1 mL) and CH₂Cl₂ (2 mL) were added. The reaction mixture was extracted with ethyl acetate, and the organic layer was washed with brine, dried over sodium sulfate, filtered, and concentrated. The residue was purified by silica gel flash column chromatography (1:10 MeOH/EtOAc) to yield 146 (5 mg, 13% yield) as a brown solid. ¹H NMR LRMS (ESI pos) m/e 488 (M+1).

### Example 47

### Preparation of 4-(2-fluoro-4-(4-(phenylsulfonyl)piperazin-1-yl)phenoxy)-6,7-dimethoxyquinoline 147

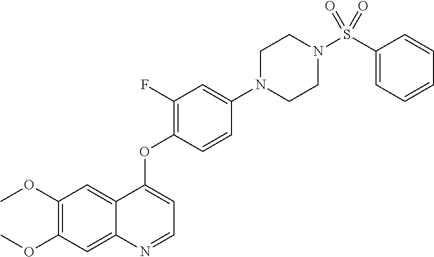

Prepared from benzenesulfonyl chloride (14 mg, 0.078 mmol) and 4-(2-fluoro-4-(piperazin-1-yl)phenoxy)-6,7-dimethoxyquinoline (Example 46, step A, 30 mg, 0.078 mmol) according to the procedure described for Example 46, Step B. The crude product was purified by silica gel flash column chromatography (1:10 MeOH/EtOAc) to yield 147 (3.2 mg, 7.8% yield) as white solid. ¹H NMR (DMSO-d₆, 400 MHz) δ 8.42 (d, 1H), 7.74-7.84 (m, 2H), 7.65-7.74 (m, 2H), 7.50 (s, 1H), 7.39 (s, 1H), 7.25-7.32 (m, 1H), 7.00-7.08 (m, 1H), 6.80-6.88 (m, 1H), 6.35 (d, 1H), 5.75 (s, 1H), 3.95 (s, 6H), 3.00-3.08 (m, 4H). LRMS (ESI pos) m/e 524 (M+1).

### Example 48

### Preparation of 3-benzyl-5-(4-(6,7-dimethoxyquinolin-4-yloxy)-3-fluorophenyl)pyrimidine-4(3H)-thione 148

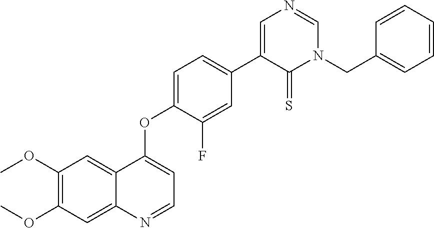

Step A: Preparation of 3-benzyl-5-(4-(benzyloxy)-3-fluorophenyl)pyrimidine-4(3H)-thione: Lawesson's reagent (262 mg, 0.647 mmol) was added into a suspension of 3-benzyl-5-(4-(benzyloxy)-3-fluorophenyl)pyrimidin-4(3H)-one (Example 7, Step A, 100 mg, 0.26 mmol) in toluene. The reaction was heated at 120° C. for 12 hours. LCMS indicated the reaction was complete. The reaction mixture was concentrated and the crude product was purified by silica gel flash column chromatography (EtOAc) to yield the product (70 mg, 67%) as a brown solid. LRMS (ESI pos) m/e 403 (M+1).

Step B: 3-benzyl-5-(3-fluoro-4-hydroxyphenyl)pyrimidine-4(3H)-thione: Prepared from 3-benzyl-5-(4-(benzyloxy)-3-fluorophenyl)pyrimidine-4(3H)-thione (70 mg, 0.17 mmol) according to the procedure described for Example 1, Step C to yield the product (50 mg, 92%) as brown solid. LRMS (ESI pos) m/e 313 (M+1).

Step C: Preparation of 3-benzyl-5-(4-(6,7-dimethoxyquinolin-4-yloxy)-3-fluorophenyl)pyrimidine-4(3H)-thione: Prepared from 4-chloro-6,7-dimethoxyquinoline (prepared according to reference procedure in Example 5) (40 mg, 0.18 mmol) and 3-(4-chlorobenzyl)-5-(3-fluoro-4-hydroxyphenyl)pyrimidine-4(3H)-thione (62 mg, 0.18 mmol) according to the procedure described for Example 1, Step E to yield 148 (40 mg, 45%) as yellow solid. ¹H NMR (DMSO-d₆, 400 MHz) δ 9.08 (s, 1H), 8.54 (d, 1H), 8.11 (s, 1H), 7.70 (d, 1H), 7.54 (s, 1H), 7.47-7.50 (m, 2H), 7.43 (s, 1H), 7.35-7.40 (m, 4H), 7.26-7.33 (m, 1H), 6.53 (d, 1H), 5.76 (s, 2H), 3.96 (s, 3H), 3.96 (s, 3H). LRMS (ESI pos) m/e 500 (M+1).

### Example 49

### Preparation of 3-benzyl-5-(4-(7-(benzyloxy)-6-methoxyquinolin-4-yloxy)-3-fluorophenyl)pyrimidin-4(3H)-one 149

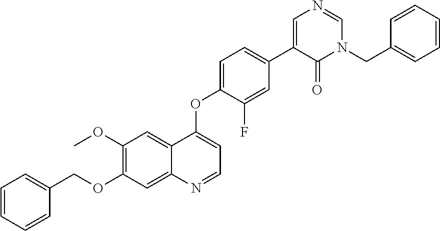

Step A: Preparation of 3-benzyl-5-(4-(benzyloxy)-3-fluorophenyl)pyrimidin-4(3H)-one: Tetrakis(triphenylphosphine)palladium(0) (0.65 g, 0.57 mmol) was added into a suspension of 3-benzyl-5-bromopyrimidin-4(3H)-one (prepared according to Gurnos Jones described in Journal of the Chemical Society, Perkin Transactions 1: Organic and Bio-Organic Chemistry (1972-1999) 1983, 11:2645-8, 3.0 g, 11 mmol), 4-benzyloxy-3-fluorobenzeneboronic acid (3.3 g, 14 mmol) and lithium chloride (2.4 g, 57 mmol) in dioxane (100 mL) and 2M aqueous sodium carbonate solution (50 mL). The reaction mixture was heated at 100° C. for 2 hours, cooled and poured into water (10 mL). The reaction mixture was extracted with ethyl acetate, and the organic layer was washed with brine, dried over sodium sulfate, filtered, and concentrated. The crude product was purified by silica gel flash column chromatography (2:1 EtOAc/Hexane) to yield the product (1.4 g, 32%) as a white solid. ¹H NMR (CDCl₃, 400 MHz) δ 8.15 (s, 1H), 8.01 (s, 1H), 7.53 (dd, J=12.5, 2.34 Hz, 1H), 7.43-7.47 (m, 2H), 7.30-7.42 (m, 9H), 7.00-7.05 (m, 1H), 5.18 (s, 2H), 5.17 (s, 2H). LRMS (ESI pos) m/e 387 (M+1).

Step B: Preparation of 3-benzyl-5-(3-fluoro-4-hydroxyphenyl)pyrimidin-4(3H)-one: Prepared from 3-benzyl-5-(4-(benzyloxy)-3-fluorophenyl)pyrimidin-4(3H)-one (0.3 g, 0.8 mmol) according to the procedure described for Example 1, Step C, to yield the product (0.2 g, 87%) as a white solid. LRMS (ESI pos) m/e 297 (M+1).

Step C: Preparation of 3-benzyl-5-(4-(7-(benzyloxy)-6-methoxyquinolin-4-yloxy)-3-fluorophenyl)pyrimidin-4(3H)-one: Prepared from 7-(benzyloxy)-4-chloro-6-methoxyquinoline (prepared according to WO 2005/030140, Example 32, 200 mg, 0.67 mmol) and 3-benzyl-5-(3-fluoro-4-hydroxyphenyl)pyrimidin-4(3H)-one (198 mg, 0.48 mmol) according to the procedure described for Example 1, Step E. The crude product was purified by silica gel flash column chromatography (1:10 MeOH/EtOAc) to yield 149 (100 mg, 27%) as a white solid. LRMS (ESI pos) m/e 560 (M+1).

### Example 50

### Preparation of 3-benzyl-5-(3-fluoro-4-(7-hydroxy-6-methoxyquinolin-4-yloxy)phenyl)pyrimidin-4(3H)-one 150

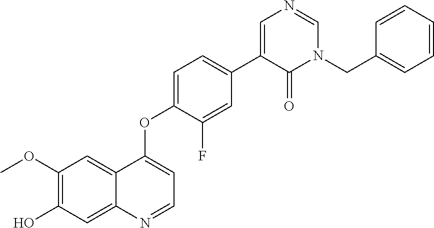

Prepared from 3-benzyl-5-(4-(7-(benzyloxy)-6-methoxyquinolin-4-yloxy)-3-fluorophenyl)pyrimidin-4(3H)-one (90 mg, 0.16 mmol) according to the procedure described for Example 1, Step C, to yield 150 (50 mg, 66%) as a white solid. LRMS (ESI pos) m/e 470 (M+1).

### Example 51

### Preparation of 3-(4-tert-butylbenzyl)-5-(4-(6,7-dimethoxyquinolin-4-yloxy)-3-fluorophenyl)pyrimidin-4(3H)-one 151

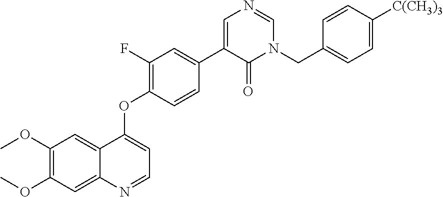

Step A: Preparation of 5-bromo-3-(4-tert-butylbenzyl)pyrimidin-4(3H)-one: Prepared from 1-(bromomethyl)-4-tert-butylbenzene (1.9 g, 8.4 mmol) according to the procedure described for Example 13, Step A. The crude product was purified by silica gel flash column chromatography (1:1 EtOAc/Hexane) to yield the product (0.65 g, 24%) as a white solid. LRMS (ESI pos) m/e 323 (M+1).

Step B: Preparation of 5-(4-(benzyloxy)-3-fluorophenyl)-3-(4-tert-butylbenzyl)pyrimidin-4(3H)-one: Prepared from 5-bromo-3-(4-tert-butylbenzyl)pyrimidin-4(3H)-one (0.65 mg, 2.0 mmol) according to the procedure described for Example 7, Step A. The crude product was purified by silica gel flash column chromatography (1:1 EtOAc/Hexane) to yield the product (0.50 g, 56%) as a white solid. LRMS (ESI pos) m/e 443 (M+1).

Step C: Preparation of 3-(4-tert-butylbenzyl)-5-(3-fluoro-4-hydroxyphenyl)pyrimidin-4(3H)-one: Prepared from 5-(4-(benzyloxy)-3-fluorophenyl)-3-(4-tert-butylbenzyl)pyrimidin-4(3H)-one (0.50 g, 1.1 mmol) according to the procedure described for Example 1, Step C, to yield the product (0.30 g, 88%) as a white solid. LRMS (ESI pos) m/e 353 (M+1).

Step D: Preparation of 3-(4-tert-butylbenzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (100 mg, 0.46 mmol) and 3-(4-tert-butylbenzyl)-5-(3-fluoro-4-hydroxyphenyl)pyrimidin-4(3H)-one (130 mg, 0.30 mmol) according to the procedure described for Example 1, Step E. The crude product was purified by silica gel flash column chromatography (1:10 MeOH/EtOAc) to yield 151 (48 mg, 30%) as a white solid. ¹H NMR (DMSO-d₆, 400 MHz) δ 8.79 (s, 1H), 8.50 (d, 1H), 8.35 (s, 1H), 7.92 (dd, 1H), 7.72-7.76 (m, 1H), 7.54 (s, 1H), 7.48-7.52 (m, 1H), 7.42 (s, 1H), 7.36-7.40 (m, 2H), 7.30-7.34 (m, 2H), 6.52 (d, 1H), 5.18 (s, 2H), 3.96 (s, 3H), 3.95 (s, 3H). LRMS (ESI pos) m/e 540 (M+1).

### Example 52

### Preparation of 3-(4-chloro-3-(trifluoromethyl)benzyl)-5-(4-(6,7-dimethoxyquinolin-4-yloxy)-3-fluorophenyl)pyrimidin-4(3H)-one 152

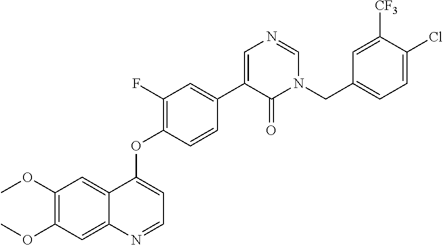

Step A: Preparation of 5-bromo-3-(4-chloro-3-(trifluoromethyl)benzyl)pyrimidin-4(3H)-one: Prepared from 4-(bromomethyl)-1-chloro-2-(trifluoromethyl)benzene (0.76 g, 2.8 mmol) according to the procedure described for Example 13, Step A. The crude product was purified by silica gel flash column chromatography (1:1 EtOAc/Hexane) to yield the product (0.41 g, 39%) as a white solid. LRMS (ESI pos) m/e 369 (M+1).

Step B: Preparation of 5-(4-(benzyloxy)-3-fluorophenyl)-3-(4-chloro-3-(trifluoromethyl)benzyl)pyrimidin-4(3H)-one: Prepared from 5-bromo-3-(4-chloro-3-fluorobenzyl)pyrimidin-4(3H)-one (0.41 g, 1.1 mmol) according to the procedure described for Example 7, Step A. The crude product was purified by silica gel flash column chromatography (1:1 EtOAc/Hexane) to yield the product (0.50 g, 92%) as a white solid. LRMS (ESI pos) m/e 489 (M+1).

Step C: Preparation of 3-(4-chloro-3-(trifluoromethyl)benzyl)-5-(3-fluoro-4-hydroxyphenyl)pyrimidin-4(3H)-one: Prepared from 5-(4-(benzyloxy)-3-fluorophenyl)-3-(4-chloro-3-(trifluoromethyl)benzyl)pyrimidin-4(3H)-one (0.50 g, 1.0 mmol) according to the procedure described for Example 1, Step C, to yield the product (0.40 g, 86%) as a white solid. LRMS (ESI pos) m/e 331 (M+1).

Step D: Preparation of 3-(4-chloro-3-(trifluoromethyl)benzyl)-5-(4-(6,7-dimethoxyquinolin-4-yloxy)-3-fluorophenyl)pyrimidin-4(3H)-one: Prepared from 4-chloro-6,7-dimethoxyquinoline (prepared according to reference procedure in Example 5) (60 mg, 0.27 mmol) and 3-(4-chloro-3-(trifluoromethyl)phenyl)-5-(3-fluoro-4-hydroxyphenyl)pyrimidin-4(3H)-one (107 mg, 0.27 mmol) according to the procedure described for Example 1, Step E. The crude product was purified by silica gel flash column chromatography (1:10 MeOH/EtOAc) to yield 152 (12 mg, 7%) as a white solid. ¹H NMR (DMSO-d₆, 400 MHz) δ 8.80 (s, 1H), 8.40 (s, 1H), 8.30 (s, 1H), 7.92 (s, 1H), 7.80-7.90 (m, 1H), 7.60-7.75 (m, 3H), 7.40-7.50 (m, 2H), 7.30-7.40 (m, 1H), 6.40-6.50 (m, 1 h), 5.20 (s, 2H), 3.96 (s, 6H). LRMS (ESI pos) m/e 586 (M+1).

### Example 53

### Preparation of (4-benzylpiperazin-1-yl)(4-(6,7-dimethoxyquinolin-4-yloxy)-3-fluorophenyl)methanone 153

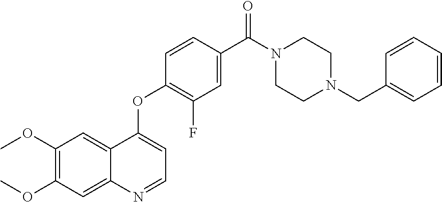

Step A: Preparation of (4-benzylpiperazin-1-yl)(3-fluoro-4-methoxyphenyl)methanone: Prepared from 1-benzylpiperazine (2.3 mg, 13 mmol)) according to the procedure described for Example 4, Step A, to yield the product (2.6 g, 65%) as a white solid. LRMS (ESI pos) m/e 329 (M+1).

Step B: Preparation of (4-benzylpiperazin-1-yl)(3-fluoro-4-hydroxyphenyl)methanone: Prepared from (4-benzylpiperazin-1-yl)(3-fluoro-4-methoxyphenyl)methanone (2.0 g, 6.1 mmol) according to the procedure described for Example 4, Step B, to yield the product (1.0 g, 53%) as a white solid. LRMS (ESI pos) m/e 315 (M+1).

Step C: Preparation of 5 (4-benzylpiperazin-1-yl)(4-(6,7-dimethoxyquinolin-4-yloxy)-3-fluorophenyl)methanone: Prepared from (4-benzylpiperazin-1-yl)(3-fluoro-4-hydroxyphenyl)methanone (18 mg, 0.059 mmol) and 4-chloro-6-methoxy-7-(3-morpholinopropoxy)quinoline (20 mg, 0.059 mmol) according to the procedure described for Example 1, Step E, to yield 153 (40 mg, 18% yield) as a white solid. ¹H NMR (DMSO-d₆, 400 MHz) δ 8.50 (d, 1H), 7.48-7.56 (m, 3H), 7.42 (s, 1H), 7.24-7.36 (m, 6H), 6.58 (d, 1H), 3.96 (s, 3H), 3.95 (s, 3H), 3.60-3.70 (m, 2H), 3.30-3.50 (m, 2H), 2.35-2.45 (m, 4H). LRMS (ESI pos) m/e 502 (M+1).

### Example 54

### Preparation of 6-benzyl-3-(4-(6-methoxy-7-(3-morpholinopropoxy)quinolin-4-yloxy)phenyl)pyrimidin-4(3H)-one 154

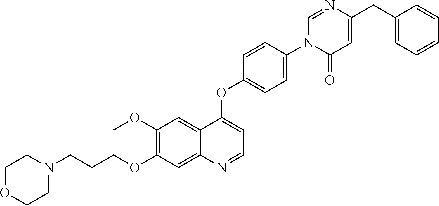

Step A: Preparation of (E)-ethyl 3-amino-2-(4-(benzyloxy)phenylcarbamoyl)-4-phenylbut-2-enoate: A suspension of (E)-ethyl 3-amino-4-phenylbut-2-enoate (1 g, 4.9 mmol) and 1-((4-isocyanatophenoxy)methyl)benzene (1.1 g, 4.9 mmol) was heated in DMF (20 mL) at 60° C. for 72 hours. The reaction mixture was poured into water (10 mL) and removed the solid by filtration. The filtrate was evaporated and the residue was purified by silica gel flash column chromatography (1:4 EtOAc/hexane) to yield the product (1.8 g, 86%) as a yellow solid. ¹H NMR (CDCl₃, 400 MHz) δ 10.90 (s, 1H), 7.20-7.42 (m, 14H), 6.90-6.95 (m, 2H), 5.04 (s, 2H), 4.24 (q, 2H), 4.15 (s, 2H), 1.29 (t, 3H).

Step B: Preparation of ethyl 4-benzyl-1-(4-(benzyloxy)phenyl)-6-oxo-1,6-dihydropyrimidine-5-carboxylate: Acetic anhydride (1 ml, 10.6 mmol) was added into a solution of (E)-ethyl 3-amino-2-((4-(benzyloxy)phenyl)carbamoyl)-4-phenylbut-2-enoate (1 g, 2.32 mmol) and triethyl orthoformate (5 ml, 30.1 mmol). The reaction was heated at 110° C. for 16 hours, cooled and solvent was evaporated. The residue was purified by silica gel flash column chromatography (1:2 EtOAc/hexane) to yield the product (0.9 g, 88%) as a yellow solid. ¹H NMR (CDCl₃, 400 MHz) δ 8.10 (s, 1H), 7.30-7.42 (m, 10H), 7.20-7.28 (m, 2H), 3.96 (s, 2H), 1.36 (q, 3H).

Step C: Preparation of 6-benzyl-3-(4-hydroxyphenyl)pyrimidin-4(3H)-one: Ethyl 4-benzyl-1-(4-(benzyloxy)phenyl)-6-oxo-1,6-dihydropyrimidine-5-carboxylate (0.87 g, 1.98 mmol) was added into concentrated HCl (20 mL) and acetic acid (20 mL) and stirred for 3 hours. The reaction mixture was extracted with CH₂Cl₂, and the organic layer was washed with brine, dried over sodium sulfate, filtered, and concentrated. The residue was purified by silica gel flash column chromatography (1:10 Et₂O/Hexane) to yield the product (0.2 g, 36% yield) as a yellow solid. ¹H NMR (CDCl₃, 400 MHz) δ 8.10 (s, 1H), 7.25-7.40 (m, 5H), 7.04-7.14 (m, 2H), 6.79-6.85 (m, 2H), 6.33 (s, 1H), 3.91 (s, 2H). LRMS (ESI pos) m/e 279 (M+1).

Step D: Preparation of 6-benzyl-3-(4-(6-methoxy-7-(3-morpholinopropoxy)quinolin-4-yloxy)phenyl)pyrimidin-4(3H)-one: Prepared from 4-chloro-6-methoxy-7-(3-morpholinopropoxy)quinoline (34 mg, 0.10 mmol) and 6-benzyl-3-(4-hydroxyphenyl)pyrimidin-4(3H)-one (28 mg, 0.10 mmol) according to the procedure described for Example 1, Step E. The residue was purified by silica gel flash column chromatography column (1:10 MeOH/EtOAc) to yield 154 (20 mg, 34%) as a yellow solid. ¹H NMR (CDCl₃, 400 MHz) δ 8.55 (d, 1H), 8.14 (s, 1H), 7.46 (d, 1H), 7.40-7.45 (m, 1H), 7.35-7.46 (m, 1H), 7.24-7.34 (m, 8H), 6.63 (d, 2H), 6.35 (s, 1H), 4.25-4.36 (m, 2H), 4.01 (s, 3H), 3.92 (s, 2H), 3.70-3.75 (m, 4H), 2.55-2.66 (m, 2H), 2.45-2.50 (m, 4H), 2.10-2.15 (m, 2H). LRMS (ESI pos) m/e 579 (M+1).

### Example 55

### Preparation of 5-benzyl-3-(3-fluoro-4-(6-methoxy-7-(3-morpholinopropoxy)quinolin-4-yloxy)phenyl)pyrimidin-4(3H)-one 155

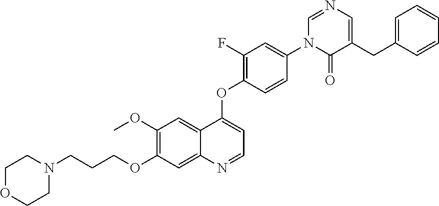

Step A: preparation of (4,6-dichloropyrimidin-5-yl)(phenyl)methanol: Phenylmagnesium bromide (11 ml, 11 mmol) was added into a solution of 4,6-Dichloro-5-pyrimidinecarbaldehyde (2 g, 11 mmol) in THF (30 mL) at −78° C. The reaction was allowed to warm to room temperature and was poured into water (10 mL). The reaction mixture was extracted with EtOAc, and the organic layer was washed with brine, dried over sodium sulfate, filtered, and concentrated. The residue was purified by silica gel flash column chromatography (1:10 Et₂O/Hexane) to yield the product (2.4 g, 83% yield) as a white solid. ¹H NMR (CDCl₃, 400 MHz) δ 8.76 (s, 1H), 7.20-7.41 (m, 5H), 6.50 (d, 1H), 3.00 (d, 1H).

Step B: Preparation of (4-(benzyloxy)-6-chloropyrimidin-5-yl)(phenyl)methanol: KOH (0.44 g, 7.8 mmol) was added into a solution of (4,6-dichloropyrimidin-5-yl)(phenyl)methanol (1.0 g, 3.9 mmol), benzyl alcohol (0.41 ml, 3.9 mmol) and 18-crown-6 (0.21 g, 0.78 mmol) in toluene (50 mL). The solution was heated to reflux for 2 hours. The reaction was allowed to warm to room temperature and was poured into water (10 mL). The reaction mixture was extracted with EtOAc, and the organic layer was washed with brine, dried over sodium sulfate, filtered, and concentrated. The residue was purified by silica gel flash column chromatography (1:10 Et2O/Hexane) to yield the product (0.5 g, 39% yield) as a white solid. LRMS (ESI pos) m/e 327 (M+1).

Step C: Preparation of 5-benzylpyrimidin-4-ol: Prepared from 5-benzylpyrimidin-4-ol (0.5 g, 1.5 mmol) according to the procedure described for Example 14, Step B, to yield the product (0.17 g, 60%) as a white solid. LRMS (ESI pos) m/e 187 (M+1).

Step D: Preparation of 5-benzyl-3-(3-fluoro-4-hydroxyphenyl)pyrimidin-4(3H)-one: Prepared from 5-benzylpyrimidin-4-ol (0.1 g, 0.54 mmol) according to the procedure described for Example 1, Step D, to yield the product (20 mg, 13% yield) as a brown solid. LRMS (ESI pos) m/e 279 (M+1).

Step E: Preparation of 5-benzyl-3-(3-fluoro-4-(6-methoxy-7-(3-morpholinopropoxy)quinolin-4-yloxy)phenyl)pyrimidin-4(3H)-one: Prepared from 5-benzyl-3-(3-fluoro-4-hydroxyphenyl)pyrimidin-4(3H)-one (18 mg, 0.059 mmol) according to the procedure described for Example 1, Step E, to yield 155 (1 mg, 2.8% yield) as a light brown solid. LRMS (ESI pos) m/e 597 (M+1).

### Example 56

### Preparation of 2-benzyl-5-(3-fluoro-4-(6-methoxy-7-(3-morpholinopropoxy)quinolin-4-yloxy)phenyl)pyrimidin-4(3H)-one 156

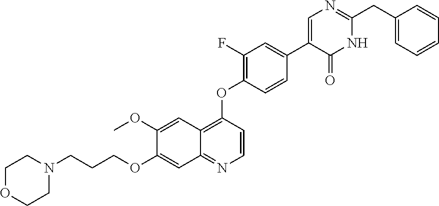

Step A: Preparation of 2-benzyl-4-methoxypyrimidine: A solution of 2-chloro-4-methoxypyrimidine (0.500 g, 3.46 mmol) and PdCl₂(PPh₃)₂ (0.121 g, 0.173 mmol) in THF (10 mL) was sparged with N₂. Benzylzinc(II) bromide (8.30 ml, 4.15 mmol; 0.5 M solution in THF) was added and the reaction mixture was stirred at reflux for 1 hour. The reaction mixture was cooled to room temperature and then partitioned between EtOAc and H₂O. The phases were separated, and the aqueous phase was re-extracted with EtOAc (1×). The combined organic layers were dried (Na₂SO₄), filtered and concentrated to yield a crude dark brown oil. The crude product was purified by silica gel flash column chromatography, eluting with 20:1 dichloromethane/EtOAc. The desired product (0.676 g, 98%) was obtained as a dark yellow oil. ¹H-NMR (400 MHz, CDCl₃) δ 8.34 (d, 1H), 7.41-7.25 (m, 4H), 7.21 (m, 1H), 6.53 (d, 1H), 4.16 (s, 2H), 3.95 (s, 3H). LRMS (ESI pos) m/e 201 (M+1).

Step B: Preparation of 2-benzylpyrimidin-4(3H)-one: To a solution of 2-benzyl-4-methoxypyrimidine (0.675 g, 3.37 mmol) in AcOH (15 mL) was added HBr (2.28 ml, 20.2 mmol; 48 wt % in H₂O). The reaction mixture was stirred at 95° C. for 2 hours. The reaction mixture was cooled to room temperature and diluted with H₂O. The pH of the reaction mixture was adjusted to 5-6 with 6 M aqueous NaOH and then partitioned between EtOAc and H₂O. The phases were separated, and the aqueous phase was re-extracted with EtOAc (1×). The combined organic layers were dried (Na₂SO₄), filtered and concentrated to yield a crude yellow solid. Purification of the crude product was achieved by trituration with dichloromethane and diethyl ether. The resulting solid was filtered, washed with diethyl ether, collected and dried under vacuum to yield the desired product (0.531 g, 85%) as an off-white solid. ¹H-NMR (400 MHz, DMSO-d₆) δ 7.79 (d, 1H), 7.31 (m, 4H), 7.23 (m, 1H), 6.10 (d, 1H), 3.83 (s, 2H). LRMS (ESI pos) m/e 187 (M+1).

Step C: Preparation of 2-benzyl-5-bromopyrimidin-4(3H)-one: To a solution of 2-benzylpyrimidin-4(3H)-one (0.531 g, 2.85 mmol) in CHCl₃ (15 mL) and methanol (3 mL) was added bromine (0.146 ml, 2.85 mmol). The reaction mixture was stirred at room temperature for 3 hours and then quenched with 10% sodium bisulfate solution. The reaction mixture was partitioned between EtOAc and H₂O. The phases were separated, and the aqueous phase was re-extracted with EtOAc (1×). The combined organic layers were dried (Na₂SO₄), filtered and concentrated to yield a crude yellow solid. Purification of the crude product was achieved by trituration with dichloromethane. The resulting solid was filtered, washed with dichloromethane, collected and dried under vacuum to yield the desired product (0.302 g, 40%) as an off-white solid. ¹H-NMR (400 MHz, DMSO-d₆) δ 13.23 (br s, 1H), 8.25 (s, 1H), 7.35-7.28 (m, 4H), 7.27-7.22 (m, 1H), 3.87 (s, 2H). LRMS (ESI pos) m/e 265, 267 (M+, Br pattern).

Step D: Preparation of 2-benzyl-5-(4-(benzyloxy)-3-fluorophenyl)pyrimidin-4(3H)-one: A solution of 2-benzyl-5-bromopyrimidin-4(3H)-one (0.300 g, 1.13 mmol), 4-(benzyloxy)-3-fluorophenylboronic acid (0.334 g, 1.36 mmol), Pd(PPh₃)₄ (0.065 g, 0.057 mmol) and lithium chloride (0.240 g, 5.66 mmol) in dioxane (3 mL) and 2 M aqueous Na₂CO₃ (0.3 mL) was stirred at 100° C. for 18 hours. After cooling, the reaction mixture was partitioned between EtOAc and H₂O. The phases were separated, and the aqueous phase was re-extracted with EtOAc (3×). The combined organic layers were dried (Na₂SO₄), filtered and concentrated to yield a crude dark yellow solid. Purification of the crude product was achieved by trituration with dichloromethane. The resulting solid was filtered, washed with dichloromethane, collected and dried under vacuum. The filtrate was concentrated and the trituration procedure was repeated (2×) with EtOAc. The solids were combined to yield the desired product (0.284 g, 65%) as a pale yellow solid. ¹H-NMR (400 MHz, DMSO-d₆) δ 12.93 (br s, 1H), 8.13 (s, 1H), 7.66 (dd, 1H), 7.53-7.22 (s, 12H), 5.21 (s, 2H), 3.90 (s, 2H). LRMS (APCI pos) m/e 387 (M+1).

Step E: Preparation of 2-benzyl-5-(3-fluoro-4-hydroxyphenyl)pyrimidin-4(3H)-one: A suspension of 2-benzyl-5-(4-(benzyloxy)-3-fluorophenyl)pyrimidin-4(3H)-one (0.284 g, 0.735 mmol) in trifluoroacetic acid (3 mL) was stirred at 40° C. for 2 hours and then at room temperature for 16 hours. The reaction mixture was concentrated to dryness and then purified by silica gel flash column chromatography, eluting with 20:1 dichloromethane/MeOH. The desired product (0.177 g, 81%) was obtained as a white solid. ¹H-NMR (400 MHz, DMSO-d₆) δ 12.89 (br s, 1H), 9.98 (s, 1H), 8.08 (s, 1H), 7.58 (dd, 1H), 7.39-7.30 (m, 5H), 7.28-7.22 (m, 1H), 6.96 (dd, 1H), 3.89 (s, 2H). LRMS (ESI pos) m/e 297 (M+1).

Step F: Preparation of 2-benzyl-5-(3-fluoro-4-(6-methoxy-7-(3-morpholinopropoxy)quinolin-4-yloxy)phenyl)pyrimidin-4(3H)-one: To a solution of 2-benzyl-5-(3-fluoro-4-hydroxyphenyl)pyrimidin-4(3H)-one (0.027 g; 0.091 mmol) in toluene (500 uL) in a microwave tube was added 4-(3-(4-chloro-6-methoxyquinolin-7-yloxy)propyl)morpholine (prepared according to the reference in Example 1, step E) (0.031 g, 0.091 mmol) and DMAP (0.011 g, 0.091 mmol). The reaction was stirred at 180° C. in the microwave for 2 hours. The mixture was solubilized with a small amount of MeOH and purified by silica gel flash column chromatography, eluting with 9:1 EtOAc/MeOH. The desired product eluted along with residual 4-(3-(4-chloro-6-methoxyquinolin-7-yloxy)propyl)morpholine. The mixture was concentrated and repurified by silica gel flash column chromatography, eluting with a step gradient of CH₂Cl₂ (150 mL), 2.5/97.5 MeOH/CH₂Cl₂ (200 mL) and 5/95 MeOH/CH₂Cl₂ (500 mL) to give 156 (0.022 g, 40%) as a tan foamy solid. ¹H-NMR (400 MHz, CDCl₃) δ 8.50 (d, 1H), 8.22 (s, 1H), 7.77 (dd, 1H), 7.57 (s, 1H), 7.55 (d, 1H), 7.45 (s, 1H), 7.42-7.28 (m, 6H), 6.50 (m, 1H), 4.28 (t, 2H), 4.08 (s, 2H), 4.04 (s, 3H), 3.73 (t, 4H), 2.58 (t, 2H), 2.49 (m, 4H), 2.14 (m, 2H). LRMS (APCI pos) m/e 597 (M+1).

### Example 57

### Preparation of 2-benzyl-5-(4-(6,7-dimethoxyquinolin-4-yloxy)-3-fluorophenyl)pyrimidin-4(3H)-one 157

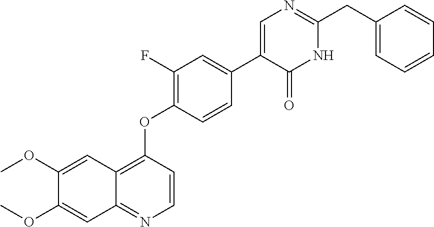

Prepared according to the method of Example 56, step F, substituting 4-chloro-6,7-dimethoxyquinoline (reference for preparation given in Example 5)(6.8 mg, 0.03 mmol) for 4-(3-(4-chloro-6-methoxyquinolin-7-yloxy)propyl)morpholine to provide 157 (3.3 mg, 34%). ¹H-NMR (400 MHz, CDCl₃) δ 8.52 (d, 1H), 8.22 (s, 1H), 7.74 (m, 1H), 7.59 (s, 1H), 7.54 m, 1H), 7.44 (s, 1H), 7.40-7.30 (m, 6H), 6.51 (d, 1H), 4.08 (s, 2H), 4.06 (s, 3H), 4.05 (s, 3H). LRMS (ESI pos) m/e 484 (M+1).

### Example 58

### Preparation of 2-benzyl-5-(4-(6-methoxy-7-(3-morpholinopropoxy)quinolin-4-yloxy)phenyl)pyrimidin-4(3H)-one 158

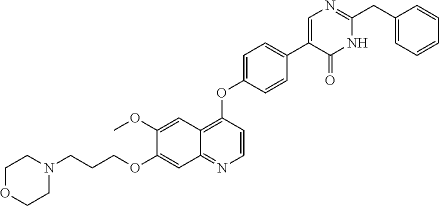

Step A: Preparation of 2-benzyl-5-(4-(benzyloxy)phenyl)pyrimidin-4(3H)-one: Prepared from 2-benzyl-5-bromopyrimidin-4(3H)-one (0.100 g, 0.377 mmol; obtained from Example 56, Step C) according to the procedure described in Step D of Example 56, substituting 4-(benzyloxy)phenylboronic acid in place of 4-(benzyloxy)-3-fluorophenylboronic acid. The desired product (0.116 g, 84%) was obtained as a white solid. ¹H-NMR (400 MHz, DMSO-d₆) δ 12.84 (br s, 1H), 8.04 (s, 1H), 7.64 (m, 2H), 7.45 (m, 2H), 7.42-7.29 (m, 7H), 7.25 (m, 1H), 7.03 (m, 2H), 5.13 (s, 2H), 3.89 (s, 2H). LRMS (APCI pos) m/e 369 (M+1).

Step B: Preparation of 2-benzyl-5-(4-hydroxyphenyl)pyrimidin-4(3H)-one: Prepared from 2-benzyl-5-(4-(benzyloxy)phenyl)pyrimidin-4(3H)-one (0.116 g, 0.315 mmol) according to the procedure described in Step E of Example 56. The desired product (0.042 g, 48%) was obtained as a white solid. ¹H-NMR (400 MHz, DMSO-d₆) δ 9.54 (br s, 1H), 7.99 (s, 1H), 7.52 (m, 2H), 7.37-7.30 (m, 4H), 7.25 (m, 1H), 6.77 (m, 2H), 3.89 (s, 2H). LRMS (ESI pos) m/e 279 (M+1).

Step C: Preparation of 2-benzyl-5-(4-(6-methoxy-7-(3-morpholinopropoxy)quinolin-4-yloxy)phenyl)pyrimidin-4(3H)-one: To a stirred suspension of 2-benzyl-5-(4-hydroxyphenyl)pyrimidin-4(3H)-one (0.021 g, 0.076 mmol) in bromobenzene (700 at room temperature under N₂ was added 4-(3-(4-chloro-6-methoxyquinolin-7-yloxy)propyl)morpholine (prepared according to the referenced procedure in Example 1, step E) (0.028 g, 0.083 mmol) followed by DMAP (0.001 g, 0.008 mmol). The reaction mixture was stirred at 150° C. for 12 hours and then stirred at room temperature for an additional 9 hours. The reaction mixture was directly purified by silica gel flash column chromatography, eluting with 10:1 dichloromethane/MeOH, to give 158 (0.023 g, 53%) as a pale yellow foamy solid. ¹H-NMR (400 MHz, DMSO-d₆) δ 12.99 (br s, 1H), 8.49 (d, 1H), 8.18 (s, 1H), 7.86 (m, 2H), 7.50 (s, 1H), 7.43-7.22 (m, 8H), 6.54 (d, 1H), 4.21 (t, 2H), 3.93 (s, 5H), 3.59 (m, 4H), 2.47 (m, 2H), 2.39 (m, 4H), 1.98 (m, 2H). LRMS (ESI pos) m/e 579 (M+1).

### Example 59

### Preparation of 2-benzyl-5-(3-fluoro-4-(6-methoxy-7-(3-morpholinopropoxy)quinolin-4-yloxy)phenyl)-3-methylpyrimidin-4(3H)-one 159

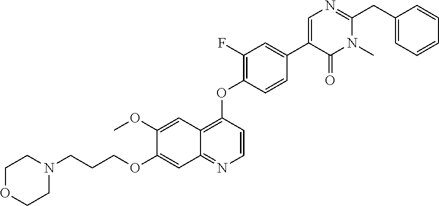

Step A: Preparation of 5-bromo-2-chloropyrimidin-4(3H)-one: Prepared from 5-bromo-2,4-dichloropyrimidine (10.00 g, 43.88 mmol) according to the procedure described in EP 1506967. The desired product (4.59 g, 50%) was obtained as a pale yellow solid. ¹H-NMR (400 MHz, DMSO-d₆) δ 8.36 (s, 1H). LRMS (ESI neg) m/e 207, 209 (M−, Br pattern).

Step B: Preparation of 5-bromo-2-chloro-3-methylpyrimidin-4(3H)-one: To a solution of 5-bromo-2-chloropyrimidin-4(3H)-one (1.00 g, 4.78 mmol) in DME (12 mL)/DMF (3 mL) under N₂ at 0° C. was added LiH (0.044 g, 5.25 mmol) and the reaction was stirred at room temperature for 15 minutes. Iodomethane (0.589 ml, 9.45 mmol) was then added and the reaction was stirred at room temperature for 30 minutes and then at 60° C. for 1.5 hours. The reaction mixture was quenched with H₂O and then partitioned between EtOAc and saturated aqueous NaCl. The phases were separated, and the aqueous phase was re-extracted with EtOAc (1×). The combined organic layers were dried (Na₂SO₄), filtered and concentrated to yield a crude yellow oil. The crude product was purified by silica gel flash column chromatography, eluting with 25:1 dichloromethane/EtOAc. The desired product (0.764 g, 72%) was obtained as a yellow crystalline solid. ¹H-NMR (400 MHz, DMSO-d₆) δ 8.26 (s, 1H), 3.59 (s, 3H). LRMS (ESI pos) m/e 223, 225 (M+, Br pattern).

Step C: Preparation of 2-benzyl-5-bromo-3-methylpyrimidin-4(3H)-one: A solution of 5-bromo-2-chloro-3-methylpyrimidin-4(3H)-one (0.100 g, 0.448 mmol) and PdCl₂(PPh₃)₂ (0.016 g, 0.022 mmol) in THF (4 mL) was sparged with N₂. Benzylzinc(II) bromide (0.904 ml, 0.452 mmol; 0.5 M solution in THF) was added and the reaction mixture was stirred at reflux for 30 minutes. The reaction mixture was cooled to room temperature and then partitioned between EtOAc and H₂O. The phases were separated, and the aqueous phase was re-extracted with EtOAc (1×). The combined organic layers were dried (Na₂SO₄), filtered and concentrated to yield a crude yellow gum. The crude product was purified by silica gel flash column chromatography, eluting with 20:1 dichloromethane/EtOAc. The desired product (0.067 g, 54%) was obtained as a colorless gum. ¹H-NMR (400 MHz, CDCl₃) δ 8.21 (s, 1H), 7.38-7.27 (m, 3H), 7.19 (m, 2H), 4.14 (s, 2H), 3.50 (s, 3H). LRMS (ESI pos) m/e 279, 281 (M+, Br pattern).

Step D. Preparation of 2-benzyl-5-(4-(benzyloxy)-3-fluorophenyl)-3-methylpyrimidin-4(3H)-one: Prepared from 2-benzyl-5-bromo-3-methylpyrimidin-4(3H)-one (0.067 g, 0.240 mmol) according to the procedure described in Step D of Example 56. The crude product was purified by silica gel flash column chromatography, eluting with 10:1 dichloromethane/EtOAc. The desired product (0.082 g, 85%) was obtained as an off-white solid. ¹H-NMR (400 MHz, DMSO-d₆) δ 8.15 (s, 1H), 7.66 (dd, 1H), 7.51 (m, 1H), 7.47 (m, 2H), 7.43-7.38 (m, 2H), 7.38-7.32 (m, 3H), 7.30-7.25 (m, 4H), 5.22 (s, 2H), 4.24 (s, 2H), 3.48 (s, 3H). LRMS (APCI pos) m/e 401 (M+1).

Step E. Preparation of 2-benzyl-5-(3-fluoro-4-hydroxyphenyl)-3-methylpyrimidin-4(3H)-one: Prepared from 2-benzyl-5-(4-(benzyloxy)-3-fluorophenyl)-3-methylpyrimidin-4(3H)-one (0.082 g, 0.20 mmol), according to the procedure described in Step E of Example 56. The desired product (0.064 g, 100%) was obtained as a pale yellow foamy solid. ¹H-NMR (400 MHz, DMSO-d₆) δ 10.01 (br s, 1H), 8.10 (s, 1H), 7.58 (dd, 1H), 7.40-7.32 (m, 3H), 7.30-7.23 (m, 3H), 6.97 (dd, 1H), 4.24 (s, 2H), 3.47 (s, 3H). LRMS (ESI pos) m/e 311 (M+1).

Step F. Preparation of 2-benzyl-5-(3-fluoro-4-(6-methoxy-7-(3-morpholinopropoxy)quinolin-4-yloxy)phenyl)-3-methylpyrimidin-4(3H)-one: Prepared from 2-benzyl-5-(3-fluoro-4-hydroxyphenyl)-3-methylpyrimidin-4(3H)-one (0.025 g, 0.081 mmol), 4-(3-(4-chloro-6-methoxyquinolin-7-yloxy)propyl)morpholine (0.030 g, 0.089 mmol) and catalytic DMAP according to the procedure described in Step C of Example 58, to give 159 (0.006 g, 12%) as a yellow solid. ¹H-NMR (400 MHz, DMSO-d₆) δ 8.50 (d, 1H), 8.29 (s, 1H), 7.92 (dd, 1H), 7.74 (m, 1H), 7.53 (s, 1H), 7.50 (t, 1H), 7.42 (s, 1H), 7.39-7.34 (m, 2H), 7.32-7.26 (m, 3H), 6.52 (dd, 1H), 4.28 (s, 2H), 4.21 (t, 2H), 3.95 (s, 3H), 3.59 (m, 4H), 3.52 (s, 3H), 2.47 (m, 2H), 2.39 (m, 4H), 1.99 (m, 2H). LRMS (APCI pos) m/e 611 (M+1).

### Example 60

### Preparation of 3-(4-(6-methoxy-7-(3-morpholinopropoxy)quinolin-4-yloxy)phenyl)-5-methyl-6-(phenylamino)pyrimidin-4(3H)-one 160

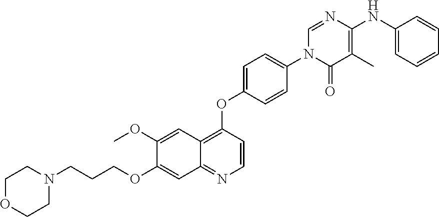

Step A: Preparation of 4-chloro-6-methoxy-5-methylpyrimidine: To a solution of 4,6-dichloro-5-methylpyrimidine (1.00 g, 6.13 mmol) in MeOH (50 mL) at 0° C. was added solid sodium methoxide (0.348 g, 6.44 mmol) in portions. The reaction mixture was allowed to warm to room temperature and stirred at room temperature for 4 hours and then at 50° C. for 12 hours. Additional sodium methoxide (0.348 g, 6.44 mmol) was added and the reaction mixture was stirred at 50° C. for 4 hours. Additional sodium methoxide (0.348 g, 6.44 mmol; 3 eq total) was added and the reaction mixture was stirred at 50° C. for 20 minutes, when HPLC showed the reaction was complete. The reaction mixture was concentrated and then partitioned between EtOAc and saturated aqueous NH₄Cl. The phases were separated, and the aqueous phase was re-extracted with EtOAc (1×). The combined organic layers were dried (Na₂SO₄), filtered and concentrated to yield the desired product (0.829 g, 85%) as a colorless oil that was used without further purification. ¹H-NMR (400 MHz, CDCl₃) δ 8.42 (s, 1H), 4.02 (s, 3H). LRMS (ESI pos) m/e 159 (M+1).

Step B: Preparation of 5-methyl-6-(phenylamino)pyrimidin-4(3H)-one: In a sealed tube was a mixture of 4-chloro-6-methoxy-5-methylpyrimidine (0.973 g, 6.14 mmol) and aniline (1.679 ml, 18.41 mmol) in n-BuOH (10 mL). The reaction mixture was stirred at reflux for 5 days and then at room temperature for 5 days. The reaction mixture was a purple suspension after cooling. The resulting solid was filtered and washed with Et₂O, collected and dried under vacuum. The filtrate was concentrated, dried under vacuum and the trituration procedure was repeated with dichloromethane/Et₂O. The solids were combined to yield the desired product (0.611 g, 49%) as a lavender solid. ¹H-NMR (400 MHz, DMSO-d₆) δ 11.88 (br s, 1H), 8.07 (s, 1H), 7.85 (s, 1H), 7.40 (m, 2H), 7.25 (m, 2H), 6.97 (m, 1H), 1.91 (s, 3H). LRMS (ESI pos) m/e 202 (M+1).

Step C: Preparation of 3-(4-(benzyloxy)phenyl)-5-methyl-6-(phenylamino)pyrimidin-4(3H)-one: To a mixture of 5-methyl-6-(phenylamino)pyrimidin-4(3H)-one (0.025 g, 0.124 mmol) and 1-(benzyloxy)-4-iodobenzene (0.046 g, 0.149 mmol) in dioxane (500 μL) and DMF (˜4 drops) was added copper (I) iodide (0.005 g, 0.024 mmol), (1S,2S)-cyclohexane-1,2-diamine (0.006 ml, 0.050 mmol), and K₃PO₄ (0.053 g, 0.248 mmol). The mixture was flushed with N₂ and stirred at 110° C. for 5 hours, when LC-MS showed some formation of desired product as well as formation of (1S,2S)—N-1-(4-(benzyloxy)phenyl)cyclohexane-1,2-diamine from coupling of ligand to 1-(benzyloxy)-4-iodobenzene. Because the reaction had stalled from ligand and 1-(benzyloxy)-4-iodobenzene being depleted, the secondary ligand N1,N2-dimethylethane-1,2-diamine (0.0132 ml, 0.124 mmol) and additional 1-(benzyloxy)-4-iodobenzene (0.020 g, 0.065 mmol) were added. The reaction mixture was then stirred at 110° C. for an additional 16 hours. The reaction mixture was partitioned between EtOAc and saturated aqueous NaCl. The phases were separated, and the aqueous phase was re-extracted with EtOAc (1×). The combined organic layers were dried (Na₂SO₄), filtered and concentrated. The crude product was purified by silica gel flash column chromatography, eluting with 10:1 dichloromethane/EtOAc. The desired product (0.030 g, 63%) was obtained as a white foamy solid. ¹H-NMR (400 MHz, DMSO-d₆) δ 8.25 (s, 1H), 8.11 (s, 1H), 7.49-7.38 (m 6H), 7.37-7.25 (m, 5H), 7.11 (m, 2H), 7.01 (m, 1H), 5.17 (s, 2H), 1.98 (s, 3H). LRMS (ESI pos) m/e 384 (M+1).

Step D: Preparation of 3-(4-hydroxyphenyl)-5-methyl-6-(phenylamino)pyrimidin-4(3H)-one: Prepared from 3-(4-(benzyloxy)phenyl)-5-methyl-6-(phenylamino)pyrimidin-4(3H)-one (0.029 g, 0.074 mmol) according to the procedure described in Step E of Example 56. The desired product (0.020 g, 89%) was obtained as a white solid. ¹H-NMR (400 MHz, DMSO-d₆) δ 9.73 (s, 1H), 8.23 (br s, 1H), 8.08 (s, 1H), 7.43 (m, 2H), 7.28 (m, 2H), 7.18 (m, 2H), 7.00 (m, 1H), 6.84 (m, 2H), 1.98 (s, 3H). LRMS (ESI pos) m/e 294 (M+1).

Step E: Preparation of 3-(4-(6-methoxy-7-(3-morpholinopropoxy)quinolin-4-yloxy)phenyl)-5-methyl-6-(phenylamino)pyrimidin-4(3H)-one: Prepared from 3-(4-hydroxyphenyl)-5-methyl-6-(phenylamino)pyrimidin-4(3H)-one (0.019 g, 0.063 mmol), 4-(3-(4-chloro-6-methoxyquinolin-7-yloxy)propyl)morpholine (0.023 g, 0.069 mmol) and catalytic DMAP according to the procedure described in Step C of Example 58, to give 160 (0.026 g, 69%) as a pale yellow foamy solid. ¹H-NMR (400 MHz, DMSO-d₆) δ 8.54 (d, 1H), 8.32 (br s, 1H), 8.21 (s, 1H), 7.56 (m, 2H), 7.49 (s, 1H), 7.47-7.36 (m, 5H), 7.29 (m, 2H), 7.02 (m, 1H), 6.62 (d, 1H), 4.21 (t, 2H), 3.93 (s, 3H), 3.59 (t, 4H), 2.48 (m, 2H), 2.39 (m, 4H), 2.02 (s, 3H), 1.98 (m, 2 h). LRMS (APCI pos) m/e 594 (M+1).

### Example 61

### Preparation of 3-(3-fluoro-4-(6-methoxy-7-(3-morpholinopropoxy)quinolin-4-yloxy)phenyl)-5-methyl-6-(phenylamino)pyrimidin-4(3H)-one 161

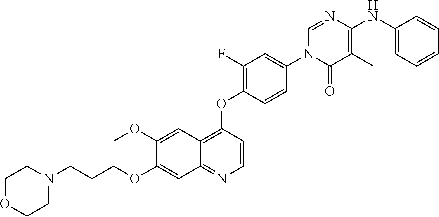

Step A: Preparation of 3-(3-fluoro-4-hydroxyphenyl)-5-methyl-6-(phenylamino)pyrimidin-4(3H)-one: To a mixture of 5-methyl-6-(phenylamino)pyrimidin-4(3H)-one (0.100 g, 0.497 mmol; obtained from Example XX, Step B) and 4-bromo-2-fluorophenol (0.0765 ml, 0.745 mmol) in dioxane (2 mL) and DMF (˜12 drops) was added copper(I) iodide (0.019 g, 0.099 mmol), N1,N2-dimethylethane-1,2-diamine (0.0214 ml, 0.199 mmol) and K₃PO₄ (0.211 g, 0.994 mmol). The mixture was flushed with N₂ and stirred at 110° C. for 16 hours. The reaction mixture was partitioned between EtOAc and saturated aqueous NaCl. The phases were separated, and the aqueous phase was re-extracted with EtOAc (2×). The combined organic layers were dried (Na₂SO₄), filtered and concentrated. The crude product was purified by silica gel flash column chromatography, eluting with 20:1 dichloromethane/MeOH. The desired product (0.069 g, 45%) was obtained as a brown solid. ¹H-NMR (400 MHz, DMSO-d₆) δ 10.22 (s, 1H), 8.26 (s, 1H), 8.11 (s, 1H), 7.42 (m, 2H), 7.33-7.25 (m, 3H), 7.07-6.97 (m, 3H), 1.98 (s, 3H). LRMS (ESI pos) m/e 312 (M+1).

Step B: Preparation of 3-(3-fluoro-4-(6-methoxy-7-(3-morpholinopropoxy)quinolin-4-yloxy)phenyl)-5-methyl-6-(phenylamino)pyrimidin-4(3H)-one: Prepared from 3-(3-fluoro-4-hydroxyphenyl)-5-methyl-6-(phenylamino)pyrimidin-4(3H)-one (0.025 g, 0.0803 mmol), 4-(3-(4-chloro-6-methoxyquinolin-7-yloxy)propyl)morpholine (0.0298 g, 0.0883 mmol) and catalytic DMAP according to the procedure described in Step C of Example 58, to give 161 (0.035 g, 71%) as a pale yellow foamy solid. ¹H-NMR (400 MHz, DMSO-d₆) δ 8.54 (d, 1H), 8.36 (br s, 1H), 8.24 (s, 1H), 7.74 (dd, 1H), 7.59 (t, 1H), 7.54 (s, 1H), 7.46-7.42 (m, 4H), 7.30 (m, 2H), 7.04 (m, 1H), 6.55 (dd, 1H), 4.22 (t, 2H), 3.96 (s, 3H), 3.59 (t, 4H), 2.47 (m, 2H), 2.39 (m, 4H), 2.02 (s, 3H), 1.99 (m, 2H). LRMS (ESI pos) m/e 612 (M+1).

### Example 62

### Preparation of 5-(3-fluoro-4-(6-methoxy-7-(3-morpholinopropoxy)quinolin-4-yloxy)phenyl)-3-methyl-2-(phenylamino)pyrimidin-4(3H)-one 162

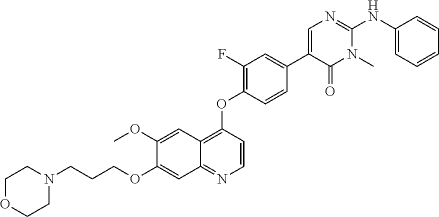

Step A: Preparation of 4-methoxy-N-phenylpyrimidin-2-amine: In a sealed tube was 2-chloro-4-methoxypyrimidine (1.00 g, 6.92 mmol) in 2-propanol (5 mL). Aniline (0.757 ml, 8.30 mmol) and DIEA (1.45 ml, 8.30 mmol) were added and the reaction mixture was heated at 100° C. until the reaction was complete by HPLC. The reaction mixture was cooled to room temperature. The resulting thick suspension was filtered, washed with ethanol, collected and dried under vacuum to yield the desired product (0.164 g) as a white solid. The filtrate was concentrated and then partitioned between EtOAc and saturated aqueous NaCl. The phases were separated, and the aqueous phase was re-extracted with EtOAc (1×). The combined organic layers were dried (Na₂SO₄), filtered and concentrated to yield a yellow solid. The crude product was purified by silica gel flash column chromatography, eluting with 25:1 dichloromethane/EtOAc. The desired product (0.548 g) was obtained as a white solid which was combined with the filtered product to yield 0.712 g (51%) total desired product. ¹H-NMR (400 MHz, DMSO-d₆) δ 9.51 (s, 1H), 8.20 (d, 1H), 7.77 (d, 2H), 7.27 (t, 2H), 6.94 (t, 1H), 6.28 (d, 1H), 3.91 (s, 3H). LRMS (ESI pos) m/e 202 (M+1).

Step B: Preparation of 2-(phenylamino)pyrimidin-4(3H)-one: To a solution of 4-methoxy-N-phenylpyrimidin-2-amine (0.632 g, 3.14 mmol) in acetic acid (20 mL) was added HBr (2.132 ml, 18.84 mmol; 48 wt % in H₂O). The reaction mixture was heated at 90-95° C. for 3 hours. The reaction mixture was cooled to room temperature and diluted with H₂O. The pH of the reaction mixture was adjusted to 5-6 with 6 M aqueous NaOH which resulted in the formation of a solid precipitate. The solid was filtered, washed with H₂O, collected and dried under vacuum to yield the desired product (0.553 g, 94%) as a white solid. ¹H-NMR (400 MHz, DMSO-d₆) δ 10.74 (br s, 1H), 8.81 (br s, 1H), 7.76 (s, 1H), 7.60 (d, 2H), 7.31 (t, 2H), 7.02 (t, 1H), 5.81 (s, 1H). LRMS (ESI pos) m/e 188 (M+1).

Step C: Preparation of 3-methyl-2-(phenylamino)pyrimidin-4(3H)-one. To a solution of 2-(phenylamino)pyrimidin-4(3H)-one (0.250 g, 1.34 mmol) in DMF (10 mL) was added LiH (0.012 g, 1.47 mmol). The reaction mixture was stirred for 25 minutes and then iodomethane (0.166 ml, 2.67 mmol) was added. The reaction was stirred at room temperature for 18 hours. The reaction mixture was quenched with H₂O and then partitioned between EtOAc and saturated aqueous NaCl. The phases were separated, and the aqueous phase was re-extracted with EtOAc (1×). The combined organic layers were dried (Na₂SO₄), filtered and concentrated to yield a crude yellow oil. The crude product was purified by silica gel flash column chromatography, eluting with 30:1 dichloromethane/methanol. The desired product (0.166 g, 62%) was obtained as a white crystalline solid. ¹H-NMR (400 MHz, CDCl₃) δ 7.68 (d, 1H), 7.46 (m, 2H), 7.39 (t, 2H), 7.19 (t, 1H), 6.48 (s, 1H), 6.01 (d, 1H), 3.58 (s, 3H). LRMS (ESI pos) m/e 202 (M+1).

Step D: Preparation of 5-bromo-3-methyl-2-(phenylamino)pyrimidin-4(3H)-one: To a solution of 3-methyl-2-(phenylamino)pyrimidin-4(3H)-one (0.104 g, 0.517 mmol) in CHCl₃ (5 mL)/MeOH (1 mL) at 0° C. was added bromine (0.027 ml, 0.517 mmol). The reaction mixture was stirred for 30 minutes at room temperature and then quenched with 10% aqueous sodium bisulfate solution. The reaction mixture was partitioned between EtOAc and H₂O. The phases were separated, and the aqueous phase was re-extracted with EtOAc (1×). The combined organic layers were dried (Na₂SO₄), filtered and concentrated to yield the desired product (0.145 g; 100%) as a white solid that was used without further purification. ¹H-NMR (400 MHz, DMSO-d₆) δ 8.95 (s, 1H), 7.94 (s, 1H), 7.47 (m, 2H), 7.34 (t, 2H), 7.14 (t, 1H), 3.53 (s, 3H). LRMS (ESI pos) m/e 280, 282 (M+1, Br pattern).

Step E. Preparation of 5-(4-(benzyloxy)-3-fluorophenyl)-3-methyl-2-(phenylamino)pyrimidin-4(3H)-one: A suspension of 5-bromo-3-methyl-2-(phenylamino)pyrimidin-4(3H)-one (0.145 g, 0.518 mmol), 4-(benzyloxy)-3-fluorophenylboronic acid (0.153 g, 0.621 mmol), Pd(PPh₃)₄ (0.030 g, 0.026 mmol) and lithium chloride (0.110 g, 2.59 mmol) in dioxane (1.5 mL) and 2 M aqueous Na₂CO₃ (1.5 mL) was stirred at 100° C. for 20 minutes. The reaction mixture was cooled to room temperature and then partitioned between EtOAc and H₂O. The phases were separated, and the aqueous phase was re-extracted with EtOAc (1×). The combined organic layers were dried (Na₂SO₄), filtered and concentrated to yield a crude black solid. The crude product was purified by silica gel flash column chromatography, eluting with 10:1 dichloromethane/EtOAc. The desired product (0.133 g, 64%) was obtained as a grey waxy solid. ¹H-NMR (400 MHz, DMSO-d₆) δ 8.90 (br s, 1H), 7.93 (s, 1H), 7.59 (dd, 1H), 7.55-7.31 (m, 10H), 7.22 (t, 1H), 7.14 (t, 1H), 5.20 (s, 2H), 3.55 (s, 3H). LRMS (ESI pos) m/e 402 (M+1).

Step F: Preparation of 5-(3-fluoro-4-hydroxyphenyl)-3-methyl-2-(phenylamino)pyrimidin-4(3H)-one: A solution of 5-(4-(benzyloxy)-3-fluorophenyl)-3-methyl-2-(phenylamino)pyrimidin-4(3H)-one (0.133 g, 0.331 mmol) in TFA (1.5 mL) was stirred at 40° C. for 3.5 hours The reaction mixture was concentrated to dryness and then purified by silica gel flash column chromatography, eluting with 20:1 dichloromethane/MeOH. The desired product (0.103 g, 100%) was obtained as a foamy white solid. ¹H-NMR (400 MHz, DMSO-d₆) δ 9.81 (br s, 1H), 8.96 (br s, 1H), 7.86 (s, 1H), 7.56-7.45 (m, 3H), 7.37 (t, 2H), 7.27 (m, 1H), 7.15 (t, 1H), 6.92 (t, 1H), 3.54 (s, 3H). LRMS (APCI pos) m/e 312 (M+1).

Step G. Preparation of 5-(3-fluoro-4-(6-methoxy-7-(3-morpholinopropoxy)quinolin-4-yloxy)phenyl)-3-methyl-2-(phenylamino)pyrimidin-4(3H)-one: Prepared from 5-(3-fluoro-4-hydroxyphenyl)-3-methyl-2-(phenylamino)pyrimidin-4(3H)-one (0.025 g, 0.0803 mmol), 4-(3-(4-chloro-6-methoxyquinolin-7-yloxy)propyl)morpholine (0.0298 g, 0.0883 mmol) and catalytic DMAP according to the procedure described in Step C of Example 58, to give 162 (0.029, 59%) as a pale yellow foamy solid. ¹H-NMR (400 MHz, DMSO-d₆) 9.02 (br s, 1H), 8.49 (d, 1H). 8.10 (s, 1H), 7.88 (dd, 1H), 7.68 (m, 1H), 7.57-7.51 (m, 3H), 7.47-7.35 (m, 4H), 7.16 (t, 1H), 6.49 (dd, 1H), 4.21 (t, 2H), 3.95 (s, 3H), 3.62-3.56 (m, 7H), 2.47 (m, 2H), 2.39 (m, 4H), 1.99 (m, 2H). LRMS (APCI pos) m/e 612 (M+1).

### Example 63

### Preparation of 2-(cyclopropylmethylamino)-5-(3-fluoro-4-(6-methoxy-7-(3-morpholinopropoxy)quinolin-4-yloxy)phenyl)-3-methylpyrimidin-4(3H)-one 163

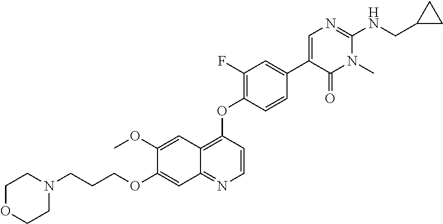

Step A: Preparation of 5-bromo-2-(cyclopropylmethylamino)-3-methylpyrimidin-4(3H)-one: A mixture of 5-bromo-2-chloro-3-methylpyrimidin-4(3H)-one (0.100 g, 0.45 mmol; obtained from Example 59, Step B), cyclopropylmethanamine (0.051 ml, 0.58 mmol) and NaHCO₃ (0.150 g, 1.79 mmol) in n-BuOH (3 mL) was stirred at 60° C. for 1 hour. The reaction mixture was cooled to room temperature and then diluted with EtOAc. The EtOAc layer was washed with H₂O and saturated aqueous NaCl. The aqueous phase was re-extracted with EtOAc (1×). The combined EtOAc layers were dried (Na₂SO4), filtered and concentrated to yield the desired product (0.114 g, 98%) as a pale yellow solid that was used without further purification. ¹H-NMR (400 MHz, DMSO-d₆) δ 7.93 (s, 1H), 7.46 (t, 1H), 3.33 (s, 3H), 3.19 (t, 2H), 1.12 (m, 1H), 0.43 (m, 2H), 0.24 (m, 2H). LRMS (ESI pos) m/e 258, 260 (M+, Br pattern).

Step B: Preparation of 5-(4-(benzyloxy)-3-fluorophenyl)-2-(cyclopropylmethylamino)-3-methylpyrimidin-4(3H)-one: A suspension of 5-bromo-2-(cyclopropylmethylamino)-3-methylpyrimidin-4(3H)-one (0.112 g, 0.434 mmol), 4-(benzyloxy)-3-fluorophenylboronic acid (0.128 g, 0.521 mmol), Pd(PPh₃)₄ (0.025 g, 0.022 mmol) and lithium chloride (0.092 g, 2.17 mmol) in dioxane (1.5 mL) and 2 M aqueous Na₂CO₃ (1.5 mL) was stirred at 100° C. for 30 minutes. The reaction mixture was cooled to room temperature and then partitioned between EtOAc and H₂O. The phases were separated, and the aqueous phase was re-extracted with EtOAc (1×). The combined organic layers were dried (Na₂SO₄), filtered and concentrated to yield a crude black solid. The crude product was purified by silica gel flash column chromatography, eluting with 10:1 dichloromethane/EtOAc. The desired product (0.128 g, 78%) was obtained as a foamy off-white solid. ¹H-NMR (400 MHz, DMSO-d₆) δ 7.93 (s, 1H), 7.57 (dd, 1H), 7.49-7.31 (m, 7H), 7.19 (t, 1H), 5.19 (s, 2H), 3.35 (s, 3H), 3.24 (t, 2H), 1.16 (m, 1H), 0.44 (m, 2H), 0.25 (m, 1H). LRMS (APCI pos) m/e 380 (M+1).

Step C. Preparation of 2-(cyclopropylmethylamino)-5-(3-fluoro-4-hydroxyphenyl)-3-methylpyrimidin-4(3H)-one: A solution of 5-(4-(benzyloxy)-3-fluorophenyl)-2-(cyclopropylmethylamino)-3-methylpyrimidin-4(3H)-one (0.128 g, 0.337 mmol) in TFA (2 mL) was stirred at 40° C. for 2 hours and 45 minutes. The reaction mixture was concentrated to dryness and then purified by silica gel flash column chromatography, eluting with 20:1 dichloromethane/MeOH. The desired product (0.080 g, 82%) was obtained as a colorless glassy solid. ¹H-NMR (400 MHz, DMSO-d₆) δ 9.71 (s, 1H), 7.87 (s, 1H), 7.46 (dd, 1H), 7.35 (t, 1H), 7.24 (dd, 1H), 6.90 (dd, 1H), 3.34 (s, 3H), 3.24 (t, 2H), 1.16 (m, 1H), 0.44 (m, 2H), 0.26 (m, 2H). LRMS (ESI pos) m/e 290 (M+1).

Step D. Preparation of 2-(cyclopropylmethylamino)-5-(3-fluoro-4-(6-methoxy-7-(3-morpholinopropoxy)quinolin-4-yloxy)phenyl)-3-methylpyrimidin-4(3H)-one: Prepared from 2-(cyclopropylmethylamino)-5-(3-fluoro-4-hydroxyphenyl)-3-methylpyrimidin-4(3H)-one (0.025 g, 0.0864 mmol), 4-(3-(4-chloro-6-methoxyquinolin-7-yloxy)propyl)morpholine (0.0320 g, 0.0951 mmol) and catalytic DMAP according to the procedure described in Step C of Example 58, to give 163 (0.030, 60%) as a pale yellow solid. ¹H-NMR (400 MHz, DMSO-d₆) δ 8.48 (d, 1H), 8.11 (s, 1H), 7.85 (dd, 1H), 7.66 (m, 1H), 7.56-7.50 (m, 2H), 7.44-7.39 (m, 2H), 6.48 (dd, 1H), 4.21 (t, 2H), 3.96 (s, 3H), 3.59 (t, 4H), 3.38 (s, 3H), 3.28 (m, 2H), 2.47 (m, 2H), 2.39 (m, 4H), 1.98 (m, 2H), 1.18 (m, 1H), 0.46 (m, 2H), 0.28 (m, 2H). LRMS (APCI pos) m/e 590 (M+1).

### Example 64

### Preparation of 5-(3-fluoro-4-(6-methoxy-7-(3-morpholinopropoxy)quinolin-4-yloxy)phenyl)-2-(4-fluorophenylamino)-3-methylpyrimidin-4(3H)-one 164

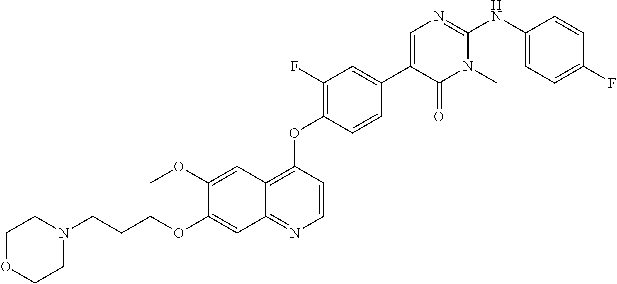

Step A: Preparation of 5-bromo-2-(4-fluorophenylamino)-3-methylpyrimidin-4(3H)-one: Prepared from 5-bromo-2-chloro-3-methylpyrimidin-4(3H)-one (0.100 g, 0.45 mmol; obtained from Example 59, Step B), according to the procedure described in Step A of Example 63, substituting 4-fluorobenzenamine in place of cyclopropylmethanamine. The desired product (0.132, 99%) was obtained as a pale yellow solid. ¹H-NMR (400 MHz, DMSO-d₆) δ 8.97 (br s, 1H), 7.93 (s, 1H), 7.47 (m, 2H), 7.19 (m, 2H), 3.51 (s, 3H). LRMS (ESI pos) m/e 298, 300 (M+, Br pattern).

Step B: Preparation of 5-(4-(benzyloxy)-3-fluorophenyl)-2-(4-fluorophenylamino)-3-methylpyrimidin-4(3H)-one: Prepared from 5-bromo-2-(4-fluorophenylamino)-3-methylpyrimidin-4(3H)-one (0.130 g, 0.436 mmol) according to the procedure described in Step B of Example 63. The desired product (0.139 g, 76%) was obtained as a grey/white solid. ¹H-NMR (400 MHz, DMSO-d₆) δ 8.93 (br s, 1H), 7.92 (s, 1H), 7.58 (dd, 1H), 7.52 (m, 2H), 7.49-7.44 (m, 2H), 7.44-7.39 (m, 3H), 7.37-7.31 (m, 1H), 7.24-7.16 (m, 3H), 5.19 (s, 2H), 3.53 (s, 3H). LRMS (ESI pos) m/e 420 (M+1).

Step C. Preparation of 5-(3-fluoro-4-hydroxyphenyl)-2-(4-fluorophenylamino)-3-methylpyrimidin-4(3H)-one: Prepared from 5-(4-(benzyloxy)-3-fluorophenyl)-2-(4-fluorophenylamino)-3-methylpyrimidin-4(3H)-one (0.139 g, 0.331 mmol) according to the procedure described in Step C of Example 63. The desired product (0.089 g, 82%) was obtained as a white solid. ¹H-NMR (400 MHz, DMSO-d₆) δ 9.78 (s, 1H), 8.89 (s, 1H), 7.86 (s, 1H), 7.56-7.46 (m, 3H), 7.27 (m, 1H), 7.19 (m, 2H), 6.92 (dd, 1H), 3.53 (s, 3H). LRMS (ESI pos) m/e 330 (M+1).

Step D. Preparation of 5-(3-fluoro-4-(6-methoxy-7-(3-morpholinopropoxy)quinolin-4-yloxy)phenyl)-2-(4-fluorophenylamino)-3-methylpyrimidin-4(3H)-one: Prepared from 5-(3-fluoro-4-hydroxyphenyl)-2-(4-fluorophenylamino)-3-methylpyrimidin-4(3H)-one (0.025 g, 0.0759 mmol), 4-(3-(4-chloro-6-methoxyquinolin-7-yloxy)propyl)morpholine (0.0281 g, 0.0835 mmol) and catalytic DMAP according to the procedure described in Step C of Example 58, to give 164 (0.026 g, 54%) as a pale yellow solid. ¹H-NMR (400 MHz, DMSO-d₆) δ 9.05 (br s, 1H), 8.49 (d, 1H), 8.09 (s, 1H), 7.87 (dd, 1H), 7.67 (m, 1H), 7.58-7.50 (m, 3H), 7.44 (t, 1H), 7.41 (s, 1H), 7.22 (m, 2H), 6.49 (dd, 1H), 4.21 (t, 2H), 3.95 (s, 3H), 3.59 (t, 4H), 3.57 (s, 3H), 2.47 (m, 2H), 2.39 (m, 4H), 1.98 (m, 2H). LRMS (APCI pos) m/e 630 (M+1).

### Example 65

### Preparation of 3-ethyl-5-(3-fluoro-4-(6-methoxy-7-(3-morpholinopropoxy)quinolin-4-yloxy)phenyl)-2-(phenylamino)pyrimidin-4(3H)-one 165

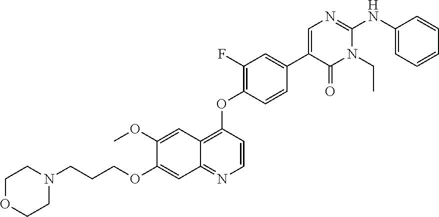

Step A: Preparation of 5-bromo-2-chloro-3-ethylpyrimidin-4(3H)-one: Prepared from 5-bromo-2-chloropyrimidin-4(3H)-one (1.00 g, 4.775 mmol; obtained from Example 59, Step A) according to the procedure described in Step B of Example 59, substituting iodoethane in place of iodomethane. The desired product (0.411 g, 36%) was obtained as a yellow crystalline waxy solid. ¹-NMR (400 MHz, DMSO-d₆) δ 8.26 (s, 1H), 4.16 (q, 3H), 1.25 (t, 4H). LRMS (ESI pos) m/e 237, 239 (M+, Br pattern).

Step B: Preparation of 5-bromo-3-ethyl-2-(phenylamino)pyrimidin-4(3H)-one: Prepared from 5-bromo-2-chloro-3-ethylpyrimidin-4(3H)-one (0.075 g, 0.316 mmol) according to the procedure described in Step A of Example 63, substituting aniline in place of cyclopropylmethanamine. The desired product (0.091 g, 98%) was obtained as a yellow solid. ¹H-NMR (400 MHz, DMSO-d₆) δ 9.00 (br s, 1H), 7.91 (s, 1H), 7.46-7.41 (m, 2H), 7.39-7.32 (m, 2H), 7.16 (m, 1H), 4.19 (q, 2H), 1.23 (t, 3H). LRMS (APCI pos) m/e 294, 296 (M+, Br pattern).

Step C: Preparation of 5-(4-(benzyloxy)-3-fluorophenyl)-3-ethyl-2-(phenylamino)pyrimidin-4(3H)-one: Prepared from 5-bromo-3-ethyl-2-(phenylamino)pyrimidin-4(3H)-one (0.086 g, 0.292 mmol) according to the procedure described in Step B of Example 63. The desired product (0.073 g, 60%) was obtained as a white foamy solid. ¹H-NMR (400 MHz, DMSO-d₆) δ 8.94 (br s, 1H), 7.90 (s, 1H), 7.59 (dd, 1H), 7.52-7.31 (m, 9H), 7.21 (t, 1H), 7.16 (t, 1H), 5.20 (s, 2H), 4.22 (q, 2H), 1.25 (t, 3H). LRMS (APCI pos) m/e 416 (M+1).

Step D: Preparation of 3-ethyl-5-(3-fluoro-4-hydroxyphenyl)-2-(phenylamino)pyrimidin-4(3H)-one: Prepared from 5-(4-(benzyloxy)-3-fluorophenyl)-3-ethyl-2-(phenylamino)pyrimidin-4(3H)-one (0.072 g, 0.17 mmol) according to the procedure described in Step C of Example 63. The desired product (0.056 g, 100%) was obtained as a white solid. ¹H-NMR (400 MHz, DMSO-d₆) δ 9.80 (br s, 1H), 8.98 (br s, 1H), 7.82 (s, 1H), 7.54-7.42 (m, 3H), 7.37 (m, 2H), 7.27 (dd, 1H), 7.16 (t, 1H), 6.92 (dd, 1H), 4.22 (q, 2H), 1.25 (t, 3H). LRMS (APCI pos) m/e 326 (M+1).

Step E: Preparation of 3-ethyl-5-(3-fluoro-4-(6-methoxy-7-(3-morpholinopropoxy)quinolin-4-yloxy)phenyl)-2-(phenylamino)pyrimidin-4(3H)-one: Prepared from 3-ethyl-5-(3-fluoro-4-hydroxyphenyl)-2-(phenylamino)pyrimidin-4(3H)-one (0.025 g, 0.0768 mmol), 4-(3-(4-chloro-6-methoxyquinolin-7-yloxy)propyl)morpholine (0.0285 g, 0.0845 mmol) and catalytic DMAP according to the procedure described in Step C of Example 58, to give 165 (0.016 g, 33%) as a pale yellow solid. ¹H-NMR (400 MHz, DMSO-d₆) δ 9.07 (br s, 1H), 8.49 (d, 1H), 8.07 (s, 1H), 7.88 (dd, 1H), 7.68 (m, 1H), 7.55-7.35 (m, 7H), 7.19 (m, 1H), 6.49 (dd, 1H), 4.30-4.18 (m, 4H), 3.95 (s, 3H), 3.60 (t, 4H), 2.49 (m, 2H), 2.40 (m, 4H), 1.99 (m, 2H), 1.29 (t, 3H). LRMS (APCI pos) m/e 626 (M+1).

### Example 66

### Preparation of 5-(3-fluoro-4-(6-methoxy-7-(3-morpholinopropoxy)quinolin-4-yloxy)phenyl)-3-methyl-2-phenoxypyrimidin-4(3H)-one 166

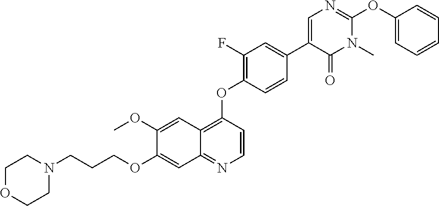

Step A: Preparation of 5-bromo-3-methyl-2-phenoxypyrimidin-4(3H)-one: Prepared from 5-bromo-2-chloro-3-methylpyrimidin-4(3H)-one (0.075 g, 0.336 mmol; obtained from Example 59, Step B), according to the procedure described in Step A of Example 63, substituting phenol in place of cyclopropylmethanamine. The desired product (0.058, 62%) was obtained as a white solid. ¹H-NMR (400 MHz, DMSO-d₆) δ 8.02 (s, 1H), 7.49-7.43 (m, 2H), 7.34-7.27 (m, 3H), 3.54 (s, 3H). LRMS (ESI pos) m/e 281, 283 (M+, Br pattern).

Step B: Preparation of 5-(4-(benzyloxy)-3-fluorophenyl)-3-methyl-2-phenoxypyrimidin-4(3H)-one: Prepared from 5-bromo-3-methyl-2-phenoxypyrimidin-4(3H)-one (0.056 g, 0.199 mmol) according to the procedure described in Step B of Example 63. The desired product (0.079 g, 99%) was obtained as a white/grey solid. ¹H-NMR (400 MHz, DMSO-d₆) δ 7.90 (s, 1H), 7.57 (dd, 1H), 7.51-7.29 (m, 1H), 7.26 (t, 1H), 5.21 (s, 2H), 3.57 (s, 3H). LRMS (ESI pos) m/e 403 (M+1).

Step C: Preparation of 5-(3-fluoro-4-hydroxyphenyl)-3-methyl-2-phenoxypyrimidin-4(3H)-one: Prepared from 5-(4-(benzyloxy)-3-fluorophenyl)-3-methyl-2-phenoxypyrimidin-4(3H)-one (0.078 g, 0.19 mmol) according to the procedure described in Step C of Example 63. The desired product (0.065 g, 82%) was obtained as a pale yellow foamy solid. ¹H-NMR (400 MHz, DMSO-d₆) δ 9.94 (br s, 1H), 7.85 (s, 1H), 7.52-7.44 (m, 3H), 7.35-7.26 (m, 4H), 6.95 (dd, 1H), 3.56 (s, 3H). LRMS (APCI pos) m/e 313 (M+1).

Step D: Preparation of 5-(3-fluoro-4-(6-methoxy-7-(3-morpholinopropoxy)quinolin-4-yloxy)phenyl)-3-methyl-2-phenoxypyrimidin-4(3H)-one: Prepared from 5-(3-fluoro-4-hydroxyphenyl)-3-methyl-2-phenoxypyrimidin-4(3H)-one (0.029 g, 0.086 mmol), 4-(3-(4-chloro-6-methoxyquinolin-7-yloxy)propyl)morpholine (0.0247 g, 0.0733 mmol) and catalytic DMAP according to the procedure described in Step C of Example 58, to give 166 (0.030 g, 66%) as a pale yellow foamy solid. ¹H-NMR (400 MHz, DMSO-d₆) δ 8.49 (d, 1H), 8.07 (s, 1H), 7.84 (dd, 1H), 7.67 (m, 1H), 7.54-7.46 (m, 4H), 7.41 (s, 1H), 7.37-7.31 (m, 3H), 6.51 (dd, 1H), 4.21 (t, 2H), 3.95 (s, 3H), 3.60 (s, 3H), 3.59 (m, 4H), 2.47 (m, 2H), 2.39 (m, 4H), 1.98 (m, 2H). LRMS (APCI pos) m/e 613 (M+1).

### Example 67

### Preparation of 5-(3-fluoro-4-(6-methoxy-7-(3-morpholinopropoxy)quinolin-4-yloxy)phenyl)-3-methyl-2-(methyl(phenyl)amino)pyrimidin-4(3H)-one

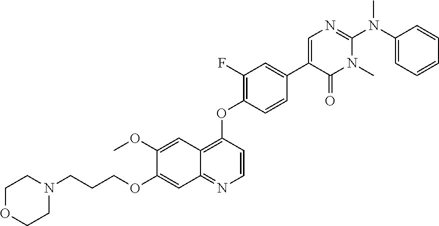

Step A: Preparation of 5-bromo-3-methyl-2-(methyl(phenyl)amino)pyrimidin-4(3H)-one: Prepared from 5-bromo-2-chloro-3-methylpyrimidin-4(3H)-one (0.100 g, 0.448 mmol; obtained from Example 59, Step B), according to the procedure described in Step A of Example 63, substituting N-methylaniline in place of cyclopropylmethanamine. The desired product (0.085, 65%) was obtained as a white solid. ¹H-NMR (400 MHz, DMSO-d₆) δ 8.23 (s, 1H), 7.42-7.36 (m, 2H), 7.20 (m, 1H), 7.15-7.10 (m, 2H), 3.32 (s, 3H), 2.92 (s, 3H). LRMS (ESI pos) m/e 294, 296 (M+, Br pattern).

Step B: 5-(4-(benzyloxy)-3-fluorophenyl)-3-methyl-2-(methyl(phenyl)amino)pyrimidin-4(3H)-one: Prepared from 5-bromo-3-methyl-2-(methyl(phenyl)amino)pyrimidin-4(3H)-one (0.083 g, 0.282 mmol) according to the procedure described in Step B of Example 63. The desired product (0.109 g, 93%) was obtained as a pale yellow solid. ¹H-NMR (400 MHz, DMSO-d₆) δ 8.17 (s, 1H), 7.66 (dd, 1H), 7.53-7.32 (m, 8H), 7.27 (t, 1H), 7.19 (m, 1H), 7.12-7.08 (m, 2H), 5.22 (s, 2H), 3.36 (s, 3H), 2.96 (s, 3H). LRMS (APCI pos) m/e 416 (M+1).

Step C: Preparation of 5-(3-fluoro-4-hydroxyphenyl)-3-methyl-2-(methyl(phenyl)amino)pyrimidin-4(3H)-one: Prepared from 5-(4-(benzyloxy)-3-fluorophenyl)-3-methyl-2-(methyl(phenyl)amino)pyrimidin-4(3H)-one (0.109 g, 0.262 mmol) according to the procedure described in Step C of Example 63. The desired product (0.082 g, 71%) was obtained as a pale yellow foamy solid. ¹H-NMR (400 MHz, DMSO-d₆) δ 9.92 (br s, 1H), 8.12 (s, 1H), 7.58 (dd, 1H), 7.44-7.36 (m, 3H), 7.18 (m, 1H), 7.09 (m, 2H), 6.97 (dd, 1H), 3.35 (s, 3H), 2.97 (s, 3H). LRMS (ESI pos) m/e 326 (M+1).

Step D: Preparation of 5-(3-fluoro-4-(6-methoxy-7-(3-morpholinopropoxy)quinolin-4-yloxy)phenyl)-3-methyl-2-(methyl(phenyl)amino)pyrimidin-4(3H)-one: Prepared from 5-(3-fluoro-4-hydroxyphenyl)-3-methyl-2-(methyl(phenyl)amino)pyrimidin-4(3H)-one (0.025 g, 0.078 mmol), 4-(3-(4-chloro-6-methoxyquinolin-7-yloxy)propyl)morpholine (0.025 g, 0.074 mmol) and catalytic DMAP according to the procedure described in Step C of Example 58, to give 167 (0.021, 45%) as a pale yellow foamy solid. ¹H-NMR (400 MHz, DMSO-d₆) 8.50 (d, 1H). 8.33 (s, 1H), 7.94 (dd, 1H), 7.76 (m, 1H), 7.55 (s, 1H), 7.50 (t, 1H), 7.46-7.40 (m, 3H), 7.22 (m, 1H), 7.18-7.33 (m, 2H), 6.51 (dd, 1H), 4.21 (t, 2H), 3.96 (s, 3H), 3.59 (t, 4H), 3.40 (s, 3H), 2.98 (s, 3H), 2.48 (m, 2H), 2.40 (m, 4H), 1.99 (m, 2H). LRMS (APCI pos) m/e 626 (M+1).

### Example 68

### Preparation of 6-benzyl-3-(3-fluoro-4-(6-methoxy-7-(3-morpholinopropoxy)quinolin-4-yloxy)phenyl)-1-methylpyridin-2(1H)-one 168

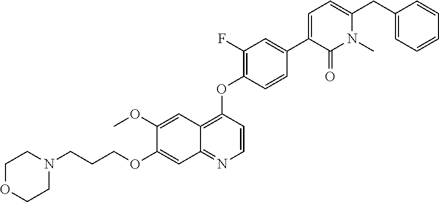

Step A: Preparation of 6-chloro-1-methylpyridin-2(1H)-one: To a solution of 6-chloropyridin-2-ol (10.00 g, 77.19 mmol) in acetone (350 mL) was added K₂CO₃ (37.34 g, 270.2 mmol) and iodomethane (17.37 ml, 270.2 mmol). The reaction mixture was stirred at room temperature for 1 hour and then at reflux for 16 hours. The reaction mixture was cooled to room temperature and the K₂CO₃ was filtered off and washed with acetone. The filtrate was then concentrated and the residue was partitioned between H₂O and CH₂Cl₂. The phases were separated, and the aqueous phase was re-extracted with CH₂Cl₂ (1×). The combined CH₂Cl₂ layers were dried (Na₂SO₄), filtered and concentrated to yield a yellow oil. The crude product was purified by silica gel flash column chromatography, eluting with 10:1 CH₂Cl₂/EtOAc. The desired product (7.38 g, 67%) was obtained as a white solid. ¹H-NMR (400 MHz, CDCl₃) δ 7.23 (dd, 1H), 6.50 (dd, 1H), 6.30 (m, 1H), 3.69 (s, 3H). LRMS (ESI pos) m/e 144 (M+1).

Step B: Preparation of 6-benzyl-1-methylpyridin-2(1H)-one: A mixture of 6-chloro-1-methylpyridin-2(1H)-one (0.200 g, 1.39 mmol) and PdCl₂(PPh₃)₂ (0.049 g, 0.070 mmol) in THF (8 mL) was sparged with N₂. Benzylzinc (II) bromide (3.06 ml, 1.53 mmol; 0.5 M solution in THF) was added and the reaction mixture was stirred at reflux for 1 hour and then at room temperature for 16 hours. The reaction mixture partitioned between H₂O and EtOAc. The phases were separated, and the aqueous phase was re-extracted with EtOAc (1×). The combined EtOAc layers were dried (Na₂SO₄), filtered and concentrated to yield a yellow oil. The crude product was purified by silica gel flash column chromatography, eluting with 20:1 CH₂Cl₂/MeOH. The desired product (0.144 g, 52%) was obtained as a yellow oil that crystallized to a waxy solid under vacuum. ¹H-NMR (400 MHz, CDCl₃) δ 7.37-7.31 (m, 3H), 7.30-7.23 (m, 1H), 7.16-7.11 (m, 2H), 6.53 (dd, 1H), 5.99 (m, 1H), 3.98 (s, 2H), 3.43 (s, 3H). LRMS (APCI pos) m/e 200 (M+1).

Step C: Preparation of 6-benzyl-3-bromo-1-methylpyridin-2(1H)-one: To a solution of 6-benzyl-1-methylpyridin-2(1H)-one (0.144 g, 0.723 mmol) in CHCl₃ (5 mL) was added Br₂ (0.037 ml, 0.72 mmol). The reaction mixture was stirred at room temperature for 2 hours and then quenched with 10% sodium bisulfite solution. The reaction mixture was partitioned between EtOAc and H₂O. The phases were separated, and the aqueous phase was re-extracted with EtOAc (1×). The combined organic layers were dried (Na₂SO₄), filtered and concentrated to yield a yellow oil. The crude product was purified by silica gel flash column chromatography, eluting with 20:1 CH₂Cl₂/EtOAc. The desired product (0.087 g, 43%) was obtained as a yellow gum. ¹H-NMR (400 MHz, CDCl₃) δ 7.38-7.31 (m, 3H), 7.31-7.28 (m, 1H), 7.14-7.10 (m, 2H), 5.91 (d, 1H), 3.96 (s, 2H), 3.50 (s, 3H). LRMS (APCI pos) m/e 278, 280 (M+, Br pattern).

Step D: Preparation of 6-benzyl-3-(4-(benzyloxy)-3-fluorophenyl)-1-methylpyridin-2(1H)-one: Prepared from 6-benzyl-3-bromo-1-methylpyridin-2(1H)-one (0.087 g, 0.313 mmol) according to the procedure described in Step B of Example 63. The desired product (0.071 g, 57%) was obtained as yellow gum that crystallized to a waxy solid under vacuum. ¹H-NMR (400 MHz, DMSO-d₆) δ 7.55 (dd, 1H), 7.48-7.27 (m, 10H), 7.17 (m, 2H), 7.00 (t, 1H), 6.08 (d, 1H), 5.17 (s, 2H), 4.01 (s, 2H), 3.49 (s, 3H). LRMS (ESI pos) m/e 400 (M+1).

Step E: 6-benzyl-3-(3-fluoro-4-hydroxyphenyl)-1-methylpyridin-2(1H)-one: Prepared from 6-benzyl-3-(4-(benzyloxy)-3-fluorophenyl)-1-methylpyridin-2(1H)-one (0.071 g, 0.18 mmol) according to the procedure described in Step C of Example 63. The desired product (0.047 g, 85%) was obtained as a pale yellow foamy solid. ¹H-NMR (400 MHz, DMSO-d₆) δ 9.87 (s, 1H), 7.60 (dd, 1H), 7.56 (d, 1H), 7.40-7.33 (m, 3H), 7.28 (m, 1H), 7.26-7.21 (m, 2H), 6.93 (dd, 1H), 6.08 (d, 1H), 4.12 (s, 2H), 3.42 (s, 3H). LRMS (ESI pos) m/e 310 (M+1).

Step F: Preparation of 6-benzyl-3-(3-fluoro-4-(6-methoxy-7-(3-morpholinopropoxy)quinolin-4-yloxy)phenyl)-1-methylpyridin-2(1H)-one: Prepared from 6-benzyl-3-(3-fluoro-4-hydroxyphenyl)-1-methylpyridin-2(1H)-one (0.027 g, 0.087 mmol), 4-(3-(4-chloro-6-methoxyquinolin-7-yloxy)propyl)morpholine (0.032 g, 0.096 mmol) and catalytic DMAP according to the procedure described in Step C of Example 58, to give 168 (0.037 g, 70%) as a pale yellow foamy solid. ¹H-NMR (400 MHz, DMSO-d₆) δ 8.49 (d, 1H), 7.95 (dd, 1H), 7.77 (d, 1H), 7.72 (m, 1H), 7.54 (s, 1H), 7.46 (t, 1H), 7.43-7.36 (m, 3H), 7.33-7.24 (m, 3H), 6.50 (dd, 1H), 6.16 (d, 1H), 4.21 (t, 2H), 4.17 (s, 2H), 3.96 (s, 3H), 3.59 (t, 4H), 3.47 (s, 3H), 2.47 (m, 2H), 2.39 (m, 4H), 1.98 (m, 2H). LRMS (ESI pos) m/e 610 (M+1).

### Example 69

### Preparation of 3-(3-fluoro-4-(6-methoxy-7-(3-morpholinopropoxy)quinolin-4-yloxy)phenyl)-1-methyl-6-(phenylamino)pyridin-2(1H)-one 169

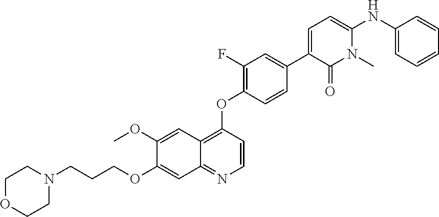

Step A: Preparation of 3-bromo-6-chloro-1-methylpyridin-2(1H)-one: To a solution of 6-chloro-1-methylpyridin-2(1H)-one (0.500 g, 3.48 mmol; obtained from Example 68, Step A) in DMF (15 mL) was added N-bromosuccinimide (0.620 g, 3.48 mmol). The reaction was stirred at room temperature for 2 hours and then quenched with 10% sodium bisulfate solution. The reaction mixture was partitioned between EtOAc and H₂O. The phases were separated, and the aqueous phase was re-extracted with EtOAc (1×). The combined organic layers were dried (Na₂SO₄), filtered and concentrated to yield a yellow oil. The crude product was purified by silica gel flash column chromatography, eluting with 20:1 CH₂Cl₂/EtOAc. The desired product (0.424 g, 55%) was obtained as a white crystalline solid. ¹H-NMR (400 MHz, DMSO-d₆) δ 7.90 (d, 1H), 6.48 (d, 1H), 3.63 (s, 3H). LRMS (ESI pos) m/e 222, 224 (M+, Br pattern). Also isolated was 5-bromo-6-chloro-1-methylpyridin-2(1H)-one (0.233 g, 30%) as a white crystalline solid. ¹H-NMR (400 MHz, DMSO-d₆) δ 7.68 (d, 1H), 6.42 (d, 1H), 3.61 (s, 3H).

Step B: Preparation of 3-(4-(benzyloxy)-3-fluorophenyl)-6-chloro-1-methylpyridin-2(1H)-one: Prepared from 3-bromo-6-chloro-1-methylpyridin-2(1H)-one (0.050 g, 0.225 mmol) according to the procedure described in Step B of Example 63. The desired product (0.059 g, 76%) was obtained as a pale yellow waxy crystalline solid. ¹H-NMR (400 MHz, DMSO-d₆) δ 7.68-7.62 (m, 2H), 7.50-7.45 (m, 3H), 7.44-7.32 (m, 3H), 7.26 (t, 1H), 6.60 (d, 1H), 5.22 (s, 2H), 3.64 (s, 3H). LRMS (ESI pos) m/e 344 (M+1).

Step C: Preparation of 3-(4-(benzyloxy)-3-fluorophenyl)-1-methyl-6-(phenylamino)pyridin-2(1H)-one: To a solution of aniline (0.018 ml, 0.203 mmol) in THF (1 mL) at −78° C. is added LiHMDS (0.203 ml, 0.203 mmol; 1 M soln in hexanes) dropwise. The reaction mixture is stirred for 30 minutes at −78° C. after addition is complete. 3-(4-(benzyloxy)-3-fluorophenyl)-6-chloro-1-methylpyridin-2(1H)-one (0.058 g, 0.169 mmol) is then added dropwise as a solution in THF (1 mL). The reaction mixture is stirred at −78° C. and slowly warmed to room temperature and stirred for 16 hours. The reaction mixture is quenched with H₂O and then partitioned between EtOAc and H₂O. The phases are separated, and the aqueous phase is re-extracted with EtOAc (1×). The combined organic layers are dried (Na₂SO₄), filtered and concentrated to yield the crude product. The crude product is purified by silica gel flash column chromatography, eluting with 20:1 CH₂Cl₂/EtOAc to obtain the desired product.

Step D: Preparation of 3-(3-fluoro-4-hydroxyphenyl)-1-methyl-6-(phenylamino)pyridin-2(1H)-one: The title compound is prepared from 3-(4-(benzyloxy)-3-fluorophenyl)-1-methyl-6-(phenylamino)pyridin-2(1H)-one according to the procedure described in Step C of Example 63.

Step E: Preparation of 3-(3-fluoro-4-(6-methoxy-7-(3-morpholinopropoxy)quinolin-4-yloxy)phenyl)-1-methyl-6-(phenylamino)pyridin-2(1H)-one: Compound 169 is prepared from 3-(3-fluoro-4-hydroxyphenyl)-1-methyl-6-(phenylamino)pyridin-2(1H)-one, 4-(3-(4-chloro-6-methoxyquinolin-7-yloxy)propyl)morpholine and catalytic DMAP according to the procedure described in Step C of Example 58.

### Example 70

### Preparation of 1-(4-(6,7-dimethoxyquinolin-4-yloxy)-3-fluorophenyl)-N-(4-fluorophenyl)-2-oxopyrrolidine-3-carboxamide 170

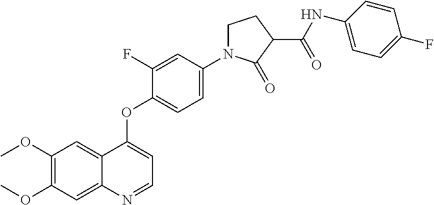

Step A: Preparation of ethyl 1-(4-(6,7-dimethoxyquinolin-4-yloxy)-3-fluorophenyl)-2-oxopyrrolidine-3-carboxylate: A mixture of 4-(4-bromo-2-fluorophenoxy)-6,7-dimethoxyquinoline (0.172 g, 0.19 mmol, Example 34), ethyl 2-oxopyrrolidine-3-carboxylate (0.025 g, 0.16 mmol), (1R,2R)-cyclohexane-1,2-diamine (0.011 g, 0.60 mmol), CuI (0.009 g, 0.30 mmol), and K₃PO₄ (0.068 g, 0.32 mmol) was placed in a sealed vial with dioxane (4 mL). The reaction mixture was then flushed with nitrogen, capped and placed in an oil bath at 110° C., and stirred for 20 hours. After the reaction was cooled to room temperature, the mixture was filtered through a pad of celite with EtOAc. After evaporation of the solvent, the crude was purified by silica gel flash column chromatography (1.5% MeOH in CH₂Cl₂) to afford 7.7 mg (11%) of the desired product. LRMS (ESI pos) m/e 455.2 (M+1).

Step B: Preparation of 1-(4-(6,7-dimethoxyquinolin-4-yloxy)-3-fluorophenyl)-2-oxopyrrolidine-3-carboxylic acid: LiOH (0.034 mL, 0.034 mmol, 1.0 M in H₂O) was added to a solution of ethyl 1-(4-(6,7-dimethoxyquinolin-4-yloxy)-3-fluorophenyl)-2-oxopyrrolidine-3-carboxylate in 2 mL (4:1 ratio of THF:MeOH) at room temperature and stirred for 1 hour. The reaction mixture was acidified to pH 1 with aq. 1 N HCl solution and treated with water (5 mL), extracted with EtOAc, washed with brine, dried over MgSO₄, and concentrated to afford 5.0 mg (69%) of the desired product.

Step C: Preparation of 1-(4-(6,7-dimethoxyquinolin-4-yloxy)-3-fluorophenyl)-N-(4-fluorophenyl)-2-oxopyrrolidine-3-carboxamide: EDCI (6.7 mg, 0.035 mmol) was added to a mixture of 1-(4-(6,7-dimethoxyquinolin-4-yloxy)-3-fluorophenyl)-2-oxopyrrolidine-3-carboxylic acid (5.0 mg, 0.012 mmol) and HOBt (4.8 mg, 0.035 mmol) in DMF (2 mL) and was stirred at room temperature for 30 min. 4-Fluoroaniline (2.6 mg, 0.023 mmol) was then added followed by Et₃N (0.005 mL, 0.035 mmol). After stirring 3 days, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material that was purified by silica gel flash column chromatography (1% MeOH in CH₂Cl₂) to afford 0.9 mg (15%) of 170. ¹H-NMR (400 MHz, CD₃OD) δ 8.44 (d, 1H), 7.93 (dd, 1H), 7.63 (s, 1H), 7.62 (m, 2H), 7.55 (d, 1H), 7.42 (t, 1H), 7.37 (s, 1H), 7.08 (t, 1H), 6.51 (d, 1H), 4.45 (m, 1H), 4.02 (s, 6H), 3.99 (m, 1H), 3.81 (m, 1H), 2.59 (m, 1H), 2.51 (m, 1H); ¹⁹F NMR (376 MHz, CD₃OD) δ−120.6, −129.8. LRMS (ESI pos) m/e 520 (M+1).

### Example 71

### Preparation of N-(1-(4-(6,7-dimethoxyquinolin-4-yloxy)-3-fluorophenyl)-2-oxo-1,2-dihydropyridin-3-yl)-4-fluorobenzamide 171

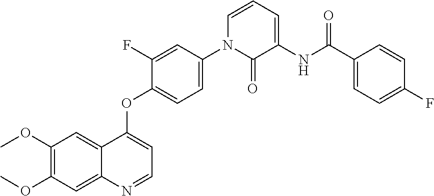

Step A: Preparation of 4-fluoro-N-(2-oxo-1,2-dihydropyridin-3-yl)benzamide: EDCI (0.52 g, 2.70 mmol) was added to a mixture of 4-fluorobenzoic acid (0.25 g, 1.80 mmol) and HOBt (0.37 g, 2.70 mmol) in DMF (5 mL) and was stirred at room temperature for 30 minutes. 3-Aminopyridin-2(1H)-one (0.10 g, 0.91 mmol) was added followed by Et₃N (0.38 mL, 2.70 mmol). After stirring 17 hours, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material that was purified by silica gel flash column chromatography (1% MeOH in CH₂Cl₂) to afford 0.11 g (52%) of the desired product. ¹H-NMR (400 MHz, CDCl₃) δ 11.62 (br. s, 1H), 9.03 (br. s, 1H), 8.63 (dd, 1H), 7.96 (m, 2H), 7.19 (t, 2H), 7.11 (dd, 1H), 6.41 (t, 1H); ¹⁹F NMR (376 MHz, CD₃OD) δ−107.4. LRMS (ESI pos) m/e 233 (M+1).

Step B: Preparation of N-(1-(4-(6,7-dimethoxyquinolin-4-yloxy)-3-fluorophenyl)-2-oxo-1,2-dihydropyridin-3-yl)-4-fluorobenzamide: A mixture of 4-(4-bromo-2-fluorophenoxy)-6,7-dimethoxyquinoline (60 mg, 0.158 mmol, Example 34), 4-fluoro-N-(2-oxo-1,2-dihydropyridin-3-yl)benzamide (35 mg, 0.151 mmol), (1R,2R)-cyclohexane-1,2-diamine (6.9 mg, 0.060 mmol), CuI (5.7 mg, 0.030 mmol), and K₃PO₄ (64 mg, 0.30 mmol) was placed in a sealed vial with dioxane (3 mL). The reaction mixture was then flushed with nitrogen, capped and placed in an oil bath at 110° C., and stirred for 17 hours. After the reaction was cooled to room temperature, the mixture was filtered through a pad of celite with EtOAc. After evaporation of the solvent, the crude was purified by silica gel flash column chromatography (1% MeOH in CH₂Cl₂ and then 100% Et₂₀ to 3:1 Et₂O:EtOAc) to afford 14.2 mg (18%) of 171. ¹H-NMR (400 MHz, CD₃OD) δ 9.21 (br. s, 1H), 8.64 (dd, 1H), 8.55 (d, 1H), 7.95 (m, 2H), 7.58 (s, 1H), 7.50 (s, 1H), 7.46 (dd, 1H), 7.43 (t, 1H), 7.33 (d, 1H), 7.18 (m, 3H), 6.56 (d, 1H), 6.47 (t, 1H), 4.08 (s, 3H), 4.07 (s, 3H); ¹⁹F NMR (376 MHz, CD₃OD) δ−107.1, −125.8. LRMS (ESI pos) m/e 530 (M+1).

### Example 72

### Preparation of N-(3-fluoro-4-(6-methoxy-7-(3-morpholinopropoxy)quinolin-4-yloxy)phenyl)-1-(4-fluorophenyl)-2-oxopiperidine-3-carboxamide 172

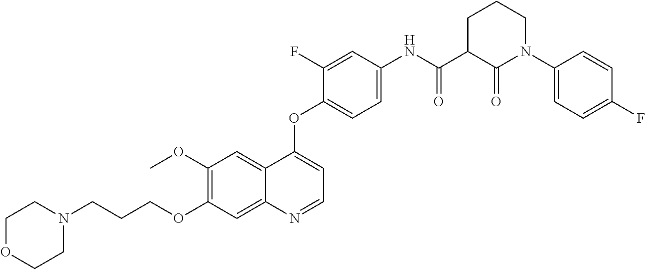

Step A: Preparation of ethyl 1-(4-fluorophenyl)-2-oxopiperidine-3-carboxylate: A mixture of 1-fluoro-4-iodobenzene (1.95 g, 8.76 mmol), ethyl 2-oxopiperidine-3-carboxylate (1.0 g, 5.84 mmol), (1R,2R)-cyclohexane-1,2-diamine (0.27 g, 2.34 mmol), CuI (0.22 g, 1.17 mmol), and K₃PO₄ (2.48 g, 11.68 mmol) was placed in a sealed vial with dioxane (20 mL). The reaction mixture was then flushed with nitrogen, capped and placed in an oil bath at 110° C., and stirred for 15 hours. After the reaction was cooled to room temperature, the mixture was filtered through a pad of celite with EtOAc. After evaporation of the solvent, the crude was purified by silica gel flash column chromatography (2:1=CH₂Cl₂:Et₂O) to afford 1.067 g (69%) of the desired product. ¹H-NMR (400 MHz, CDCl₃) δ 7.24 (m, 2H), 7.13 (m, 2H), 4.24 (m, 2H), 3.67 (m, 2H), 3.57 (t, 1H), 2.27 (m, 1H), 2.21 (m, 1H), 2.10 (m, 1H), 1.95 (m, 1H), 1.31 (t, 3H); ¹⁹F NMR (376 MHz, CDCl₃) δ−115.4. LRMS (ESI pos) m/e 266 (M+1).

Step B: Preparation of 1-(4-fluorophenyl)-2-oxopiperidine-3-carboxylic acid: LiOH (0.87 mL, 0.87 mmol, 1.0 M in H₂O) was added to a solution of ethyl 1-(4-fluorophenyl)-2-oxopiperidine-3-carboxylate in a mixture of THF (3 mL) and MeOH (1 mL) at room temperature for 1 hour. The reaction mixture was acidified to pH 1 with aq. 1 N HCl solution (0.9 mL) and then concentrated to afford the desired product salt. ¹H-NMR (400 MHz, CD₃OD) δ 7.31 (m, 2H), 7.14 (m, 2H), 3.69 (m, 2H), 3.54 (t, 1H), 2.24 (m, 2H), 2.09 (m, 1H), 1.99 (m, 1H); ¹⁹F NMR (376 MHz, CD₃OD) δ−117.3. LRMS (ESI neg) m/e 236 (M−1).

Step C: Preparation of 7-(benzyloxy)-4-(2-fluoro-4-nitrophenoxy)-6-methoxyquinoline: To a stirred solution of 7-(benzyloxy)-6-methoxyquinolin-4-ol (prepared according to the method of WO 2005/030140) (2.81 g, 10 mmol) in 30 mL of 1:1 CH₃CN:DMF at room temperature under nitrogen was added cesium carbonate (6.52 g, 20 mmol). After 30 minutes, 1,2-difluoro-4-nitrobenzene (1.22 mL, 11 mmol) was added. After 3 hours, the reaction was partially concentrated by rotovap and then diluted to 60 mL with EtOAc and washed 4×50 mL with a brine/H₂O mix. The organics were dried (MgSO₄), filtered and concentrated to a residue that was purified by silica gel flash column chromatography (2:3 EtOAc/hexanes). Product containing fractions were pooled and concentrated to a brown solid (1.56 g, 37%). ¹H NMR (400 MHz, CDCl₃) δ 8.56 (d, 1H), 8.19 (dd, 1H), 8.13 (m, 1H), 7.51 (m, 3H), 7.46 (s, 1H), 7.40 (m, 2H), 7.33 (m, 2H), 6.54 (d, 1H), 5.34 (s, 2H), 4.04 (s, 3H).

Step D: Preparation of 4-(2-fluoro-4-nitrophenoxy)-6-methoxyquinolin-7-ol: A solution of 7-(benzyloxy)-4-(2-fluoro-4-nitrophenoxy)-6-methoxyquinoline (1.56 g, 3.71 mmol) was stirred in 11 mL of 33% wt HBr in acetic acid at room temperature under a drying tube. After 4 hours the reaction was diluted with 100 mL Et₂O and filtered. The isolated tan solid was washed with Et₂O and then dried under high vacuum to give 1.45 g (89%) of the HBr salt. This material was stirred as a suspension in 100 mL 4:1 CH₂Cl₂:MeOH. 100 mL of H₂O was added and then solid NaHCO₃ added until pH=7. More MeOH was added until the mixture was a two phase solution. The organics were isolated and the aqueous phase extracted 2×50 mL with CH₂Cl₂. The combined organics were dried (MgSO₄), filtered and concentrated to a yellow solid (1.08 g, 88%). HBr Salt ¹H NMR (400 MHz, CDCl₃/CD₃OD) δ 8.60 (d, 1H), 8.30 (m, 2H), 7.80 (s, 1H), 7.64 (m, 2H), 7.33 (s, 1H), 6.76 (dd, 1H), 4.13 (s, 3H).

Step E: Preparation of 4-(3-(4-(2-fluoro-4-nitrophenoxy)-6-methoxyquinolin-7-yloxy)propyl)morpholine: To a stirred suspension of 4-(2-fluoro-4-nitrophenoxy)-6-methoxyquinolin-7-ol: A solution of 7-(benzyloxy)-4-(2-fluoro-4-nitrophenoxy)-6-methoxyquinoline (610 mg, 1.85 mmol) in 9.2 mL CH₂Cl₂ at room temperature under nitrogen was added 3-morpholinopropan-1-ol (307 uL, 2.22 mmol) followed by triphenylphosphine (775 mg, 2.96 mmol) and finally DEAD (465 uL, 2.96 mmol). After stirring overnight, the reaction was concentrated to a residue by rotovap and purified directly by silica gel flash column chromatography (9/1 EtOAc/MeOH). Product containing fractions were pooled and concentrated to a yellow solid (650 mg, 77%). ¹H NMR (400 MHz, CDCl₃) δ 8.58 (d, 1H), 8.19 (dd, 1H), 8.14 (m, 1H), 7.48 (s, 1H), 7.43 (s, 1H), 7.33 (dd, 1H), 6.55 (d, 1H), 4.29 (dd, 2H), 4.01 (s, 3H), 3.73 (m, 4H), 2.58 (dd, 2H), 2.49 (br m, 4H), 2.14 (m, 2H).

Step F: Preparation of 3-fluoro-4-(6-methoxy-7-(3-morpholinopropoxy)quinolin-4-yloxy)aniline: A solution of 4-(3-(4-(2-fluoro-4-nitrophenoxy)-6-methoxyquinolin-7-yloxy)propyl)morpholine (620 mg, 1.36 mmol) was formed in 75 mL of 95% EtOH and 75 mL EtOAc in a 250 mL Parr Bottle. Pearlman's catalyst (20 wt %, 95 mg, 0.14 g/atom palladium) was added and the reaction put through a vacuum/purge cycle three times with hydrogen gas and then held under 50 psi hydrogen and shaken overnight. The reaction was filtered through GF/F filter paper with 95% EtOH and concentrated to a yellow foam (560 mg, 96%). ¹H NMR (400 MHz, CDCl₃) δ 8.47 (d, 1H), 7.58 (s, 1H), 7.43 (s, 1H), 7.04 (m, 1H), 6.57 (m, 1H), 6.51 (m, 1H), 6.40 (m, 1H), 4.27 (m, 2H), 4.04 (s, 3H), 3.73 (m, 4H), 2.58 (m, 2H), 2.49 (br m, 4H), 2.13 (m, 2H).

Step G: Preparation of N-(3-fluoro-4-(6-methoxy-7-(3-morpholinopropoxy)quinolin-4-yloxy)phenyl)-1-(4-fluorophenyl)-2-oxopiperidine-3-carboxamide: EDCI (29.6 mg, 0.154 mmol) was added to a mixture of 1-(4-fluorophenyl)-2-oxopiperidine-3-carboxylic acid (15.3 mg, 0.064 mmol) and HOBt (20.9 mg, 0.154 mmol) in DMF (2 mL) was stirred at room temperature for 30 minutes. 3-Fluoro-4-(6-methoxy-7-(3-morpholinopropoxy)quinolin-4-yloxy)aniline (11 mg, 0.026 mmol) was added followed by Et₃N (0.022 mL, 0.154 mmol). After stirring 17 hours, the reaction mixture was diluted with EtOAc and washed with saturated aq. NH₄Cl, saturated aq. NaHCO₃, and brine. The organic layer was dried over MgSO₄ and concentrated under reduced pressure to give the crude material that was purified by silica gel flash column chromatography (7% MeOH in CH₂Cl₂) to afford 4.9 mg (29%) of 172. ¹H-NMR (400 MHz, CD₃OD) δ 8.41 (d, 1H), 7.85 (dd, 1H), 7.64 (s, 1H), 7.42 (m, 1H), 7.33 (m, 4H), 7.16 (t, 2H), 6.49 (d, 1H), 4.25 (t, 2H), 4.01 (s, 3H), 3.65-3.78 (m, 7H), 2.64 (t, 2H), 2.54 (m, 3H), 2.0-2.32 (m, 7H); ¹⁹F NMR (376 MHz, CD₃OD) δ−117.2. LRMS (ESI pos) m/e 647 (M+1).

### Example 73

### Preparation of 1-(3-fluoro-4-(6-methoxy-7-(3-morpholinopropoxy)quinolin-4-yloxy)phenyl)-N-(4-fluorophenyl)-2-oxo-1,2-dihydropyridine-3-carboxamide 173

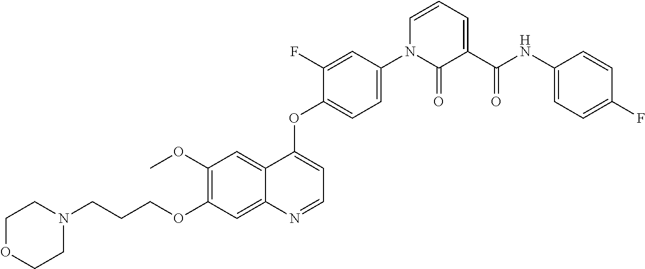

Step A: Preparation of methyl 1-(3-fluoro-4-(6-methoxy-7-(3-morpholinopropoxy)quinolin-4-yloxy)phenyl)-2-oxo-1,2-dihydropyridine-3-carboxylate: To a solution of methyl 2-oxo-2H-pyran-3-carboxylate (9.7 mg, 0.042 mmol) in a mixture of THF (2 mL) and DMF (0.5 mL) at room temperature was added 3-fluoro-4-(6-methoxy-7-(3-morpholinopropoxy)quinolin-4-yloxy)aniline (prepared according to Example 72, steps C-F) (18 mg, 0.042 mmol), and the reaction mixture was stirred for 2.5 hours. To the aniline adduct intermediate formed via Michael addition was added in situ EDCI (13 mg, 0.066 mmol) and DMAP (0.57 mg, 0.0047 mmol) at room temperature. The reaction mixture was stirred at room temperature for 5 days. To the reaction mixture were added aqueous 1 N NaHCO₃, extracted with EtOAc, dried over MgSO₄, and concentrated to give the crude material that was purified by silica gel flash column chromatography (10% MeOH in CH₂Cl₂) to afford 3.5 mg (13%) of the desired product. ¹H-NMR (400 MHz, CD₃OD) δ 8.47 (d, 1H), 8.35 (dd, 1H), 7.98 (dd, 1H), 7.65 (s, 1H), 7.59 (m, 2H), 7.40 (m, 2H), 6.65 (d, 1H), 6.57 (t, 1H), 4.27 (t, 2H), 4.02 (s, 3H), 3.87 (s, 3H), 3.75 (t, 4H), 2.74 (t, 2H), 2.64 (m, 4H), 2.17 (m, 2H); ¹⁹F NMR (376 MHz, CD₃OD) δ−129.1. LRMS (ESI pos) m/e 564 (M+1).

Step B: Preparation of 1-(3-fluoro-4-(6-methoxy-7-(3-morpholinopropoxy)quinolin-4-yloxy)phenyl)-2-oxo-1,2-dihydropyridine-3-carboxylic acid: LiOH (0.012 mL, 0.012 mmol, 1.0 M in H₂O) was added to a solution of methyl 1-(3-fluoro-4-(6-methoxy-7-(3-morpholinopropoxy)quinolin-4-yloxy)phenyl)-2-oxo-1,2-dihydropyridine-3-carboxylate in a mixture of THF (1.5 mL) and MeOH (0.5 mL) at room temperature for 6 hours. The reaction mixture was acidified to pH 1 with aqueous 1 N HCl solution (0.012 mL) and then concentrated to afford the desired product salt. LRMS (ESI pos) m/e 550 (M+1).

Step C: Preparation of 1-(3-fluoro-4-(6-methoxy-7-(3-morpholinopropoxy)quinolin-4-yloxy)phenyl)-N-(4-fluorophenyl)-2-oxo-1,2-dihydropyridine-3-carboxamide: EDCI (3.6 mg, 0.019 mmol) was added to a mixture of 1-(3-fluoro-4-(6-methoxy-7-(3-morpholinopropoxy)quinolin-4-yloxy)phenyl)-2-oxo-1,2-dihydropyridine-3-carboxylic acid (3.4 mg, 0.0062 mmol) and HOBt (2.5 mg, 0.019 mmol) in DMF (0.5 mL) and was stirred at room temperature for 1 hour. 4-Fluoroaniline (2.1 mg, 0.019 mmol) was added followed by Et₃N (1.9 mg, 0.019 mmol). After stirring 17 hours, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material that was purified by silica gel flash column chromatography (5% MeOH in CH₂Cl₂) to afford 0.7 mg (18%) of 173. LRMS (ESI pos) m/e 643 (M+1).

### Example 74

### Preparation of N-(3-fluoro-4-(6-methoxy-7-(3-morpholinopropoxy)quinolin-4-yloxy)phenyl)quinoline-8-carboxamide 174

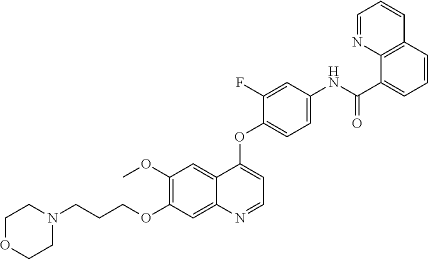

EDCI (27 mg, 0.14 mmol) was added to a mixture of quinoline-8-carboxylic acid (8.1 mg, 0.047 mmol) and HOBt (19 mg, 0.14 mmol) in DMF (2 mL) was stirred at room temperature for 1 hour. 3-Fluoro-4-(6-methoxy-7-(3-morpholinopropoxy)quinolin-4-yloxy)aniline (prepared in Example 72, steps C-F) (10 mg, 0.023 mmol) was added followed by Et₃N (0.020 mL, 0.14 mmol). After stirring 17 hours, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material that was purified by silica gel flash column chromatography (5% MeOH in CH₂Cl₂) to afford 9.6 mg (70%) of 174. ¹H-NMR (400 MHz, CD₃OD/CDCl₃) δ 9.11 (dd, 1H), 8.83 (dd, 1H), 8.50 (dd, 1H), 8.42 (d, 1H), 8.18 (d, 1H), 8.13 (dd, 1H), 7.79 (t, 1H), 7.66 (m, 3H), 7.39 (t, 1H), 7.36 (s, 1H), 6.54 (d, 1H), 4.27 (t, 2H), 4.04 (s, 3H), 3.75 (t, 4H), 2.66 (t, 2H), 2.56 (m, 4H), 2.15 (m, 2H); ¹⁹F NMR (376 MHz, CD₃OD/CDCl₃) δ−129.0. LRMS (ESI pos) m/e 583 (M+1).

### Example 75

### Preparation of N-(3-fluoro-4-(6-methoxy-7-(3-morpholinopropoxy)quinolin-4-yloxy)phenyl)-1-(4-fluorophenyl)-2-oxopyrrolidine-3-carboxamide 175

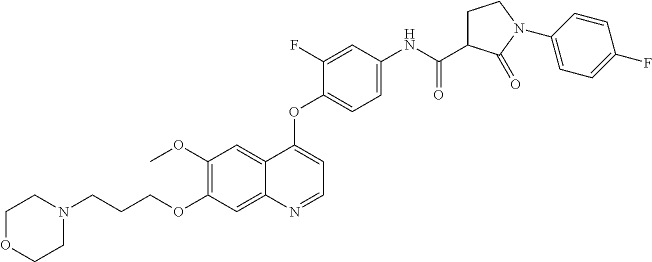

EDCI (67 mg, 0.35 mmol) was added to a mixture of 1-(4-fluorophenyl)-2-oxopyrrolidine-3-carboxylic acid (31 mg, 0.14 mmol) and HOBt (47 mg, 0.35 mmol) in DMF (3 mL) and was stirred at room temperature for 30 minutes. 3-fluoro-4-(6-methoxy-7-(3-morpholinopropoxy)quinolin-4-yloxy)aniline (prepared in Example 72, steps C-F) (30 mg, 0.070 mmol) was added followed by Et₃N (0.049 mL, 0.35 mmol). After stirring 2 hours, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material that was purified by silica gel flash column chromatography (7% MeOH in CH₂Cl₂) to afford 29 mg (65%) of 175. ¹H-NMR (400 MHz, CDCl₃) δ 9.85 (s, 1H), 8.48 (d, 1H), 7.82 (dd, 1H), 7.57 (m, 3H), 7.44 (s, 1H), 7.32 (m, 1H), 7.22 (t, 1H), 7.13 (t, 2H), 6.39 (d, 1H), 4.28 (t, 2H), 4.04 (s, 3H), 3.91 (m, 2H), 3.72 (m, 5H), 2.71 (m, 1H), 2.58 (m, 3H), 2.49 (m, 4H), 2.14 (m, 2H); ¹⁹F NMR (376 MHz, CDCl₃) δ−115.9, −127.1. LRMS (ESI pos) m/e 633 (M+1).

### Example 76

### Preparation of 1-(4-chlorophenyl)-N-(3-fluoro-4-(6-methoxy-7-(3-morpholinopropoxy)quinolin-4-yloxy)phenyl)-2-oxopyrrolidine-3-carboxamide 176

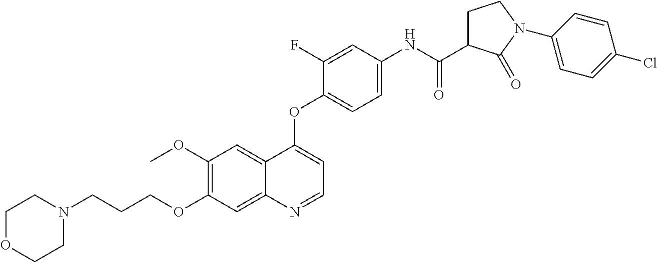

Prepared from 3-fluoro-4-(6-methoxy-7-(3-morpholinopropoxy)quinolin-4-yloxy)aniline (prepared in Example 72, steps C-F) and 1-(4-chlorophenyl)-2-oxopyrrolidine-3-carboxylic acid according to the procedure for Example 75. The crude was purified by silica gel flash column chromatography (5% MeOH in CH₂Cl₂) to afford 25 mg (55%) of 176. ¹H-NMR (400 MHz, CDCl₃) δ 9.85 (s, 1H), 8.48 (d, 1H), 7.82 (dd, 1H), 7.57 (m, 3H), 7.40 (m, 3H), 7.32 (m, 1H), 7.22 (t, 1H), 6.39 (d, 1H), 4.28 (t, 2H), 4.04 (s, 3H), 3.91 (m, 2H), 3.73 (m, 5H), 2.71 (m, 1H), 2.58 (m, 3H), 2.49 (m, 4H), 2.13 (m, 2H); ¹⁹F NMR (376 MHz, CDCl₃) δ−127.0. LRMS (ESI pos) m/e 649, 650 (M+, Cl pattern).

### Example 77

### Preparation of N-(3-fluoro-4-(6-methoxy-7-(3-morpholinopropoxy)quinolin-4-yloxy)phenyl)-2-oxo-1-phenylpyrrolidine-3-carboxamide 177

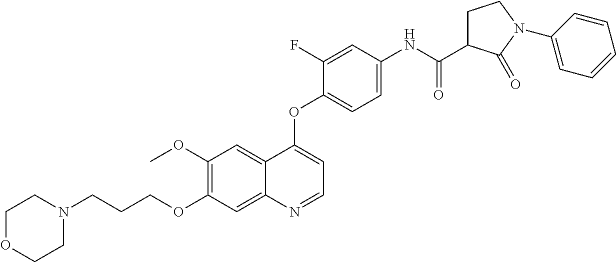

Prepared from 3-fluoro-4-(6-methoxy-7-(3-morpholinopropoxy)quinolin-4-yloxy)aniline (prepared in Example 72, steps C-F) and 2-oxo-1-phenylpyrrolidine-3-carboxylic acid according to the procedure for Example 75. The crude was purified by silica gel flash column chromatography (7% MeOH in CH₂Cl₂) to afford 6.5 mg (5%) of 177. ¹H-NMR (400 MHz, CD₃OD) δ 8.42 (d, 1H), 7.88 (d, 1H), 7.63 (m, 3H), 7.40 (m, 5H), 7.22 (t, 1H), 6.50 (d, 1H), 4.24 (t, 2H), 4.0 (m, 5H), 3.72 (t, 4H), 2.64 (t, 2H), 2.54 (m, 6H), 2.13 (m, 2H); ¹⁹F NMR (376 MHz, CD₃OD) δ−130.1. LRMS (ESI neg) m/e 613 (M−1).

### Example 78

### Preparation of N-(3-fluoro-4-(6-methoxy-7-(3-morpholinopropoxy)quinolin-4-yloxy)phenyl)-1-(4-fluorophenyl)-3-methyl-2-oxopyrrolidine-3-carboxamide 178

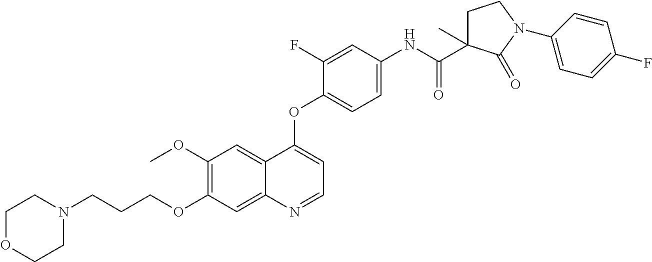

Step A: Preparation of methyl 1-(4-fluorophenyl)-2-oxopyrrolidine-3-carboxylate: To a solution of 1-(4-fluorophenyl)-2-oxopyrrolidine-3-carboxylic acid (0.20 g, 0.90 mmol) in a mixture of Et₂O (6 mL), MeOH (2 mL), and THF (2 mL) was added (diazomethyl)trimethylsilane (1.1 mL, 2.0 M) at 0° C. The resulting mixture was stirred for 30 minutes at room temperature, quenched with AcOH, and diluted with EtOAc. The organic layer was washed with water, NaHCO₃ solution (2×), and brine, dried over MgSO₄, and concentrated under reduced pressure to give the desired product (0.206 g, 98%). LRMS (ESI pos) m/e 238 (M+1).

Step B: Preparation of methyl 1-(4-fluorophenyl)-3-methyl-2-oxopyrrolidine-3-carboxylate: LiH (13.8 mg, 1.737 mmol) was added to the solution of methyl 1-(4-fluorophenyl)-2-oxopyrrolidine-3-carboxylate (0.206 g, 0.868 mmol) in DMF (5 mL) at 0° C. After 30 minutes stirring, iodomethane (0.16 mL, 2.61 mmol) was added to the reaction mixture at 0° C., and then the reaction was warmed to room temperature. The reaction mixture was stirred 17 hours and heated at 40° C. for 3 hours. After cooled to room temperature, the mixture was treated with EtOAc, quenched with ice water, extracted with EtOAc, washed with brine, dried over MgSO₄, and concentrated to give the crude material that was purified by silica gel flash column chromatography (19:1 CH₂Cl₂/EtOAc) to afford 0.149 g (68%) of the desired product. ¹H-NMR (400 MHz, CDCl₃) δ 7.61 (m, 2H), 7.07 (m, 2H), 3.94 (m, 1H), 3.78 (m, 1H), 3.75 (s, 3H), 2.68 (m, 1H), 2.06 (m, 1H), 1.55 (s, 3H); ¹⁹F NMR (376 MHz, CDCl₃) δ−117.6. LRMS (ESI pos) m/e 252 (M+1).

Step C: Preparation of 1-(4-fluorophenyl)-3-methyl-2-oxopyrrolidine-3-carboxylic acid: LiOH (1.2 mL, 1.19 mmol, 1.0 M in H₂O) was added to a solution of methyl 1-(4-fluorophenyl)-3-methyl-2-oxopyrrolidine-3-carboxylate (0.149 g, 0.593 mmol) in a mixture of THF (4.5 mL) and MeOH (1.5 mL) at room temperature for 1 hour. The reaction mixture was acidified with aqueous 1 N HCl solution (1.4 mL), extracted with EtOAc, washed with brine, dried over MgSO₄, and concentrated to afford the desired product (0.13 g, 92%). ¹H-NMR (400 MHz, CD₃OD) δ 7.62 (m, 2H), 7.13 (t, 2H), 3.97 (m, 1H), 3.86 (td, 1H), 2.63 (m, 1H), 2.13 (m, 1H), 1.47 (s, 3H); ¹⁹F NMR (376 MHz, CD₃OD) δ−119.3.

Step D: Preparation of N-(3-fluoro-4-(6-methoxy-7-(3-morpholinopropoxy)quinolin-4-yloxy)phenyl)-1-(4-fluorophenyl)-3-methyl-2-oxopyrrolidine-3-carboxamide: Prepared from 3-fluoro-4-(6-methoxy-7-(3-morpholinopropoxy)quinolin-4-yloxy)aniline (prepared in Example 72, steps C-F) and 1-(4-fluorophenyl)-3-methyl-2-oxopyrrolidine-3-carboxylic acid according to the procedure for Example 75. The crude was purified by silica gel flash column chromatography (5% MeOH in CH₂Cl₂) to afford 66 mg (62%) of 178, as a racemic mixture. ¹H-NMR (400 MHz, CD₃OD) δ 8.41 (d, 1H), 7.87 (dd, 1H), 7.69 (m, 2H), 7.63 (s, 1H), 7.45 (m, 1H), 7.35 (t, 2H), 7.16 (t, 2H), 6.49 (d, 1H), 4.25 (t, 2H), 4.01 (s, 3H), 3.92 (m, 2H), 3.72 (t, 4H), 2.81 (m, 1H), 2.64 (t, 2H), 2.54 (m, 4H), 2.19 (m, 1H), 2.13 (m, 2H), 1.66 (s, 3H); ¹⁹F NMR (376 MHz, CD₃OD) δ−119.0, −130.1. LRMS (ESI pos) m/e 647 (M+1).

### Examples 79 and 80

### Preparation of (S)—N-(3-fluoro-4-(6-methoxy-7-(3-morpholinopropoxy)quinolin-4-yloxy)phenyl)-1-(4-fluorophenyl)-3-methyl-2-oxopyrrolidine-3-carboxamide 179

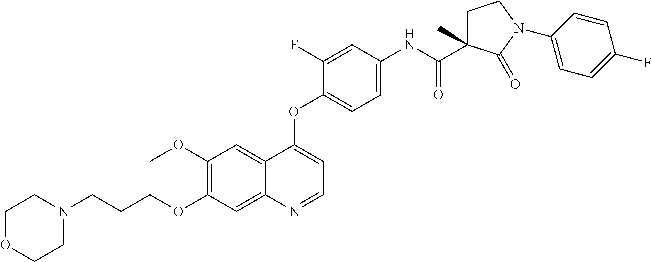

and (R)—N-(3-fluoro-4-(6-methoxy-7-(3-morpholinopropoxy)quinolin-4-yloxy)phenyl)-1-(4-fluorophenyl)-3-methyl-2-oxopyrrolidine-3-carboxamide 180

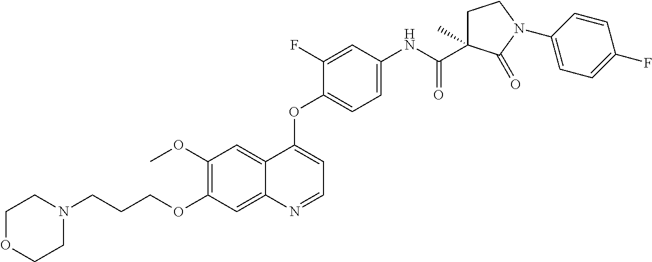

The title compounds were isolated from the racemic mixture of 178 from Example 78 N-(3-fluoro-4-(6-methoxy-7-(3-morpholinopropoxy)quinolin-4-yloxy)phenyl)-1-(4-fluorophenyl)-3-methyl-2-oxopyrrolidine-3-carboxamide by chiral Prep HPLC (Agilent 1100 MSD prep, Fifi) with 40% EtOH and 60% Hexane using Chiralpak IA 250×10 mm column.

### Example 81

### N-(3-fluoro-4-(6-methoxy-7-(3-morpholinopropoxy)quinolin-4-yloxy)phenyl)-3-(4-fluorophenyl)-2-oxo-3-azabicyclo[3.1.0]hexane-1-carboxamide 181

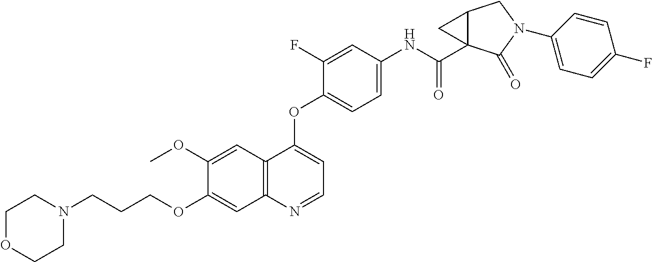

Step A: Preparation of N-allyl-4-fluoroaniline: To a stirred solution of p-fluoroaniline (1.92 mL, 20 mmol) in 60 mL THF at −78° C. was added n-BuLi (12.5 mL, 20 mmol, 1.6 M in hexanes) dropwise by syringe. After 30 minutes, allyl bromide (1.69 mL, 20 mmol) was added neat by syringe. After 2 hours at −78° C., the reaction was allowed to warm to 0° C., was quenched by pouring into 50 mL H₂O and then excess THF was removed by rotovap. The residual material was extracted 2×50 mL with EtOAc. The combined organics were dried (MgSO₄), filtered and concentrated to a crude oil that was purified by silica gel flash column chromatography (5/95 Et₂O/hexanes). Product containing fractions were pooled and concentrated to an orange oil (1.9 g, 63% yield). ¹H NMR (400 MHz, CDCl₃) δ 6.88 (m, 2H), 6.56 (m, 2H), 5.95 (m, 1H), 5.28 (m, 1H), 5.17 (m, 1H), 3.74 (br d, 2H), 3.66 (br s, 1H).

Step B: Preparation of methyl 3-(allyl(4-fluorophenyl)amino)-3-oxopropanoate: To a stirred solution of N-allyl-4-fluoroaniline (207 mg, 1.37 mmol) in 3 mL CH₂Cl₂ at 0° C. under nitrogen was added DIEA (262 uL, 1.5 mmol) followed by DMAP (17 mg, 0.14 mmol) and the methyl malonyl chloride (161 uL, 1.5 mmol) as a solution in 1 mL CH₂Cl₂ dropwise by syringe. After 1 hour at 0° C., the reaction was diluted to 30 mL with CH₂Cl₂ and washed 2×30 mL with 2N HCl and 2×30 mL with saturated NaHCO₃. The organics were dried (MgSO₄), filtered and concentrated to a yellow oil that was used as is in the next reaction (260 mg, 75%). ¹H NMR (400 MHz, CDCl₃) δ 7.18 (m, 2H), 7.09 (m, 2H), 5.85 (m, 1H), 5.13 (m, 2H), 4.29 (m, 2H), 3.68 (s, 3H), 3.19 (s, 2H).

Step C: Preparation of methyl 3-(4-fluorophenyl)-2-oxo-3-azabicyclo[3.1.0]hexane-1-carboxylate: To a stirred suspension of manganese triacetate dihydrate (557 mg, 2.08 mmol) and copper diacetate monohydrate (207 mg, 1.04 mmol) in 6 mL glacial acetic acid at room temperature under nitrogen was added a solution of methyl 3-(allyl(4-fluorophenyl)amino)-3-oxopropanoate (261 mg, 1.04 mmol) in 1 mL acetic acid. After stirring overnight at room temperature, a solution of 10% aqueous sodium bisulfate was added (40 mL). After stirring for a few minutes, the suspension was extracted 3×50 mL with EtOAc. The combined organics were washed 3×50 mL with H₂O and 3×50 mL with saturated NaHCO₃. The organics were dried (MgSO₄), filtered and concentrated. The residue was purified by silica gel flash column chromatography (3/7 EtOAc/hexanes) to give after pooling and concentration of product containing fractions a clear solid (26 mg, 10%). ¹H NMR (400 MHz, CDCl₃) δ 7.51 (m, 2H), 7.04 (m, 2H), 4.04 (m, 1H), 3.82 (s, 3H), 3.71 (d, 1H), 2.50 (m, 1H), 2.05 (m, 1H), 1.31 (m, 1H).

Step D: Preparation of 3-(4-fluorophenyl)-2-oxo-3-azabicyclo[3.1.0]hexane-1-carboxylic acid: To a stirred solution of methyl 3-(4-fluorophenyl)-2-oxo-3-azabicyclo[3.1.0]hexane-1-carboxylate (26 mg, 0.1 mmol) in 1 mL 3:2 THF:H₂O at room temperature under nitrogen was added lithium hydroxide powder (4.8 mg, 0.2 mmol). After stirring overnight, the reaction was partitioned between EtOAc (30 mL) and 2N HCl (30 mL). The organics were washed 1×30 mL with brine, was dried (MgSO₄), filtered and concentrated to a brown solid (20 mg, 85%). ¹H NMR (400 MHz, CDCl₃) δ 7.47 (m, 2H), 7.08 (m, 2H), 4.10 (m, 1H), 3.77 (m, 1H), 2.75 (m, 1H), 2.05 (m, 1H), 1.44 (m, 1H).

Step E: To a stirred solution of 3-(4-fluorophenyl)-2-oxo-3-azabicyclo[3.1.0]hexane-1-carboxylic acid (20 mg, 0.85 mmol) in 850 uL CH₂Cl₂ at room temperature under nitrogen was added DIEA (44 uL, 0.26 mmol) followed by EDCI (24 mg, 0.13 mmol) and HOBt (17 mg, 0.13 mmol). After 30 minutes, 3-fluoro-4-(6-methoxy-7-(3-morpholinopropoxy)quinolin-4-yloxy)aniline (prepared in Example 72, steps C-F) (36 mg, 0.85 mmol) was added as a solid. After stirring overnight, the reaction was diluted to 30 mL with CH₂Cl₂ and stirred with 10 mL 10% Na₂CO₃. The layers were separated and the aqueous solution extracted 1×10 mL with CH₂Cl₂. The combined organics were dried (MgSO₄), filtered and concentrated. The crude product was purified by silica gel flash column chromatography, loading with CH₂Cl₂ and eluted with 100 mL CH₂Cl₂ and then 5/95 MeOH/CH₂Cl₂. Fractions were pooled and concentrated to give 181 as a yellow oil (30 mg, 55%). ¹H NMR (400 MHz, CDCl₃) δ 10.50 (s, 1H), 8.48 (d, 1H), 7.82 (m, 1H), 7.57 (s, 1H), 7.48 (m, 2H), 7.44 (s, 1H), 7.31 (m, 1H), 7.22 (m, 1H), 7.11 (m, 2H), 6.40 (m, 1H), 5.30 (s, 1H), 4.28 (m, 2H), 4.11 (m, 1H), 4.04 (s, 3H), 3.77 (m, 1H), 3.72 (m, 4H), 2.81 (m, 1H), 2.58 (m, 2H), 2.49 (m, 4H), 2.13 (m, 2H), 2.05 (m, 1H), 1.39 (m, 1H).

### Example 82

### Preparation of N-(3-fluoro-4-(6-methoxy-7-(3-morpholinopropoxy)quinolin-4-yloxy)phenyl)-1-(4-fluorophenyl)-2-oxo-1,2-dihydropyridine-3-carboxamide 182

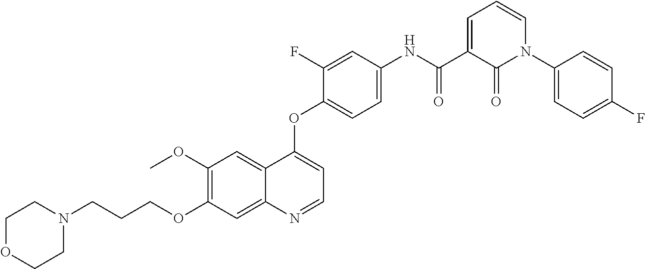

Prepared from 3-fluoro-4-(6-methoxy-7-(3-morpholinopropoxy)quinolin-4-yloxy)aniline (prepared in Example 72, steps C-F) and 1-(4-fluorophenyl)-2-oxo-1,2-dihydropyridine-3-carboxylic acid (prepared from methyl 2-oxo-2H-pyran-3-carboxylate with 4-fluoroaniline and followed by hydrolysis using the methods described in US 2005/0239820) according to the procedure for Example 75. The crude was purified by silica gel flash column chromatography (7% MeOH in CH₂Cl₂) to afford 8.1 mg (49%) of 182. ¹H-NMR (400 MHz, CD₃OD) δ 8.66 (dd, 1H), 8.42 (d, 1H), 8.01 (dd, 1H), 7.97 (dd, 1H), 7.64 (s, 1H), 7.54 (m, 2H), 7.43 (m, 1H), 7.34 (m, 4H), 6.74 (t, 1H), 6.51 (d, 1H), 4.25 (t, 2H), 4.01 (s, 3H), 3.72 (t, 4H), 2.64 (t, 2H), 2.54 (m, 4H), 2.13 (m, 2H); ¹⁹F NMR (376 MHz, CD₃OD) δ−114.5, −129.8. LRMS (ESI pos) m/e 643 (M+1).

### Example 83

### Preparation of N-(3-fluoro-4-(6-methoxy-7-(3-morpholinopropoxy)quinolin-4-yloxy)phenyl)-1-(4-fluorobenzyl)-2-oxo-1,2-dihydropyridine-3-carboxamide 183

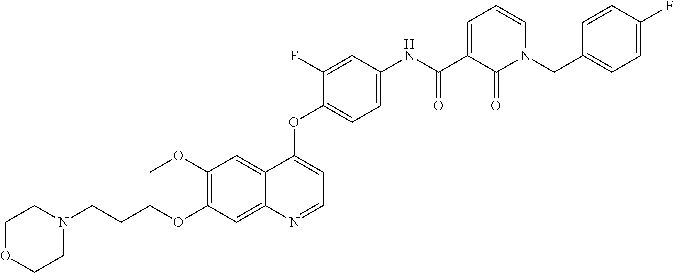

Step A: Preparation of methyl 1-(4-fluorobenzyl)-2-oxo-1,2-dihydropyridine-3-carboxylate: LiH (7.8 mg, 0.980 mmol) was added to the solution of methyl 2-oxo-1,2-dihydropyridine-3-carboxylate (50 mg, 0.327 mmol) in DMF (3 mL) at 0° C. After 30 minutes stirring, 1-(bromomethyl)-4-fluorobenzene (9.3 mg, 0.490 mmol) was added to the reaction mixture at 0° C., and then the reaction was warmed to room temperature. After 4 hours stirring, the reaction mixture was quenched with ice water, extracted with EtOAc, washed with brine, dried over MgSO₄, and concentrated to give the crude material that was purified by silica gel flash column chromatography (100% Et₂O and then 3:1=Et₂O:EtOAc) to afford both of 23.2 mg (27%) of methyl 1-(4-fluorobenzyl)-2-oxo-1,2-dihydropyridine-3-carboxylate and 25.5 mg (22%) of 4-fluorobenzyl 1-(4-fluorobenzyl)-2-oxo-1,2-dihydropyridine-3-carboxylate. For methyl 1-(4-fluorobenzyl)-2-oxo-1,2-dihydropyridine-3-carboxylate: ¹H-NMR (400 MHz, CDCl₃) δ 8.16 (dd, 1H), 7.52 (dd, 1H), 7.33 (dd, 2H), 7.02 (t, 2H), 6.22 (t, 1H), 5.13 (s, 2H), 3.91 (s, 3H); ¹⁹F NMR (376 MHz, CDCl₃) δ−113.9. LRMS (ESI pos) m/e 262 (M+1). For 4-fluorobenzyl 1-(4-fluorobenzyl)-2-oxo-1,2-dihydropyridine-3-carboxylate: ¹H-NMR (400 MHz, CDCl₃) δ 8.12 (dd, 1H), 7.51 (dd, 1H), 7.45 (m, 2H), 7.33 (m, 2H), 7.04 (m, 4H), 6.21 (t, 1H), 5.31 (s, 2H), 5.14 (s, 2H); ¹⁹F NMR (376 MHz, CDCl₃) δ−113.8, −114.4. LRMS (ESI pos) m/e 356 (M+1).

Step B: Preparation of 1-(4-fluorobenzyl)-2-oxo-1,2-dihydropyridine-3-carboxylic acid: LiOH (0.14 mL, 0.14 mmol, 1.0 M in H₂O) was added to a solution of 4-fluorobenzyl 1-(4-fluorobenzyl)-2-oxo-1,2-dihydropyridine-3-carboxylate (24 mg, 0.068 mmol) in a mixture of THF (1.5 mL) and MeOH (0.5 mL) at room temperature for 2 hours. The reaction mixture was acidified to pH 1 with aq 1 N HCl solution (0.14 mL) and then concentrated to afford the desired product salt. ¹H-NMR (400 MHz, CD₃OD) δ 8.46 (dd, 1H), 8.16 (dd, 1H), 7.45 (m, 2H), 7.10 (m, 2H), 6.68 (t, 1H), 5.31 (s, 2H); ¹⁹F NMR (376 MHz, CD₃OD) δ−115.5. LRMS (ESI neg) m/e 246 (M−1).

Step C: Preparation of N-(3-fluoro-4-(6-methoxy-7-(3-morpholinopropoxy)quinolin-4-yloxy)phenyl)-1-(4-fluorobenzyl)-2-oxo-1,2-dihydropyridine-3-carboxamide: Prepared from 3-fluoro-4-(6-methoxy-7-(3-morpholinopropoxy)quinolin-4-yloxy)aniline (prepared in Example 72, steps C-F) and 1-(4-fluorobenzyl)-2-oxo-1,2-dihydropyridine-3-carboxylic acid according to the procedure for Example 72. The crude was purified by silica gel flash column chromatography (7% MeOH in CH₂Cl₂) to afford 6.9 mg (46%) of 183. ¹H-NMR (400 MHz, CD₃OD/CDCl₃) δ 8.58 (dd, 1H), 8.41 (d, 1H), 8.07 (dd, 1H), 8.0 (dd, 1H), 7.64 (s, 1H), 7.45 (m, 3H), 7.34 (m, 2H), 7.10 (t, 2H), 6.64 (t, 1H), 6.51 (d, 1H), 5.32 (s, 2H), 4.26 (t, 2H), 4.03 (s, 3H), 3.73 (t, 4H), 2.65 (t, 2H), 2.55 (m, 4H), 2.14 (m, 2H); ¹⁹F NMR (376 MHz, CD₃OD/CDCl₃) δ−115.9, −129.3. LRMS (ESI pos) m/e 657 (M+1).

### Example 84

### Preparation of 1-(4-chlorobenzyl)-N-(3-fluoro-4-(6-methoxy-7-(3-morpholinopropoxy)quinolin-4-yloxy)phenyl)-2-oxo-1,2-dihydropyridine-3-carboxamide 184

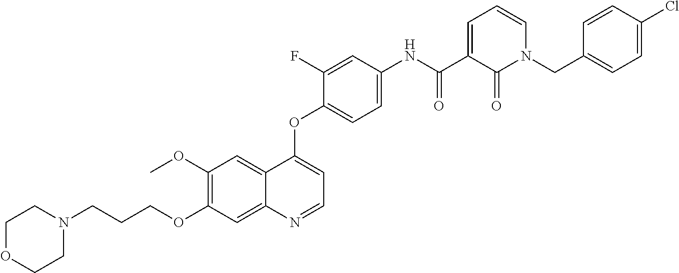

Prepared from 3-fluoro-4-(6-methoxy-7-(3-morpholinopropoxy)quinolin-4-yloxy)aniline (prepared in Example 72, steps C-F) and 1-(4-chlorobenzyl)-2-oxo-1,2-dihydropyridine-3-carboxylic acid (prepared from methyl 2-oxo-1,2-dihydropyridine-3-carboxylate with 1-(bromomethyl)-4-chlorobenzene and followed by hydrolysis using the methods described in Example 83, steps A and B) according to the procedure for Example 72. The crude was purified by silica gel flash column chromatography (7% MeOH in CH₂Cl₂) to afford 9 mg (57%) of 184. ¹H-NMR (400 MHz, CD₃OD/CDCl₃) δ 8.59 (dd, 1H), 8.41 (d, 1H), 8.05 (dd, 1H), 7.99 (dd, 1H), 7.64 (s, 1H), 7.49 (dd, 1H), 7.34 (m, 6H), 6.65 (t, 1H), 6.41 (d, 1H), 5.32 (s, 2H), 4.27 (t, 2H), 4.03 (s, 3H), 3.74 (t, 4H), 2.66 (t, 2H), 2.56 (m, 4H), 2.15 (m, 2H); ¹⁹F NMR (376 MHz, CD₃OD/CDCl₃) δ−129.0. LRMS (ESI pos) m/e 673 (M+1).

### Example 85

### Preparation of 1-benzyl-N-(3-fluoro-4-(6-methoxy-7-(3-morpholinopropoxy)quinolin-4-yloxy)phenyl)-2-oxo-1,2-dihydropyridine-3-carboxamide 185

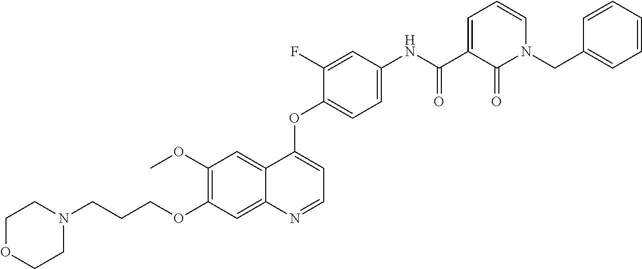

Prepared from 3-fluoro-4-(6-methoxy-7-(3-morpholinopropoxy)quinolin-4-yloxy)aniline (prepared in Example 72, steps C-F) and 1-benzyl-2-oxo-1,2-dihydropyridine-3-carboxylic acid according to the procedure for Example 72. The crude was purified by silica gel flash column chromatography (5% MeOH in CH₂Cl₂) to afford 9.2 mg (62%) of 185. ¹H-NMR (400 MHz, CD₃OD/CDCl₃) δ 8.59 (dd, 1H), 8.41 (d, 1H), 8.02 (m, 2H), 7.64 (s, 1H), 7.46 (m, 1H), 7.34 (m, 7H), 6.63 (t, 1H), 6.50 (d, 1H), 5.35 (s, 2H), 4.26 (t, 2H), 4.03 (s, 3H), 3.74 (t, 4H), 2.65 (t, 2H), 2.55 (m, 4H), 2.14 (m, 2H); ¹⁹F NMR (376 MHz, CD₃OD/CDCl₃) δ−129.1. LRMS (ESI pos) m/e 639 (M+1).

### Example 86

### Preparation of N-(3-fluoro-4-(6-methoxy-7-(3-morpholinopropoxy)quinolin-4-yloxy)phenyl)-1-methyl-2-oxo-1,2-dihydropyridine-3-carboxamide 186

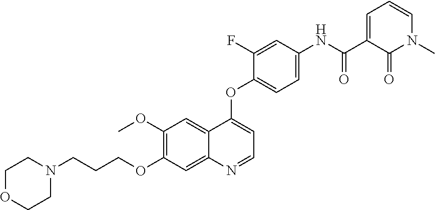

Prepared from 3-fluoro-4-(6-methoxy-7-(3-morpholinopropoxy)quinolin-4-yloxy)aniline (prepared in Example 72, steps C-F) and 1-(4-chlorobenzyl)-2-oxo-1,2-dihydropyridine-3-carboxylic acid (prepared from methyl 2-oxo-1,2-dihydropyridine-3-carboxylate with iodomethane and followed by hydrolysis using the methods described in Example 83, steps A and B) according to the procedure for Example 72. The crude was purified by silica gel flash column chromatography (7% MeOH in CH₂Cl₂) to afford 8.5 mg (65%) of 186. ¹H-NMR (400 MHz, CD₃OD/CDCl₃) δ 8.57 (dd, 1H), 8.41 (d, 1H), 8.0 (m, 1H), 7.64 (s, 1H), 7.46 (m, 1H), 7.36 (m, 2H), 6.61 (t, 1H), 6.51 (d, 1H), 4.26 (t, 2H), 4.02 (s, 3H), 3.73 (t, 4H), 3.72 (s, 3H), 2.65 (t, 2H), 2.55 (m, 4H), 2.14 (m, 2H); ¹⁹F NMR (376 MHz, CD₃OD/CDCl₃) δ−129.3. LRMS (ESI pos) m/e 563 (M+1).

### Example 87

### Preparation of N-(3-fluoro-4-(6-methoxy-7-(3-morpholinopropoxy)quinolin-4-yloxy)phenyl)-2-oxo-1-(pyrimidin-4-ylmethyl)-1,2-dihydropyridine-3-carboxamide 187

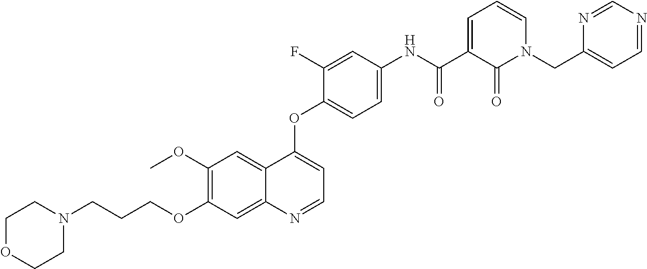

Prepared from 3-fluoro-4-(6-methoxy-7-(3-morpholinopropoxy)quinolin-4-yloxy)aniline (prepared in Example 72, steps C-F) and 1-(4-chlorobenzyl)-2-oxo-1,2-dihydropyridine-3-carboxylic acid (prepared from methyl 2-oxo-1,2-dihydropyridine-3-carboxylate with 4-(chloromethyl)pyrimidine and followed by hydrolysis using the methods described in Example 83 steps A and B) according to the procedure for Example 72. The crude was purified by silica gel flash column chromatography (7% MeOH in CH₂Cl₂) to afford 10.5 mg (70%) of 187. ¹H-NMR (400 MHz, CD₃OD/CDCl₃) δ 9.11 (s, 1H), 8.77 (d, 1H), 8.67 (dd, 1H), 8.41 (dd, 1H), 8.09 (dd, 1H), 7.96 (dd, 1H), 7.63 (s, 1H), 7.51 (d, 1H), 7.42 (d, 1H), 7.36 (s, 1H), 7.29 (t, 1H), 6.72 (t, 1H), 6.48 (d, 1H), 5.44 (s, 2H), 4.28 (t, 2H), 4.04 (s, 3H), 3.76 (t, 4H), 2.67 (t, 2H), 2.57 (m, 4H), 2.16 (m, 2H); ¹⁹F NMR (376 MHz, CD₃OD/CDCl₃) δ−128.5. LRMS (ESI pos) m/e 641 (M+1).

### Example 88

### Preparation of 4-benzyl-N-(3-fluoro-4-(6-methoxy-7-(3-morpholinopropoxy)quinolin-4-yloxy)phenyl)-3-oxo-3,4-dihydropyrazine-2-carboxamide 188

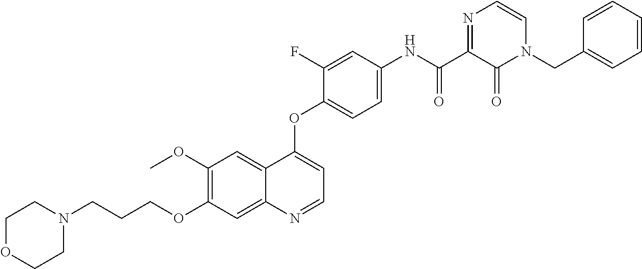

Step A: Preparation of methyl 4-benzyl-3-oxo-3,4-dihydropyrazine-2-carboxylate: LiH (7.8 mg, 0.980 mmol) was added to the solution of methyl 3-oxo-3,4-dihydropyrazine-2-carboxylate (100 mg, 0.65 mmol) in DMF (3 mL) at 0° C. After 30 minutes stirring, (chloromethyl)benzene (0.15 mL, 1.30 mmol) was added to the reaction mixture at 0° C., and then the reaction was warmed to room temperature. After 4 hours stirring, the reaction mixture was quenched with ice water, extracted with EtOAc, washed with brine, dried over MgSO₄, and concentrated to give the crude material that was purified by silica gel flash column chromatography (2% MeOH in CH₂Cl₂) to afford 0.102 g (64%) of the desired product. ¹H-NMR (400 MHz, CDCl₃) δ 7.38 (m, 6H), 7.29 (d, 1H), 5.14 (s, 2H), 3.98 (s, 3H).

Step B: Preparation of 4-benzyl-3-oxo-3,4-dihydropyrazine-2-carboxylic acid: LiOH (0.82 mL, 0.82 mmol, 1.0 M in H₂O) was added to a solution of methyl 4-benzyl-3-oxo-3,4-dihydropyrazine-2-carboxylate (100 mg, 0.41 mmol) in a mixture of THF (4.5 mL) and MeOH (1.5 mL) at room temperature for 4 hours. The reaction mixture was acidified to pH 1 with aq. 1 N HCl solution and treated with water (5 mL), extracted with EtOAc, washed with brine, dried over MgSO₄, and concentrated to afford 77 mg (82%) of the desired product. ¹H-NMR (400 MHz, CD₃OD) δ 8.0 (d, 1H), 7.68 (d, 1H), 7.36-7.42 (m, 5H), 5.29 (s, 2H).

Step C: Preparation of 4-benzyl-N-(3-fluoro-4-(6-methoxy-7-(3-morpholinopropoxy)quinolin-4-yloxy)phenyl)-3-oxo-3,4-dihydropyrazine-2-carboxamide: Prepared from 3-fluoro-4-(6-methoxy-7-(3-morpholinopropoxy)quinolin-4-yloxy)aniline (prepared in Example 72, steps C-F) and 4-benzyl-3-oxo-3,4-dihydropyrazine-2-carboxylic acid according to the procedure for Example 72. The crude was purified by silica gel flash column chromatography (7% MeOH in CH₂Cl₂) to afford 24.8 mg (83%) of 188. ¹H-NMR (400 MHz, CD₃OD) δ 8.43 (d, 1H), 8.06 (dd, 1H), 8.01 (d, 1H), 7.73 (d, 1H), 7.65 (s, 1H), 7.55 (d, 1H), 7.45 (m, 1H), 7.38 (m, 6H), 6.52 (d, 1H), 5.33 (s, 2H), 4.25 (t, 2H), 4.01 (s, 3H), 3.72 (t, 4H), 2.64 (t, 2H), 2.54 (m, 4H), 2.13 (m, 2H). LRMS (APCI pos) m/e 640 (M+1).

### Example 89

### Preparation of N-(3-fluoro-4-(6-methoxy-7-(3-morpholinopropoxy)quinolin-4-yloxy)phenyl)-2-(4-fluorophenyl)-3-oxo-2,3-dihydropyridazine-4-carboxamide 189

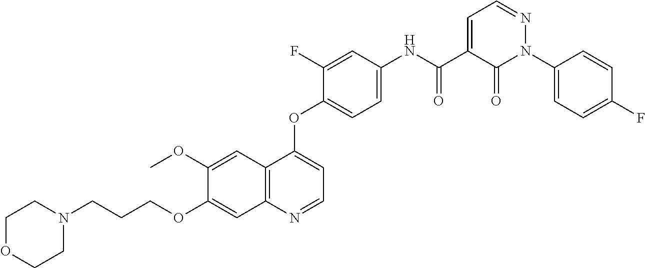

Step A: Preparation of (E)-2-(2-(4-fluorophenyl)hydrazono)acetaldehyde: A mixture of the 4-fluorophenylhydrazine HCl salt (5.0 g, 30.75 mmol), water (20 mL), and acetic acid (20 mL) was added with stirring to a 40% aqueous solution of glyoxal (17.6 mL, 153.8 mmol) during 20 minutes. Stirring was continued for 2 hours and the mixture was then filtered. The precipitate was washed with water and dried to afford 5.0 g (98%) of the desired product. ¹H-NMR (400 MHz, CDCl₃) δ 9.56 (d, 1H), 8.63 (br. s, 1H), 7.24 (m, 1H), 7.16 (m, 2H), 7.06 (m, 2H); ¹⁹F NMR (376 MHz, CDCl₃) δ−120.3. LRMS (ESI pos) m/e 151 (M−16).

Step B: Preparation of (E)-5-(2-(2-(4-fluorophenyl)hydrazono)ethylidene)-2,2-dimethyl-1,3-dioxane-4,6-dione: A suspension of the dioxan-dione (1.44 g, 10.0 mmol) and (E)-2-(2-(4-fluorophenyl)hydrazono)acetaldehyde (1.66 g, 10.0 mmol) in toluene (15 mL) was treated with acetic acid (5 drops) and with piperidine (5 drops). The reaction mixture was then stirred at room temp for 17 hours. The precipitated condensation product was filtered off and thoroughly washed with light petroleum to afford 2.87 g (98%) of the desired product. ¹H-NMR (400 MHz, CD₃OD/CDCl₃) δ 8.72 (d, 1H), 8.24 (d, 1H), 7.32 (m, 2H), 7.08 (t, 2H), 1.76 (s, 6H); ¹⁹F NMR (376 MHz, CD₃OD/CDCl₃) δ−119.1.

Step C: Preparation of 2-(4-fluorophenyl)-3-oxo-2,3-dihydropyridazine-4-carboxylic acid: A mixture of (E)-5-(2-(2-(4-fluorophenyl)hydrazono)ethylidene)-2,2-dimethyl-1,3-dioxane-4,6-dione (0.60 g, 2.05 mmol) and NaOMe (0.133 g, 2.46 mmol) in MeOH (10 mL) was heated under reflux for 15 hours. The salt was treated with cold 1 N HCl solution, extracted with DCM, dried over MgSO₄, and concentrated to afford 0.42 g (87%) of the desired product. ¹H-NMR (400 MHz, CDCl₃) δ 13.57 (br. s, 1H), 8.29 (m, 2H), 7.63 (m, 2H), 7.24 (m, 2H); ¹⁹F NMR (376 MHz, CDCl₃) δ−110.7. LRMS (ESI pos) m/e 235 (M+1).

Step D: Preparation of N-(3-fluoro-4-(6-methoxy-7-(3-morpholinopropoxy)quinolin-4-yloxy)phenyl)-2-(4-fluorophenyl)-3-oxo-2,3-dihydropyridazine-4-carboxamide: Prepared from 3-fluoro-4-(6-methoxy-7-(3-morpholinopropoxy)quinolin-4-yloxy)aniline (prepared in Example 72, steps C-F) and 2-(4-fluorophenyl)-3-oxo-2,3-dihydropyridazine-4-carboxylic acid according to the procedure for Example 72. The crude was purified by silica gel flash column chromatography (5% MeOH in CH₂Cl₂) to afford 10 mg (66%) of 189. ¹H-NMR (400 MHz, CDCl₃/CD₃OD) δ 8.41 (d, 1H), 8.38 (d, 1H), 8.32 (d, 1H), 8.01 (dd, 1H), 7.66 (m, 2H), 7.63 (s, 1H), 7.43 (m, 1H), 7.34 (m, 2H), 7.28 (t, 2H), 6.50 (d, 1H), 4.26 (t, 2H), 4.03 (s, 3H), 3.74 (t, 4H), 2.65 (t, 2H), 2.56 (m, 4H), 2.15 (m, 2H); ¹⁹F NMR (376 MHz, CDCl₃/CD₃OD) δ−113.7, −128.6. LRMS (ESI pos) m/e 644 (M+1).

### Example 90

### Preparation of N-(3-fluoro-4-(6-methoxy-7-(3-(4-methylpiperazin-1-yl)propoxy)quinolin-4-yloxy)phenyl)-2-(4-fluorophenyl)-3-oxo-2,3-dihydropyridazine-4-carboxamide 190

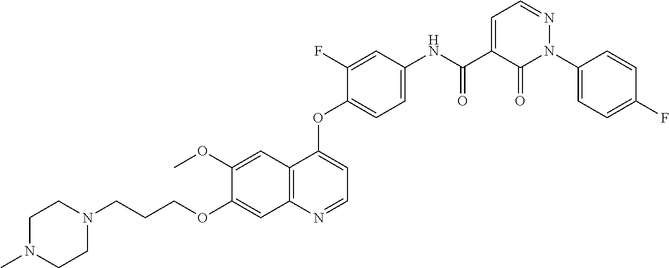

Step A: Preparation of 4-(2-fluoro-4-nitrophenoxy)-6-methoxy-7-(3-(4-methylpiperazin-1-yl)propoxy)quinoline: To a stirred suspension of the 4-(2-fluoro-4-nitrophenoxy)-6-methoxyquinolin-7-ol (Example 72, step D, 0.15 g, 0.454 mmol) in CH₂Cl₂ (4 mL) at room temperature under nitrogen was added the 3-(4-methylpiperazin-1-yl)propan-1-ol (0.086 g, 0.545 mmol) followed by PPh₃ (0.191 g, 0.727 mmol) and (E)-diethyl diazene-1,2-dicarboxylate (0.127 g, 0.727 mmol). After 17 hours stirring, the reaction was concentrated to a residue under reduced pressure. The crude was purified by silica gel flash column chromatography (10% MeOH in CH₂Cl₂) to afford 0.185 mg (87%) of the desired product. LRMS (ESI pos) m/e 471 (M+1).

Step B: Preparation of 3-fluoro-4-(6-methoxy-7-(3-(4-methylpiperazin-1-yl)propoxy)quinolin-4-yloxy)aniline: 10% Pd/C (0.105 g, 0.197 mmol, 20% Wt) was added to a solution of 4-(2-fluoro-4-nitrophenoxy)-6-methoxy-7-(3-(4-methylpiperazin-1-yl)propoxy)quinoline in a mixture of THF (6 mL) and EtOH (3 mL) at room temperature and then the mixture was held under 1 atmosphere of hydrogen gas pressure. After 17 hours stirring, the mixture was filtered with MeOH and concentrated under reduced pressure to give the desired product (0.17 g, 98%). ¹H-NMR (400 MHz, CD₃OD) δ 8.39 (d, 1H), 7.63 (s, 1H), 7.33 (s, 1H), 7.04 (t, 1H), 6.62 (dd, 1H), 6.57 (m, 1H), 6.46 (d, 1H), 4.24 (t, 2H), 4.0 (s, 3H), 2.68 (t, 2H), 2.62 (m, 8H), 2.35 (s, 3H), 2.12 (m, 2H); ¹⁹F NMR (376 MHz, CD₃OD) δ−132.4. LRMS (ESI pos) m/e 441 (M+1).

Step C: Preparation of N-(3-fluoro-4-(6-methoxy-7-(3-(4-methylpiperazin-1-yl)propoxy)quinolin-4-yloxy)phenyl)-2-(4-fluorophenyl)-3-oxo-2,3-dihydropyridazine-4-carboxamide: Prepared from 3-fluoro-4-(6-methoxy-7-(3-(4-methylpiperazin-1-yl)propoxy)quinolin-4-yloxy)aniline and 2-(4-fluorophenyl)-3-oxo-2,3-dihydropyridazine-4-carboxylic acid (Example 89, step C) according to the procedure for Example 72. The crude was purified by silica gel flash column chromatography (10 to 20% MeOH in CH₂Cl₂) to afford 9.2 mg (31%) of 190. ¹H-NMR (400 MHz, CD₃OD) δ 8.42 (d, 1H), 8.35 (d, 1H), 8.30 (d, 1H), 8.04 (dd, 1H), 7.68 (m, 2H), 7.64 (s, 1H), 7.50 (m, 1H), 7.38 (t, 1H), 7.35 (s, 1H), 7.29 (t, 2H), 6.51 (d, 1H), 4.24 (t, 2H), 4.0 (s, 3H), 2.65 (t, 2H), 2.55 (m, 6H), 2.30 (s, 3H), 2.12 (m, 2H); ¹⁹F NMR (376 MHz, CD₃OD) δ−114.8, −129.6. LRMS (APCI pos) m/e 657 (M+1).

### Example 91

### Preparation of N-(3-fluoro-4-(6-methoxy-7-(3-(piperidin-1-yl)propoxy)quinolin-4-yloxy)phenyl)-2-(4-fluorophenyl)-3-oxo-2,3-dihydropyridazine-4-carboxamide 191

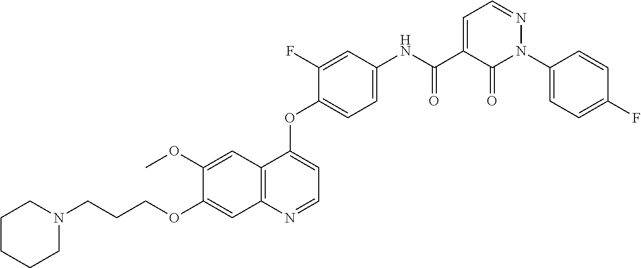

Prepared from 3-fluoro-4-(6-methoxy-7-(3-(piperidin-1-yl)propoxy)quinolin-4-yloxy)aniline (prepared from 4-(2-fluoro-4-nitrophenoxy)-6-methoxyquinolin-7-ol with 3-(piperidin-1-yl)propan-1-ol and followed by hydrogenation using the methods described in Example 90, steps A and B) and Example 89C 2-(4-fluorophenyl)-3-oxo-2,3-dihydropyridazine-4-carboxylic acid according to the procedure for Example 72. The crude was purified by silica gel flash column chromatography (10 to 20% MeOH in CH₂Cl₂) to afford 20 mg (66%) of 191. ¹H-NMR (400 MHz, CDCl₃/CD₃OD) δ 8.43 (d, 1H), 8.38 (d, 1H), 8.32 (d, 1H), 8.03 (dd, 1H), 7.68 (m, 2H), 7.65 (s, 1H), 7.49 (m, 1H), 7.36 (t, 2H), 7.29 (t, 2H), 6.52 (d, 1H), 4.27 (t, 2H), 4.03 (s, 3H), 2.89 (m, 2H), 2.80 (m, 4H), 2.23 (m, 2H), 1.74 (m, 4H), 1.59 (m, 2H); ¹⁹F NMR (376 MHz, CDCl₃/CD₃OD) δ−114.0, −128.8. LRMS (APCI pos) m/e 642 (M+1).

### Example 92

### Preparation of 2-(4-fluorophenyl)-N-(6-(6-methoxy-7-(3-morpholinopropoxy)quinolin-4-yloxy)pyridin-3-yl)-3-oxo-2,3-dihydropyridazine-4-carboxamide 192

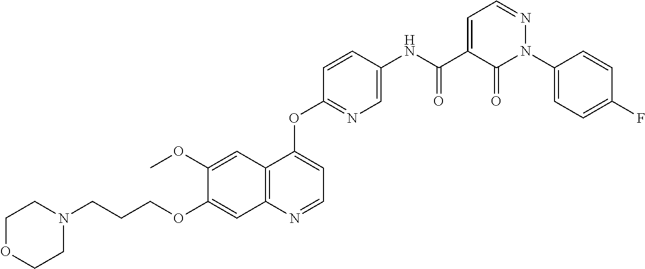

Step A: Preparation of 7-(benzyloxy)-6-methoxy-4-(5-nitropyridin-2-yloxy)quinoline: To a stirred solution of 7-(benzyloxy)-6-methoxyquinolin-4-ol (562 mg, 2 mmol) (reference for preparation given in Example 73, step C) in 20 mL CH₃CN at room temperature under nitrogen was added cesium carbonate (716 mg, 2.2 mmol). After 5 minutes 2-chloro-5-nitropyridine (384 mg, 2.2 mmol) was added. The reaction was allowed to proceed overnight. The reaction was diluted to 30 mL with EtOAc and washed 4×30 mL with H₂O/brine and then 1×30 mL with brine. The organics were dried (MgSO₄), filtered and concentrated. The residue was purified by silica gel flash column chromatography (1/1 EtOAc/hexanes). Product containing fractions were pooled and concentrated to a brown solid (177 mg, 22%). ¹H-NMR (400 MHz, CDCl₃) δ 9.06 (d, 1H), 8.71 (d, 1H), 8.58 (m, 1H), 7.52 (s, 1H), 7.50 (d, 1H), 7.39 (m, 2H), 7.33 (m, 1H), 7.24 (d, 1H), 7.16 (s, 1H), 7.05 (d, 1H), 5.32 (s, 2H), 3.94 (s, 3H).

Step B: Preparation of 6-methoxy-4-(5-nitropyridin-2-yloxy)quinolin-7-ol: A suspension of 7-(benzyloxy)-6-methoxy-4-(5-nitropyridin-2-yloxy)quinoline (130 mg, 0.32 mmol) was stirred in 300 uL 33 wt % HBr in acetic acid. After 4 hours a tan precipitate had formed. The reaction was diluted with 5 mL diethyl ether and filtered, rinsing with ether. The isolated solid is presumably the di HBr salt. This material was dissolved in 20 mL 4:1 CH₂Cl₂:MeOH and stirred with 20 mL of water (pH was <3). The pH was raised to approximately 6-7 with saturated NaHCO₃. A little more MeOH was added to make the mixture a biphasic solution with no precipitate present. The organics were isolated, dried (MgSO₄), filtered and concentrated to a yellow solid (80 mg, 79%). HBr Salt: ¹H-NMR (400 MHz, d₆-DMSO) δ 9.18 (d, 1H), 9.00 (d, 1H), 8.89 (m, 1H), 7.76 (d, 1H), 7.66 (d, 1H), 7.65 (s, 1H), 7.56 (s, 1H), 4.00 (s, 3H).

Step C: Preparation of 6-methoxy-7-(3-morpholinopropoxy)-4-(5-nitropyridin-2-yloxy)quinoline: To a stirred suspension of 6-methoxy-4-(5-nitropyridin-2-yloxy)quinolin-7-ol (150 mg, 0.48 mmol) in 1.5 mL CH₂Cl₂ at room temperature under nitrogen was added 3-morpholinopropan-1-ol (106 uL, 0.77 mmol) followed by triphenylphosphine (201 mg, 0.77 mmol) and finally DEAD (121 uL, 0.77 mmol). After stirring overnight, the reaction was concentrated to a residue by rotovap and purified directly by silica gel flash column chromatography (9/1 EtOAc/MeOH). Product containing fractions were pooled and concentrated to a tan foam (100 mg, 47%). ¹H-NMR (400 MHz, CDCl₃) δ 9.07 (d, 1H), 8.73 (d, 1H), 8.58 (m, 1H), 7.50 (s, 1H), 7.25 (d, 1H), 7.15 (s, 1H), 7.06 (d, 1H), 4.28 (m, 2H), 3.92 (s, 3H), 3.72 (m, 4H), 2.57 (m, 2H), 2.48 (m, 4H), 2.13 (m, 2H).

Step D: Preparation of 6-(6-methoxy-7-(3-morpholinopropoxy)quinolin-4-yloxy)pyridin-3-amine: A solution of 6-methoxy-7-(3-morpholinopropoxy)-4-(5-nitropyridin-2-yloxy)quinoline (100 mg, 0.23 mmol) was formed in 25 mL of 95% EtOH and 25 mL EtOAc in a 250 mL Parr bottle. Pearlman's catalyst (160 mg, 0.23 g/atom) was added and the reaction put through a vacuum/purge cycle three times with hydrogen gas and then held under 50 psi hydrogen and shaken overnight. The reaction was filtered through GF/F filter paper with 95% EtOH and concentrated to a yellow foam (93 mg, 100%). ¹H-NMR (400 MHz, CDCl₃) δ 8.49 (d, 1H), 7.84 (d, 1H), 7.54 (s, 1H), 7.44 (s, 1H), 7.21 (m, 1H), 6.98 (d, 1H), 6.63 (d, 1H), 4.28 (m, 2H), 4.00 (s, 3H), 3.81 (m, 4H), 2.73 (m, 2H), 2.65 (m, 4H), 2.22 (m, 2H).

Step E: Prepared from 6-(6-methoxy-7-(3-morpholinopropoxy)quinolin-4-yloxy)pyridin-3-amine and 2-(4-fluorophenyl)-3-oxo-2,3-dihydropyridazine-4-carboxylic acid (Example 89C) according to the procedure for Example 72. The crude was purified by silica gel flash column chromatography (7% MeOH in CH₂Cl₂) to afford 10 mg (33%) of 192. ¹H-NMR (400 MHz, CD₃OD) δ 8.63 (d, 1H), 8.53 (d, 1H), 8.41 (d, 1H), 8.38 (dd, 1H), 8.32 (d, 1H), 7.64 (m, 2H), 7.47 (s, 1H), 7.38 (s, 1H), 7.27 (m, 3H), 6.87 (d, 1H), 4.28 (t, 2H), 3.99 (s, 3H), 3.76 (t, 4H), 2.66 (t, 2H), 2.57 (m, 4H), 2.16 (m, 2H); ¹⁹F NMR (376 MHz, CD₃OD) δ−112.4. LRMS (APCI pos) m/e 627 (M+1).

### Example 93

### Preparation of N-(3-fluoro-4-(6-methoxy-7-(3-(4-methylpiperazin-1-yl)propoxy)quinolin-4-yloxy)phenyl)-2-(4-fluorophenyl)-6-methyl-3-oxo-2,3-dihydropyridazine-4-carboxamide 193

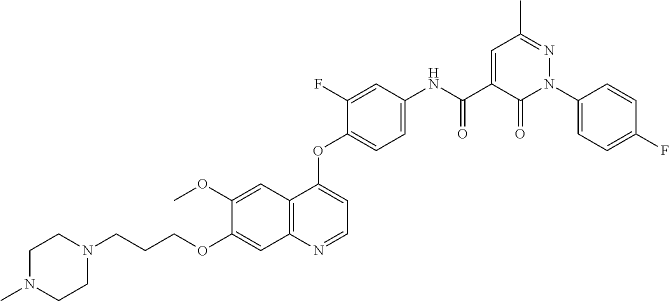

Step A: Preparation of (E)-2-(2-(4-fluorophenyl)hydrazono)propanal and 1-(2-(4-fluorophenyl)hydrazono)propan-2-one: A mixture of (4-fluorophenyl)hydrazine HCl salt (2.0 g, 12.30 mmol), water (10 mL), and acetic acid (10 mL) was added with stirring to a 40% aqueous solution of 2-oxopropanal (9.41 mL, 61.5 mmol) during 20 minutes. Stirring was continued for 4 hours and the mixture was then filtered. The precipitate was washed with water and dried to afford the desired products. The crude was purified by silica gel flash column chromatography (1:50 to 1:10 EtOAc/CH₂Cl₂) to afford 2.05 g (93%) of both desired products.

(E)-2-(2-(4-fluorophenyl)hydrazono)propanal: ¹H-NMR (400 MHz, CDCl₃) δ 9.47 (s, 1H), 8.09 (br. s, 1H), 7.24 (m, 2H), 7.06 (t, 2H), 1.98 (s, 3H); ¹⁹F NMR (376 MHz, CDCl₃) δ−121.0.

1-(2-(4-fluorophenyl)hydrazono)propan-2-one (two isomers—cis and trans): ¹H-NMR (400 MHz, CDCl₃) δ 8.23 (br. s, 1H), 7.22 (m, 2H isomer b), 7.13 (m, 2H isomer b), 7.04 (m, 4H isomer a), 6.96 (s, 1H), 2.44 (s, 3H isomer a), 2.67 (s, 3H isomer b); ¹⁹F NMR (376 MHz, CDCl₃) δ−120.2, −121.4.

Step B: Preparation of 2-(4-fluorophenyl)-6-methyl-3-oxo-2,3-dihydropyridazine-4-carboxylic acid: A suspension of 2,2-dimethyl-1,3-dioxane-4,6-dione (0.71 g, 4.93 mmol) and (E)-2-(2-(4-fluorophenyl)hydrazono)propanal (0.889 g, 4.934 mmol) in toluene (20 mL) was treated with acetic acid (5 drops) and with piperidine (5 drops). The reaction mixture was then stirred at room temperature for 17 hours. The precipitated condensation-cyclization product (2 steps in one pot reaction) was filtered off and thoroughly washed with light petroleum to afford 0.709 g (58%) of the desired product. ¹H-NMR (400 MHz, CD₃OD) δ 7.96 (s, 1H), 7.61 (m, 2H), 7.24 (t, 2H), 2.45 (s, 3H); ¹⁹F NMR (376 MHz, CD₃OD) δ−115.1. LRMS (ESI pos) m/e 249 (M+1).

Step C: Preparation of N-(3-fluoro-4-(6-methoxy-7-(3-(4-methylpiperazin-1-yl)propoxy)quinolin-4-yloxy)phenyl)-2-(4-fluorophenyl)-6-methyl-3-oxo-2,3-dihydropyridazine-4-carboxamide: Prepared from 3-fluoro-4-(6-methoxy-7-(3-(4-methylpiperazin-1-yl)propoxy)quinolin-4-yloxy)aniline (prepared in example 72, steps C-F) and 2-(4-fluorophenyl)-6-methyl-3-oxo-2,3-dihydropyridazine-4-carboxylic acid according to the procedure for Example 72. The crude was purified by silica gel flash column chromatography (10% MeOH in CH₂Cl₂) to afford 7.4 mg (19%) of 193. ¹H-NMR (400 MHz, CD₃OD) δ 8.40 (d, 1H), 8.26 (s, 1H), 8.02 (dd, 1H), 7.65 (m, 2H), 7.62 (s, 1H), 7.46 (m, 1H), 7.37 (t, 1H), 7.34 (s, 1H), 7.27 (t, 2H), 6.49 (d, 1H), 4.23 (t, 2H), 3.99 (s, 3H), 2.65 (t, 2H), 2.55 (m, 6H), 2.49 (s, 3H), 2.31 (s, 3H), 2.11 (m, 2H); ¹⁹F NMR (376 MHz, CD₃OD) δ−115.0, −129.5. LRMS (ESI pos) m/e 671 (M+1).

### Example 94

### N-(3-Fluoro-4-(6-methoxy-7-(3-morpholinopropoxy)quinolin-4-yloxy)phenyl)pyridin-2-amine 194

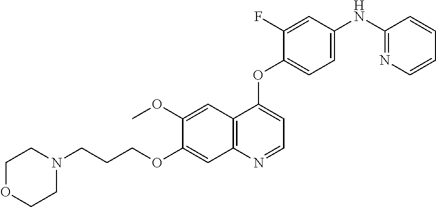

Step A: Preparation of N-(3-fluoro-4-methoxyphenyl)pyridin-2-amine: A mixture of pyridin-2-amine (0.433 g, 4.60 mmol), 4-bromo-2-fluoro-1-methoxybenzene (1.23 g, 5.98 mmol), Pd2(dba)3 (0.421 g, 0.460 mmol), 4,5-bis(diphenylphosphino)-9,9-dimethyl-9H-xanthene (0.799 g, 1.38 mmol), Cs₂CO₃ (3.00 g, 9.20 mmol), in dioxane (25 mL) was stirred at 100° C. for 16 hours. Water (25 mL) was added and extracted with CH₂Cl₂ (3×100 mL). The combined organic layers were dried over Na₂SO₄. Concentration and purification by silica gel flash column chromatography afforded the desired product (0.59 g, 59%). ¹H NMR (400 MHz, CDCl₃) δ 8.17 (m, 1H), 7.48 (m, 1H), 7.22 (dd, J=13.0, 2.6 Hz, 1H), 7.01 (m, 1H), 6.92 (t, J=9.0 Hz, 1H), 6.82 (s, br, 1H, NH), 6.69-6.75 (m, 2H). LRMS (ESI pos) m/e 219 (M+1).

Step B: Preparation of 2-fluoro-4-(pyridin-2-ylamino)phenol: A mixture of N-(3-fluoro-4-methoxyphenyl)pyridin-2-amine (0.587 g, 2.690 mmol) and tribromoborane (3.369 g, 13.45 mmol) in CH₂Cl₂ (50 mL) was stirred at 0° C. for 4 hours. Saturated NaHCO₃ was added and then extracted with CH₂Cl₂ (3×100 mL). The combined organic layers were dried over Na₂SO₄. Concentration afforded the crude product (0.48 g, 88%), which was used in the next step without further purification. ¹H NMR (400 MHz, CDCl₃) δ 8.17 (m, 1H), 7.48 (m, 1H), 7.17 (dd, J=12.0, 2.4 Hz, 1H), 6.88-6.97 (m, 2H), 6.70-6.74 (m, 2H), 6.45 (br s, 1H). LRMS (ESI pos) m/e 205(M+1).

Step C: Preparation of N-(3-fluoro-4-(6-methoxy-7-(3-morpholinopropoxy)quinolin-4-yloxy)phenyl)pyridin-2-amine: A mixture of 4-chloro-6-methoxy-7-(3-morpholinopropoxy)quinoline (prepared according to WO 01/55116, Example 2, 20 mg, 0.059 mmol), 2-fluoro-4-(pyridin-2-ylamino)phenol (14.6 mg, 0.0713 mmol) and N,N-dimethylpyridin-4-amine (0.725 mg, 0.00594 mmol) in bromobenzene (10 mL) was stirred at 150° C. for 2 days. Water (10 mL) was added and the aqueous extracted with CH₂Cl₂ (3×50 mL). The organic layers were combined and dried over Na₂SO₄. The crude product was concentrated and purified by silica gel flash column chromatography to afford 194 (15.8 mg, 53%). ¹H NMR (400 MHz, CDCl₃), δ 8.49 (d, J=5.2 Hz, 1H), 8.27 (dd, J=5.2, 1.6 Hz, 1H), 7.63 (dd, J=12.4, 2.4 Hz, 1H), 7.59 (s, 1H), 7.55-7.58 (m, 1H), 7.44 (s, 1H), 7.14-7.22 (m, 2H), 6.82-6.85 (m, 2H), 6.58 (s, 1H, NH), 6.45 (d, J=5.2 Hz, 1H), 4.28 (t, J=6.8 Hz, 2H), 4.05 (s, 3H), 3.73 (t, J=4.6 Hz, 4H), 2.58 (t, J=7.2 Hz, 2H), 2.49 (m, 4H), 2.10-2.17 (m, 2H). LRMS (APCI neg) m/z 503 (M−1).

### Example 95

### N-(3-Fluoro-4-(6-methoxy-7-(3-morpholinopropoxy)quinolin-4-yloxy)phenyl)-1-methyl-2-oxopyrrolidine-3-carboxamide 195

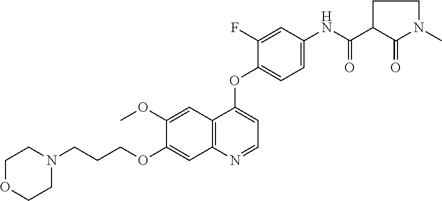

Step A: Preparation of methyl 1-methyl-2-oxopyrrolidine-3-carboxylate: LDA (43.77 mL, 78.79 mmol) was added into a solution of 1-methylpyrrolidin-2-one (5.20 g, 52.53 mmol) in THF (125 mL) under −78° C. The mixture was stirred for 30 minutes, and then methyl carbonochloridate (7.45 g, 78.79 mmol) was added. The mixture was stirred for four hours at room temperature. Water (150 mL) was added and the aqueous phase was extracted with CH₂Cl₂ (3×150 mL). The organic layers were combined and dried over Na₂SO₄. Concentration to afford the crude desired product (7.35 g, 89%) without further purification. ¹H NMR (400 MHz, CD₃Cl). δ 3.37 (s, 3H), 3.28-3.25 (m, 2H), 2.85 (s, 3H), 2.62-2.67 (m, 1H), 2.13-2.22 (m, 1H), 1.99-2.06 (m, 1H).

Step B: Preparation of 1-methyl-2-oxopyrrolidine-3-carboxylic acid: A mixture of methyl 1-methyl-2-oxopyrrolidine-3-carboxylate (1.89 g, 12.04 mmol) and TMSOK (4.64 g, 36.13 mmol) in THF (100 mL) was stirred overnight at room temperature. HCl (50 mL, 100 mmol, 2.0 M in Et₂O) was added and the mixture was stirred for 20 minutes. The solid was removed by filtration with Et₂O. The filtrated was then concentrated under reduced pressure to afford the crude product (1.28 g, 74.2%), which was used for next step without further purification. ¹H NMR (400 MHz, CDCl₃). δ 3.40-3.49 (m, 3H), 2.94 (s, 3H), 2.37-2.47 (m, 2H).

Step C: Preparation of N-(3-fluoro-4-(6-methoxy-7-(3-morpholinopropoxy)quinolin-4-yloxy)phenyl)-1-methyl-2-oxopyrrolidine-3-carboxamide: A mixture of 3-fluoro-4-(6-methoxy-7-(3-morpholinopropoxy)quinolin-4-yloxy)aniline (prepared in Example 72, steps C-F) (10.0 mg, 0.0234 mmol), 1-methyl-2-oxopyrrolidine-3-carboxylic acid (16.7 mg, 0.117 mmol), N¹-((ethylimino)methylene)-N³,N³-dimethylpropane-1,3-diamine hydrochloride (22.4 mg, 0.117 mmol), 1H-benzo[d][1,2,3]triazol-1-ol (15.8 mg, 0.117 mmol) and N-ethyl-N-isopropylpropan-2-amine (0.0204 ml, 0.117 mmol) in CH₂Cl₂ (10 mL) was stirred at room temperature for 2 days. Water (10 mL) was added and the aqueous phase was extracted with CH₂Cl₂ (3×50 mL). The organic layers were combined and dried over Na₂SO₄. Concentration and purification by silica gel flash column chromatography afforded 195 (12.4 mg, 96%). ¹H NMR (400 MHz, CDCl₃) δ 10.02 (s, 1H, NH), 8.48 (d, J=5.2 Hz, 1H), 7.80 (dd, J=12.0, 2.4 Hz, 1H), 7.57 (s, 1H), 7.44 (s, 1H), 7.30 (m, 1H), 7.21 (t, J=8.8 Hz, 1H), 6.39 (d, J=5.2 Hz, 1H), 4.28 (t, J=6.8 Hz, 2H), 4.04 (s, 3H), 3.73 (t, J=4.8 Hz, 4H), 3.43-3.49 (m, 3H), 2.95 (s, 3H), 2.58 (t, J=7.2 Hz, 2H), 2.40-2.54 (m, 6H), 2.10-2.17 (m, 2H). LRMS (APCI neg) m/z 551 (M−1).

### Example 96

### N-(3-Fluoro-4-(6-methoxy-7-(3-morpholinopropoxy)quinolin-4-yloxy)phenyl)-2-oxopyrrolidine-3-carboxamide 196

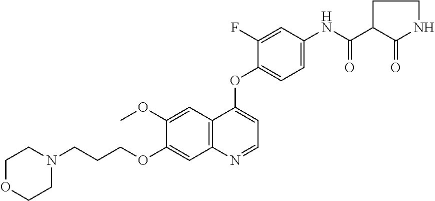

Step A: Preparation of 2-oxopyrrolidine-3-carboxylic acid: A mixture of ethyl 1-methyl-2-oxopyrrolidine-3-carboxylate (0.50 g, 3.18 mmol) and TMSOK (1.34 g, 10.48 mmol) in THF (10 mL) was stirred overnight at room temperature. HCl (20 mL, 100 mmol, 2.0 M in Et2O) was added and the mixture stirred for 20 minutes. The solid was removed by filtration with Et₂O and the filtrate was concentrated under reduced pressure to afford the crude product (0.19 g, 42%), which was used for next step without further purification.

Step B: Preparation of N-(3-fluoro-4-(6-methoxy-7-(3-morpholinopropoxy)quinolin-4-yloxy)phenyl)-2-oxopyrrolidine-3-carboxamide: A mixture of 3-fluoro-4-(6-methoxy-7-(3-morpholinopropoxy)quinolin-4-yloxy)benzenamine (prepared in Example 72, steps C-F) (15.0 mg, 0.0351 mmol), 2-oxopyrrolidine-3-carboxylic acid (22.7 mg, 0.175 mmol), N¹-((ethylimino)methylene)-N³,N³-dimethylpropane-1,3-diamine hydrochloride (33.6 mg, 0.175 mmol), ¹H-benzo[d][1,2,3]triazol-1-ol (23.7 mg, 0.175 mmol) and N-ethyl-N-isopropylpropan-2-amine (22.7 mg, 0.175 mmol) in THF (10 mL) was stirred at room temperature for 16 hours. Water (10 mL) was added and the aqueous phase extracted with CH₂Cl₂ (3×50 mL). The combined organic layers were dried over Na₂SO₄. Concentration and purification by silica gel flash column chromatography afforded 196 (17.3 mg, 92%). ¹H NMR (400 MHz, CDCl₃). δ 9.84 (s, 1H, NH), 8.48 (d, J=5.6 Hz, 1H), 7.80 (dd, J=12.0, 2.4 Hz, 1H), 7.57 (s, 1H), 7.44 (s, 1H), 7.30 (m, 1H), 7.21 (t, J=8.8 Hz, 1H), 6.39 (d, J=5.6 Hz, 1H), 5.77 (s, 1H, NH), 4.28 (t, J=6.6 Hz, 2H), 4.04 (s, 3H), 3.73 (t, J=4.6 Hz, 4H), 3.38-3.51 (m, 3H), 2.53-2.73 (m, 2H), 2.58 (t, J=7.2 Hz, 2H), 2.49 (m, 4H), 2.10-2.17 (m, 2H). LRMS (APCI neg) m/z 537 (M−1).

### Example 97

### N-(3-fluoro-4-(6-methoxy-7-(3-morpholinopropoxy)quinolin-4-yloxy)phenyl)-7,7-dimethyl-2-oxobicyclo[2.2.1]heptane-1-carboxamide 197

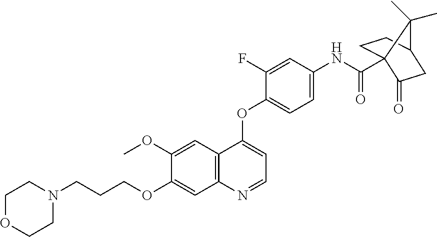

A mixture of 3-fluoro-4-(6-methoxy-7-(3-morpholinopropoxy)quinolin-4-yloxy)benzenamine (prepared in Example 72, steps C-F) (10.0 mg, 0.0234 mmol), (1S)-7,7-dimethyl-2-oxobicyclo[2.2.1]heptane-1-carboxylic acid (21.3 mg, 0.117 mmol), N¹-((ethylimino)methylene)-N³,N³-dimethylpropane-1,3-diamine hydrochloride (22.4 mg, 0.117 mmol), ¹H-benzo[d][1,2,3]triazol-1-ol (15.8 mg, 0.117 mmol) and N-ethyl-N-isopropylpropan-2-amine (0.0204 ml, 0.117 mmol) in THF (10 mL) was stirred at room temperature for 4 days. Water (10 mL) was added and the aqueous phase extracted with CH₂Cl₂ (3×50 mL). The organic layers were combined and dried over Na₂SO₄. Concentration and purification by silica gel flash column chromatography afforded 197 (2.4 mg, 17%). ¹H NMR (400 MHz, CDCl₃). δ 9.94 (s, 1H, NH), 8.47 (d, J=5.2 Hz, 1H), 7.84 (dd, J=12.4, 2.4 Hz, 1H), 7.58 (s, 1H), 7.43 (s, 1H), 7.32 (m, 1H), 7.21 (t, J=8.8 Hz, 1H), 6.39 (d, J=5.2 Hz, 1H), 4.28 (t, J=6.6 Hz, 2H), 4.04 (s, 3H), 3.73 (t, J=4.6 Hz, 4H), 2.58 (t, J=7.2 Hz, 2H), 2.49 (m, 4H), 2.12-2.17 (m, 2H), 1.56 (s, 3H), 1.18-1.32 (m, 3H), 1.26 (s, 3H), 0.78-0.94 (m, 4H). LRMS (APCI neg) m/z 590 (M−1).

### Example 98

### N-(3-Fluoro-4-(6-methoxy-7-(3-morpholinopropoxy)quinolin-4-yloxy)phenyl)-3-(pyridin-2-yloxy)pyridin-2-amine 198

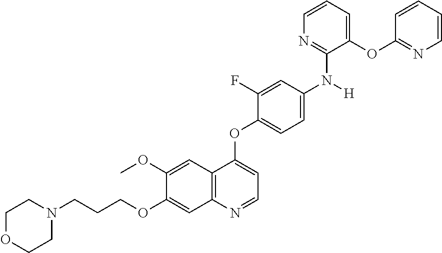

Step A: Preparation of 3-(pyridin-2-yloxy)pyridin-2-amine: A mixture of 2-aminopyridin-3-ol (0.50 g, 4.54 mmol), Cs₂CO₃ (4.44 g, 13.6 mmol) and 2-fluoropyridine (0.441 g, 4.54 mmol) in DMF (25 mL) was stirred at 100° C. for 4 hours. The reaction mixture was cooled to room temperature, water (25 mL) was added and then the aqueous phase extracted with CH₂Cl₂ (3×100 mL). The combined organic layers were dried over Na₂SO₄. Concentration and purification by silica gel flash column chromatography afforded the desired product (0.823 g, 97%). ¹H NMR (400 MHz, CDCl₃) δ 8.20 (m, 1H), 7.95 (dd, J=5.0, 1.4 Hz, 1H), 7.71 (m, 1H), 7.29 (dd, J=7.8, 1.4 Hz, 1H), 7.03 (m, 1H), 6.94 (d, J=8.0 Hz, 1H), 6.71 (dd, J=8.0, 4.8, 1H), 4.63 (s, 2H). LRMS (ESI pos) m/e 188 (M+1).

Step B: Preparation of N-(3-fluoro-4-(6-methoxy-7-(3-morpholinopropoxy)quinolin-4-yloxy)phenyl)-3-(pyridin-2-yloxy)pyridin-2-amine: A mixture of 4-(4-bromo-2-fluorophenoxy)-6-methoxy-7-(3-morpholinopropoxy)quinoline (Example 45) (20.0 mg, 0.0407 mmol), 3-(pyridin-2-yloxy)pyridin-2-amine (22.9 mg, 0.122 mmol), Pd₂(dba)₃ (7.45 mg, 0.00814 mmol), 4,5-bis(diphenylphosphino)-9,9-dimethyl-9H-xanthene (14.1 mg, 0.0244 mmol) and Cs₂CO₃ (39.8 mg, 0.122 mmol) in dioxane (10 mL) was stirred at 100° C. for 1 hour. The reaction mixture was cooled to room temperature, water (10 mL) was added and the aqueous layer extracted with CH₂Cl₂ (3×100 mL). The combined organic layers were dried over Na₂SO₄. Concentration and purification by silica gel flash column chromatography afforded 198 (13.8 mg, 57%). ¹H NMR (400 MHz, CDCl₃). δ 8.50 (d, J=5.2 Hz, 1H), 8.29 (dd, J=4.8, 1.2 Hz, 1H), 8.05 (dd, J=4.4, 1.6 Hz, 1H), 7.63 (m, 1H), 7.56 (s, 1H), 7.43 (m, 2H), 7.16-7.24 (m, 3H), 7.08-7.11 (m, 1H), 6.95-6.98 (m, 1H), 6.85 (d, J=8.4 Hz, 1H), 6.52 (d, J=5.2 Hz, 1H), 4.27 (t, J=6.6 Hz, 2H), 4.03 (s, 3H), 3.72 (t, J=4.4 Hz, 4H), 2.58 (t, J=7.0 Hz, 2H), 2.48 (m, 4H), 2.10-2.18 (m, 2H). LRMS (APCI neg) m/z 596 (M−1).

### Example 99

### 3-(Benzyloxy)-N-(3-fluoro-4-(6-methoxy-7-(3-morpholinopropoxy)quinolin-4-yloxy)phenyl)pyridin-2-amine 199

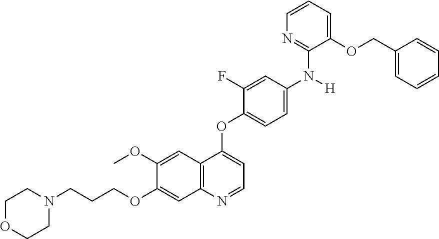

A mixture of 4-(4-bromo-2-fluorophenoxy)-6-methoxy-7-(3-morpholinopropoxy)quinoline (Example 45) (20.0 mg, 0.0407 mmol), 3-(benzyloxy)pyridin-2-amine (40.8 mg, 0.204 mmol), Pd₂(dba)₃ (7.45 mg, 0.00814 mmol), 4,5-bis(diphenylphosphino)-9,9-dimethyl-9H-xanthene (14.1 mg, 0.0244 mmol) and Cs₂CO₃ (39.8 mg, 0.122 mmol) in dioxane (10 mL) was stirred at 100° C. for 1 hour. The reaction mixture was cooled to room temperature, water (10 mL) was added and the aqueous layer was extracted with CH₂Cl₂ (3×50 mL). The combined organic layers were dried over Na₂SO₄. Concentration and purification by silica gel chromatography afforded 199 (16.8 mg, 68%). ¹H NMR (400 MHz, CDCl₃) δ 8.47 (d, J=5.2 Hz, 1H), 8.07 (d, J=12.4 Hz, 1H), 7.89 (d, J=4.4 Hz, 1H), 7.60 (s, 1H), 7.45 (m, 5H), 7.31 (d, J=8.8 Hz, 1H), 7.16 (m, 2H), 7.09 (d, J=7.2 Hz, 1H), 6.76 (m, 1H), 6.44 (d, J=4.8 Hz, 1H), 5.18 (s, 2H), 4.28 (t, J=6.4 Hz, 2H), 4.05 (s, 3H), 3.73 (m, 4H), 2.58 (t, J=6.8 Hz, 2H), 2.49 (m, 4H), 2.13 (m, 2H). LRMS (APCI neg) m/z 609 (M−1).

### Example 100

### 3-Fluoro-4-(6-methoxy-7-(3-morpholinopropoxy)quinolin-4-yloxy)-N-phenylaniline 200

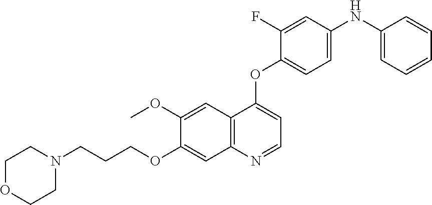

A mixture of 4-(4-bromo-2-fluorophenoxy)-6-methoxy-7-(3-morpholinopropoxy)quinoline (Example 45) (10.0 mg, 0.0204 mmol), aniline (9.48 mg, 0.102 mmol), Pd₂(dba)₃ (3.73 mg, 0.00407 mmol), 4,5-bis(diphenylphosphino)-9,9-dimethyl-9H-xanthene (7.07 mg, 0.0122 mmol) and Cs₂CO₃ (33.2 mg, 0.102 mmol) in dioxane (10 mL) was stirred at 100° C. for 4 hours. The reaction mixture was cooled to room temperature, water (10 mL) was added and the aqueous phase was extracted with CH₂Cl₂ (3×50 mL). The combined organic layers were dried over Na₂SO₄. Concentration and purification by silica gel flash column chromatography afforded 200 (9.9 mg, 97%). ¹H NMR (400 MHz, CDCl₃). δ 8.49 (d, J=5.2 Hz, 1H), 7.59 (s, 1H), 7.43 (s, 1H), 7.34 (m, 2H), 7.12-7.16 (m, 3H), 7.04 (t, J=7.2 Hz, 1H), 6.97 (dd, J=12.4, 2.8 Hz, 1H), 6.85 (m, 1H), 6.45 (dd, J=5.2, 0.8 Hz, 1H), 5.83 (s, 1H, NH), 4.28 (t, J=6.8 Hz, 2H), 4.04 (s, 3H), 3.73 (t, J=4.6 Hz, 4H), 2.58 (t, J=7.2 Hz, 2H), 2.49 (m, 4H), 2.07-2.17 (m, 2H). LRMS (APCI neg) m/z 502 (M−1).

### Example 101

### N-(3-Fluoro-4-(6-methoxy-7-(3-morpholinopropoxy)quinolin-4-yloxy)phenyl)-2,3′-bipyridin-2′-amine 201

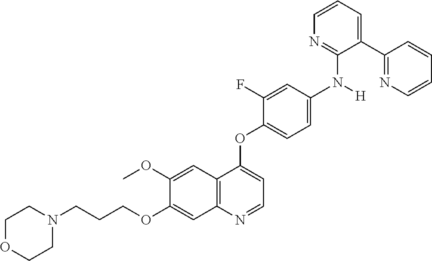

Step A: Preparation of 2′-fluoro-2,3′-bipyridine: A mixture of 2-fluoropyridin-3-ylboronic acid (200 mg, 1.419 mmol), 2-iodopyridine (291.0 mg, 1.419 mmol) and Tetrakis(triphenylphosphine)palladium(0) (328.0 mg, 0.2839 mmol) in 2 M aqueous Na₂CO₃ (3.5 mL) and DME (10 mL) was stirred at 80° C. for 8 hours. Water (10 mL) was added and the aqueous phase was extracted with CH₂Cl₂ (3×50 mL). The combined organic layers were dried over Na₂SO₄. Concentration and purification by silica gel chromatography afforded the product (124.6 mg, 50%). ¹H NMR (400 MHz, CDCl₃), δ8.72-8.75 (m, 1H), 8.8.50-8.57 (m, 1H), 8.24-8.27 (m, 1H), 7.88-7.92 (m, 1H), 7.77-7.82 (m, 1H), 7.28-7.36 (m, 2H). LRMS (ESI pos) m/e 175 (M+1).

Step B: Preparation of N-(3-fluoro-4-(6-methoxy-7-(3-morpholinopropoxy)quinolin-4-yloxy)phenyl)-2,3′-bipyridin-2′-amine: A mixture of 3-fluoro-4-(6-methoxy-7-(3-morpholinopropoxy)quinolin-4-yloxy)benzenamine (prepared in Example 72, steps C-F) (10.0 mg, 0.0234 mmol), NaH (2.81 mg, 0.117 mmol) and 2-fluoro-3-(pyridin-2-yl)pyridine (4.89 mg, 0.0281 mmol) in DMF (10 mL) was stirred at 70° C. for 16 hours. Water (10 mL) was added and the aqueous phase was extracted with CH₂Cl₂ (3×50 mL). The combined organic layers were dried over Na₂SO₄. Concentration and purification by silica gel flash column chromatography afforded 201 (10.8 mg, 79%). ¹H NMR (400 MHz, CDCl₃) δ 8.72 (m, 1H), 8.47 (d, J=5.2 Hz, 1H), 8.34 (dd, J=4.8, 1.6 Hz, 1H), 8.07 (dd, J=13.2, 2.8 Hz, 1H), 8.03 (dd, J=8.0, 2.0 Hz, 1H), 7.83-7.89 (m, 2H), 7.62 (s, 1H), 7.44 (s, 1H), 7.40-7.43 (m, 1H), 7.31-7.34 (m, 1H), 7.18 (t, J=8.8 Hz, 1H), 6.90 (m, 1H), 6.47 (m, 1H), 4.28 (t, J=6.8 Hz, 2H), 4.05 (s, 3H), 3.73 (t, J=4.6 Hz, 4H), 2.58 (t, J=7.2 Hz, 2H), 2.49 (m, 4H), 2.10-2.17 (m, 2H). LRMS (APCI neg) m/z 580 (M−1).

### Example 102

### Preparation of 6-(4-(6,7-dimethoxyquinolin-4-yloxy)-3-fluorophenyl)-N-phenylpyridin-3-amine 202

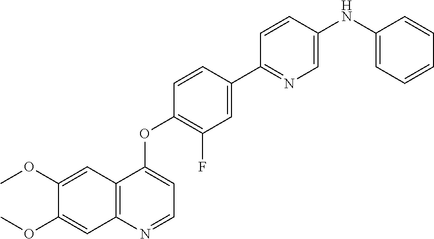

Step A: Preparation of 2-(4-(benzyloxy)-3-fluorophenyl)-5-bromopyridine: To a mixture of 2,5-dibromopyridine (3.00 g, 12.7 mmol), 4-(benzyloxy)-3-fluorophenylboronic acid (3.74 g, 15.2 mmol), Pd(PPh₃)₄ (0.73 g, 0.63 mmol) in a 3:1 mixture of toluene:ethanol (60 mL total) was added a 2M aqueous solution of Na₂CO₃ (15 mL). The reaction mixture was stirred at 105° C. for 3 hours. After cooling to room temperature, the mixture was partitioned between ethyl acetate (100 mL) and water (50 mL). The organic layer was separated, washed with brine, dried over Na₂SO₄, and evaporated to dryness. The crude material was purified by silica gel flash column chromatography (70:30 hexanes:EtOAc). Insoluble material was removed by filtration prior to chromatography. The desired product (200 mg, 0.4%) was obtained as an off-white solid. ¹H-NMR (400 MHz; DMSO-d₆) δ 8.74 (d, 1H), 8.11 (d, 1H), 7.92-7.98 (m, 3H), 7.89 (d, 1H), 7.49 (d, 2H) 7.34-7.44 (m, 3H), 5.26 (s, 2H). LRMS (ESI pos) m/e 360 (M+1).

Step B: Preparation of 6-(4-(benzyloxy)-3-fluorophenyl)-N-phenylpyridin-3-amine: To a mixture of 2-(4-(benzyloxy)-3-fluorophenyl)-5-bromopyridine (200 mg, 0.56 mmol), Pd₂dba₃ (5.11 mg, 0.0056 mmol), (S)(−)2,2′-Bis(diphenylphosphino)-1,1′-binaphthyl (5.22 mg, 0.0084 mmol) in dry THF (2 mL) was added NaOtBu (75.12 mg, 0.78 mmol) and aniline (0.053 ml, 0.586 mmol). The mixture was degassed by successive evacuation and back-filling with nitrogen (3×). The mixture was heated to reflux temperature and stirred under nitrogen for 18 hours. The reaction mixture was cooled to room temperature and was partitioned between ethyl acetate (15 mL) and water (15 mL). The organic layer was separated, washed with brine, dried over Na₂SO₄, and evaporated to give a crude black oil. The crude product was purified by silica gel flash column chromatography loading with dichloromethane and eluting with 80:20 hexanes:EtOAc to give the desired product (87 mg, 42%) as an off-white crystalline solid. ¹H-NMR (400 MHz, DMSO-d₆) δ 8.50 (s, 1H), 8.40 (d, 1H), 7.86 (d, 1H), 7.79 (m, 2H), 7.53-7.47 (m, 3H), 7.42 (t, 2H), 7.36 (t, 1H), 7.29 (t, 3H), 7.12 (d, 2H), 6.90 (t, 1H), 5.23 (s, 2H). LRMS (APCI pos) m/e 371 (M+1).

Step C: Preparation of 2-fluoro-4-(5-(phenylamino)pyridin-2-yl)phenol: 6-(4-(benzyloxy)-3-fluorophenyl)-N-phenylpyridin-3-amine (62 mg, 0.167 mmol) was dissolved in TFA (1.29 ml, 16.7 mmol) and the reaction mixture was stirred at 70° C. for 18 hours. The mixture was cooled to room temperature and the excess TFA was removed by evacuation. The crude product was partitioned between dichloromethane and saturated Na₂CO₃ solution. The layers were separated and the organic layer was dried over Na₂SO₄ and evaporated to give a dark yellow oil which was purified by silica gel flash column chromatography, loading with DCM and eluting with 80/20 hexanes/EtOAc to give the desired product (42 mg, 89%) as yellow glass. LRMS (APCI pos) m/e 281 (M+1).

Step D: Preparation of 6-(4-(6,7-dimethoxyquinolin-4-yloxy)-3-fluorophenyl)-N-phenylpyridin-3-amine: 2-fluoro-4-(5-(phenylamino)pyridin-2-yl)phenol (42 mg, 0.150 mmol), 4-chloro-6,7-dimethoxyquinoline (Example 5) (40.2 mg, 0.180 mmol) and DMAP (5.49 mg, 0.045 mmol) were added to a small sealable glass reaction tube. The mixture was suspended in bromobenzene (1.5 mL) and stirred at 150° C. for 36 hours. The reaction mixture was cooled to room temperature, the solvent was evaporated, and the crude dissolved in MeOH and absorbed onto silica gel and purified by silica gel flash column chromatography, eluting with 60/40 hexanes/EtOAc to yield 202 (29 mg, 36%) as a pale yellow solid. ¹H-NMR (400 MHz, CDCl₃) δ 8.49 (d, 2H), 7.92-7.79 (m, 2H), 7.62 (s, 1H), 7.55-7.41 (m, 2H), 7.39-7.24 (m, 3H), 7.15 (d, 2H), 7.05 (t, 1H), 6.49 (s, 1H), 5.98 (s, 1H), 4.06 (d, 6H). LRMS (APCI pos) m/e 468 (M+1).

### Example 103

### 6-(4-(6,7-dimethoxyquinolin-4-yloxy)-3-fluorophenyl)-N-methyl-N-phenylpyridin-3-amine 203

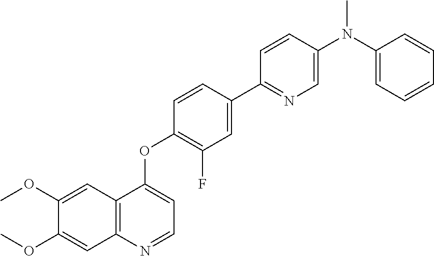

Step A: Preparation of 6-(4-(6,7-dimethoxyquinolin-4-yloxy)-3-fluorophenyl)-N-methyl-N-phenylpyridin-3-amine: 6-(4-(6,7-dimethoxyquinolin-4-yloxy)-3-fluorophenyl)-N-phenylpyridin-3-amine (Example 102, 22 mg, 0.047 mmol) was dissolved in DMF (0.5 mL) and cooled to 0° C. NaH, 60% dispersion in oil (26 mg, 0.056 mmol) was added and the mixture was allowed to warm to room temperature and stirred for 10 minutes. Iodomethane (0.012 ml, 0.188 mmol) was added and the reaction was stirred at 25° C. for 18 hours. The solvent was evaporated and the crude product purified by silica gel flash column chromatography, loading with DCM and eluting with 80/20 EtOAc/hexane to give 203 (5 mg, 19%) as pale yellow glass. ¹H-NMR (400 MHz, CDCl₃) δ 8.50 (s, 1H), 8.37 (s, 1H), 7.89 (d, 1H), 7.79 (d, 1H), 7.61-7.58 (m, 2H), 7.45 (s, 1H), 7.39 (t, 2H), 7.33-7.14 (m, 5H), 6.49 (d, 1H), 4.07 (s, 3H), 4.06 (s, 3H), 3.41 (s, 3H). LRMS (APCI pos) m/e 482 (M+1).

### Example 104

### 5-(4-(6,7-dimethoxyquinolin-4-yloxy)-3-fluorophenyl)-N-phenylpyrimidin-2-amine 204

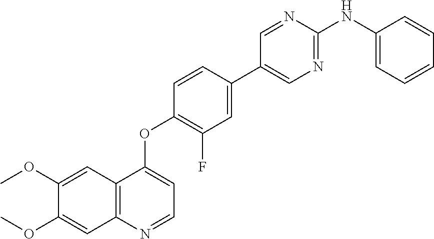

Step A: Preparation of 5-bromo-N-phenylpyrimidin-2-amine: To a sealable glass reaction tube was 5-bromo-2-chloropyrimidine (1.00 g, 5.17 mmol) dissolved in 1-propanol (10 mL). DIEA (1.08 ml, 6.20 mmol) and aniline (0.565 ml, 6.20 mmol) were added and the tube was sealed and stirred at 100° C.-120° C. for 18 hours. The mixture was cooled in an ice-water bath and a white precipitate formed. The mixture was diluted with ethyl acetate (10 mL), washed with brine (20 mL), water (20 mL), and brine (20 mL). The organic layer was isolated and evaporated to give 1.18 g of crude material. The crude product was triturated with 90/10 hexane/EtOAc and the solid isolated by filtration to give the desired product (0.74 g, 57%) as a pale brown solid. ¹H-NMR (400 MHz, DMSO-d₆) δ 9.85 (s, 1H), 8.59 (s, 2H), 7.70 (d, J=7.8 Hz, 2H), 7.29 (t, J=8.0 Hz, 2H), 6.98 (t, J=7.2 Hz, 1H). LRMS (ESI pos) m/e 252 (M+1).

Step B: Preparation of 5-(4-(benzyloxy)-3-fluorophenyl)-N-phenylpyrimidin-2-amine: A mixture of 5-bromo-N-phenylpyrimidin-2-amine (0.500 g, 2.00 mmol), 4-(benzyloxy)-3-fluorophenylboronic acid (0.984 g, 4.00 mmol), Pd(PPh₃)₄ (0.116 g, 0.1000 mmol) and lithium chloride (0.170 g, 4.00 mmol) in dioxane (10 mL) and 2M aqueous Na₂CO₃ (2 mL) was stirred at 90° C. for 2 hours and then at room temperature for 1 hour. Water (50 mL) and ethyl acetate (100 mL) were added and the organic layer was separated, washed with brine (100 mL), dried over MgSO₄, filtered and evaporated to give 1.3 g crude solid. The crude material was triturated with dichloromethane:MeOH and the resulting white solid was filtered and washed with dichloromethane to give the desired product (480 mg, 65%) as a white solid. ¹H-NMR (400 MHz, DMSO-d₆) δ 9.77 (s, 1H), 8.82 (s, 2H), 7.79 (d, 2H), 7.67 (d, 1H), 7.50-7.47 (m, 3H), 7.42 (t, 3H), 7.37-7.27 (m, 3H), 6.96 (t, 1H), 5.24 (s, 2H). LRMS (ESI pos) m/e 372 (M+1).

Step C: Preparation of 2-fluoro-4-(2-(phenylamino)pyrimidin-5-yl)phenol: 5-(4-(benzyloxy)-3-fluorophenyl)-N-phenylpyrimidin-2-amine (280 mg, 0.754 mmol) was suspended in TFA (5 mL) and the suspension was stirred at 70° C. for 18 hours. The excess solvent was removed under reduced pressure and the residue dissolved in dichloromethane (15 mL). The organic layer was washed with water (15 mL), aqueous NaHCO₃ solution, separated and evaporated to give the desired product (120 mg, 57%) as a yellow solid. ¹H-NMR (400 MHz, DMSO-d₆) δ 10.01 (s, 1H), 9.73 (s, 1H), 8.78 (s, 2H), 7.79 (d, 2H), 7.56 (d, 1H), 7.36 (d, 1H), 7.29 (t, 2H), 7.04 (t, 1H), 6.96 (t, 1H). LRMS (ESI pos) m/e 282 (M+1).

Step D: Preparation of 5-(4-(6,7-dimethoxyquinolin-4-yloxy)-3-fluorophenyl)-N-phenylpyrimidin-2-amine: 2-fluoro-4-(2-(phenylamino) pyrimidin-5-yl)phenol (51.0 mg, 0.181 mmol), 4-chloro-6,7-dimethoxyquinoline (Example 5, 48.7 mg, 0.218 mmol), and DMAP (6.65 mg, 0.054 mmol) were added to a sealable glass reaction tube. The mixture was suspended in bromobenzene (2 mL) and stirred at 150° C. for 18 hours. The reaction mixture was cooled to room temperature and the solvent was evaporated. The crude product was triturated with EtOAc:MeOH and the resulting solid filtered to give 204 (52 mg, 61%) as a white solid. ¹H-NMR (400 MHz, DMSO-d₆) δ 9.88 (s, 1H), 8.94 (s, 2H), 8.51 (d, 1H), 7.96 (d, 1H), 7.80 (s, 2H), 7.75 (d, 1H), 7.57 (m, 1H), 7.43 (s, 1H), 7.32 (t, 2H), 6.99 (t, 1H), 6.55 (d, 1H), 3.96 (s, 6H). LRMS (APCI pos) m/e 469 (M+1).

### Example 105

### 5-(4-(6,7-dimethoxyquinolin-4-yloxy)-3-fluorophenyl)-N-methyl-N-phenylpyrimidin-2-amine 205

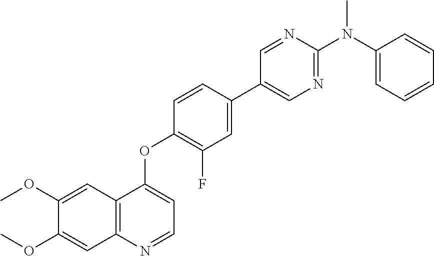

Step A: Preparation of 5-(4-(6,7-dimethoxyquinolin-4-yloxy)-3-fluorophenyl)-N-methyl-N-phenylpyrimidin-2-amine: 5-(4-(6,7-dimethoxyquinolin-4-yloxy)-3-fluorophenyl)-N-phenylpyrimidin-2-amine (Example 104, 35 mg, 0.0747 mmol) was dissolved in DMF (0.5 mL) and cooled to 0° C. NaH, 60% dispersion in oil (3.59 mg, 0.0897 mmol) was added and the mixture was allowed to warm to room temperature and stirred overnight. The mixture was partitioned between ethyl acetate (10 mL) and water (10 mL). The organic layer was washed with brine, dried over Na₂SO₄, and evaporated to give 205 (32 mg, 89%) as an off-white glass. ¹H-NMR (400 MHz, DMSO-d₆) δ 8.82 (s, 2H), 8.50 (d, 1H), 7.89 (d, 1H), 7.66 (d, 1H), 7.58-7.52 (m, 2H), 7.45-7.37 (m, 5H), 7.25 (t, 1H), 6.52 (d, 1H), 3.96 (s, 6H), 3.54 (s, 3H). LRMS (APCI pos) m/e 483 (M+1).

### Example 106

### c-Met Enzyme Assay

The assay for the determination of c-Met kinase activity is based on an enzyme linked immunosorbant assay (ELISA). A compound of Formula I, 50 pM c-Met (His-tagged recombinant human Met (amino acids 974-end), expressed by baculovirus), and 5 μM ATP in assay buffer (25 mM MOPS, pH 7.4, 5 mM MgCl₂, 0.5 mM MnCl₂, 100 μM Sodium Orthovanadate, 0.01% Triton X-100, 1 mM DTT, final DMSO concentration 1% (v/v)) are incubated on a 0.25 mg/mL PGT coated plates for 20 minutes at room temperature. The reaction mixture is removed by washing and the phosphorylated polymer substrate is detected with 0.2 μg/mL phosphotyrosine specific monoclonal antibody (PY20) conjugated to horseradish peroxidase (HRP). After the addition of 1M phosphoric acid to stop the development, the chromogenic substrate (TMB) color is quantitated by spectrophotometry at 450 nm.

### Example 107

### In Vitro Cell Proliferation Assay

The cellular activity of the compounds of the present invention may be determined by the following procedure. MKN45 cells were plated in Costar 3904 96-well plates in growth media (RPMI, 10% FBS) at a density of 15000 cells/well and incubated at 37° C., 5% CO₂ overnight. The following day, one-tenth volume of a 10× concentration of compounds was added to the wells in a 11-point dilution series. The dilutions series was composed of an initial 1:3 dilution in DMSO, followed by a 1:20 dilution in HBSS, for a final DMSO concentration on cells of 0.5%. Control wells were treated with 0.5% DMSO. The typical range of dilution was 5 μM to 0.3 nM, which was expanded to 25 μM depending on the potency of the compound. Once compound was added to the cells, plates were incubated for one hour at 37° C., 5% CO₂. Plates were then washed in PBS, fixed in 4% formaldehyde and rehydrated with a 10% methanol solution. The plates were then blocked with Licor blocking buffer. The total phosphorylated c-Met levels were measured by incubating with a rabbit polyclonal antibody against phosphorylated c-Met followed by an anti-rabbit antibody conjugated to Alexa Fluor 680. Signal was normalized for differences in cell number by reference to the levels of the housekeeping protein GAPDH. Cells were incubated with a mouse monoclonal antibody against GAPDH followed by an anti-mouse antibody labeled with IRdye 800. Signal was measured on the Licor. The overall fluorescent signal from the Alexa Fluor 680 is normalized by dividing the value by the fluorescent value of the IRdye 800 signal. The fluorescent signal of the control wells was defined as 100% and the percent of inhibition of phosphorylated c-Met was expressed as percent of control. IC₅₀ values were determined from the percent of control data using a standard 4-parameter logistical model.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A compound having Formula I: 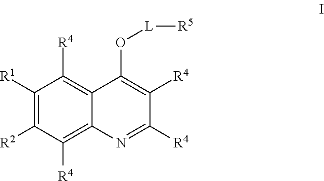
and stereoisomers, geometric isomers, tautomers, solvates, metabolites, and salts thereof, wherein:
R¹, R² and R⁴ are independently selected from H, F, Cl, Br, I, CN, —(CR¹⁴R¹⁵)ₜNR¹⁰R¹¹, —C(═Y)R¹⁰, —C(═Y)OR¹⁰, —C(═Y)NR¹⁰R¹¹, —C(═O)NR¹²(CR¹⁴R¹⁵)NR¹⁰R¹¹, —NO₂, —NR¹⁰R¹¹, —NR¹⁰C(═Y)R¹¹, —NR¹⁰C(═Y)OR¹¹, —NR¹²C(═Y)NR¹⁰R¹¹, —NR¹²SO₂NR¹⁰R¹¹, OR¹⁰, —OC(═Y)OR¹⁰, OC(═Y)OR¹⁰, OC(═Y)NR¹¹, —OP(═Y)(OR¹⁰)(OR¹¹), —OP(OR¹⁰)(OR¹¹), —P(═Y)(OR¹⁰ (OR¹¹), —SR¹⁰, —S(O)R¹⁰, —S(O)₂R¹⁰, —S(O)₂NR¹⁰R¹¹, —SC(═Y)R¹⁰, —SC(═Y)OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl, where said alkyl, alkenyl, alkynyl, carbocyclyl, heterocyclyl, aryl and heteroaryl are optionally substituted with one or more groups independently selected from F, Cl, Br, I, CN, CF₃, —NO₂, oxo, —C(═Y)R¹⁰, —C(═Y)OR¹⁰, —C(═Y)NR¹⁰R¹¹, —(CR¹⁴R¹⁵)ₙ—NR¹⁰R¹¹, —NR¹⁰C(═Y)R¹⁰, —NR¹⁰C(═Y)OR¹¹, —NR¹²C(═Y)NR¹⁰R¹¹, —NR¹²SO₂R¹⁰, ═NR¹⁰, OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰, —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, C₁-C₂₀ heteroaryl, —(CR¹⁴R¹⁵)ₜ—NR¹²C(═O)(CR¹⁴R¹⁵)NR¹⁰R¹¹, and (CR⁴R⁵)ₜ—NR¹⁰R¹¹,
with the proviso that at least one of R¹ and R² is not H;
L is C₃-C₁₂ carbocyclyl, C₂-C₂₀ heterocyclyl, C₆-C₂₀ aryl or C₁-C₂₀ heteroaryl, wherein said carbocyclyl, heterocyclyl, aryl and heteroaryl are optionally substituted with one or more groups independently selected from R⁴ and R¹⁰, with the proviso that L is not naphthyl;
R⁵ is —C(═Y)R¹³, —C(═Y)NR¹⁰R¹³, —NR¹⁰R¹³, —NR¹⁰C(═Y)R¹³, —NR¹⁰C(═Y)OR¹³, —NR¹²SO₂R¹⁰, —NR¹²C(═Y¹)(CR¹⁴R¹⁵)C(═Y²)NR¹⁰R¹¹, C₃-C₁₂ carbocyclyl, C₂-C₂₀ heterocyclyl, C₆-C₂₀ aryl, or C₁-C₂₀ heteroaryl, wherein said carbocyclyl, heterocyclyl, aryl, and heteroaryl are optionally substituted with one or more groups independently selected from oxo, F, Cl, Br, I, SO₂R^{c}, CN, OR^{a}, (CH₂)ₙ—NR^{a}R^{b}, C(═O)NR^{a}R^{b}, C(═O)R^{a}, CR^{a}C(═O)R^{b}, NHSO₂R^{c}, CF₃, C₁-C₁₂ alkyl, C₂-C₈ alkenyl, C₂-C₈ alkynyl, (CH₂)ₙ—(C₆-C₂₀ aryl), (CH₂)ₙ-cycloalkyl, (CH₂)ₙ-cycloalkyl, CH(OH)-aryl, CH(CO₂CH₃)aryl, and (CH₂)ₙ—(C₁-C₂₀ heteroaryl), and wherein any aryl or heteroaryl of the one or more groups is optionally substituted with one or more R^{d};
R¹⁰, R¹¹ and R¹² are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more groups independently selected from F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₃-C₁₂ carbocyclyl, C₂-C₂₀ heterocyclyl optionally substituted with C₁-C₆ alkyl, CH₂OH or SO₂Me, C₆-C₂₀ aryl, and C₁-C₂₀ heteroaryl optionally substituted with C₁-C₆ alkyl,
or R¹⁰ and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₙOR^{a}, NR^{a}R^{b}, CF₃, F, Cl, Br, I, SO₂R^{a}, C(═O)R^{a}, NR¹⁰C(═Y)R¹¹, C(═Y)NR¹⁰R¹¹, C₁₋₁₂ alkyl, C₂-C₈ alkenyl, C₂-C₈ alkynyl, C₃-C₁₂ carbocyclyl, C₂-C₂₀ heterocyclyl, C₆-C₂₀ aryl and C₁-C₂₀ heteroaryl;
R¹³ is H, C₁-C₆ alkyl, —(CR¹⁴R¹⁵)ₙ-cycloalkyl, —(CR¹⁴R¹⁵)ₙ-heterocyclyl, —(CR¹⁴R¹⁵)ₙ-aryl, —(CR¹⁴R¹⁵)ₙ-heteroaryl, (CR¹⁴R¹⁵)ₙ—O—(CR¹⁴R¹⁵)ₘ-aryl, (CR¹⁴R¹⁵)—N(SO₂R^{a})—(CR¹⁴R¹⁵)R¹¹, (CR¹⁴R¹⁵)ₙ-heterocyclyl-(CR¹⁴R¹⁵)ₜ-aryl, or (CR¹⁴R¹⁵)—NR¹⁰C(═O)aryl, where said cycloalkyl, heterocyclyl, aryl, and heteroaryl portions are optionally substituted with one or more groups independently selected from F, Cl, Br, I, oxo, SO₂R^{c}, CN, OR^{a}, C(═O)R^{a}, C(═O)OR^{a}, NR^{a}R^{b}, NR^{a}C(═O)R^{b}, O—(CH₂)-aryl, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;
each R¹⁴ and R¹⁵ is independently H, C₁-C₁₂ alkyl, or (CH₂)ₜ-aryl,
or R¹⁴ and R¹⁵ together with the atoms to which they are attached form a saturated or partially unsaturated C₃-C₁₂ carbocyclic ring,
or R¹⁰ and R¹⁵ together with the atoms to which they are attached form a saturated or partially unsaturated C₂-C₁₂ heterocyclic ring,
or R¹⁴ is null and R¹⁰ and R¹⁵ together with the atoms to which they are attached form a 5-6 membered heteroaryl ring,
or R¹² and R¹⁴ together with the atoms to which they are attached form a saturated or partially unsaturated C₂-C₁₂ heterocyclic ring;
R^{a} and R^{b} are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more alkyl groups;
R^{c} is C₁-C₁₂ alkyl or C₆-C₂₀ aryl, wherein said alkyl and aryl are optionally substituted with one or more groups independently selected from F, Cl, Br, I, OR^{a} and C(═O)NR^{a}R^{b};
R^{d} is F, Cl, Br, I, CF₃, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, or C₁-C₂₀ heteroaryl;
Y, Y¹ and Y² are independently O or S;
t is 1, 2, 3, 4, 5 or 6; and
n and m are independently 0, 1, 2, 3, 4, 5 or 6.

2. The compound of claim 1, wherein one or both of R¹ and R² is —OR¹⁰ where R¹⁰ is C₁-C₁₂ alkyl.

3. The compound of claim 1, wherein one of R¹ and R² is methoxy.

4. The compound of claim 1, wherein both of R¹ and R² are methoxy.

5. The compound of claim 1, wherein one or both of R¹ and R² is —OR¹⁰ where R¹⁰ is C₁-C₁₂ alkyl substituted with NR^{a}R^{b}.

6. The compound of claim 1, wherein one or both of R¹ and R² is —OR¹⁰ where R¹⁰ is C₁-C₁₂ alkyl substituted with C₂-C₂₀ heterocyclyl optionally substituted with C₁-C₆ alkyl, CH₂OH or SO₂Me.

7. The compound of claim 6 wherein —OR¹⁰ is selected from the structures: 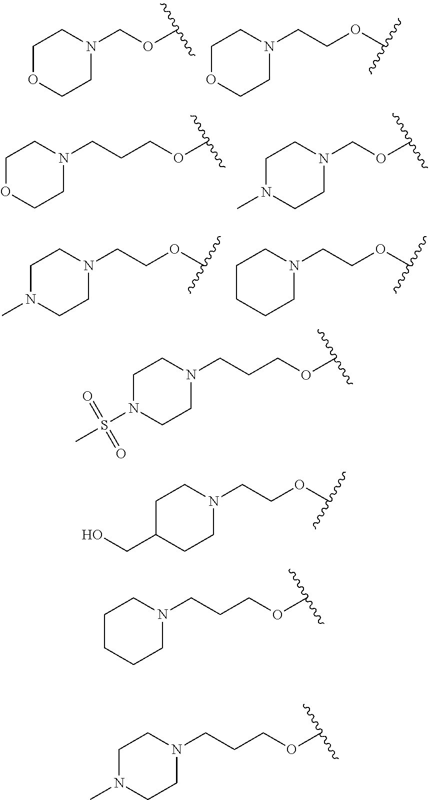
where the wavy line is the attachment site to the quinoline ring.

8. The compound of claim 1 wherein R¹ is methoxy and R² is 3-morpholinopropoxy.

9. The compound of claim 1, wherein one or both of R¹ and R² is —OR¹⁰ where R¹⁰ is C₁-C₁₂ alkyl substituted with C₁-C₂₀ heteroaryl, wherein said heteroaryl is optionally substituted with C₁-C₆ alkyl.

10. The compound of claim 9 wherein —OR¹⁰ is selected from the structures: 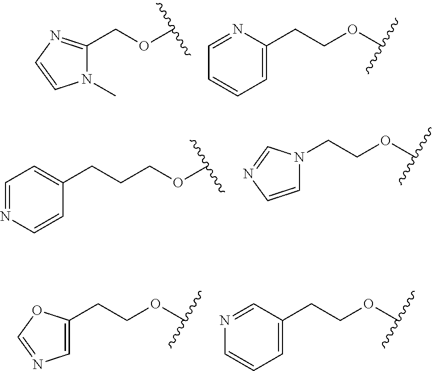
where the wavy line is the attachment site to the quinoline ring.

11. The compound of claim 1, wherein one or both of R¹ and R² are independently selected from alkynyl substituted by —(CR¹⁴R¹⁵)ₜ—NR¹²C(═O)(CR¹⁴R¹⁵)NR¹⁰R¹¹ or —(CR⁴R⁵)ₜNR¹⁰R¹¹.

12. The compound of claim 11 wherein one or both of R¹ and R² are independently selected from the structures:

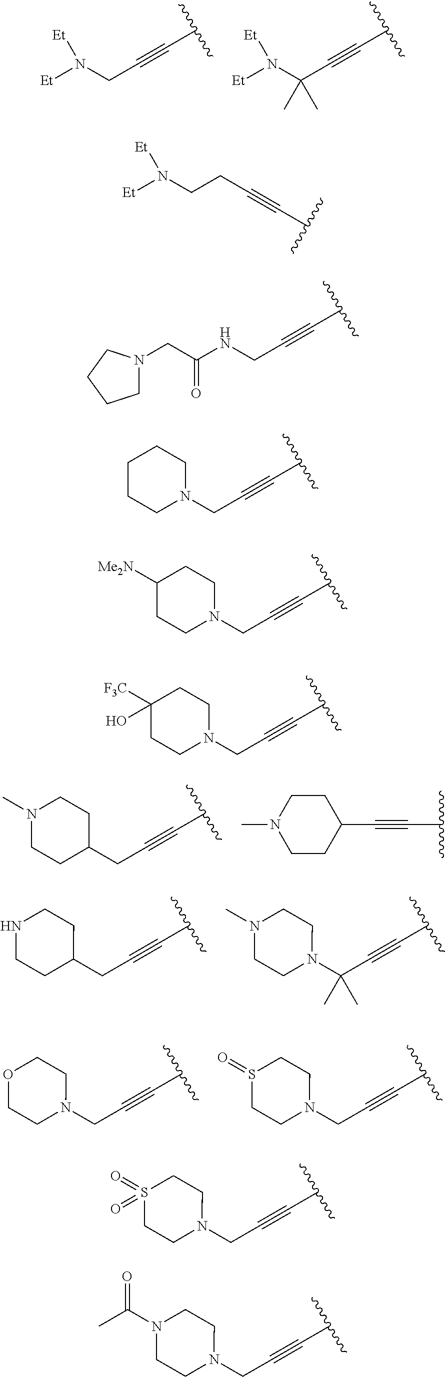

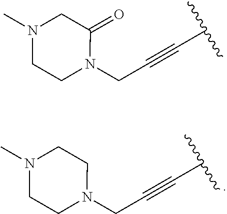

13. The compound of claim 1 wherein one or both of R¹ and R² are independently selected from optionally substituted aryl or heteroaryl.

14. The compound of claim 13 wherein one or both of R¹ and R² are independently selected from:

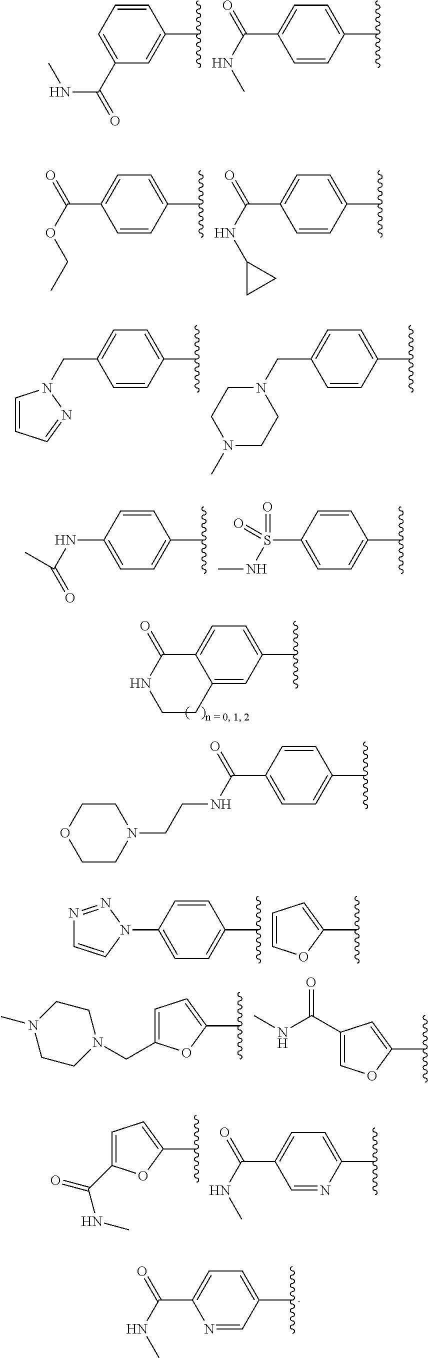

15. The compound of claim 1, wherein one or both of R¹ and R² are independently selected from —C(═O)NR¹⁰R¹¹ or —(CR¹⁴R¹⁵)ₜNR¹⁰R¹¹.

16. The compound of claim 1 wherein one or both of R¹ and R² are independently selected from alkyl optionally substituted with one or more groups independently selected from OR¹⁰, NR¹⁰R¹¹, heterocyclyl and heteroaryl.

17. The compound of claim 16 wherein one or both of R¹ and R² are independently selected from methyl, —CH₂OH, —CH₂CH₂OH, —CH₂CH₂CH₂OH, and —CH(OH)CH₂OH.

18. The compound of claim 1 wherein each R⁴ is H.

19. The compound of claim 1 where L-R⁵ is (C₃-C₁₂ carbocyclyl)-R⁵.

20. The compound of claim 19 wherein L-R⁵ is selected from the structures: 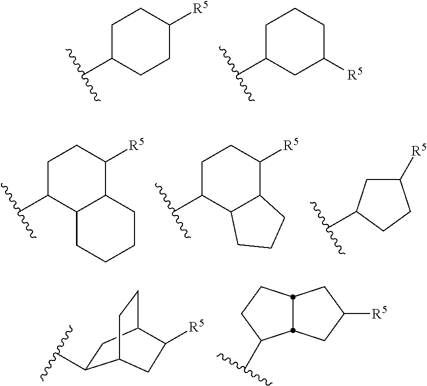
where the wavy line indicates the point of attachment to the 4-oxy position of the quinoline ring.

21. The compound of claim 1 where L-R⁵ is (C₂-C₂₀ heterocyclyl)-R⁵.

22. The compound of claim 21 wherein L-R⁵ is selected from the structures: 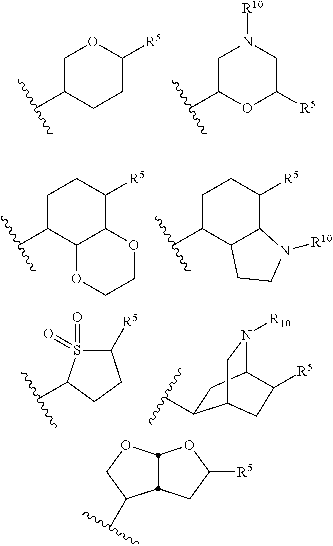
where the wavy line indicates the point of attachment to the 4-oxy position of the quinoline ring.

23. The compound of claim 1 where L-R⁵ is (C₆-C₂₀ aryl)-R⁵.

24. The compound of claim 23 wherein L-R⁵ is selected from the structures: 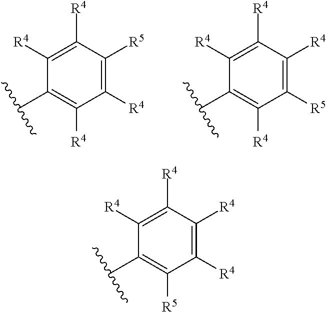
where the wavy line indicates the point of attachment to the 4-oxy position of the quinoline ring and each R⁴ is independent of the other.

25. The compound of claim 24 where L-R⁵ is selected from the structures:

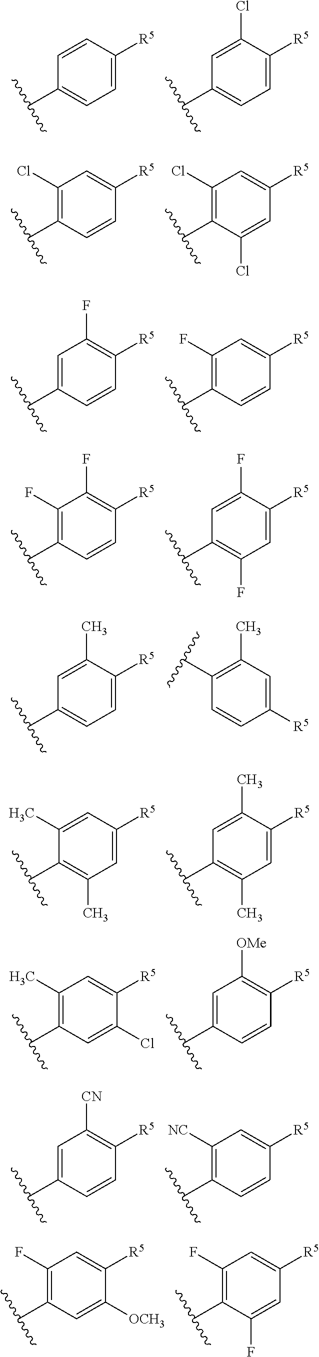

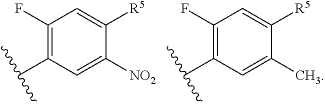

26. The compound of claim 24 where L-R⁵ is selected from the structures:

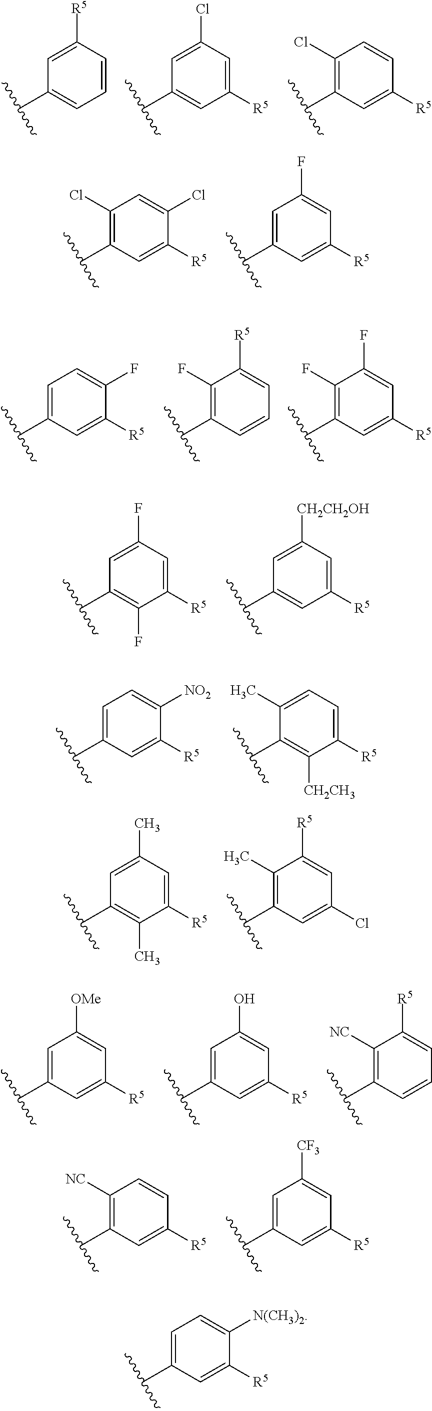

27. The compound of claim 24 where L-R⁵ is selected from the structures:

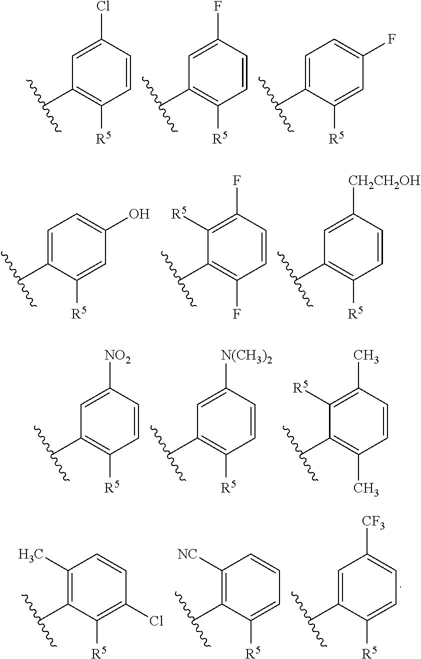

28. The compound of claim 23 where L-R⁵ is selected from the structures:

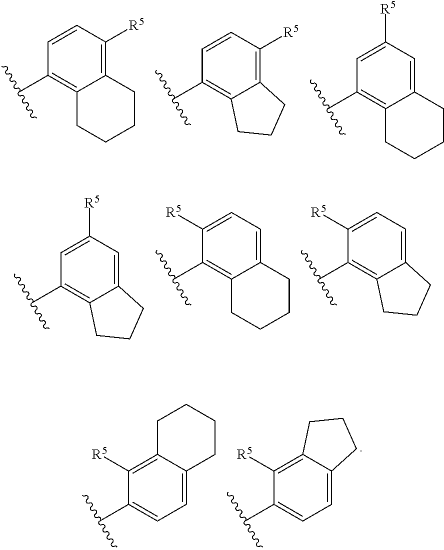

29. The compound of claim 1 where L-R⁵ is (C₁-C₂₀ heteroaryl)-R⁵.

30. The compound of claim 29 wherein L-R⁵ is selected from the structures: 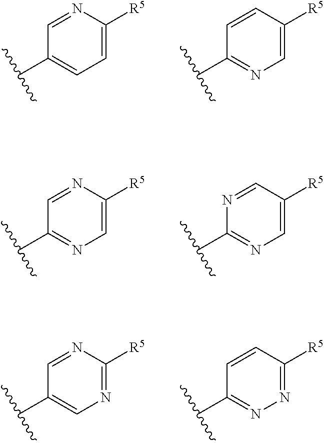
where the wavy line indicates the point of attachment to the 4-oxy position of the quinoline ring.

31. The compound of claim 30 wherein L-R⁵ is selected from the structures

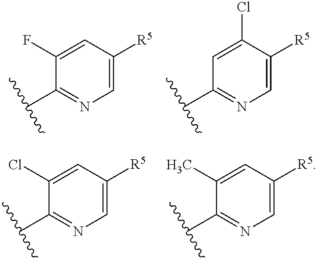

32. The compound of claim 29 wherein L-R⁵ is selected from the structures:

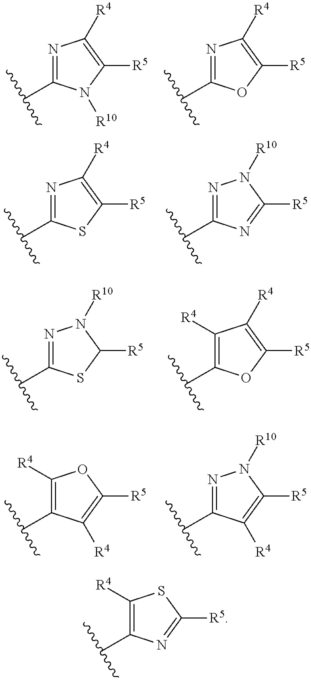

33. The compound of claim 29 wherein L is selected from the structures:

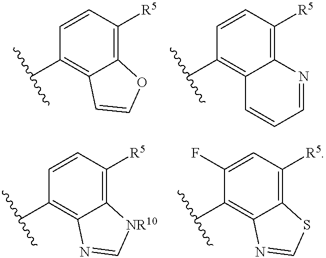

34. The compound of claim 24 wherein R⁵ is —C(═Y)R¹³.

35. The compound of claim 34 wherein R⁵ is selected from the structures: 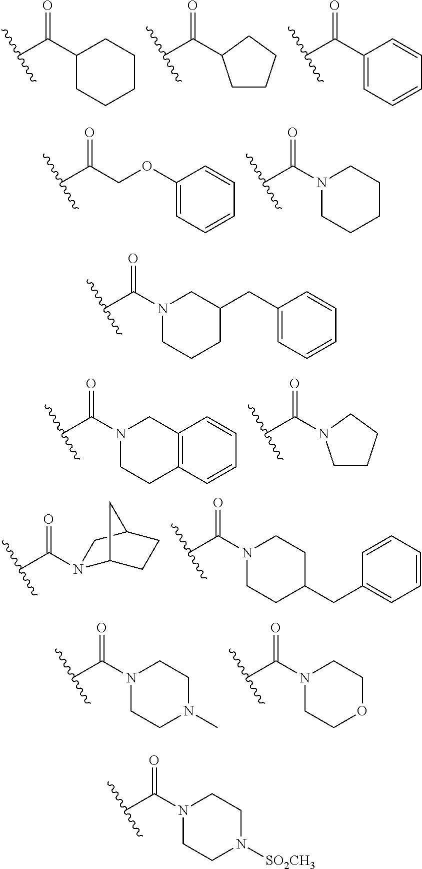
where the wavy line indicates the point of attachment to L.

36. The compound of claim 24 wherein R⁵ is —C(═Y)NR¹⁰R¹³.

37. The compound of claim 36 wherein R⁵ is selected from the structures: 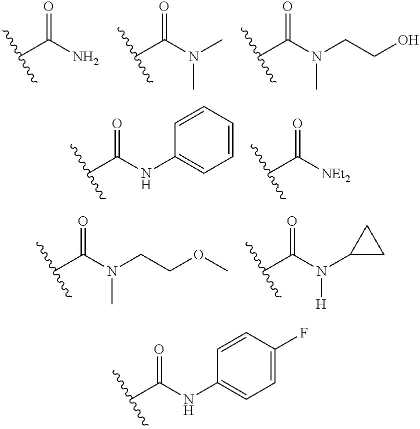
where the wavy line indicates the point of attachment to L.

38. The compound of claim 1 wherein R⁵ is —NR¹⁰R¹³.

39. The compound of claim 38 wherein R⁵ is selected from the structures: 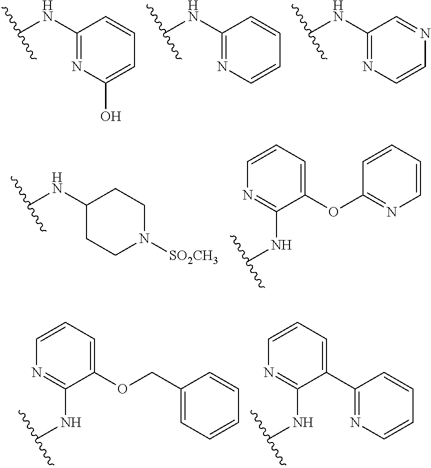
where the wavy line indicates the point of attachment to L.

40. The compound of claim 24 wherein R⁵ is —NR¹²C(═Y¹)(CR¹⁴R¹⁵)C(═Y²)NR¹⁰R¹¹, wherein R¹⁵ and R¹⁰ optionally together with the atoms to which they are attached form a 5-6 membered heterocyclic ring, and wherein R¹⁴ and the adjacent saturated ring carbon together with the atoms to which they are attached optionally form a fused cyclopropyl ring.

41. The compound of claim 40 wherein R⁵ is selected from the structures: 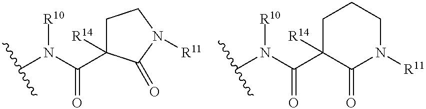
where the wavy line indicates the point of attachment to L.

42. The compound of claim 41 wherein R⁵ is selected from the structures:

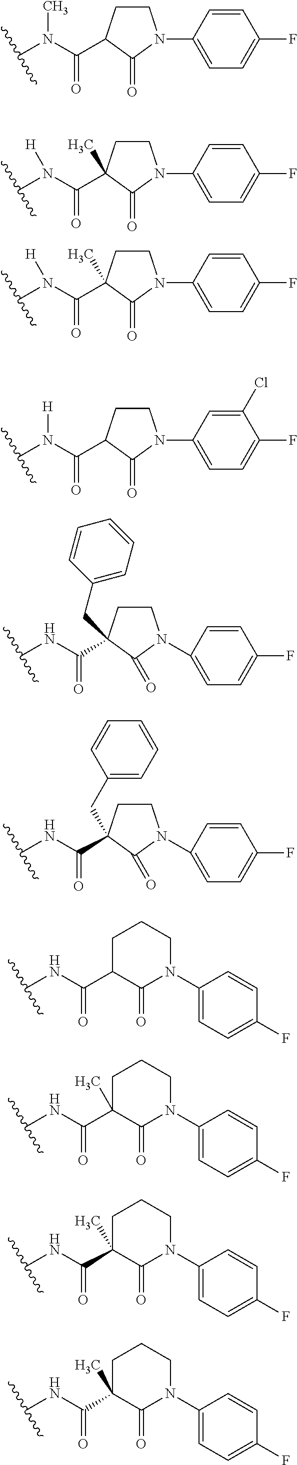

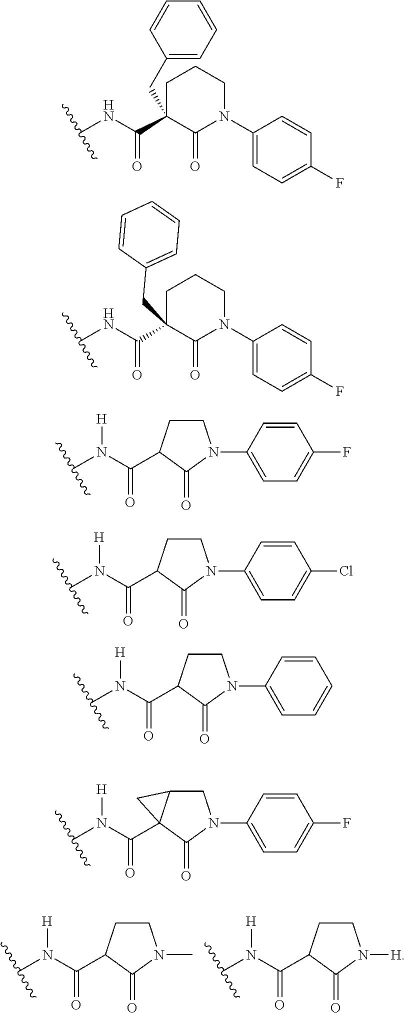

43. The compound of claim 40 wherein R⁴ is null and R¹⁰ and R¹⁵ together with the nitrogen atom to which they are attached form a heteroaryl ring optionally having an additional ring nitrogen atom.

44. The compound of claim 43, wherein R⁵ is selected from the structures: 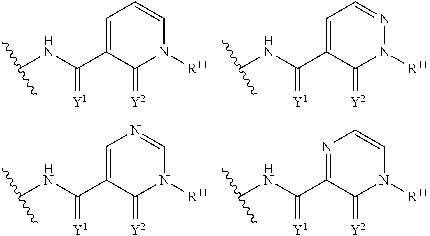
where Y¹ and Y² are independently selected from O and S; and where the wavy line indicates the point of attachment to L.

45. The compound of claim 43 wherein R⁵ is selected from the structures: 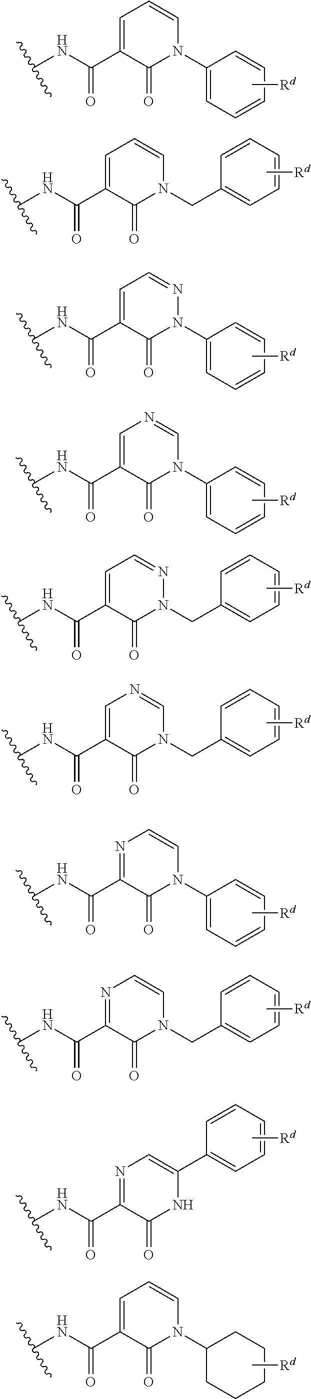 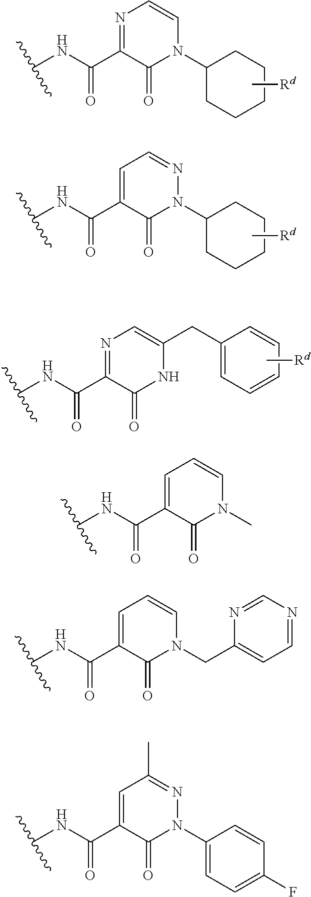
wherein the cyclohexyl and phenyl groups are optionally substituted with one or more R^{d} groups independently selected from F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, and C₁-C₂₀ heteroaryl.

46. The compound of claim 24, wherein R⁵ is —NR¹²C(═Y¹)(CR¹⁴R¹⁵)C(═Y²)NR¹⁰R¹¹, wherein R¹² and R¹⁴ together with the atoms to which they are attached form a 5-6 membered heterocyclic ring.

47. The compound of claim 46, wherein R⁵ is

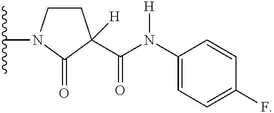

48. The compound of claim 1, wherein R⁵ is —NR¹⁰C(═Y)R¹³, wherein R¹³ is C₁-C₆ alkyl, (CR¹⁴R¹⁵)ₙ—O—(CR¹⁴R¹⁵)ₘ-aryl, (CR¹⁴R¹⁵)-aryl, (CR¹⁴R¹⁵)-heteroaryl, (CR¹⁴R¹⁵)-heterocyclyl, (CR¹⁴R¹⁵)—N(SO₂R^{a})(CR¹⁴R¹⁵)R¹¹, or (CR¹⁴R¹⁵)NR¹⁰C(═O)-aryl, wherein said alkyl, aryl, heteroaryl and heterocyclyl portions are optionally substituted.

49. The compound of claim 48 wherein R⁵ is selected from the structures: 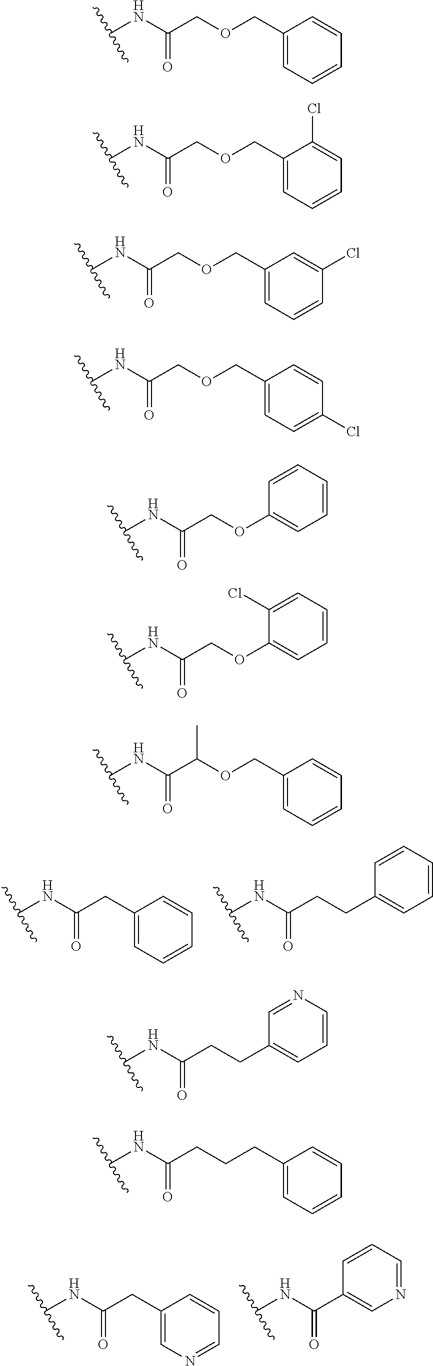 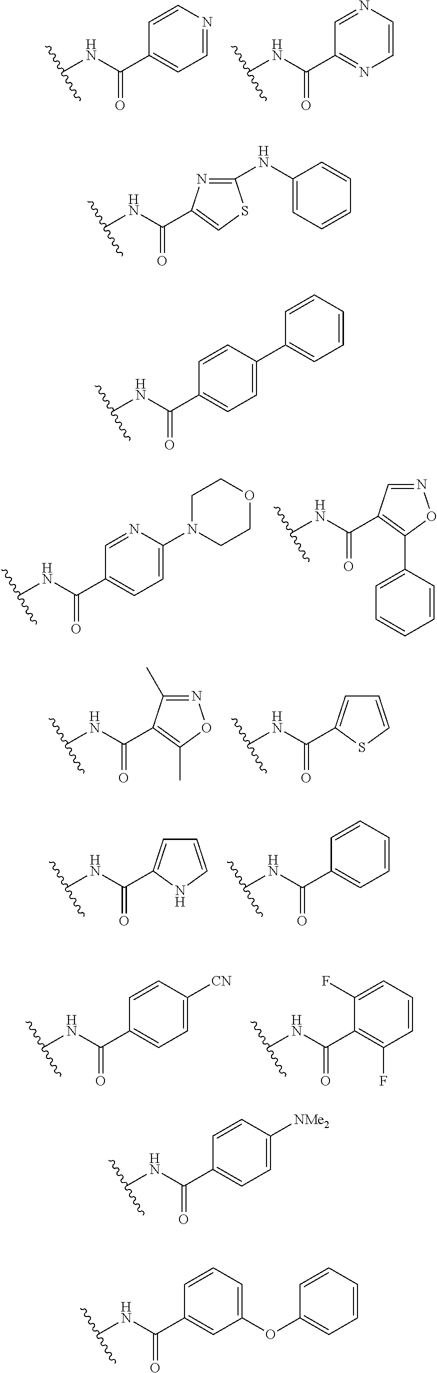 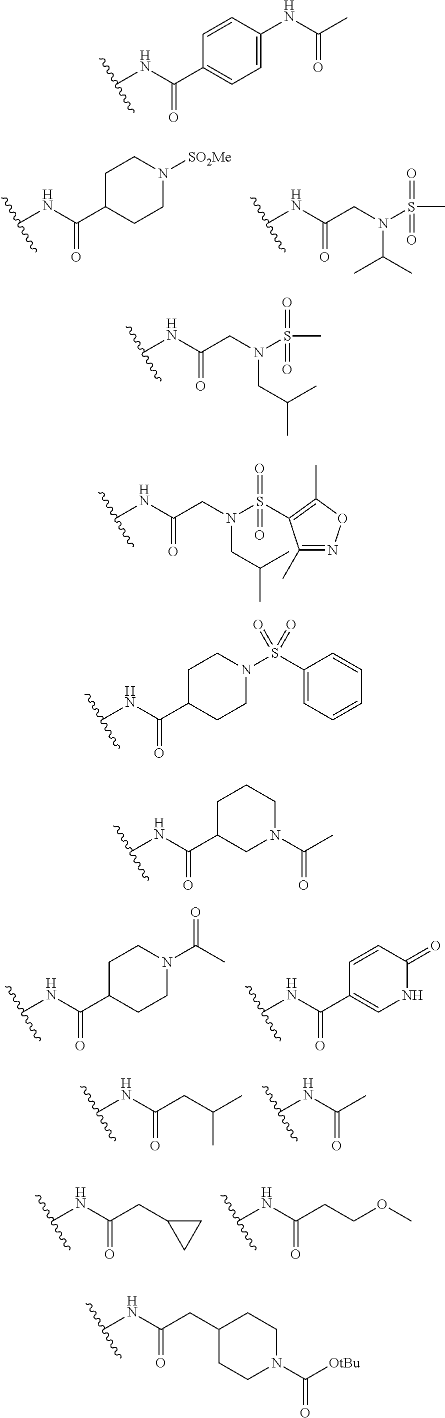 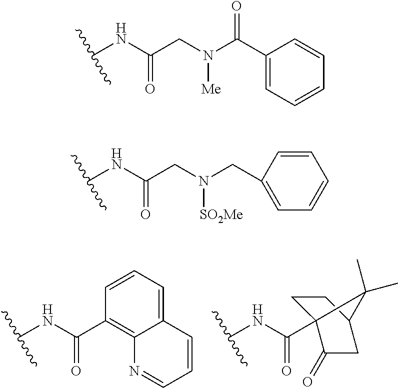
where the wavy line indicates the point of attachment to L.

50. The compound of claim 1 wherein R⁵ is —NR¹⁰C(═Y)OR¹³.

51. The compound of claim 50 wherein R⁵ is selected from the structures: 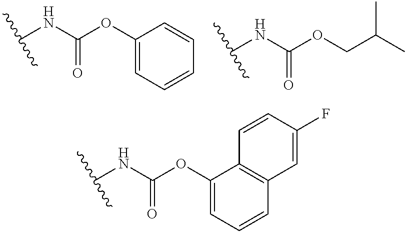
where the wavy line indicates the point of attachment to L.

52. The compound of claim 1 wherein R⁵ is —NR¹²SO₂R¹⁰.

53. The compound of claim 52 wherein R¹⁰ is alkyl or optionally substituted aryl.

54. The compound of claim 53 wherein R⁵ is selected from the structures: 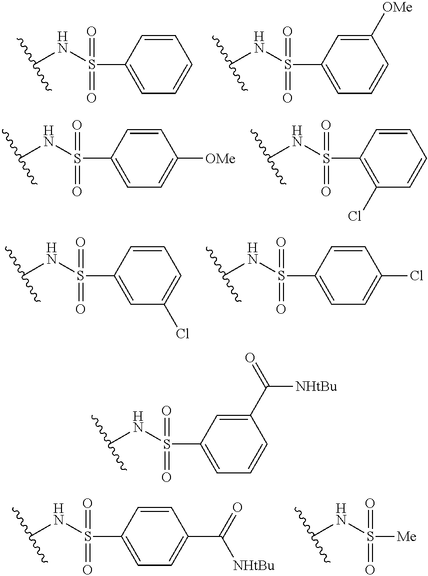
where the wavy line indicates the point of attachment to L.

55. The compound of claim 1 wherein R⁵ is a substituted carbocyclyl.

56. The compound of claim 55 wherein R⁵ is selected from the structures: 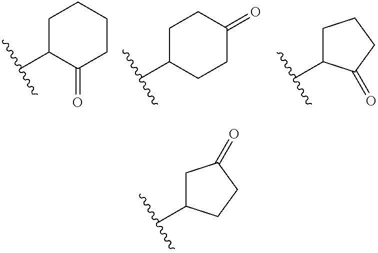
where the wavy line indicates the point of attachment to L.

57. The compound of claim 1 wherein R⁵ is a substituted heterocyclyl.

58. The compound of claim 57 wherein R⁵ is selected from the structures: 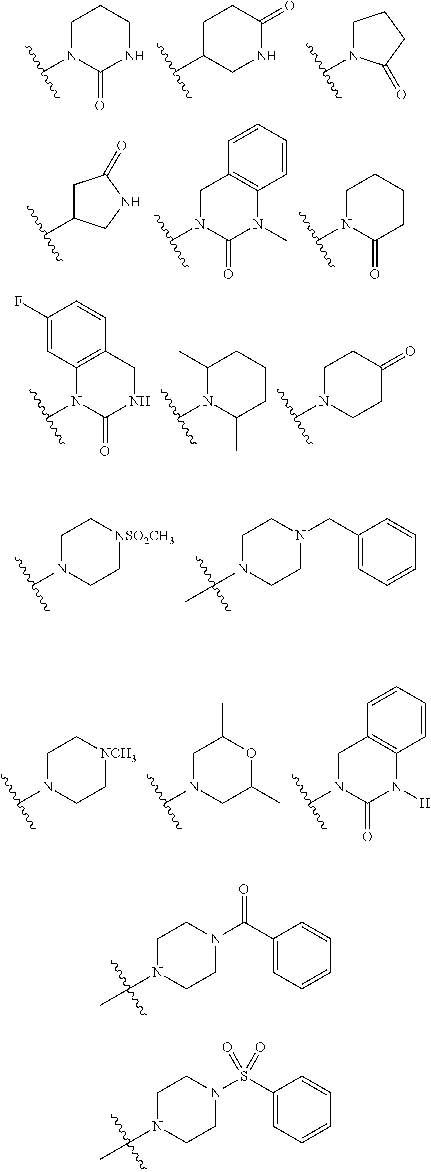
where the wavy line indicates the point of attachment to L.

59. The compound of claim 1 wherein R⁵ is an optionally substituted aryl.

60. The compound of claim 59 wherein R⁵ is selected from the structures: 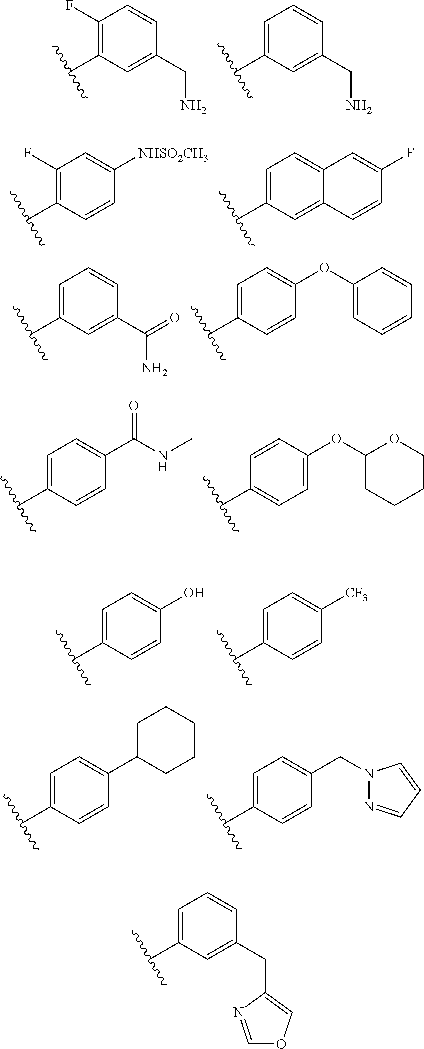
where the wavy line indicates the point of attachment to L.

61. The compound of claim 1 wherein R⁵ is an optionally substituted heteroaryl.

62. The compound of claim 61 wherein R⁵ is selected from the structures: 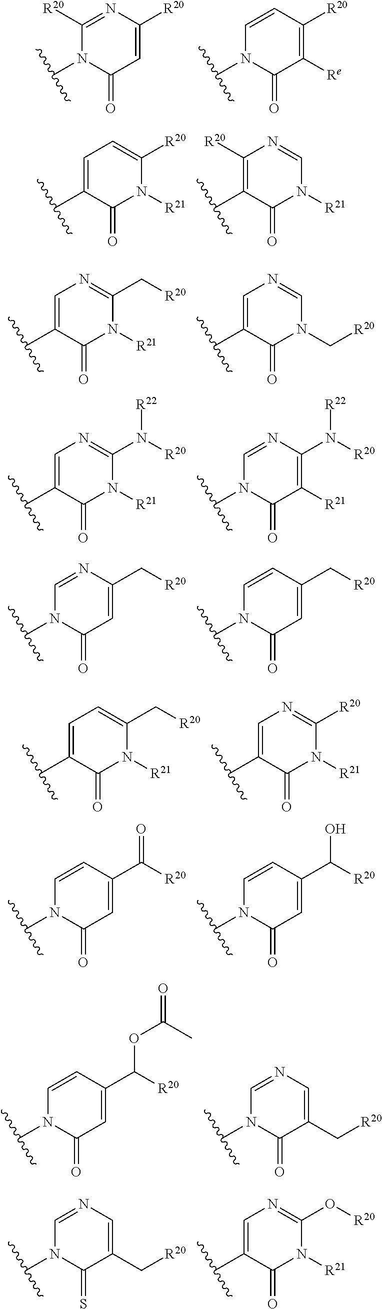 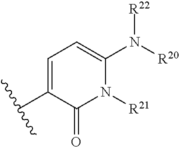
where R²⁰ is H, C₁-C₁₂ alkyl, C₃-C₁₂ cycloalkyl, C₆-C₂₀ aryl, or C₁-C₂₀ heteroaryl, and R²¹ and R²² are independently selected from H or C₁-C₁₂ alkyl, wherein said alkyl, cycloalkyl, aryl, heteroaryl are optionally substituted with one or more groups independently selected from F, Cl, Br, I and C₁-C₁₂ alkyl; each R^{e} is independently H or C₁-C₄ alkyl; and where the wavy line indicates the point of attachment to L.

63. The compound of claim 62 wherein R²⁰ is H.

64. The compound of claim 61 wherein R⁵ is selected from the structures: 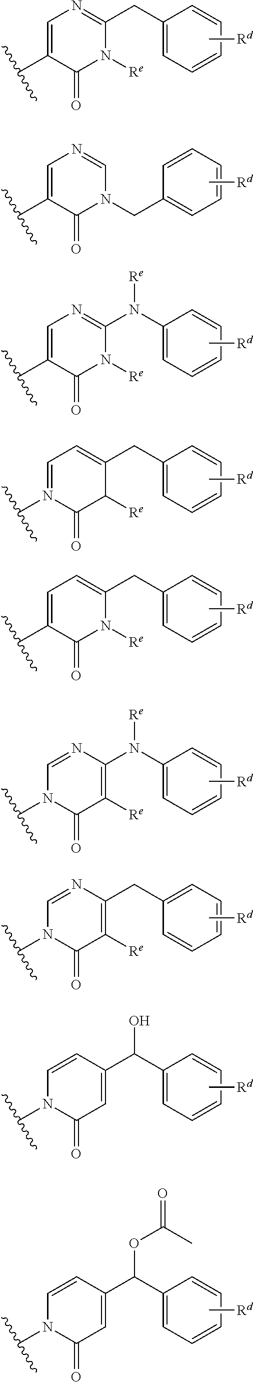 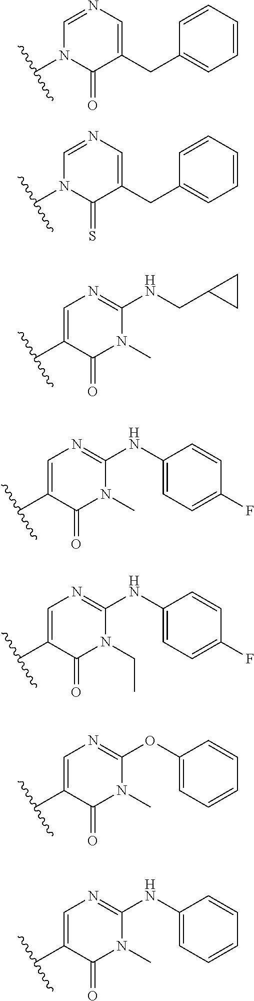
where the phenyl groups are optionally substituted with one or more R^{d} groups independently selected from F, Cl, Br, I, CF₃, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, and C₁-C₂₀ heteroaryl; and each R^{e} is independently H or C₁-C₄ alkyl.

65. The compound of claim 61, wherein R⁵ is selected from:

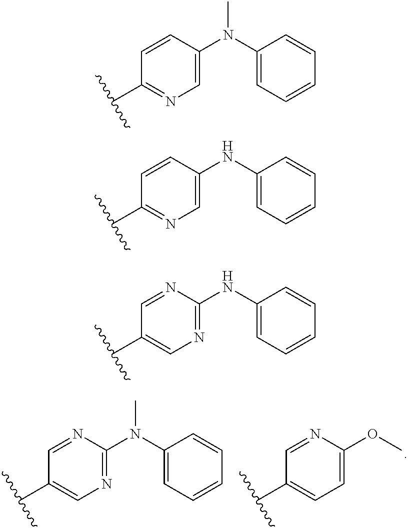

66. The compound of claim 1 selected from:
N-(4-(7-(3-morpholinopropoxy)-6-methoxyquinolin-4-yloxy)-3-fluorophenyl)pyridin-2-amine;
3-(4-(7-(3-morpholinopropoxy)-6-methoxyquinolin-4-yloxy)-3-fluorophenyl)-6-benzyl-1-methylpyridin-2(1H)-one;
1-(4-(7-(3-morpholinopropoxy)-6-methoxyquinolin-4-yloxy)-3-fluorophenyl)-4-(2-methyl)benzyl)-5-methyl-pyrimidin-6-one;
5-(4-(7-(3-morpholinopropoxy)-6-methoxyquinolin-4-yloxy)-3-fluorophenyl)-3-ethyl-2-(phenylamino)pyrimidin-4(3H)-one;
N-(4-(7-(3-(piperidin-1-yl)propoxy)-6-methoxyquinolin-4-yloxy)-3-fluorophenyl)-2-(4-fluorophenyl)-2,3-dihydro-3-oxopyridazine-4-carboxamide;
N-(4-(7-(3-morpholinopropoxy)-6-methoxyquinolin-4-yloxy)-3-fluorophenyl)-1-methyl-2-oxopyrrolidine-3-carboxamide;
N-(4-(7-(3-morpholinopropoxy)-6-methoxyquinolin-4-yloxy)-3-fluorophenyl)-4-benzyl-3,4-dihydro-3-oxopyrazine-2-carboxamide;
N-(6-(7-(3-morpholinopropoxy)-6-methoxyquinolin-4-yloxy)pyridin-3-yl)-2-(4-fluorophenyl)-2,3-dihydro-3-oxopyridazine-4-carboxamide;
N-(4-(7-(3-(4-methylpiperazin-1-yl)propoxy)-6-methoxyquinolin-4-yloxy)-3-fluorophenyl)-2-(4-fluorophenyl)-2,3-dihydro-3-oxopyridazine-4-carboxamide;
2-(4-fluorophenylamino)-5-(4-(7-(3-morpholinopropoxy)-6-methoxyquinolin-4-yloxy)-3-fluorophenyl)-3-methylpyrimidin-4(3H)-one;
5-(4-(7-(3-morpholinopropoxy)-6-methoxyquinolin-4-yloxy)-3-fluorophenyl)-2-(cyclopropylmethylamino)-3-methylpyrimidin-4(3H)-one;
5-(4-(7-(3-morpholinopropoxy)-6-methoxyquinolin-4-yloxy)-3-fluorophenyl)-2-benzyl-3-methylpyrimidin-4(3H)-one;
1-(4-(7-(3-morpholinopropoxy)-6-methoxyquinolin-4-yloxy)-3-fluorophenyl)-N-(4-fluorophenyl)-1,2-dihydro-2-oxopyridine-3-carboxamide;
3-(4-(7-(3-morpholinopropoxy)-6-methoxyquinolin-4-yloxy)-3-fluorophenyl)-5-benzylpyrimidin-4(3H)-one;
N-(4-(7-(3-morpholinopropoxy)-6-methoxyquinolin-4-yloxy)-3-fluorophenyl)-2-oxopyrrolidine-3-carboxamide;
3-(4-(7-(3-morpholinopropoxy)-6-methoxyquinolin-4-yloxy)-3-fluorophenyl)-5-methyl-6-(phenylamino)pyrimidin-4(3H)-one;
3-(4-(7-(3-morpholinopropoxy)-6-methoxyquinolin-4-yloxy)-3-fluorophenyl)-6-benzylpyrimidin-4(3H)-one;
3-(4-(7-(3-morpholinopropoxy)-6-methoxyquinolin-4-yloxy)-3-fluorophenyl)-6-benzyl-5-methylpyrimidin-4(3H)-one;
(4-(7-(3-morpholinopropoxy)-6-methoxyquinolin-4-yloxy)-3-fluorophenyl)(3-benzylpiperidin-1-yl)methanone;
N-(4-(7-(3-morpholinopropoxy)-6-methoxyquinolin-4-yloxy)-3-fluorophenyl)-1,2-dihydro-1-methyl-2-oxopyridine-3-carboxamide;
N-(4-(7-(3-morpholinopropoxy)-6-methoxyquinolin-4-yloxy)-3-fluorophenyl)-7,7-dimethyl-2-oxobicyclo[2.2.1]heptane-1-carboxamide;
N-(4-(7-(3-morpholinopropoxy)-6-methoxyquinolin-4-yloxy)-3-fluorophenyl)-2-(pyridin-2-yl)acetamide;
N-(4-(7-(3-morpholinopropoxy)-6-methoxyquinolin-4-yloxy)-3-fluorophenyl)-2-(4-fluorophenyl)-2,3-dihydro-3-oxopyridazine-4-carboxamide;
N-(4-(7-(3-morpholinopropoxy)-6-methoxyquinolin-4-yloxy)-3-fluorophenyl)quinoline-8-carboxamide;
N-(4-(7-(3-morpholinopropoxy)-6-methoxyquinolin-4-yloxy)-3-fluorophenyl)-1,2-dihydro-2-oxo-1-((pyrimidin-4-yl)methyl)pyridine-3-carboxamide;
1-(4-chlorobenzyl)-N-(4-(7-(3-morpholinopropoxy)-6-methoxyquinolin-4-yloxy)-3-fluorophenyl)-1,2-dihydro-2-oxopyridine-3-carboxamide;
N-(4-(7-(3-morpholinopropoxy)-6-methoxyquinolin-4-yloxy)-3-fluorophenyl)-1-benzyl-1,2-dihydro-2-oxopyridine-3-carboxamide;
5-(4-(7-(3-morpholinopropoxy)-6-methoxyquinolin-4-yloxy)-3-fluorophenyl)-3-methyl-2-(phenylamino)pyrimidin-4(3H)-one;
3-(4-(7-(3-morpholinopropoxy)-6-methoxyquinolin-4-yloxy)phenyl)-5-methyl-6-(phenylamino)pyrimidin-4(3H)-one;
N-(4-(7-(3-morpholinopropoxy)-6-methoxyquinolin-4-yloxy)-3-fluorophenyl)-1-(4-fluorophenyl)-2-oxopiperidine-3-carboxamide;
N-(4-(7-(3-morpholinopropoxy)-6-methoxyquinolin-4-yloxy)-3-fluorophenyl)-1-(4-fluorophenyl)-1,2-dihydro-2-oxopyridine-3-carboxamide;
3-(4-(7-(3-morpholinopropoxy)-6-methoxyquinolin-4-yloxy)-3-fluorophenyl)-3,4-dihydroquinazolin-2(1H)-one;
N-(4-(7-(3-morpholinopropoxy)-6-methoxyquinolin-4-yloxy)-3-fluorophenyl)-1-(4-fluorophenyl)-3-methyl-2-oxopyrrolidine-3-carboxamide;
1-(4-fluorobenzyl)-N-(4-(7-(3-morpholinopropoxy)-6-methoxyquinolin-4-yloxy)-3-fluorophenyl)-1,2-dihydro-2-oxopyridine-3-carboxamide;
N-(4-(7-(3-morpholinopropoxy)-6-methoxyquinolin-4-yloxy)-3-fluorophenyl)-2-oxo-1-phenylpyrrolidine-3-carboxamide;
5-(4-(7-(3-morpholinopropoxy)-6-methoxyquinolin-4-yloxy)phenyl)-2-benzylpyrimidin-4(3H)-one;
N-(4-(7-(3-morpholinopropoxy)-6-methoxyquinolin-4-yloxy)-3-fluorophenyl)-1-(4-chlorophenyl)-2-oxopyrrolidine-3-carboxamide;
N-(4-(7-(3-morpholinopropoxy)-6-methoxyquinolin-4-yloxy)-3-fluorophenyl)-1-(4-fluorophenyl)-2-oxopyrrolidine-3-carboxamide;
5-(4-(7-(3-(piperidin-1-yl)propoxy)-6-methoxyquinolin-4-yloxy)-3-fluorophenyl)-3-benzylpyrimidin-4(3H)-one;
5-(4-(7-(3-(4-methylpiperazin-1-yl)propoxy)-6-methoxyquinolin-4-yloxy)-3-fluorophenyl)-3-benzylpyrimidin-4(3H)-one;
3-(4-chloro-2-fluorobenzyl)-5-(4-(6,7-dimethoxyquinolin-4-yloxy)-3-fluorophenyl)pyrimidin-4(3H)-one;
1-(4-(6,7-dimethoxyquinolin-4-yloxy)-3-fluorophenyl)-N-(4-fluorophenyl)-2-oxopyrrolidine-3-carboxamide;
3-(4-fluoro-3-methylbenzyl)-5-(4-(6,7-dimethoxyquinolin-4-yloxy)-3-fluorophenyl)pyrimidin-4(3H)-one;
3-(3,4-dimethylbenzyl)-5-(4-(6,7-dimethoxyquinolin-4-yloxy)-3-fluorophenyl)pyrimidin-4(3H)-one;
3-(4-chloro-2,6-difluorobenzyl)-5-(4-(6,7-dimethoxyquinolin-4-yloxy)-3-fluorophenyl)pyrimidin-4(3H)-one;
3-(2-chloro-4-fluorobenzyl)-5-(4-(6,7-dimethoxyquinolin-4-yloxy)-3-fluorophenyl)pyrimidin-4(3H)-one;
3-(3,4-dichlorobenzyl)-5-(4-(6,7-dimethoxyquinolin-4-yloxy)-3-fluorophenyl)pyrimidin-4(3H)-one;
N-(2-(7-(3-morpholinopropoxy)-6-methoxyquinolin-4-yloxy)naphthalen-6-yl)thiophene-3-carboxamide;
5-(4-(7-(2-(1H-imidazol-1-yl)ethoxy)-6-methoxyquinolin-4-yloxy)-3-fluorophenyl)-3-benzylpyrimidin-4(3H)-one;
3-(4-(trifluoromethyl)benzyl)-5-(4-(6,7-dimethoxyquinolin-4-yloxy)-3-fluorophenyl)pyrimidin-4(3H)-one;
3-(4-tolyl)-5-(4-(6,7-dimethoxyquinolin-4-yloxy)-3-fluorophenyl)pyrimidin-4(3H)-one;
3-(4-fluorobenzyl)-5-(4-(6,7-dimethoxyquinolin-4-yloxy)-3-fluorophenyl)pyrimidin-4(3H)-one;
3-(2-fluorobenzyl)-5-(4-(6,7-dimethoxyquinolin-4-yloxy)-3-fluorophenyl)pyrimidin-4(3H)-one;
3-(4-chlorobenzyl)-5-(4-(7-(3-morpholinopropoxy)-6-methoxyquinolin-4-yloxy)-3-fluorophenyl)pyrimidin-4(3H)-one;
5-(4-(7-(3-morpholinopropoxy)-6-methoxyquinolin-4-yloxy)-3-fluorophenyl)-3-benzylpyrimidin-4(3H)-one;
(4-(6,7-dimethoxyquinolin-4-yloxy)-3-fluorophenyl)(4-benzylpiperidin-1-yl)methanone;
(4-(6,7-dimethoxyquinolin-4-yloxy)-3-fluorophenyl)(3-benzylpiperidin-1-yl)methanone;
3-(4-chlorobenzyl)-5-(4-(6,7-dimethoxyquinolin-4-yloxy)-3-fluorophenyl)pyrimidin-4(3H)-one;
3-(3-chlorobenzyl)-5-(4-(6,7-dimethoxyquinolin-4-yloxy)-3-fluorophenyl)pyrimidin-4(3H)-one;
3-(2-methylbenzyl)-5-(4-(6,7-dimethoxyquinolin-4-yloxy)-3-fluorophenyl)pyrimidin-4(3H)-one;
3-(2-chlorobenzyl)-5-(4-(6,7-dimethoxyquinolin-4-yloxy)-3-fluorophenyl)pyrimidin-4(3H)-one;
5-(4-(7-(3-morpholinopropoxy)-6-methoxyquinolin-4-yloxy)-3-fluorophenyl)-2-benzylpyrimidin-4(3H)-one;
3-(4-(7-(3-morpholinopropoxy)-6-methoxyquinolin-4-yloxy)-3-fluorophenyl)-6-benzylpyridin-2(1H)-one;
1-(4-(7-(3-morpholinopropoxy)-6-methoxyquinolin-4-yloxy)-3-fluorophenyl)-4-benzylpyridin-2(1H)-one;
5-(4-(6,7-dimethoxyquinolin-4-yloxy)-3-fluorophenyl)-N-methyl-N-phenylpyrimidin-2-amine;
5-(4-(6,7-dimethoxyquinolin-4-yloxy)-3-fluorophenyl)-N-phenylpyrimidin-2-amine;
4-((6,7-dimethoxyquinolin-4-yloxy)-3-fluorophenyl)-(2-tetrahydro-2H-pyranyl)-phenol;
4-((6,7-dimethoxyquinolin-4-yloxy)-3-fluorophenyl)-N-cyclopropylbenzamide;
4-((6,7-dimethoxyquinolin-4-yloxy)-3-fluorophenyl)-benzyl-1H-pyrazole;
4-((6,7-dimethoxyquinolin-4-yloxy)-3-fluorophenyl)-cyclohexylbenzene;
4-((6,7-dimethoxyquinolin-4-yloxy)-3-fluorophenyl)-N-phenylmethylsulfonamide;
4-((6,7-dimethoxyquinolin-4-yloxy)-3-fluorophenyl)-phenoxybenzene;
4-(2-fluoro-4-(6-methoxypyridin-3-yl)phenoxy)-6,7-dimethoxyquinoline;
tert-butyl 2-(4-(6,7-dimethoxyquinolin-4-yloxy)-3-fluorophenyl)-1H-pyrrole-1-carboxylate;
5-(4-(6,7-dimethoxyquinolin-4-yloxy)-3-fluorophenyl)-2-benzylpyrimidin-4(3H)-one;
5-(4-(6,7-dimethoxyquinolin-4-yloxy)-3-fluorophenyl)-3-benzylpyrimidin-4(3H)-one;
5-(4-(6,7-dimethoxyquinolin-4-yloxy)-3-fluorophenyl)pyrimidin-4(3H)-one;
3-(4-(6,7-dimethoxyquinolin-4-yloxy)-3-fluorophenyl)-6-benzylpyridin-2(1H)-one;
(1-(4-(6,7-dimethoxyquinolin-4-yloxy)-3-fluorophenyl)-1,2-dihydro-2-oxopyridin-4-yl)(phenyl)methyl acetate;
1-(4-(6,7-dimethoxyquinolin-4-yloxy)-3-fluorophenyl)-4-(hydroxy(phenyl)methyl)pyridin-2(1H)-one;
1-(4-(6,7-dimethoxyquinolin-4-yloxy)-3-fluorophenyl)-(4-phenylmethanone) pyridin-2(1H)-one;
1-(4-(6,7-dimethoxyquinolin-4-yloxy)-3-fluorophenyl)-3-methylpyridin-2(1H)-one;
1-(4-(6,7-dimethoxyquinolin-4-yloxy)-3-fluorophenyl)-4-methylpyridin-2(1H)-one;
1-(4-(6,7-dimethoxyquinolin-4-yloxy)-3-fluorophenyl)-4-benzylpyridin-2(1H)-one; and
1-(4-(6,7-dimethoxyquinolin-4-yloxy)-3-fluorophenyl)pyridin-2(1H)-one.

67. A pharmaceutical composition comprised of a compound of claim 1.

68. The composition according to claim 67, further comprising an additional therapeutic agent selected from an anti-proliferative agent, an anti-inflammatory agent, an immunomodulatory agent, a neurotropic factor, an agent for treating cardiovascular disease, an agent for treating liver disease, an anti-viral agent, an agent for treating blood disorders, an agent for treating diabetes, or an agent for treating immunodeficiency disorders.

69. A composition comprising a compound of claim 1 in an amount to detectably inhibit Met kinase activity and a pharmaceutically acceptable carrier, adjuvant, or vehicle.

70. A method of treating or lessening the severity of a disease or condition selected from the group consisting of cancer, stroke, diabetes, hepatomegaly, cardiovascular disease, Alzheimer's disease, cystic fibrosis, viral disease, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders in a patient, comprising the step of administering to said patient a compound of claim 1.

71. A method of treating cancer in a mammal in need of such treatment which is comprised of administering to said mammal a therapeutically effective amount of a compound of claim 1.

72. The method of claim 71 wherein the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

73. A process for making a pharmaceutical composition which comprises combining a compound of claim 1 with a pharmaceutically acceptable carrier.

74. (canceled)

75. (canceled)

76. A method for inhibiting or modulating receptor tyrosine kinase activity, comprising contacting the kinase with an effective inhibitory amount of a compound of claim 1.

77. The method of claim 76 wherein the kinase is c-Met.

78. A method for inhibiting or modulating receptor tyrosine kinase activity in a mammal, comprising administering to the mammal a therapeutically effective amount of a compound of claim 1.

79. The method according to claim 78 wherein the receptor tyrosine kinase is c-Met.

80. A kit for treating a c-Met-mediated condition, comprising:
a) a first pharmaceutical composition comprising a compound of claim 1; and
b) instructions for use.

81. The kit of claim 80 further comprising (c) a second pharmaceutical composition, wherein the second pharmaceutical composition comprises a second compound having anti-hyperproliferative activity.

82. The compound of claim 1 wherein —OR¹⁰ is selected from the structure:

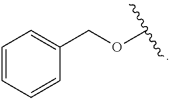

83. The compound of claim 1 selected from 3-benzyl-5-(4-(7-(benzyloxy)-6-methoxyquinolin-4-yloxy)-3-fluorophenyl)pyrimidin-4(3H)-one.
